(19) United States
(12) Patent Application Publication
(10) Pub. No.: US 2019/0288215 A1
(43) Pub. Date: Sep. 19, 2019

(54) COMPOUND AND ORGANIC LIGHT EMITTING DEVICE COMPRISING THE SAME

(71) Applicant: SOULBRAIN CO., LTD., Seongnam-si (KR)

(72) Inventors: Seok-Jong LEE, Seongnam-si (KR); Jin-Hee KIM, Seongnam-si (KR); Eun-Chul SHIN, Seongnam-si (KR); Jae-Kyo PARK, Seongnam-si (KR); Hyun-Young YOO, Seongnam-si (KR); Ah Rang LEE, Seongnam-si (KR)

(21) Appl. No.: 16/192,788

(22) Filed: Nov. 15, 2018

(30) Foreign Application Priority Data
Nov. 30, 2017 (KR) 10-2017-0163645

(51) Int. Cl. H01L 51/00; C07D 403/10

(52) U.S. Cl. CPC H01L 51/0067; H01L 51/5096; H01L 51/0072; C07D 403/10

(57) ABSTRACT

The present invention provides a compound of Chemical Formula 1 and a light emitting device comprising the same.

### CROSS-REFERENCE TO RELATED APPLICATIONS

This application claims priority under 35 U.S.C. § 119 to Korean Patent Application No. 10-2017-0163645, filed on Nov. 30, 2017, in the Korean Intellectual Property Office, the disclosure of which is incorporated herein by reference in its entirety.

### TECHNICAL FIELD

The following disclosure relates to a compound and an organic light emitting device including the same.

### BACKGROUND

In general, an organic light emitting phenomenon refers to a phenomenon in which electric energy is converted into light energy through use of an organic material. The fields of application of devices manufactured for making organic electroluminescent devices using organic materials has been gradually expanded to the fields of display and illumination for various electronic products. However, efficiency and lifetime are limiting factors in expanding the fields of use, and thus a number of studies are underway in view of both the material as well as the device in order to improve the efficiency and the lifetime. The host material being used simultaneously with a dopant material is also important to obtain high light emitting efficiency and lifetime characteristics. As a light emitting host material, in view of the light emitting mechanism, phosphorescent materials, rather than fluorescent materials, have been actively studied as a method for improving the efficiency. For example, there are carbazole derivatives including 4,4′-bis(9-carbazolyl)biphenyl (CBP) material, which is a representative material being used. When a device is manufactured using a carbazole derivative material such as CBP as the phosphorescent light emitting host material, the electron or hole transporting ability is biased toward one side, and thus the efficiency of light emitting is poor, driving voltage increases, whereby there is no great benefit even in view of power efficiency, while the lifetime is also unsatisfactory. Therefore, in order for the organic electroluminescent device to sufficiently exhibit excellent characteristics thereof, a hole injecting material, a hole transport material, a light emitting material, an electron transport material, an electron injecting material, and the like, included in the device are required to be supported by stable and efficient materials.

Patent Literature: JP 2008-214244

### SUMMARY

An embodiment of the present invention is directed to providing a compound capable of being used as a material of an organic material layer of an organic light emitting device, and an organic light emitting device comprising the same.

An embodiment of the present invention provides a compound represented by Chemical Formula 1 below:

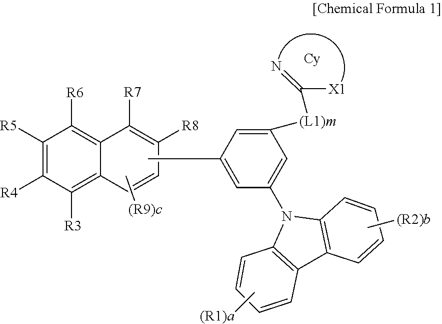

in Chemical Formula 1,

Cy is a substituted or unsubstituted monocyclic or polycyclic heterocycle group including at least one nitrogen atom,

X1 is N or CR11 when it is a member of a 6-membered ring,

X1 is NR12 or NR13R14 when it is a member of a 5-membered ring,

L1 is either a direct bond or a substituted or unsubstituted arylene group,

R1 to R9, and R11 to R14 may be the same as or different from each other, and each independently selected from the group consisting of: hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; and an amine group which is either unsubstituted or substituted with an alkyl group, an aryl group, or a heteroaryl group, and adjacent groups may be bonded to each other to form a hydrocarbon ring or a heterocycle,

a and b are each integers of 0 to 4, each R1 may be the same as or different from each other when a is an integer of 2 or more, each R2 may be the same as or different from each other when b is an integer of 2 or more, c is an integer of 0 or 1, and m is an integer of 1 or 2.

In addition, another embodiment of the present invention provides an organic light emitting diode comprising an anode, a cathode, and one or more layered organic material layers provided between the anode and the cathode, wherein one or more of the organic material layers include the compound represented by Chemical Formula 1.

### BRIEF DESCRIPTION OF THE DRAWINGS

FIG. 1 is a schematic view showing a stacked structure of an organic light emitting device according to an embodiment of the present invention.

### DETAILED DESCRIPTION OF EMBODIMENTS

Hereinafter, the present invention will be described in detail.

The term “substituted or unsubstituted” used herein means substitution or unsubstitution with one or more substituents selected from the group consisting of: deuterium; a halogen group; —CN; a C₁-C₆₀ alkyl group; a C₂-C₆₀ alkenyl group; a C₂-C₆₀ alkynyl group; a C₃-C₆₀ cycloalkyl group; a C₂-C₆₀ heterocycloalkyl group; a C₆-C₆₀ aryl group; a C₂-C₆₀ heteroaryl group; a C₁-C₂₀ alkylamine group; a C₆-C₆₀ arylamine group; and a C₂-C₆₀ heteroarylamine group, or substitution or unsubstitution with a substituent to which two or more of the substituents are bonded, or substitution or unsubstitution with a substituent linked with two or more substituents selected from among the substituents. For example, the “substituent linked with two or more substituents selected from among the substituents” may be a terphenyl group. In other words, the terphenyl group may be an aryl group, and may be interpreted as a substituent to which two phenyl groups are linked. The additional substituents may be further substituted.

According to an embodiment of the present invention, the term “substituted or unsubstituted” means substitution or unsubstitution with one or more substituents selected from the group consisting of deuterium, a halogen group, —CN, a C₁-C₂₀ linear or branched alkyl group, a C₆-C₆₀ aryl group, and a C₂-C₆₀ heteroaryl group. The term “substitution” means that a hydrogen atom bonded to a carbon atom of the compound is substituted with another substituent, and the position to be substituted is not limited if it is a position where the hydrogen atom is substituted, i.e., the position where the substituent is substitutable. When substitution is carried out with two or more substituents, the two or more substituents may be the same as or different from each other.

In the present specification, the halogen may be fluorine, chlorine, bromine or iodine.

In the present specification, the term alkyl group includes a linear or branched chain having 1 to 60 carbon atoms, which may be further substituted with other substituents. The carbon atoms of the alkyl group can be 1 to 60, preferably 1 to 40, and more preferably 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but the alkyl group is not limited thereto.

In the present specification, the alkenyl group includes a straight chain or branched chain having 2 to 60 carbon atoms, which may be additionally substituted with other substituents.

The number of carbon atoms of the alkenyl group may be 2 to 60, preferably 2 to 40, and more preferably 2 to 20.

Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but the alkenyl group is not limited thereto.

In the present specification, the alkynyl group includes a straight chain or branched chain having 2 to 60 carbon atoms, which may be additionally substituted with other substituents. The number of carbon atoms of the alkynyl group may be 2 to 60, preferably 2 to 40, and more preferably 2 to 20.

In the present specification, the cycloalkyl group includes monocyclic or polycyclic groups having 3 to 60 carbon atoms, which may be further substituted with other substituents. Herein, the term “polycyclic” means a group in which a cycloalkyl group is directly linked to another ring group or condensed therewith. Here, the other ring group may be a cycloalkyl group, but may also be other kinds of ring groups such as a heterocycloalkyl group, an aryl group, a heteroaryl group, and the like. The number of carbon atoms of the cycloalkyl group can be 3 to 60, preferably 3 to 40, and more preferably 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but the cycloalkyl group is not limited thereto.

In the present specification, the heterocycloalkyl group includes O, S, Se, N or Si as a hetero atom and includes a monocyclic or polycyclic group having 2 to 60 carbon atoms, which may be further substituted with other substituents. Herein, the term “polycyclic” means a group in which a heterocycloalkyl group is directly linked to another ring group or condensed therewith.

Here, the other cyclic group may also be a heterocycloalkyl group, but it may also be another kind of cyclic group, for example, a cycloalkyl group, an aryl group, a heteroaryl group, and the like. The number of carbon atoms of the heterocycloalkyl group may be 2 to 60, preferably 2 to 40, and more preferably 3 to 20.

In the present specification, the aryl group includes a monocycle or polycycle having 6 to 60 carbon atoms, which may be additionally substituted with other substituents. Here, polycycle means a group in which an aryl group is directly linked to or fused with another cyclic group. Here, the other cyclic group may also be an aryl group, but it may also be another kind of cyclic group, for example, a cycloalkyl group, a heterocycloalkyl group, a heteroaryl group, and the like. The term aryl group includes a spiro group. The number of carbon atoms of the aryl group may be 6 to 60, preferably 6 to 40, and more preferably 6 to 25. Specific examples of the aryl group include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but the aryl group is not limited thereto.

In the present specification, the spiro group is a group including a spiro structure and may have 15 to 60 carbon atoms.

For example, the spiro group may include a structure in which a 2,3-dihydro-1H-indene group or a cyclohexane group is spiro-bonded to a fluorenyl group.

In the present specification, the heteroaryl group includes O, S, Se, N, or Si as a heteroatom, includes a monocycle or polycycle having 2 to 60 carbon atoms, and may be additionally substituted with other substituents. Here, polycycle means a group in which a heteroaryl group is directly linked to or fused with another cyclic group. Here, the other cyclic group may also be a heteroaryl group, but it may also be another kind of cyclic group, for example, a cycloalkyl group, a heterocycloalkyl group, an aryl group, and the like. The number of carbon atoms of the heteroaryl group may be 2 to 60, preferably 2 to 40, and more preferably 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxinyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but they heteroaryl group is not limited thereto.

In the present specification, the amine group may be selected from the group consisting of a monoalkylamine group, a monoarylamine group, a monoheteroarylamine group, —NH2, a dialkylamine group, a diarylamine group, a diheteroarylamine group, an alkylarylamine group, an alkylheteroarylamine group, and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but the amine group is not limited thereto.

The compound according to an embodiment of the present invention is characterized by the representation in Chemical Formula 1 above.

More specifically, the compound may be used as a material for an organic material layer of an organic light emitting device by including three characteristic substituents, i.e., triphenylene, N-carbazole, and an N-containing ring in the benzene structure of Chemical Formula 1.

The compound according to an embodiment of the present invention has a wide band gap, and has an excellent hole block function due to having a low HOMO energy level, thereby making a large contribution to increasing the current efficiency.

Further, since the compound according to an embodiment of the present invention has a bipolar structure, there is a great advantage in hole and electron stabilization, leading to long lifetime characteristics.

In an embodiment of the present invention, L1 is a direct bond, or a substituted or unsubstituted C₆-C₂₀ arylene group.

In an embodiment of the present invention, L1 is a direct bond.

In an embodiment of the present invention, L1 is a phenylene group.

In an embodiment of the present invention, R1 and R2 are the same as or different from each other, and each independently may be hydrogen, or a substituted or unsubstituted aryl group.

In an embodiment of the present invention, R1 and R2 are the same as or different from each other, and each independently may be hydrogen, or a substituted or unsubstituted aryl group having 6 to 20 carbon atoms.

In an embodiment of the present invention, R1 and R2 are the same as or different from each other, and each independently may be hydrogen, or a substituted or unsubstituted phenyl group.

In an embodiment of the present invention, R1 and R2 are the same as or different from each other, and each independently may be hydrogen or a phenyl group.

In an embodiment of the present invention, R1 and R2 are hydrogen.

In an embodiment of the present invention, R1 is hydrogen, and R2 is a phenyl group.

In an embodiment of the present invention, Chemical Formula 1 may be represented by Chemical Formulas 2 or 3 below.

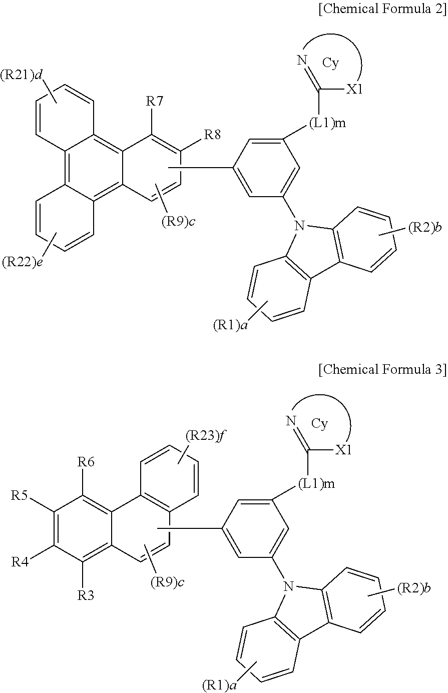

R21 and R23 are the same as or different from each other, and each, independently, may be: hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; or a substituted or unsubstituted heteroaryl group.

d, e and f are each, independently, an integer of 0 to 4, each R21 may be the same as or different from each other when d is an integer of 2 or more, each R22 may be the same as or different from each other when e is an integer of 2 or more, and each R23 may be the same as or different from each other when f is an integer of 2 or more.

In an embodiment of the present invention, R21 is hydrogen.

In an embodiment of the present invention, R22 is hydrogen.

In an embodiment of the present invention, R23 is hydrogen.

In an embodiment of the present invention, Chemical Formula 1 can be represented by any one of Chemical Formulas 4-1 to 4-3 below:

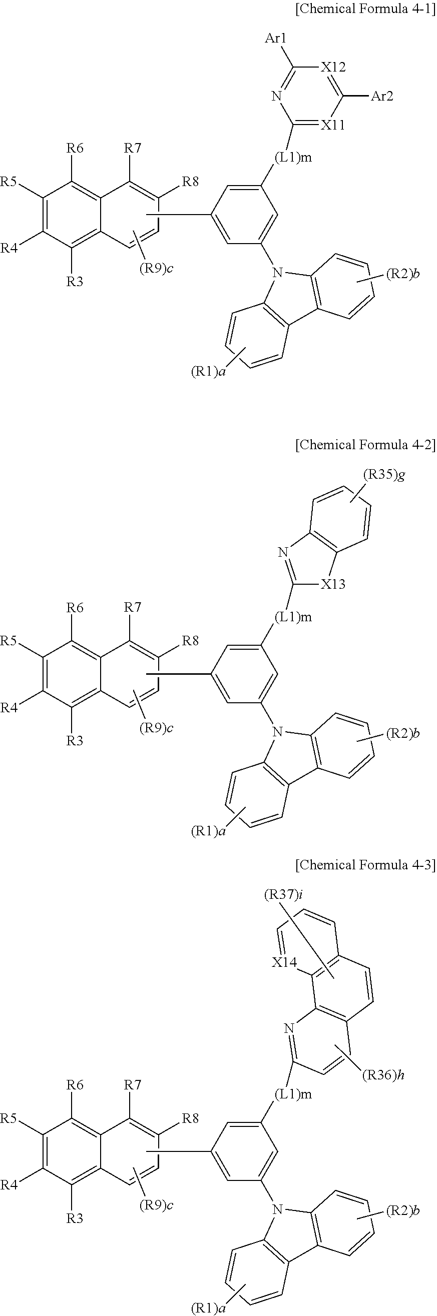

In an embodiment of the present invention, in Chemical Formulas 4-1 to 4-3, X11, X12, and X14 are the same as or different from each other, and each, independently, may be N or CR31, and X13 is NR32 or CR33R34,

R31 to R37, Ar1 and Ar2 may be the same as or different from each other, and each, independently, may be: hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; or a substituted or unsubstituted heteroaryl group; and R33 and R34 may be bonded to each other to form a hydrocarbon ring,

g is an integer of 1 to 4, each R35 may be the same as or different from each other when g is an integer of 2 or more, h is an integer of 1 or 2, each R36 may be the same as or different from each other when h is an integer of 2, i is an integer of 1 to 5, and each R37 is the same as or different from each other when g is an integer of 2 or more.

In an embodiment of the present invention, Ar1 and Ar2 may be the same as or different from each other, and each, independently, may be: hydrogen; a substituted or unsubstituted aryl group; or a substituted or unsubstituted heteroaryl group.

In an embodiment of the present invention, Ar1 and Ar2 may be the same as or different from each other, and each, independently, may be: a substituted or unsubstituted aryl group having 6 to 20 carbon atoms; or a substituted or unsubstituted heteroaryl group having 3 to 20 carbon atoms.

In an embodiment of the present invention, Ar1 and Ar2 may be the same as or different from each other, and each, independently, may be: a substituted or unsubstituted phenyl; a substituted or unsubstituted biphenyl; a substituted or unsubstituted triphenyl; a substituted or unsubstituted terphenyl; a substituted or unsubstituted naphthyl group; a substituted or unsubstituted phenanthrenyl group; a substituted or unsubstituted pyrenyl group; a substituted or unsubstituted fluorenyl group; a substituted or unsubstituted pyridyl group; a substituted or unsubstituted dibenzothiophene group; a substituted or unsubstituted dibenzofuran group; or a substituted or unsubstituted carbazole group.

In an embodiment of the present invention, Ar1 and Ar2 may be the same as or different from each other, and each, independently, may be: a phenyl group; a biphenyl group; a triphenyl group; a terphenyl group; a naphthyl group; a phenanthrenyl group; a pyrenyl group; a pyridyl group; or they may be represented by Chemical Formulas 6 or 7 below, wherein

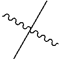

means a moiety bonded to Chemical Formula 4 and Y1 is O, S, CRaRb or NRc; and Ra to Rc are the same as or different from each other, and each, independently, may be: hydrogen; a substituted or unsubstituted alkyl group; or a substituted or unsubstituted aryl group, wherein Ra and Rb may be bonded to each other to form a hydrocarbon ring.

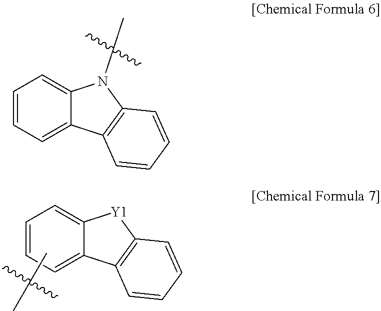

In an embodiment of the present invention, Ra to Rc may be the same as or different from each other, and each, independently, may be: hydrogen; a substituted or unsubstituted C₁-C₄ alkyl group; or a substituted or unsubstituted C₆-C₂₀ aryl group, wherein Ra and Rb may be bonded to each other to form a hydrocarbon ring.

In an embodiment of the present invention, Ra and Rb may be the same as or different from each other, and each, independently, may be: hydrogen; a substituted or unsubstituted methyl group; or a substituted or unsubstituted phenyl group, wherein Ra and Rb may be bonded to each other to form a hydrocarbon ring.

In an embodiment of the present invention, Ra and Rb may be the same as or different from each other, and each, independently, may be: hydrogen; a methyl group; or a phenyl group, wherein Ra and Rb may be bonded to each other to form a hydrocarbon ring.

In an embodiment of the present invention, Rc is hydrogen, or a substituted or unsubstituted phenyl group.

In an embodiment of the present invention, Rc is hydrogen, or a phenyl group.

In an embodiment of the present invention, Chemical Formula 7 may be represented by any one of the following Chemical Formulas, 7-1-1 to 7-2-6.

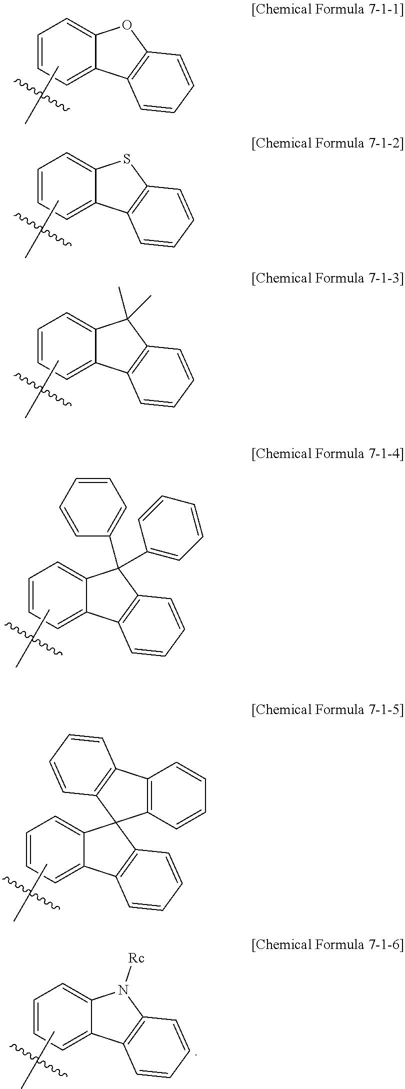

In an embodiment of the present invention, Chemical Formula 7 may be represented by any one of the following Chemical Formulas, 7-2-1 to 7-2-4.

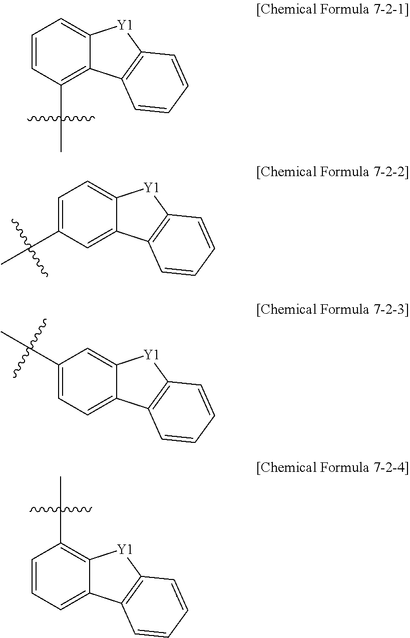

In an embodiment of the present invention, X11 and X12 are N.

In an embodiment of the present invention, X11 and X12 are the same as or different from each other, and each independently C31.

In an embodiment of the present invention, R31 is hydrogen.

In an embodiment of the present invention, X13 is NR32.

In an embodiment of the present invention, R32 is hydrogen, or a substituted or unsubstituted aryl group.

In an embodiment of the present invention, R32 is hydrogen, or a substituted or unsubstituted aryl group having 6 to 20 carbon atoms.

In an embodiment of the present invention, R32 is hydrogen, or a substituted or unsubstituted phenyl group.

In an embodiment of the present invention, R32 is a phenyl group.

In an embodiment of the present invention, R35 is hydrogen.

In an embodiment of the present invention, R36 and R37 are the same as or different from each other, and each, independently, may be hydrogen, or a substituted or unsubstituted aryl group having 6 to 20 carbon atoms.

In an embodiment of the present invention, R36 and R37 are the same as or different from each other, and each, independently, may be hydrogen, or a substituted or unsubstituted phenyl group.

In an embodiment of the present invention, R36 and R37 are the same as or different from each other, and each, independently, may be hydrogen or a phenyl group.

In an embodiment of the present invention, Chemical Formula 2 may be represented by Chemical Formula 2-2 below.

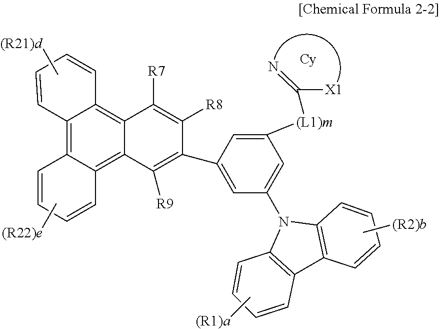

According to an embodiment of the present invention, Chemical Formula 1 may be represented by any one of the following compounds, but it is not limited thereto.

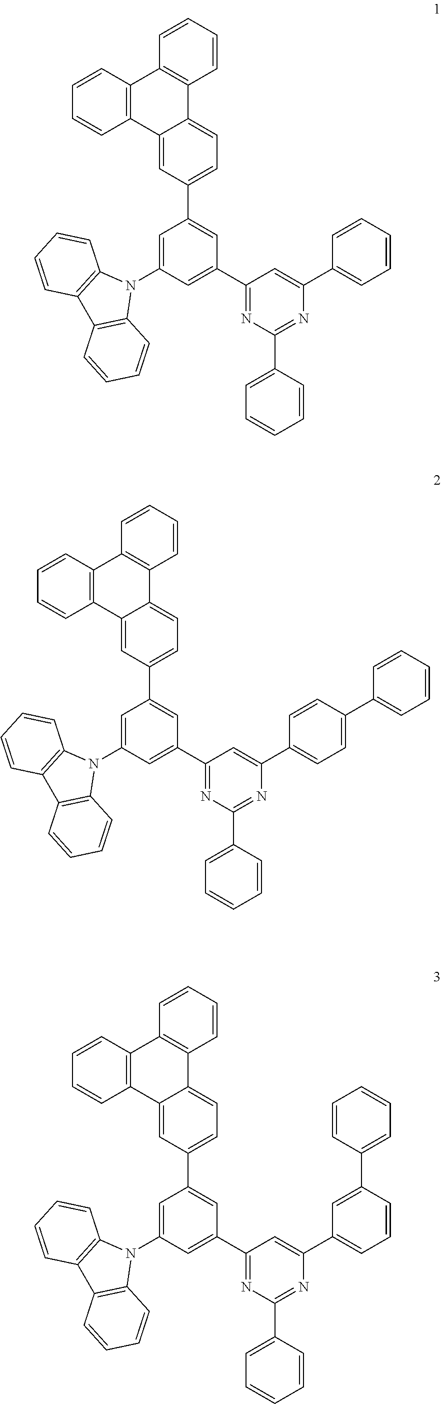 [SMILES: c1ccc(-c2cc(-c3cc(-c4ccc5c6ccccc6c6ccccc6c5c4)cc(-n4c5ccccc5c5ccccc54)c3)nc(-c3ccccc3)n2)cc1 ; c1ccc(-c2ccc(-c3cc(-c4cc(-c5ccc6c7ccccc7c7ccccc7c6c5)cc(-n5c6ccccc6c6ccccc65)c4)nc(-c4ccccc4)n3)cc2)cc1 ; c1ccc(-c2cccc(-c3cc(-c4cc(-c5ccc6c7ccccc7c7ccccc7c6c5)cc(-n5c6ccccc6c6ccccc65)c4)nc(-c4ccccc4)n3)c2)cc1]

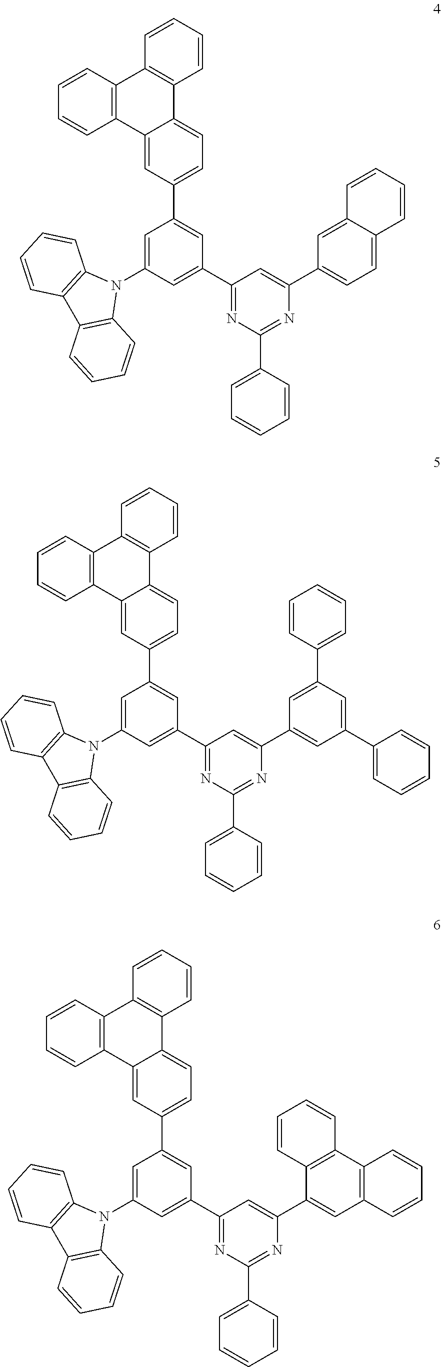 [SMILES: c1ccc(-c2nc(-c3cc(-c4ccc5c6ccccc6c6ccccc6c5c4)cc(-n4c5ccccc5c5ccccc54)c3)cc(-c3ccc4ccccc4c3)n2)cc1 ; c1ccc(-c2cc(-c3ccccc3)cc(-c3cc(-c4cc(-c5ccc6c7ccccc7c7ccccc7c6c5)cc(-n5c6ccccc6c6ccccc65)c4)nc(-c4ccccc4)n3)c2)cc1 ; c1ccc(-c2nc(-c3cc(-c4ccc5c6ccccc6c6ccccc6c5c4)cc(-n4c5ccccc5c5ccccc54)c3)cc(-c3cc4ccccc4c4ccccc34)n2)cc1]

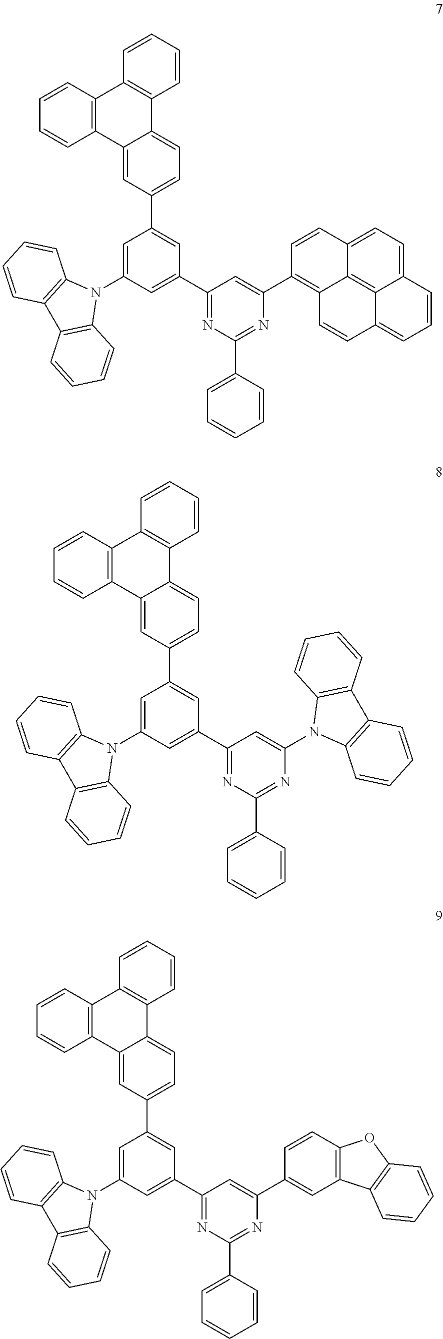 [SMILES: c1ccc(-c2nc(-c3cc(-c4ccc5c6ccccc6c6ccccc6c5c4)cc(-n4c5ccccc5c5ccccc54)c3)cc(-c3ccc4ccc5cccc6ccc3c4c56)n2)cc1 ; c1ccc(-c2nc(-c3cc(-c4ccc5c6ccccc6c6ccccc6c5c4)cc(-n4c5ccccc5c5ccccc54)c3)cc(-n3c4ccccc4c4ccccc43)n2)cc1 ; c1ccc(-c2nc(-c3cc(-c4ccc5c6ccccc6c6ccccc6c5c4)cc(-n4c5ccccc5c5ccccc54)c3)cc(-c3ccc4oc5ccccc5c4c3)n2)cc1]

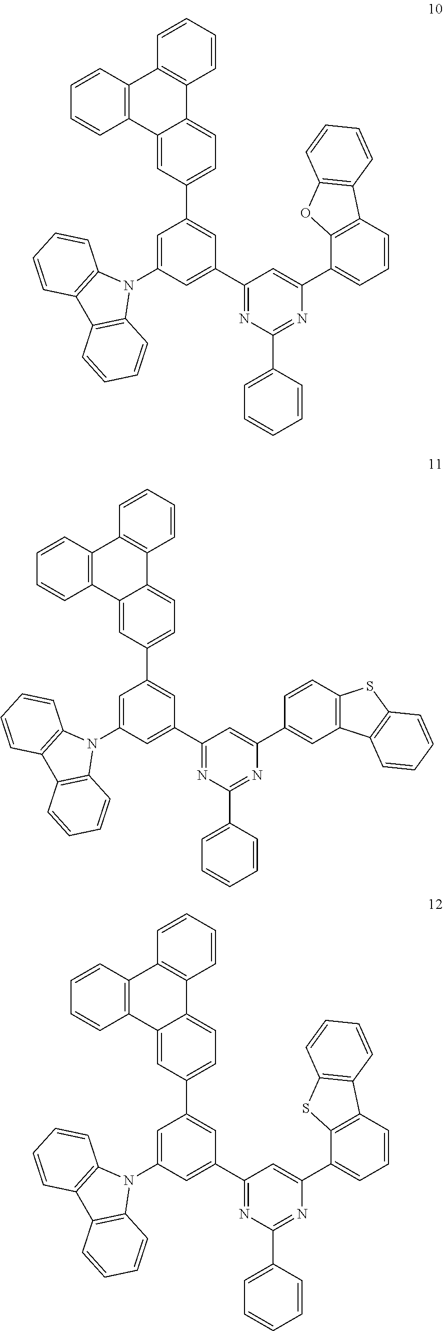

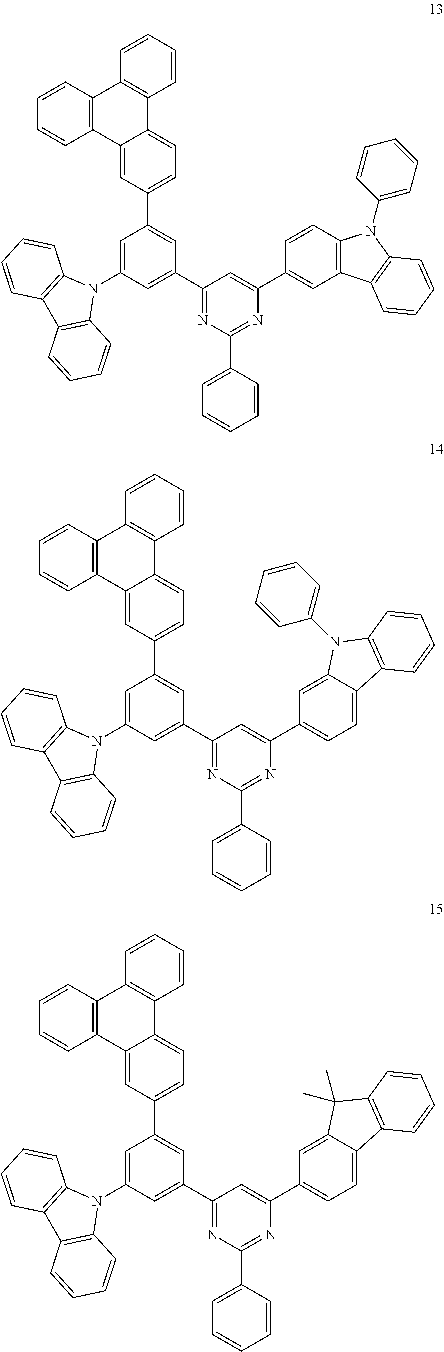 [SMILES: c1ccc(-c2nc(-c3cc(-c4ccc5c6ccccc6c6ccccc6c5c4)cc(-n4c5ccccc5c5ccccc54)c3)cc(-c3ccc4c(c3)c3ccccc3n4-c3ccccc3)n2)cc1 ; c1ccc(-c2nc(-c3cc(-c4ccc5c6ccccc6c6ccccc6c5c4)cc(-n4c5ccccc5c5ccccc54)c3)cc(-c3ccc4c5ccccc5n(-c5ccccc5)c4c3)n2)cc1 ; CC1(C)c2ccccc2-c2ccc(-c3cc(-c4cc(-c5ccc6c7ccccc7c7ccccc7c6c5)cc(-n5c6ccccc6c6ccccc65)c4)nc(-c4ccccc4)n3)cc21]

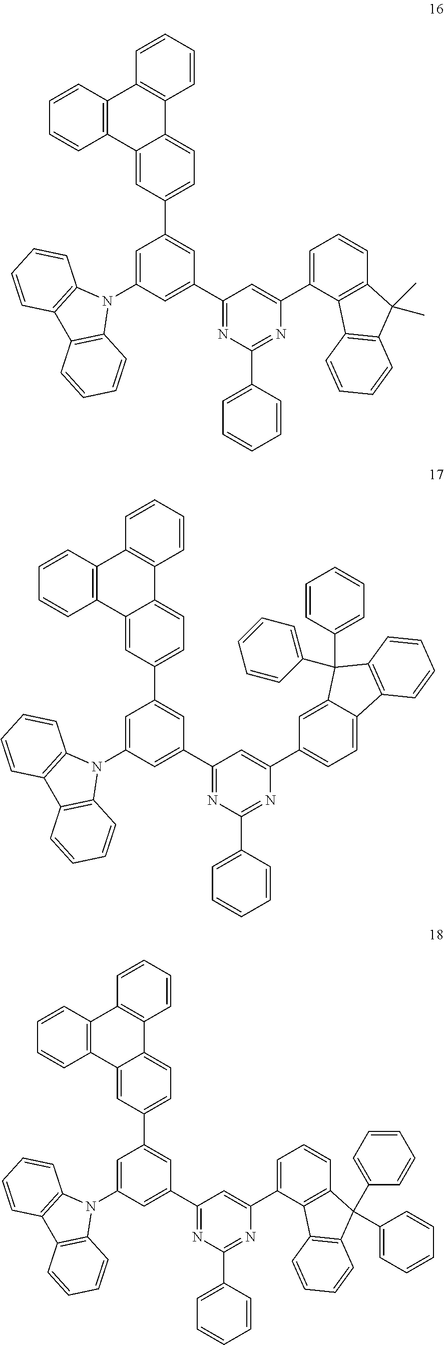 [SMILES: CC1(C)c2ccccc2-c2c(-c3cc(-c4cc(-c5ccc6c7ccccc7c7ccccc7c6c5)cc(-n5c6ccccc6c6ccccc65)c4)nc(-c4ccccc4)n3)cccc21 ; c1ccc(-c2nc(-c3cc(-c4ccc5c6ccccc6c6ccccc6c5c4)cc(-n4c5ccccc5c5ccccc54)c3)cc(-c3ccc4c(c3)C(c3ccccc3)(c3ccccc3)c3ccccc3-4)n2)cc1 ; c1ccc(-c2nc(-c3cc(-c4ccc5c6ccccc6c6ccccc6c5c4)cc(-n4c5ccccc5c5ccccc54)c3)cc(-c3cccc4c3-c3ccccc3C4(c3ccccc3)c3ccccc3)n2)cc1]

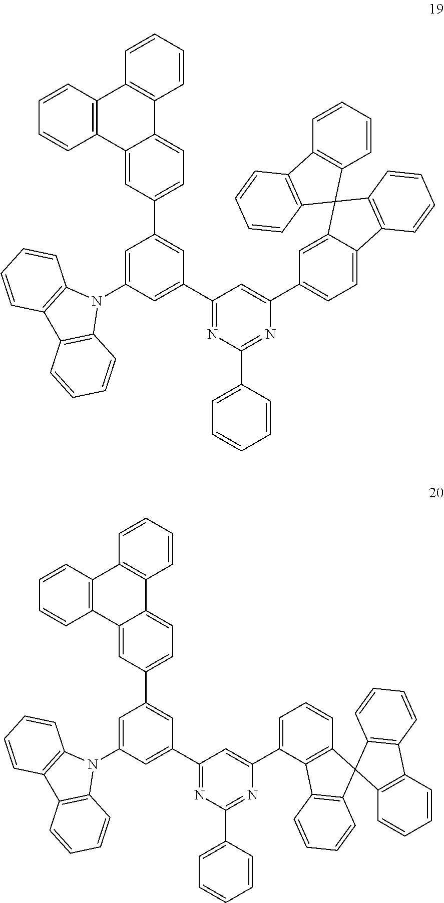 [SMILES: c1ccc(-c2nc(-c3cc(-c4ccc5c6ccccc6c6ccccc6c5c4)cc(-n4c5ccccc5c5ccccc54)c3)cc(-c3ccc4c(c3)C3(c5ccccc5-c5ccccc53)c3ccccc3-4)n2)cc1 ; c1ccc(-c2nc(-c3cc(-c4ccc5c6ccccc6c6ccccc6c5c4)cc(-n4c5ccccc5c5ccccc54)c3)cc(-c3cccc4c3-c3ccccc3C43c4ccccc4-c4ccccc43)n2)cc1]

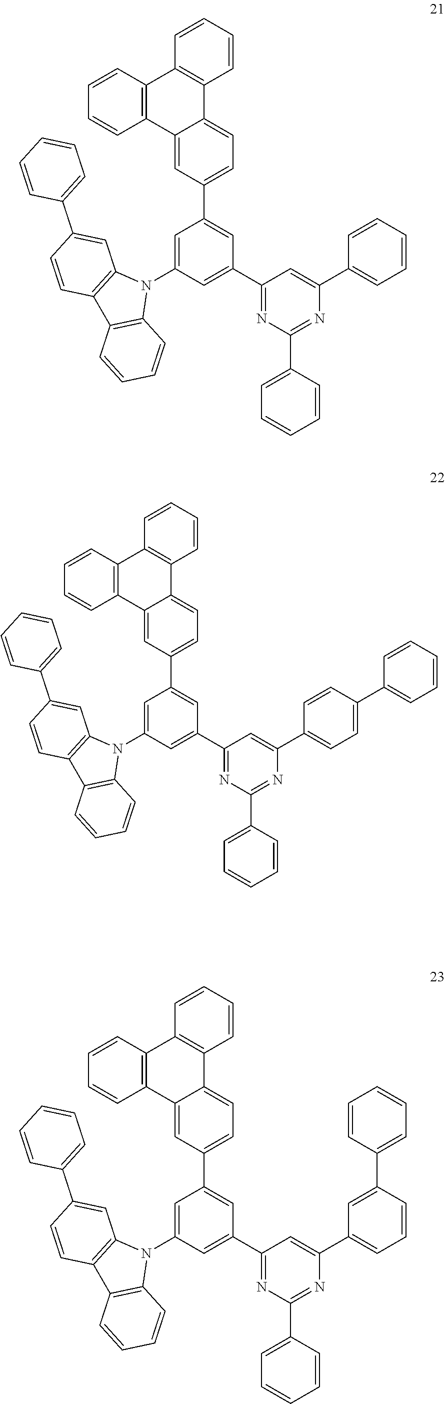 [SMILES: c1ccc(-c2ccc3c4ccccc4n(-c4cc(-c5ccc6c7ccccc7c7ccccc7c6c5)cc(-c5cc(-c6ccccc6)nc(-c6ccccc6)n5)c4)c3c2)cc1 ; c1ccc(-c2ccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cc2)cc1 ; c1ccc(-c2cccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)c2)cc1]

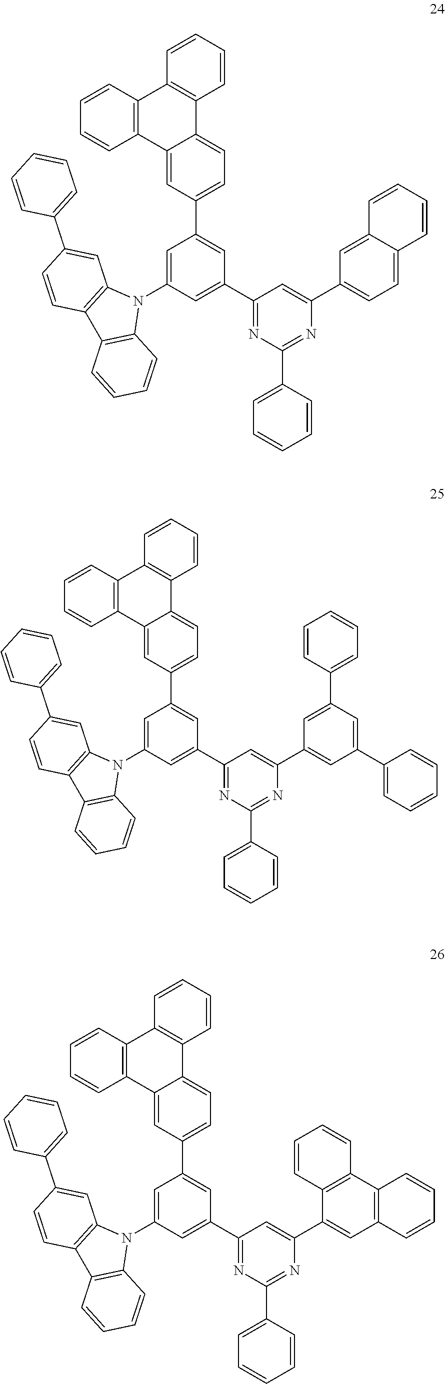 [SMILES: c1ccc(-c2ccc3c4ccccc4n(-c4cc(-c5ccc6c7ccccc7c7ccccc7c6c5)cc(-c5cc(-c6ccc7ccccc7c6)nc(-c6ccccc6)n5)c4)c3c2)cc1 ; c1ccc(-c2cc(-c3ccccc3)cc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)c2)cc1 ; c1ccc(-c2ccc3c4ccccc4n(-c4cc(-c5ccc6c7ccccc7c7ccccc7c6c5)cc(-c5cc(-c6cc7ccccc7c7ccccc67)nc(-c6ccccc6)n5)c4)c3c2)cc1]

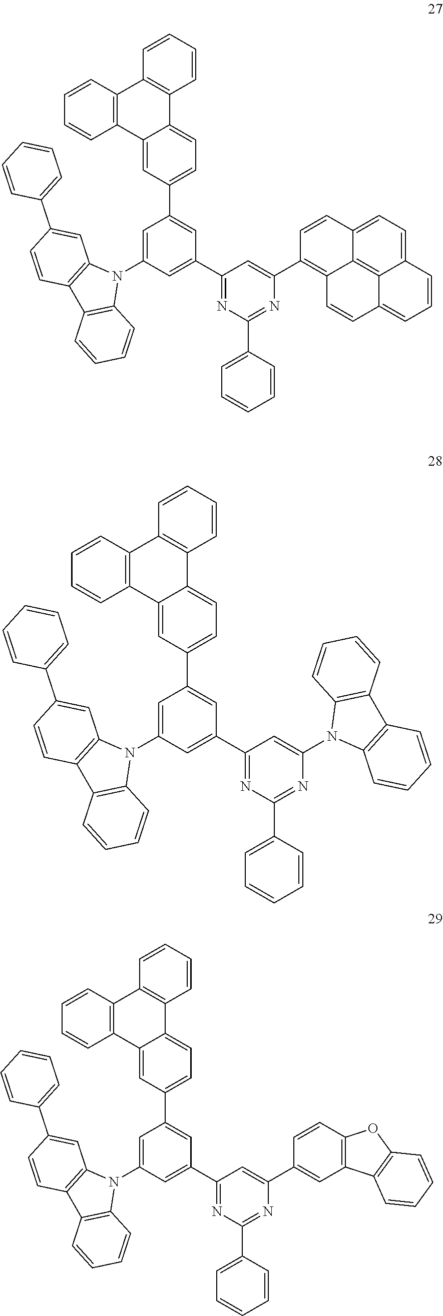 [SMILES: c1ccc(-c2ccc3c4ccccc4n(-c4cc(-c5ccc6c7ccccc7c7ccccc7c6c5)cc(-c5cc(-c6ccc7ccc8cccc9ccc6c7c89)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-n6c7ccccc7c7ccccc76)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7oc8ccccc8c7c6)nc(-c6ccccc6)n5)c4)c3c2)cc1]

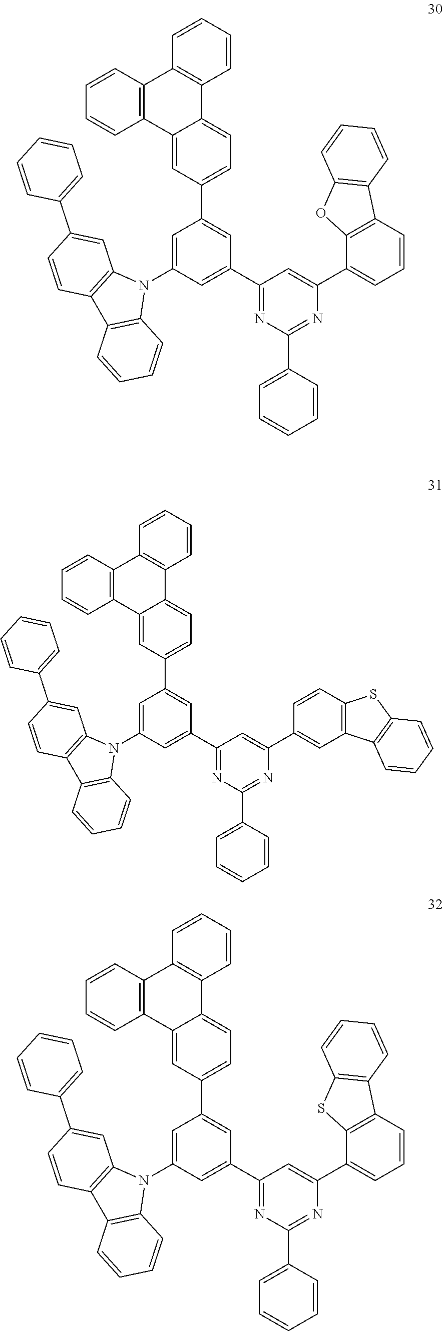 [SMILES: c1ccc(-c2ccc3c4ccccc4n(-c4cc(-c5ccc6c7ccccc7c7ccccc7c6c5)cc(-c5cc(-c6cccc7c6oc6ccccc67)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7sc8ccccc8c7c6)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6sc6ccccc67)nc(-c6ccccc6)n5)c4)c3c2)cc1]

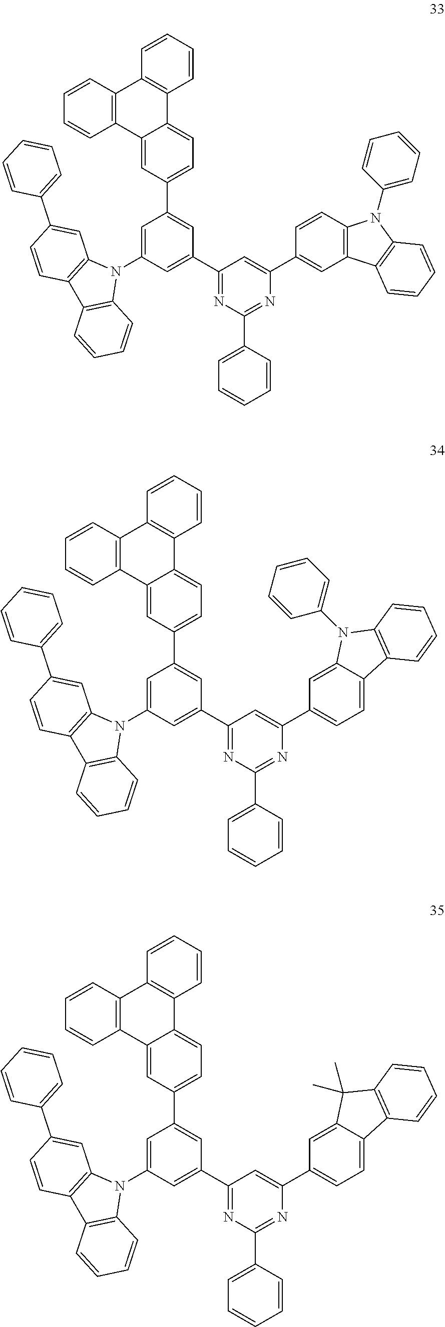 [SMILES: c1ccc(-c2ccc3c4ccccc4n(-c4cc(-c5ccc6c7ccccc7c7ccccc7c6c5)cc(-c5cc(-c6ccc7c(c6)c6ccccc6n7-c6ccccc6)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7c8ccccc8n(-c8ccccc8)c7c6)nc(-c6ccccc6)n5)c4)c3c2)cc1 ; CC1(C)c2ccccc2-c2ccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cc21]

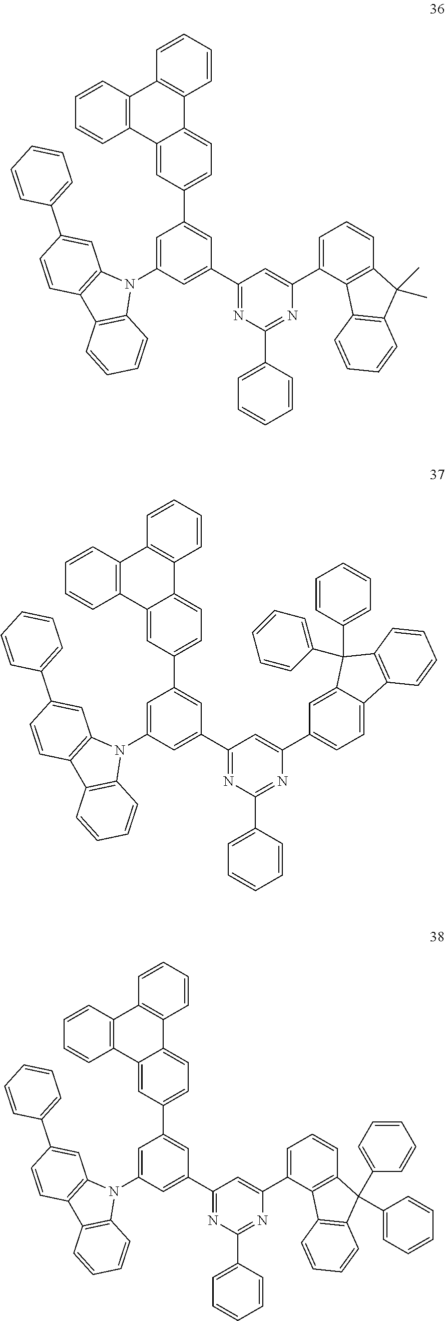 [SMILES: CC1(C)c2ccccc2-c2c(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cccc21 ; c1ccc(-c2ccc3c4ccccc4n(-c4cc(-c5ccc6c7ccccc7c7ccccc7c6c5)cc(-c5cc(-c6ccc7c(c6)C(c6ccccc6)(c6ccccc6)c6ccccc6-7)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6-c6ccccc6C7(c6ccccc6)c6ccccc6)nc(-c6ccccc6)n5)c4)c3c2)cc1]

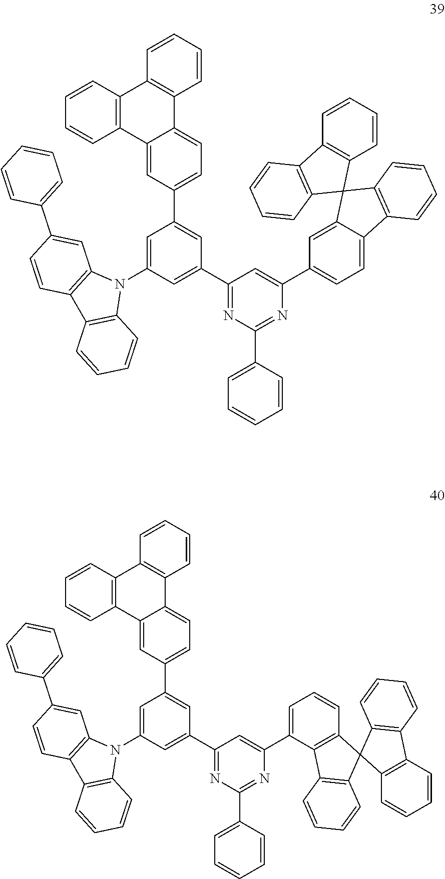 [SMILES: c1ccc(-c2ccc3c4ccccc4n(-c4cc(-c5ccc6c7ccccc7c7ccccc7c6c5)cc(-c5cc(-c6ccc7c(c6)C6(c8ccccc8-c8ccccc86)c6ccccc6-7)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6-c6ccccc6C76c7ccccc7-c7ccccc76)nc(-c6ccccc6)n5)c4)c3c2)cc1]

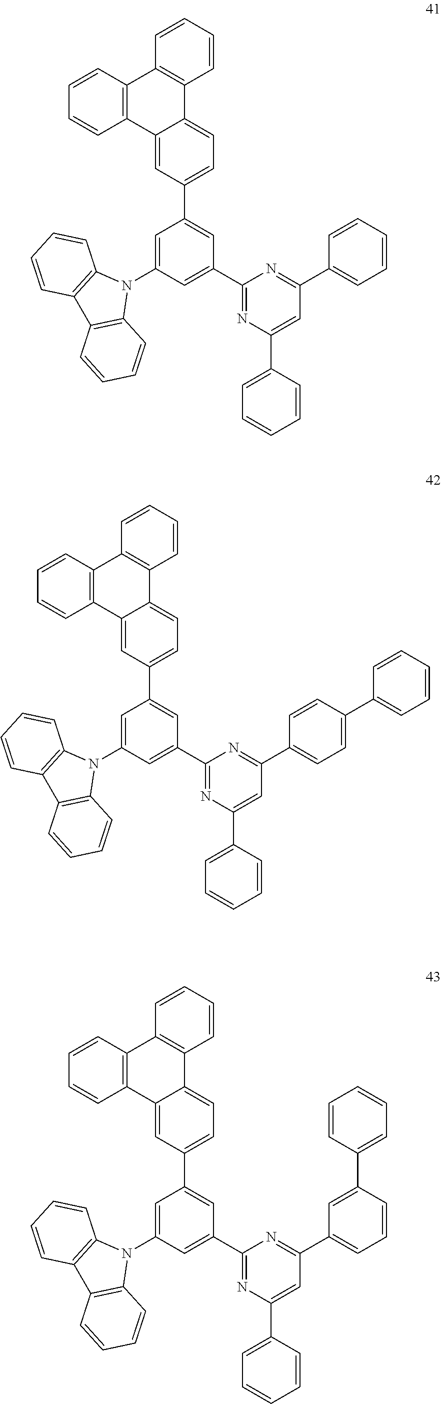 [SMILES: c1ccc(-c2cc(-c3ccccc3)nc(-c3cc(-c4ccc5c6ccccc6c6ccccc6c5c4)cc(-n4c5ccccc5c5ccccc54)c3)n2)cc1 ; c1ccc(-c2ccc(-c3cc(-c4ccccc4)nc(-c4cc(-c5ccc6c7ccccc7c7ccccc7c6c5)cc(-n5c6ccccc6c6ccccc65)c4)n3)cc2)cc1 ; c1ccc(-c2cccc(-c3cc(-c4ccccc4)nc(-c4cc(-c5ccc6c7ccccc7c7ccccc7c6c5)cc(-n5c6ccccc6c6ccccc65)c4)n3)c2)cc1]

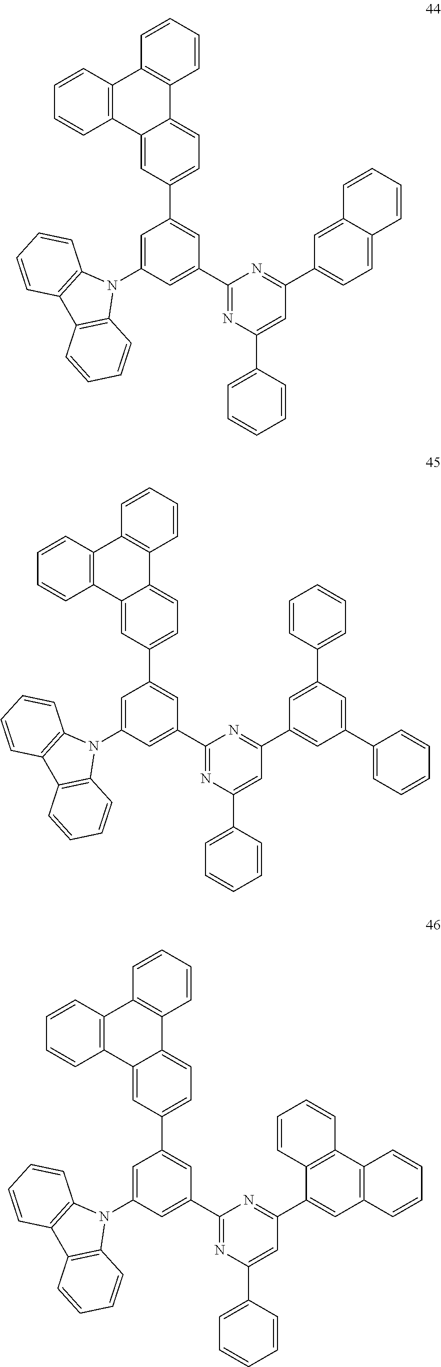 [SMILES: c1ccc(-c2cc(-c3ccc4ccccc4c3)nc(-c3cc(-c4ccc5c6ccccc6c6ccccc6c5c4)cc(-n4c5ccccc5c5ccccc54)c3)n2)cc1 ; c1ccc(-c2cc(-c3ccccc3)cc(-c3cc(-c4ccccc4)nc(-c4cc(-c5ccc6c7ccccc7c7ccccc7c6c5)cc(-n5c6ccccc6c6ccccc65)c4)n3)c2)cc1 ; c1ccc(-c2cc(-c3cc4ccccc4c4ccccc34)nc(-c3cc(-c4ccc5c6ccccc6c6ccccc6c5c4)cc(-n4c5ccccc5c5ccccc54)c3)n2)cc1]

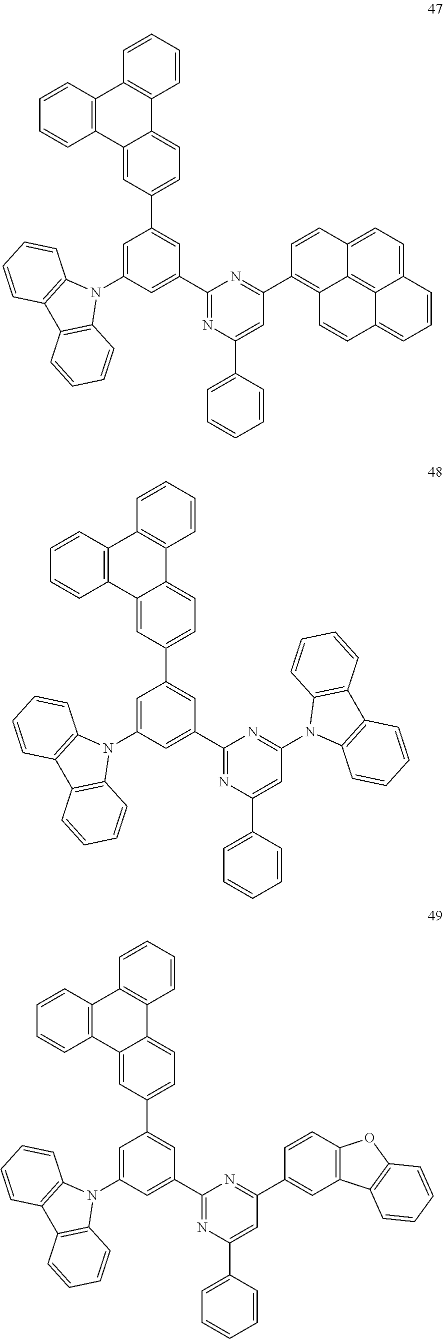 [SMILES: c1ccc(-c2cc(-c3ccc4ccc5cccc6ccc3c4c56)nc(-c3cc(-c4ccc5c6ccccc6c6ccccc6c5c4)cc(-n4c5ccccc5c5ccccc54)c3)n2)cc1 ; c1ccc(-c2cc(-n3c4ccccc4c4ccccc43)nc(-c3cc(-c4ccc5c6ccccc6c6ccccc6c5c4)cc(-n4c5ccccc5c5ccccc54)c3)n2)cc1 ; c1ccc(-c2cc(-c3ccc4oc5ccccc5c4c3)nc(-c3cc(-c4ccc5c6ccccc6c6ccccc6c5c4)cc(-n4c5ccccc5c5ccccc54)c3)n2)cc1]

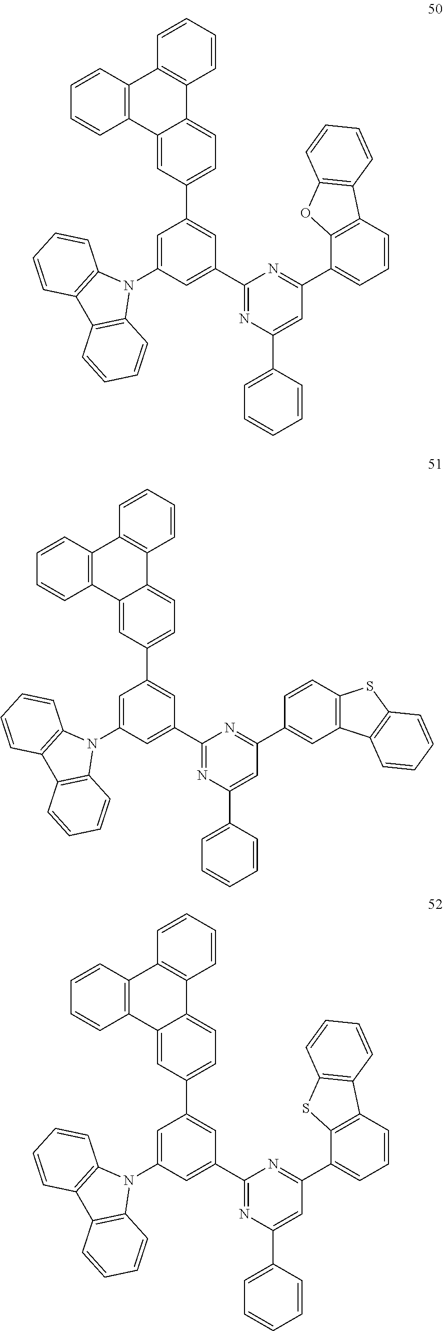

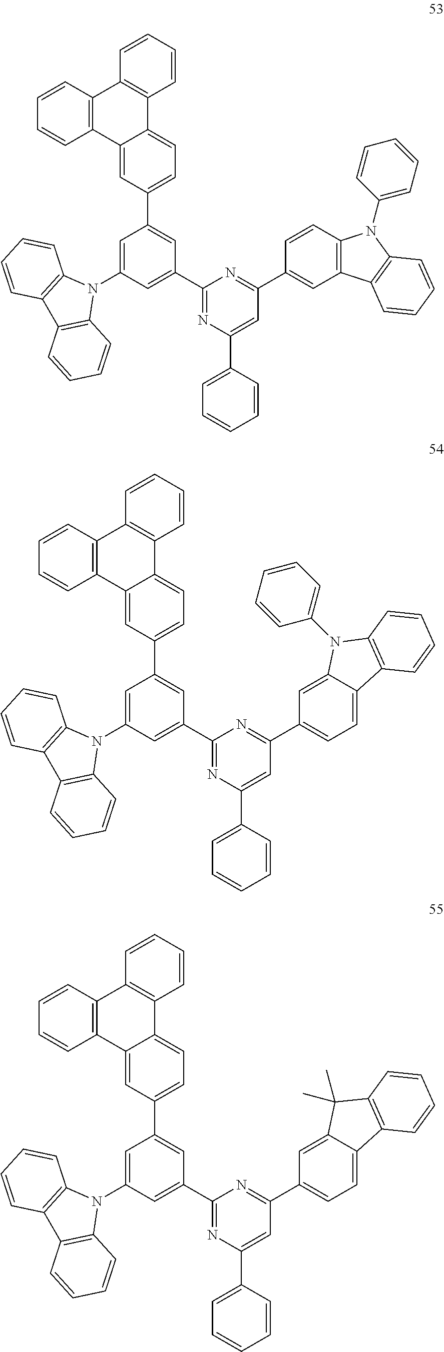 [SMILES: c1ccc(-c2cc(-c3ccc4c(c3)c3ccccc3n4-c3ccccc3)nc(-c3cc(-c4ccc5c6ccccc6c6ccccc6c5c4)cc(-n4c5ccccc5c5ccccc54)c3)n2)cc1 ; c1ccc(-c2cc(-c3ccc4c5ccccc5n(-c5ccccc5)c4c3)nc(-c3cc(-c4ccc5c6ccccc6c6ccccc6c5c4)cc(-n4c5ccccc5c5ccccc54)c3)n2)cc1 ; CC1(C)c2ccccc2-c2ccc(-c3cc(-c4ccccc4)nc(-c4cc(-c5ccc6c7ccccc7c7ccccc7c6c5)cc(-n5c6ccccc6c6ccccc65)c4)n3)cc21]

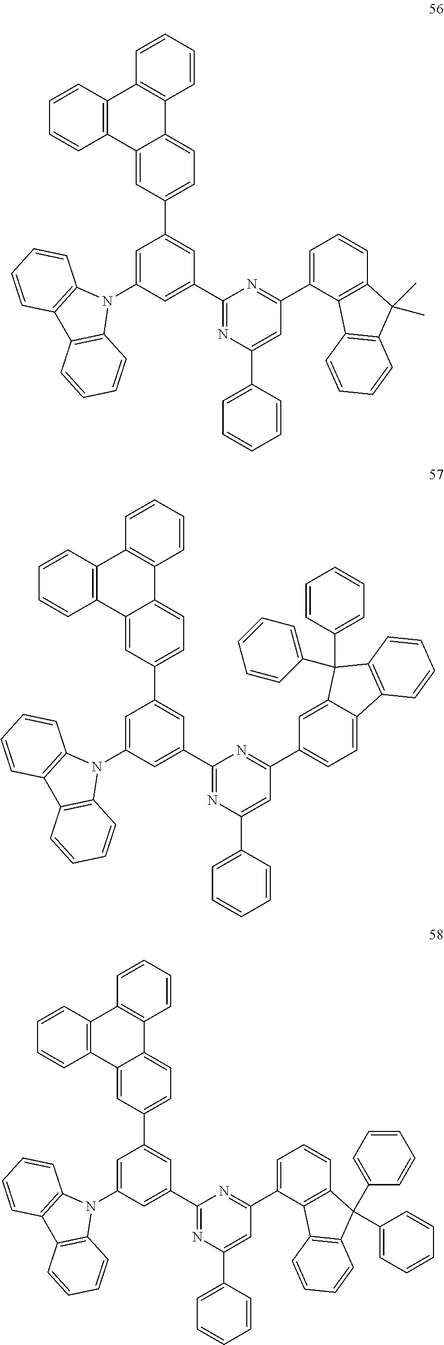 [SMILES: CC1(C)c2ccccc2-c2c(-c3cc(-c4ccccc4)nc(-c4cc(-c5ccc6c7ccccc7c7ccccc7c6c5)cc(-n5c6ccccc6c6ccccc65)c4)n3)cccc21 ; c1ccc(-c2cc(-c3ccc4c(c3)C(c3ccccc3)(c3ccccc3)c3ccccc3-4)nc(-c3cc(-c4ccc5c6ccccc6c6ccccc6c5c4)cc(-n4c5ccccc5c5ccccc54)c3)n2)cc1 ; c1ccc(-c2cc(-c3cccc4c3-c3ccccc3C4(c3ccccc3)c3ccccc3)nc(-c3cc(-c4ccc5c6ccccc6c6ccccc6c5c4)cc(-n4c5ccccc5c5ccccc54)c3)n2)cc1]

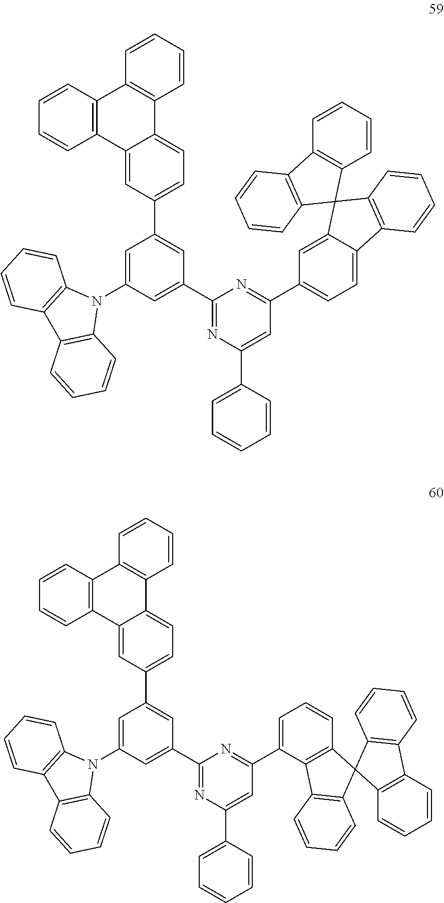 [SMILES: c1ccc(-c2cc(-c3ccc4c(c3)C3(c5ccccc5-c5ccccc53)c3ccccc3-4)nc(-c3cc(-c4ccc5c6ccccc6c6ccccc6c5c4)cc(-n4c5ccccc5c5ccccc54)c3)n2)cc1 ; c1ccc(-c2cc(-c3cccc4c3-c3ccccc3C43c4ccccc4-c4ccccc43)nc(-c3cc(-c4ccc5c6ccccc6c6ccccc6c5c4)cc(-n4c5ccccc5c5ccccc54)c3)n2)cc1]

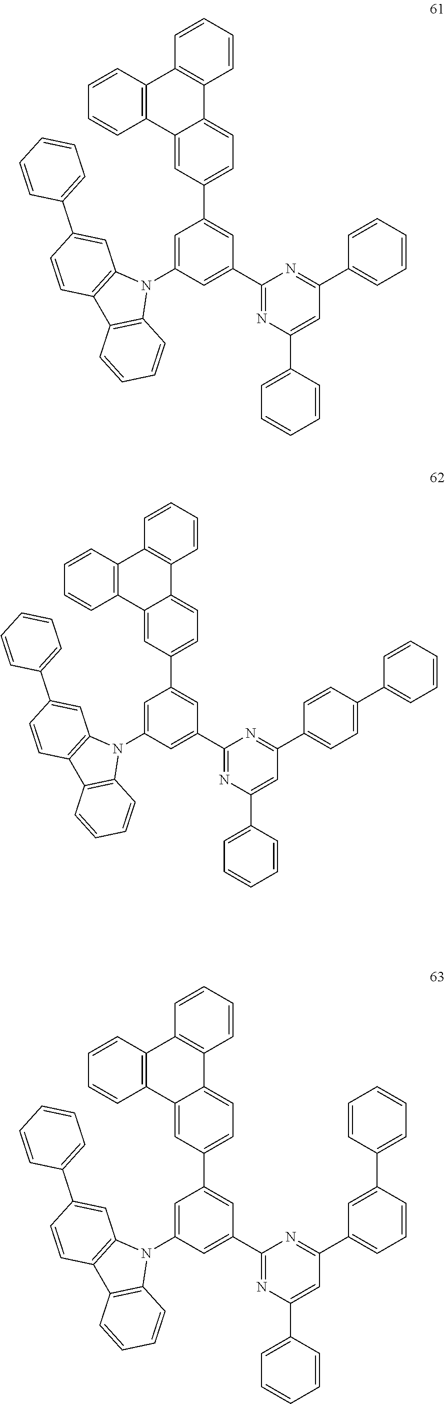 [SMILES: c1ccc(-c2ccc3c4ccccc4n(-c4cc(-c5ccc6c7ccccc7c7ccccc7c6c5)cc(-c5nc(-c6ccccc6)cc(-c6ccccc6)n5)c4)c3c2)cc1 ; c1ccc(-c2ccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cc2)cc1 ; c1ccc(-c2cccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1]

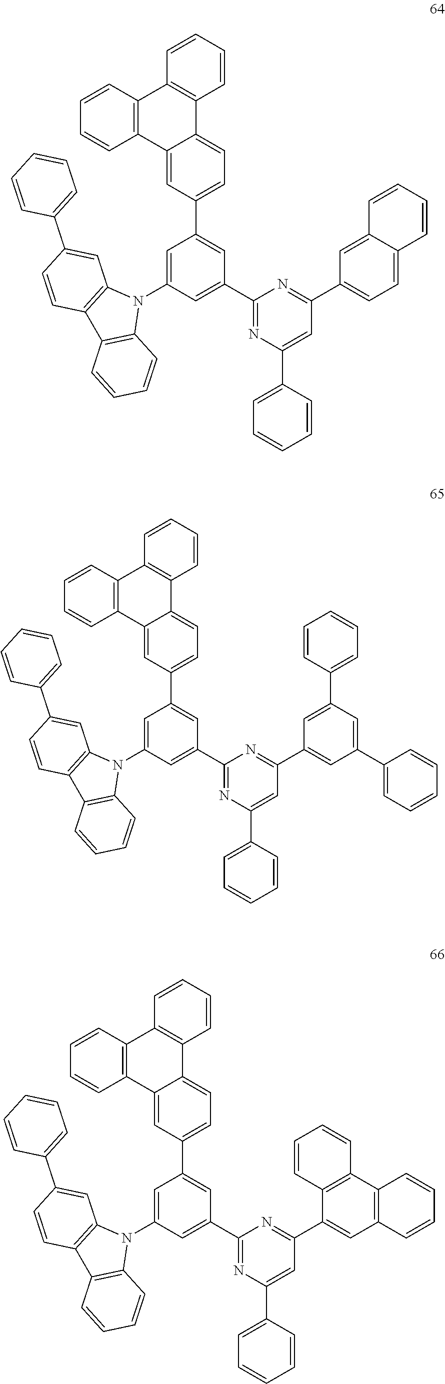 [SMILES: c1ccc(-c2ccc3c4ccccc4n(-c4cc(-c5ccc6c7ccccc7c7ccccc7c6c5)cc(-c5nc(-c6ccccc6)cc(-c6ccc7ccccc7c6)n5)c4)c3c2)cc1 ; c1ccc(-c2cc(-c3ccccc3)cc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2ccc3c4ccccc4n(-c4cc(-c5ccc6c7ccccc7c7ccccc7c6c5)cc(-c5nc(-c6ccccc6)cc(-c6cc7ccccc7c7ccccc67)n5)c4)c3c2)cc1]

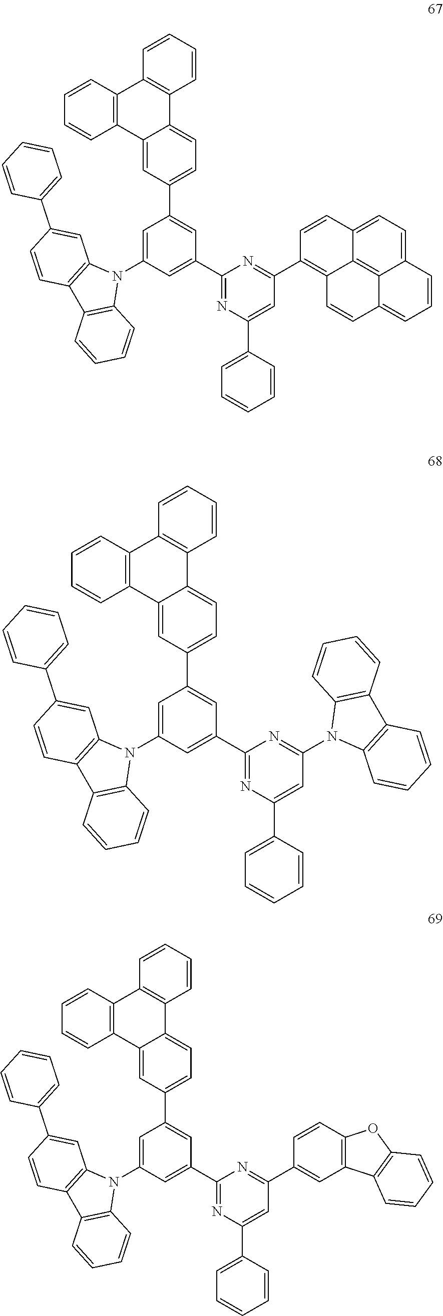 [SMILES: c1ccc(-c2ccc3c4ccccc4n(-c4cc(-c5ccc6c7ccccc7c7ccccc7c6c5)cc(-c5nc(-c6ccccc6)cc(-c6ccc7ccc8cccc9ccc6c7c89)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-n6c7ccccc7c7ccccc76)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7oc8ccccc8c7c6)n5)c4)c3c2)cc1]

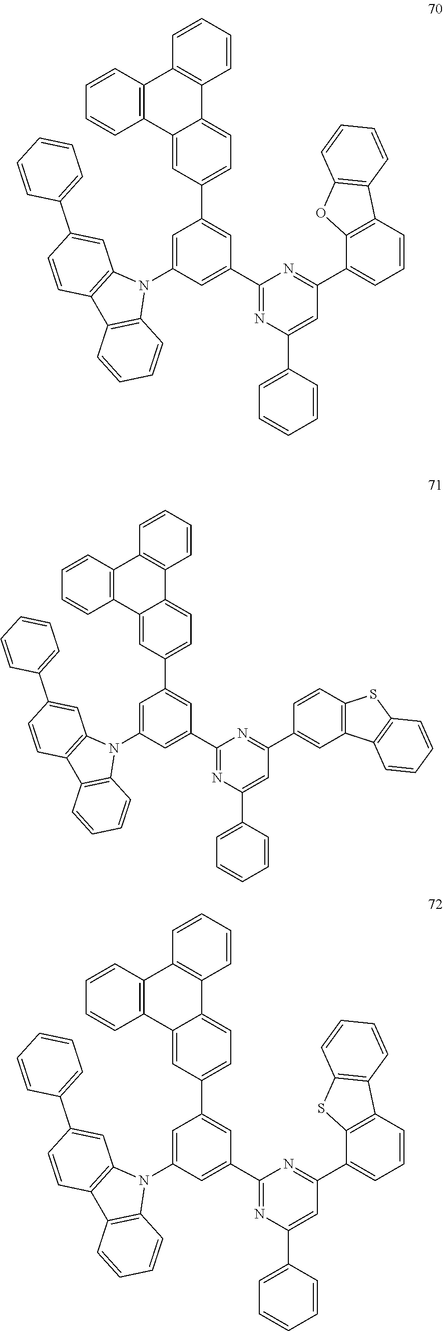 [SMILES: c1ccc(-c2ccc3c4ccccc4n(-c4cc(-c5ccc6c7ccccc7c7ccccc7c6c5)cc(-c5nc(-c6ccccc6)cc(-c6cccc7c6oc6ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7sc8ccccc8c7c6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6sc6ccccc67)n5)c4)c3c2)cc1]

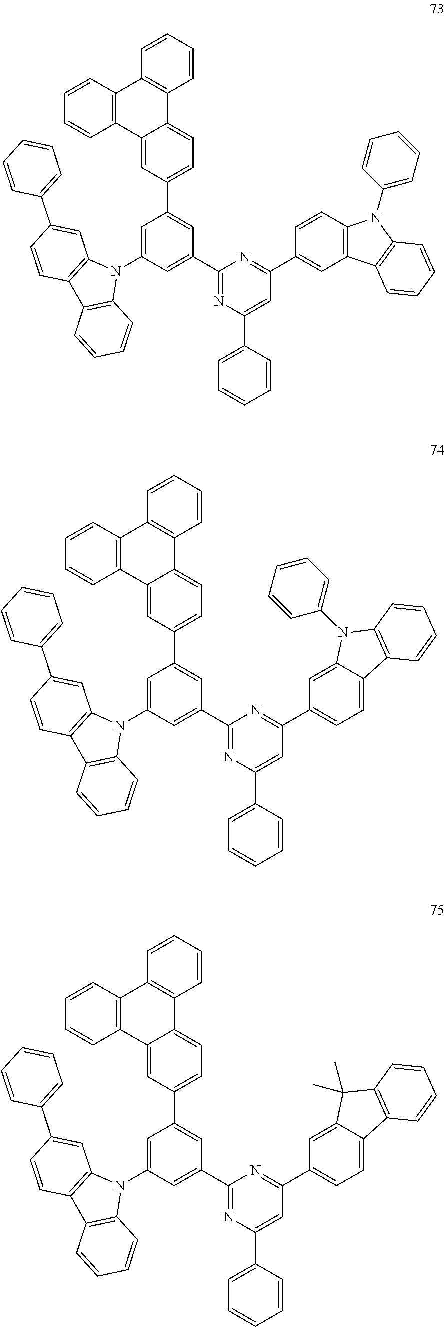 [SMILES: c1ccc(-c2ccc3c4ccccc4n(-c4cc(-c5ccc6c7ccccc7c7ccccc7c6c5)cc(-c5nc(-c6ccccc6)cc(-c6ccc7c(c6)c6ccccc6n7-c6ccccc6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7c8ccccc8n(-c8ccccc8)c7c6)n5)c4)c3c2)cc1 ; CC1(C)c2ccccc2-c2ccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cc21]

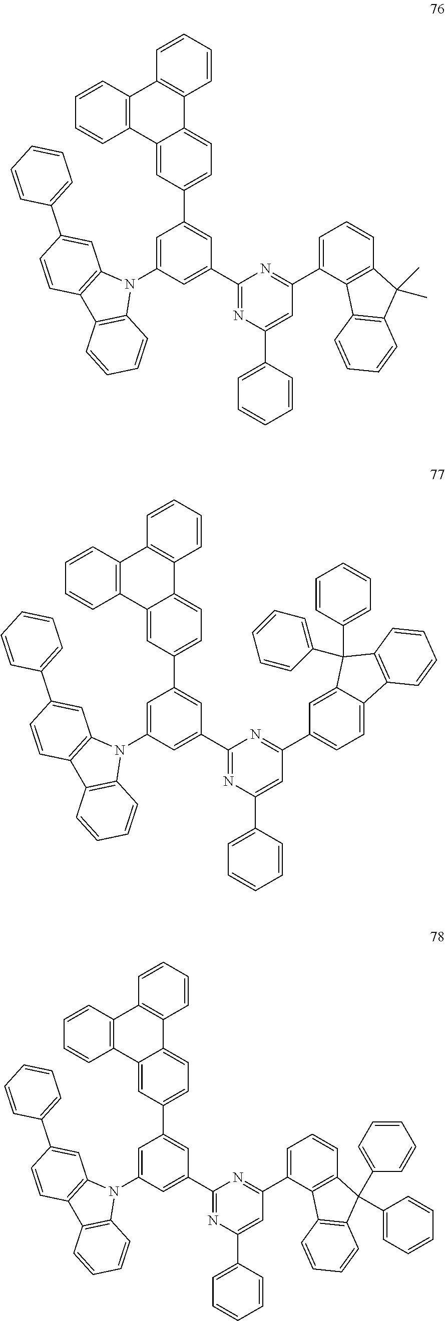 [SMILES: CC1(C)c2ccccc2-c2c(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cccc21 ; c1ccc(-c2ccc3c4ccccc4n(-c4cc(-c5ccc6c7ccccc7c7ccccc7c6c5)cc(-c5nc(-c6ccccc6)cc(-c6ccc7c(c6)C(c6ccccc6)(c6ccccc6)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6-c6ccccc6C7(c6ccccc6)c6ccccc6)n5)c4)c3c2)cc1]

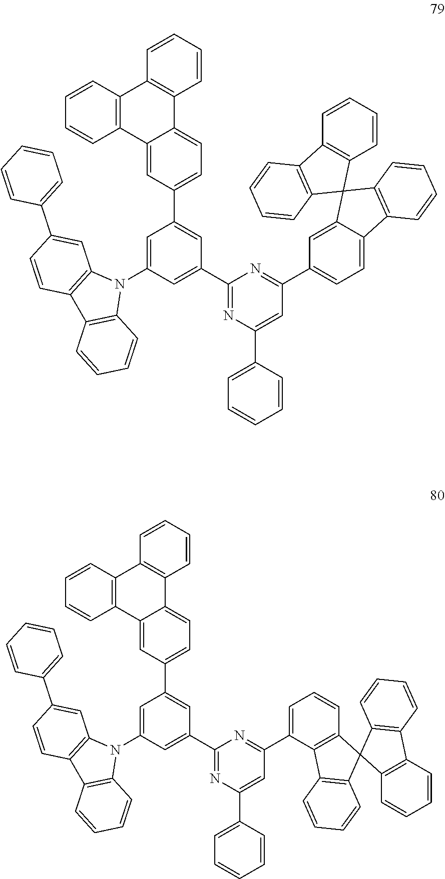 [SMILES: c1ccc(-c2ccc3c4ccccc4n(-c4cc(-c5ccc6c7ccccc7c7ccccc7c6c5)cc(-c5nc(-c6ccccc6)cc(-c6ccc7c(c6)C6(c8ccccc8-c8ccccc86)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6-c6ccccc6C76c7ccccc7-c7ccccc76)n5)c4)c3c2)cc1]

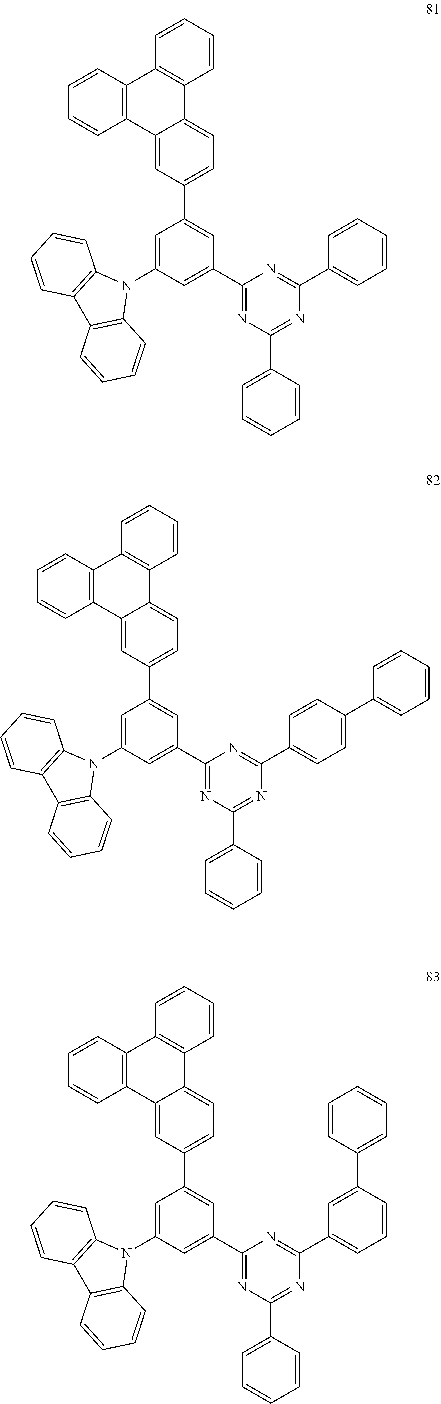 [SMILES: c1ccc(-c2nc(-c3ccccc3)nc(-c3cc(-c4ccc5c6ccccc6c6ccccc6c5c4)cc(-n4c5ccccc5c5ccccc54)c3)n2)cc1 ; c1ccc(-c2ccc(-c3nc(-c4ccccc4)nc(-c4cc(-c5ccc6c7ccccc7c7ccccc7c6c5)cc(-n5c6ccccc6c6ccccc65)c4)n3)cc2)cc1 ; c1ccc(-c2cccc(-c3nc(-c4ccccc4)nc(-c4cc(-c5ccc6c7ccccc7c7ccccc7c6c5)cc(-n5c6ccccc6c6ccccc65)c4)n3)c2)cc1]

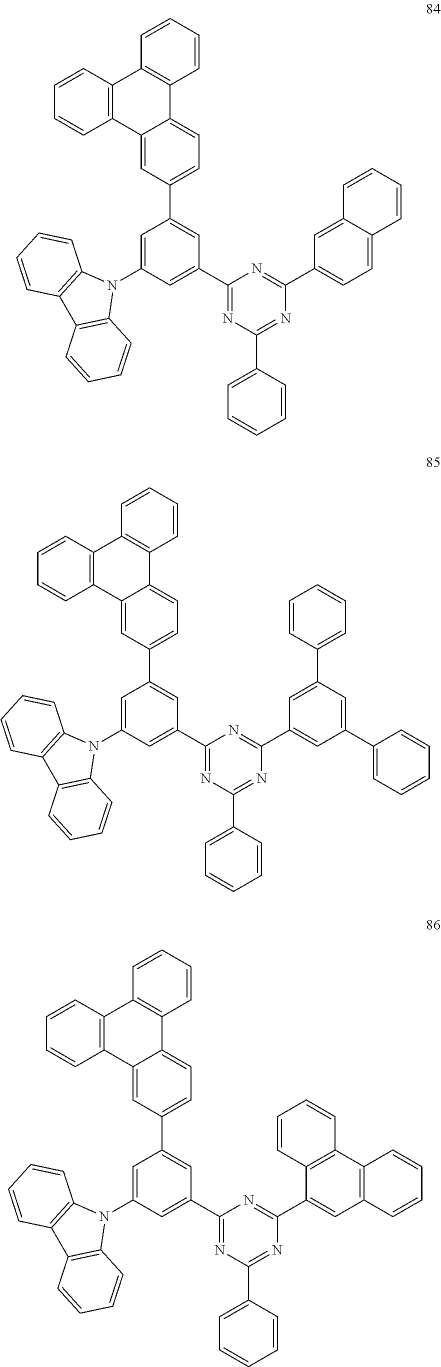 [SMILES: c1ccc(-c2nc(-c3cc(-c4ccc5c6ccccc6c6ccccc6c5c4)cc(-n4c5ccccc5c5ccccc54)c3)nc(-c3ccc4ccccc4c3)n2)cc1 ; c1ccc(-c2cc(-c3ccccc3)cc(-c3nc(-c4ccccc4)nc(-c4cc(-c5ccc6c7ccccc7c7ccccc7c6c5)cc(-n5c6ccccc6c6ccccc65)c4)n3)c2)cc1 ; c1ccc(-c2nc(-c3cc(-c4ccc5c6ccccc6c6ccccc6c5c4)cc(-n4c5ccccc5c5ccccc54)c3)nc(-c3cc4ccccc4c4ccccc34)n2)cc1]

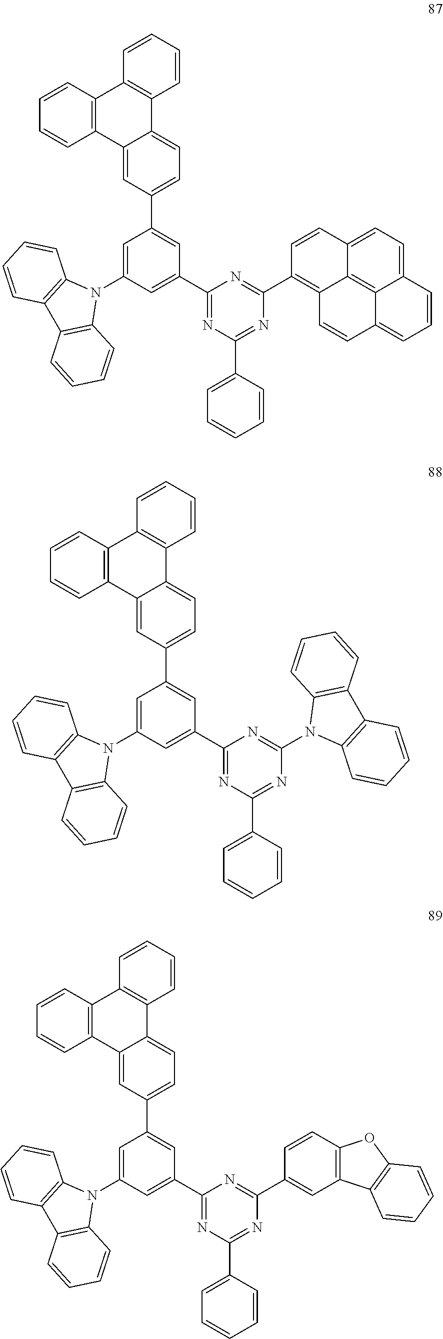 [SMILES: c1ccc(-c2nc(-c3cc(-c4ccc5c6ccccc6c6ccccc6c5c4)cc(-n4c5ccccc5c5ccccc54)c3)nc(-c3ccc4ccc5cccc6ccc3c4c56)n2)cc1 ; c1ccc(-c2nc(-c3cc(-c4ccc5c6ccccc6c6ccccc6c5c4)cc(-n4c5ccccc5c5ccccc54)c3)nc(-n3c4ccccc4c4ccccc43)n2)cc1 ; c1ccc(-c2nc(-c3cc(-c4ccc5c6ccccc6c6ccccc6c5c4)cc(-n4c5ccccc5c5ccccc54)c3)nc(-c3ccc4oc5ccccc5c4c3)n2)cc1]

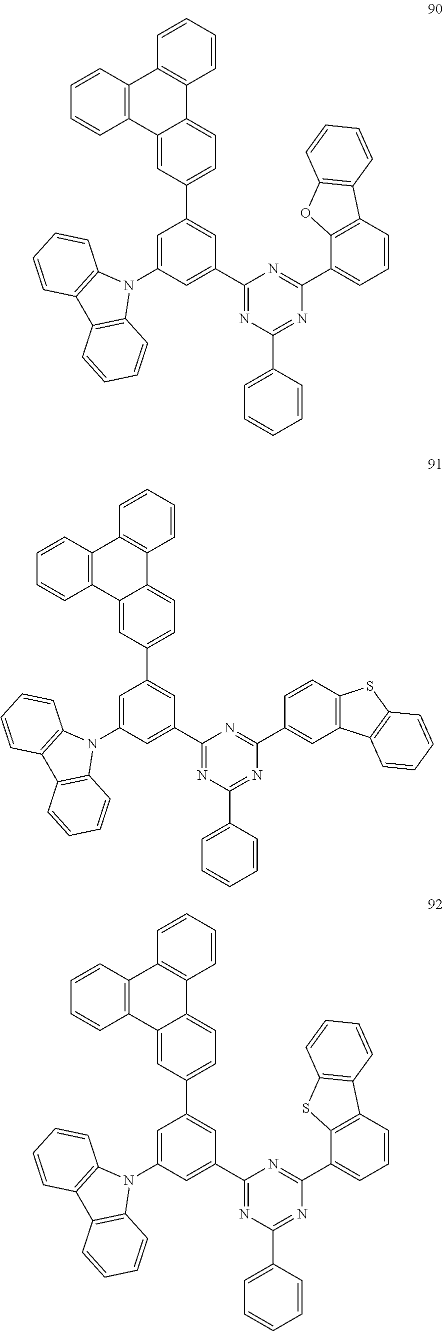

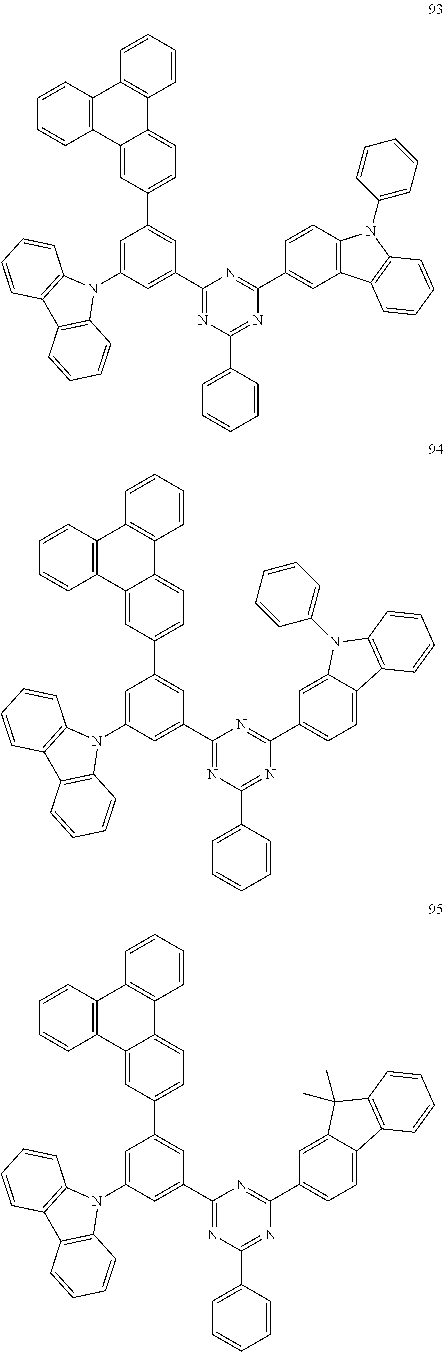 [SMILES: c1ccc(-c2nc(-c3cc(-c4ccc5c6ccccc6c6ccccc6c5c4)cc(-n4c5ccccc5c5ccccc54)c3)nc(-c3ccc4c(c3)c3ccccc3n4-c3ccccc3)n2)cc1 ; c1ccc(-c2nc(-c3cc(-c4ccc5c6ccccc6c6ccccc6c5c4)cc(-n4c5ccccc5c5ccccc54)c3)nc(-c3ccc4c5ccccc5n(-c5ccccc5)c4c3)n2)cc1 ; CC1(C)c2ccccc2-c2ccc(-c3nc(-c4ccccc4)nc(-c4cc(-c5ccc6c7ccccc7c7ccccc7c6c5)cc(-n5c6ccccc6c6ccccc65)c4)n3)cc21]

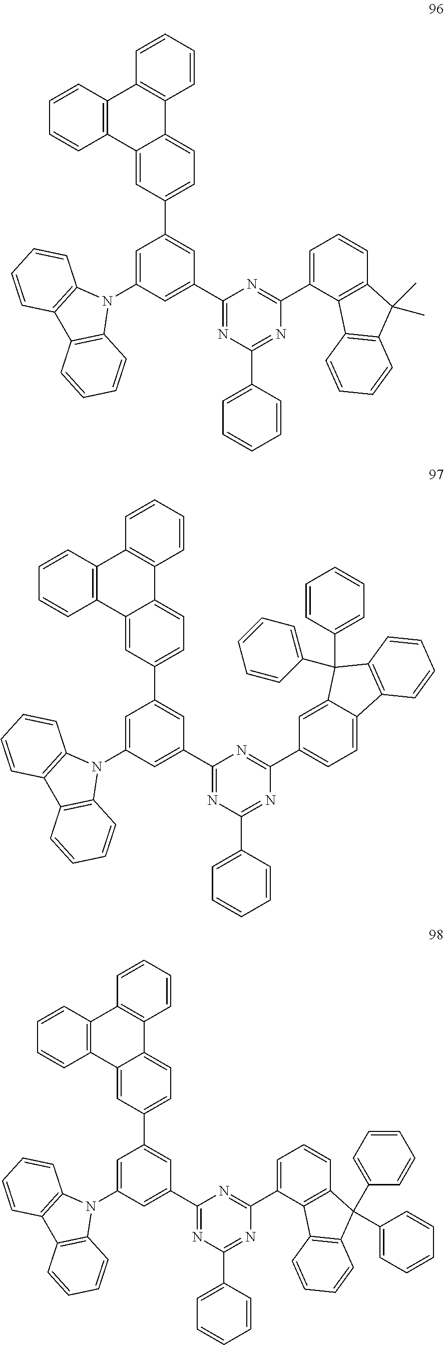 [SMILES: CC1(C)c2ccccc2-c2c(-c3nc(-c4ccccc4)nc(-c4cc(-c5ccc6c7ccccc7c7ccccc7c6c5)cc(-n5c6ccccc6c6ccccc65)c4)n3)cccc21 ; c1ccc(-c2nc(-c3cc(-c4ccc5c6ccccc6c6ccccc6c5c4)cc(-n4c5ccccc5c5ccccc54)c3)nc(-c3ccc4c(c3)C(c3ccccc3)(c3ccccc3)c3ccccc3-4)n2)cc1 ; c1ccc(-c2nc(-c3cc(-c4ccc5c6ccccc6c6ccccc6c5c4)cc(-n4c5ccccc5c5ccccc54)c3)nc(-c3cccc4c3-c3ccccc3C4(c3ccccc3)c3ccccc3)n2)cc1]

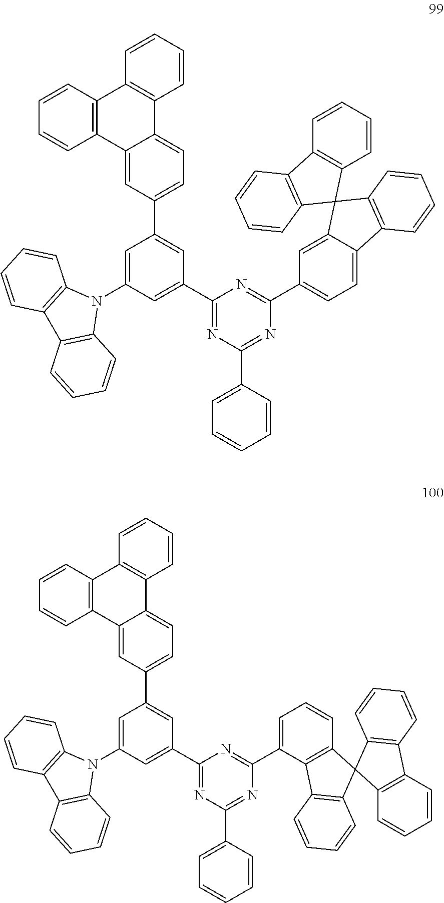 [SMILES: c1ccc(-c2nc(-c3cc(-c4ccc5c6ccccc6c6ccccc6c5c4)cc(-n4c5ccccc5c5ccccc54)c3)nc(-c3ccc4c(c3)C3(c5ccccc5-c5ccccc53)c3ccccc3-4)n2)cc1 ; c1ccc(-c2nc(-c3cc(-c4ccc5c6ccccc6c6ccccc6c5c4)cc(-n4c5ccccc5c5ccccc54)c3)nc(-c3cccc4c3-c3ccccc3C43c4ccccc4-c4ccccc43)n2)cc1]

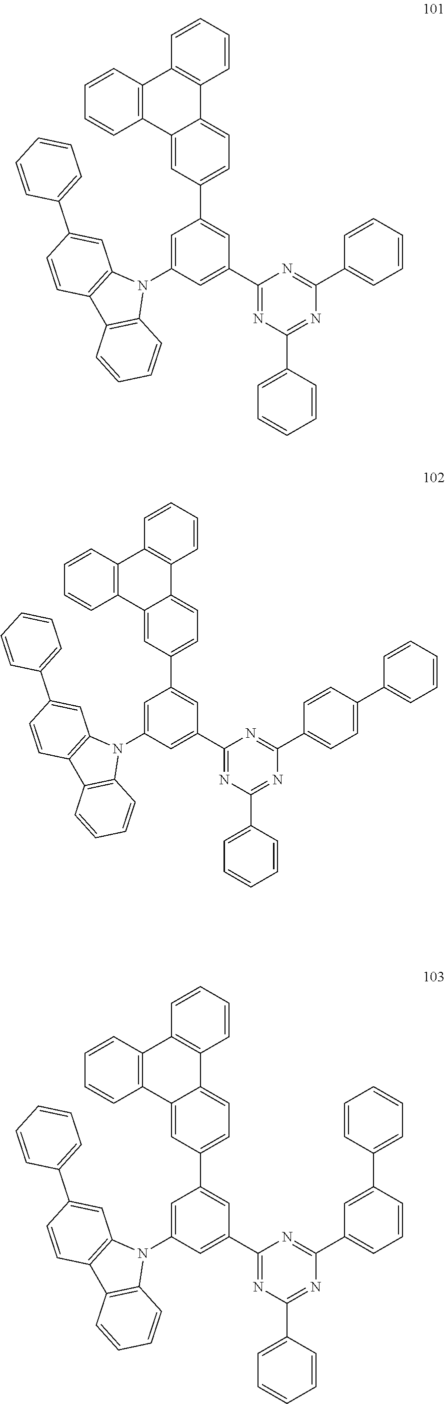 [SMILES: c1ccc(-c2ccc3c4ccccc4n(-c4cc(-c5ccc6c7ccccc7c7ccccc7c6c5)cc(-c5nc(-c6ccccc6)nc(-c6ccccc6)n5)c4)c3c2)cc1 ; c1ccc(-c2ccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cc2)cc1 ; c1ccc(-c2cccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1]

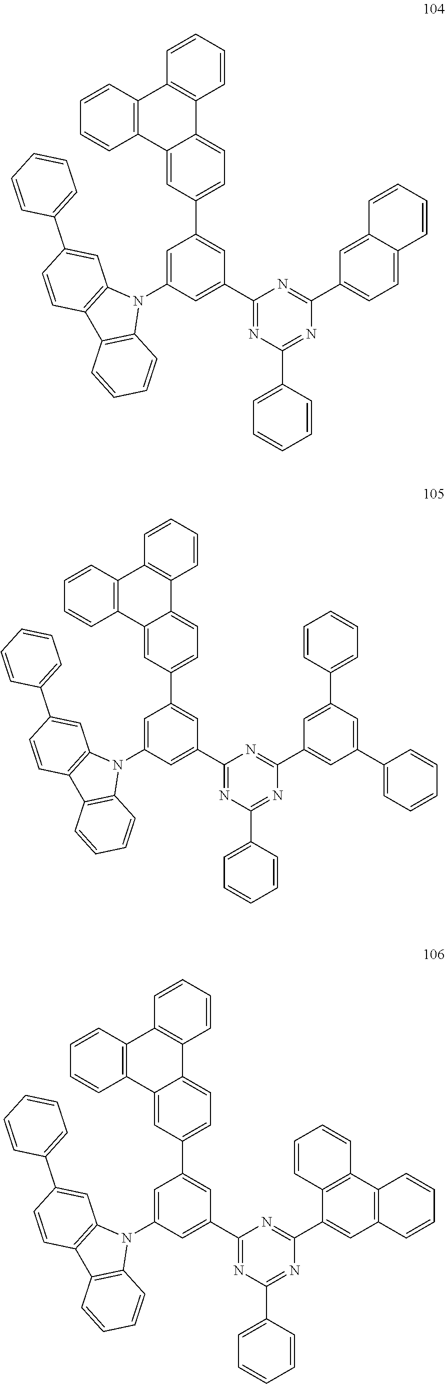 [SMILES: c1ccc(-c2ccc3c4ccccc4n(-c4cc(-c5ccc6c7ccccc7c7ccccc7c6c5)cc(-c5nc(-c6ccccc6)nc(-c6ccc7ccccc7c6)n5)c4)c3c2)cc1 ; c1ccc(-c2cc(-c3ccccc3)cc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2ccc3c4ccccc4n(-c4cc(-c5ccc6c7ccccc7c7ccccc7c6c5)cc(-c5nc(-c6ccccc6)nc(-c6cc7ccccc7c7ccccc67)n5)c4)c3c2)cc1]

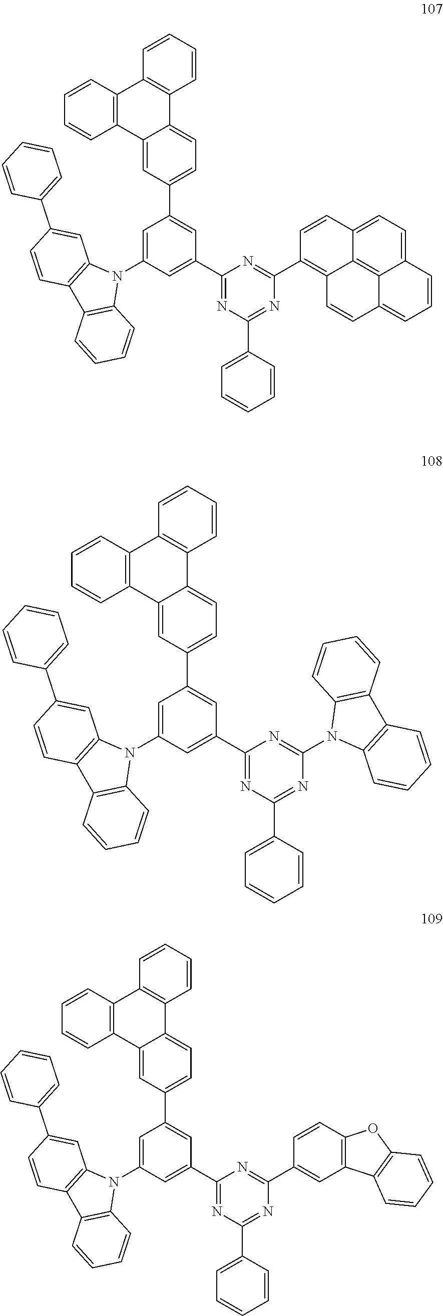 [SMILES: c1ccc(-c2ccc3c4ccccc4n(-c4cc(-c5ccc6c7ccccc7c7ccccc7c6c5)cc(-c5nc(-c6ccccc6)nc(-c6ccc7ccc8cccc9ccc6c7c89)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-n6c7ccccc7c7ccccc76)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7oc8ccccc8c7c6)n5)c4)c3c2)cc1]

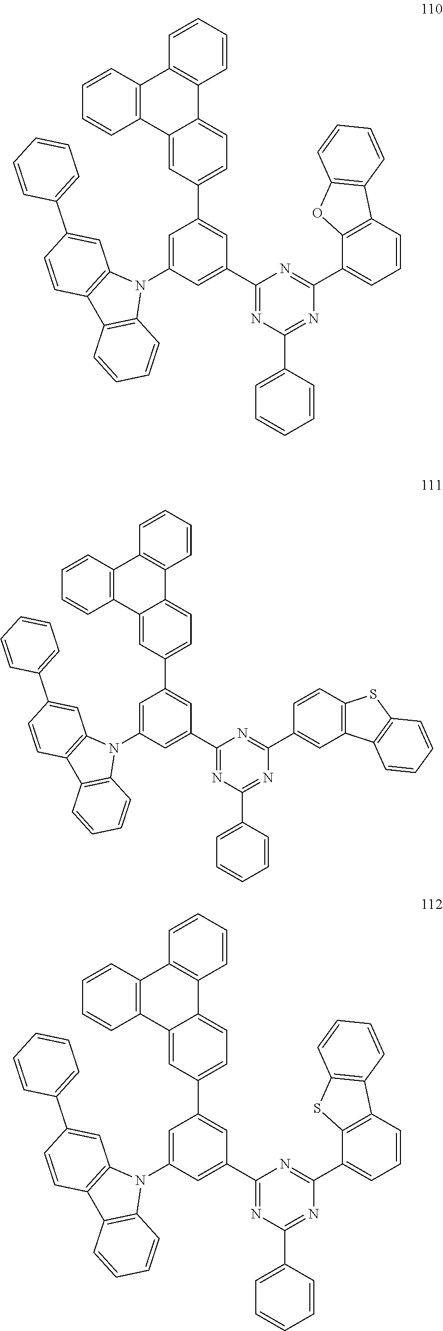 [SMILES: c1ccc(-c2ccc3c4ccccc4n(-c4cc(-c5ccc6c7ccccc7c7ccccc7c6c5)cc(-c5nc(-c6ccccc6)nc(-c6cccc7c6oc6ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7sc8ccccc8c7c6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6cccc7c6sc6ccccc67)n5)c4)c3c2)cc1]

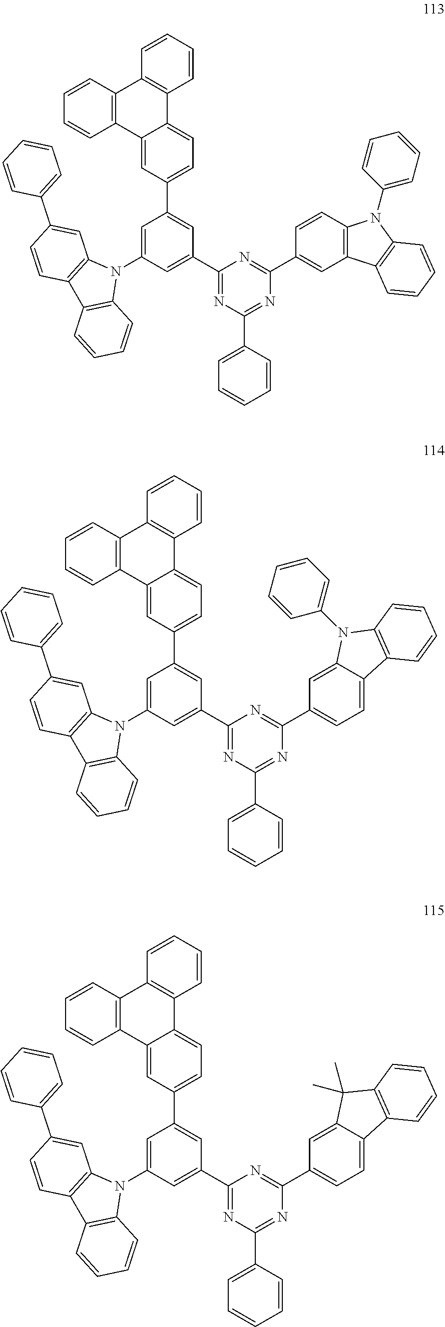 [SMILES: c1ccc(-c2ccc3c4ccccc4n(-c4cc(-c5ccc6c7ccccc7c7ccccc7c6c5)cc(-c5nc(-c6ccccc6)nc(-c6ccc7c(c6)c6ccccc6n7-c6ccccc6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7c8ccccc8n(-c8ccccc8)c7c6)n5)c4)c3c2)cc1 ; CC1(C)c2ccccc2-c2ccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cc21]

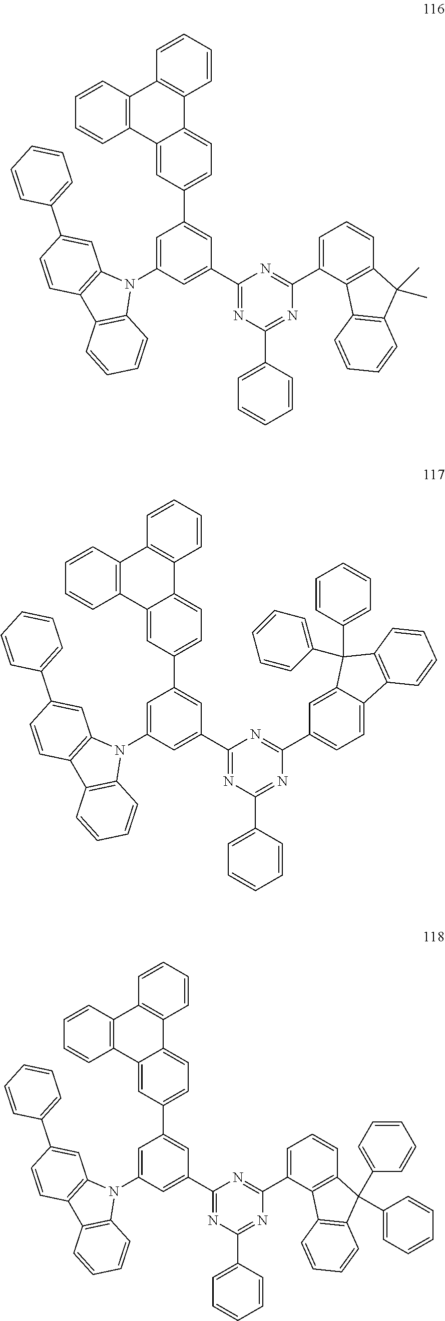 [SMILES: CC1(C)c2ccccc2-c2c(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cccc21 ; c1ccc(-c2ccc3c4ccccc4n(-c4cc(-c5ccc6c7ccccc7c7ccccc7c6c5)cc(-c5nc(-c6ccccc6)nc(-c6ccc7c(c6)C(c6ccccc6)(c6ccccc6)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6cccc7c6-c6ccccc6C7(c6ccccc6)c6ccccc6)n5)c4)c3c2)cc1]

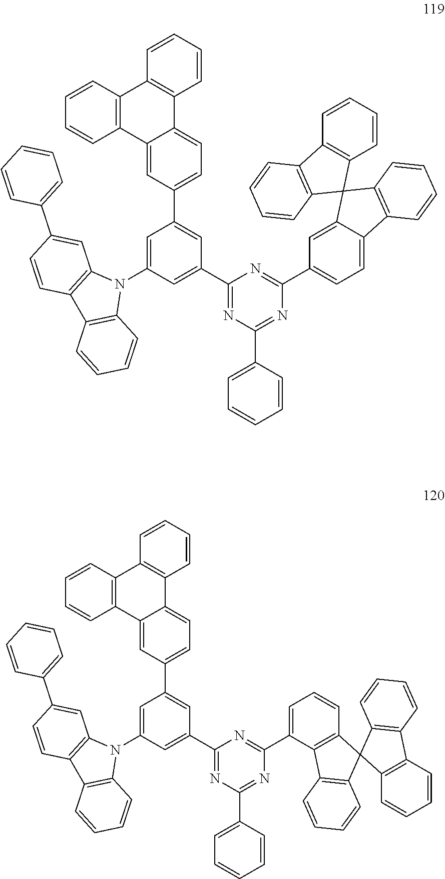 [SMILES: c1ccc(-c2ccc3c4ccccc4n(-c4cc(-c5ccc6c7ccccc7c7ccccc7c6c5)cc(-c5nc(-c6ccccc6)nc(-c6ccc7c(c6)C6(c8ccccc8-c8ccccc86)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6cccc7c6-c6ccccc6C76c7ccccc7-c7ccccc76)n5)c4)c3c2)cc1]

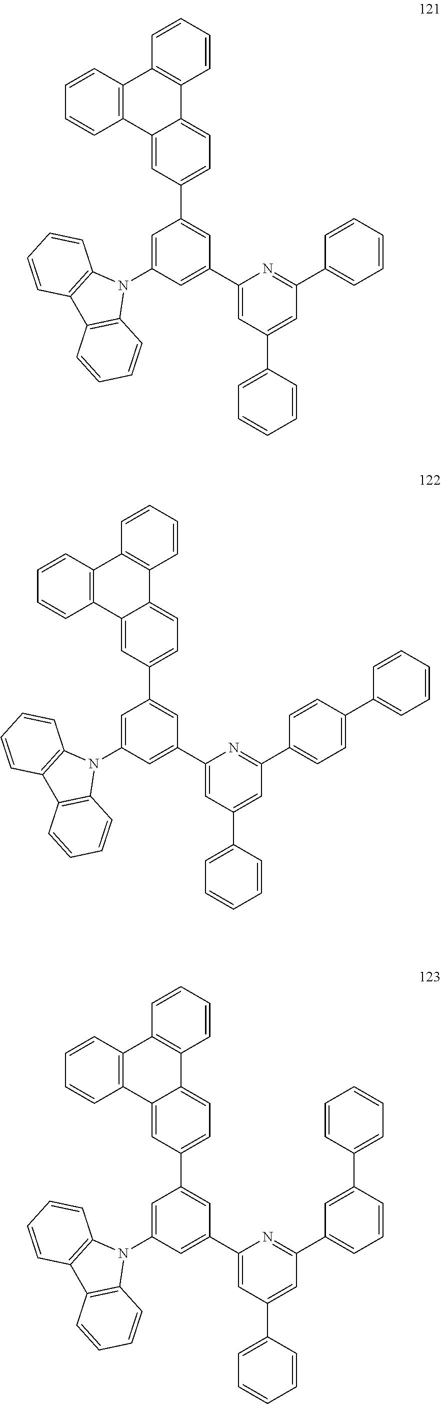 [SMILES: c1ccc(-c2cc(-c3ccccc3)nc(-c3cc(-c4ccc5c6ccccc6c6ccccc6c5c4)cc(-n4c5ccccc5c5ccccc54)c3)c2)cc1 ; c1ccc(-c2ccc(-c3cc(-c4ccccc4)cc(-c4cc(-c5ccc6c7ccccc7c7ccccc7c6c5)cc(-n5c6ccccc6c6ccccc65)c4)n3)cc2)cc1 ; c1ccc(-c2cccc(-c3cc(-c4ccccc4)cc(-c4cc(-c5ccc6c7ccccc7c7ccccc7c6c5)cc(-n5c6ccccc6c6ccccc65)c4)n3)c2)cc1]

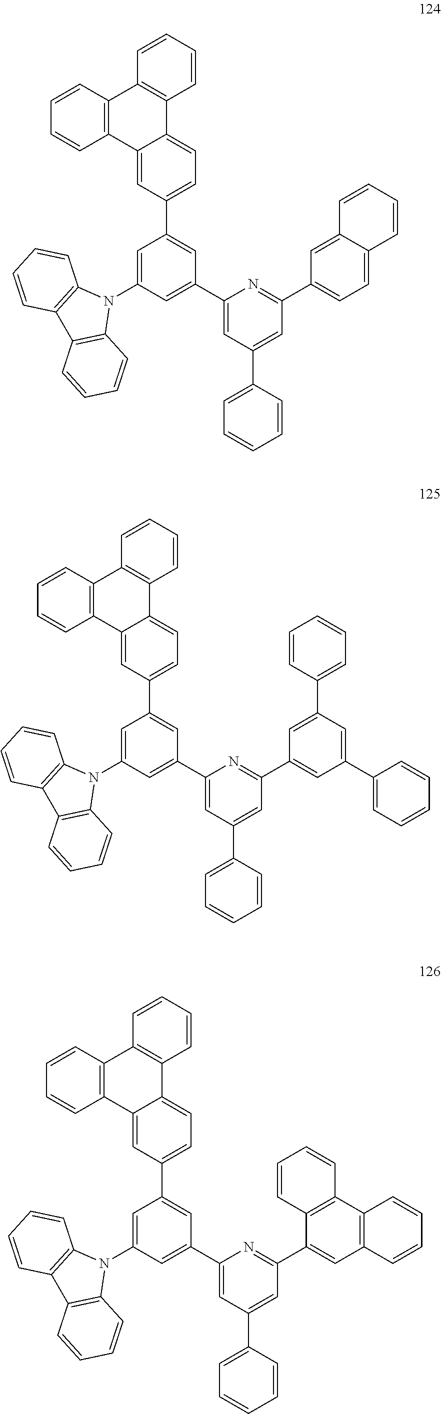 [SMILES: c1ccc(-c2cc(-c3cc(-c4ccc5c6ccccc6c6ccccc6c5c4)cc(-n4c5ccccc5c5ccccc54)c3)nc(-c3ccc4ccccc4c3)c2)cc1 ; c1ccc(-c2cc(-c3ccccc3)cc(-c3cc(-c4ccccc4)cc(-c4cc(-c5ccc6c7ccccc7c7ccccc7c6c5)cc(-n5c6ccccc6c6ccccc65)c4)n3)c2)cc1 ; c1ccc(-c2cc(-c3cc(-c4ccc5c6ccccc6c6ccccc6c5c4)cc(-n4c5ccccc5c5ccccc54)c3)nc(-c3cc4ccccc4c4ccccc34)c2)cc1]

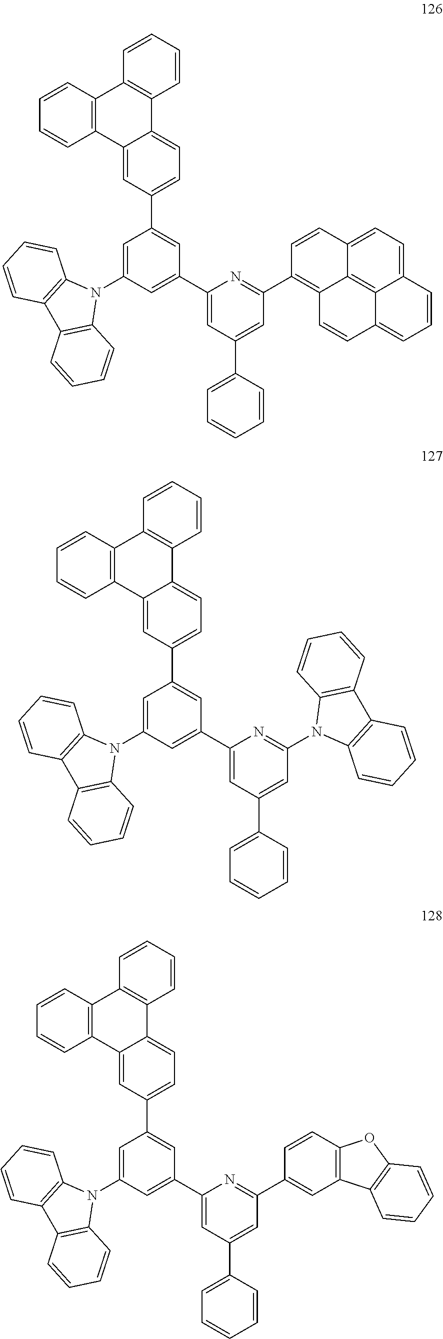 [SMILES: c1ccc(-c2cc(-c3cc(-c4ccc5c6ccccc6c6ccccc6c5c4)cc(-n4c5ccccc5c5ccccc54)c3)nc(-c3ccc4ccc5cccc6ccc3c4c56)c2)cc1 ; c1ccc(-c2cc(-c3cc(-c4ccc5c6ccccc6c6ccccc6c5c4)cc(-n4c5ccccc5c5ccccc54)c3)nc(-n3c4ccccc4c4ccccc43)c2)cc1 ; c1ccc(-c2cc(-c3cc(-c4ccc5c6ccccc6c6ccccc6c5c4)cc(-n4c5ccccc5c5ccccc54)c3)nc(-c3ccc4oc5ccccc5c4c3)c2)cc1]

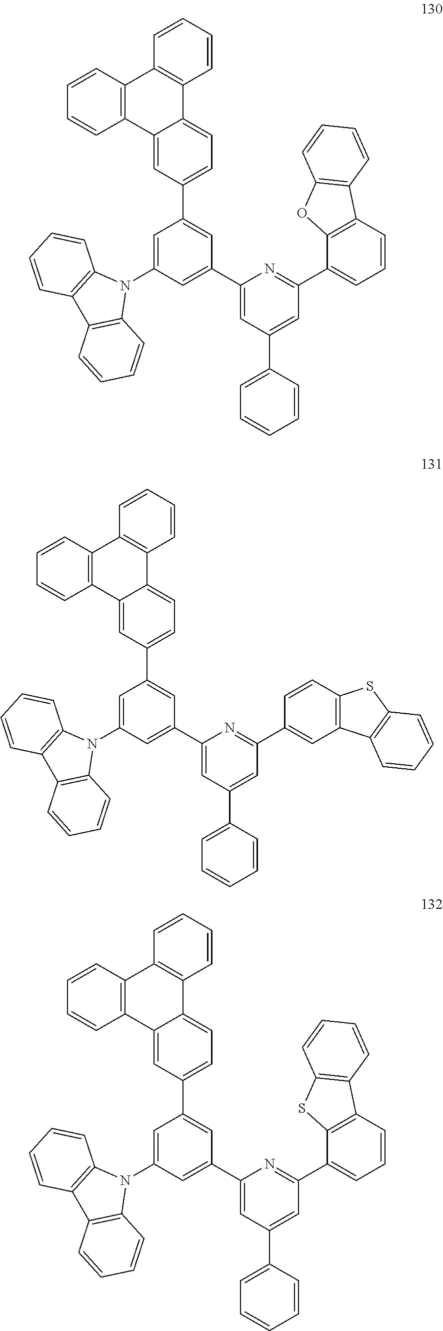 [SMILES: c1ccc(-c2cc(-c3cc(-c4ccc5c6ccccc6c6ccccc6c5c4)cc(-n4c5ccccc5c5ccccc54)c3)nc(-c3cccc4c3oc3ccccc34)c2)cc1 ; c1ccc(-c2cc(-c3cc(-c4ccc5c6ccccc6c6ccccc6c5c4)cc(-n4c5ccccc5c5ccccc54)c3)nc(-c3ccc4sc5ccccc5c4c3)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3sc3ccccc34)c2)cc1]

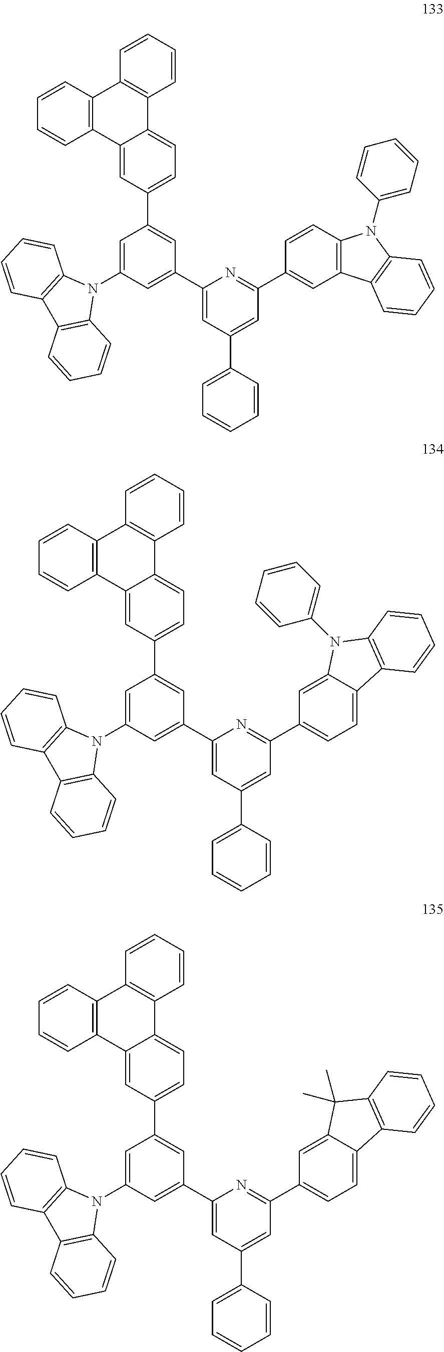 [SMILES: c1ccc(-c2cc(-c3cc(-c4ccc5c6ccccc6c6ccccc6c5c4)cc(-n4c5ccccc5c5ccccc54)c3)nc(-c3ccc4c(c3)c3ccccc3n4-c3ccccc3)c2)cc1 ; c1ccc(-c2cc(-c3cc(-c4ccc5c6ccccc6c6ccccc6c5c4)cc(-n4c5ccccc5c5ccccc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5ccc6c7ccccc7c7ccccc7c6c5)cc(-n5c6ccccc6c6ccccc65)c4)n3)cc21]

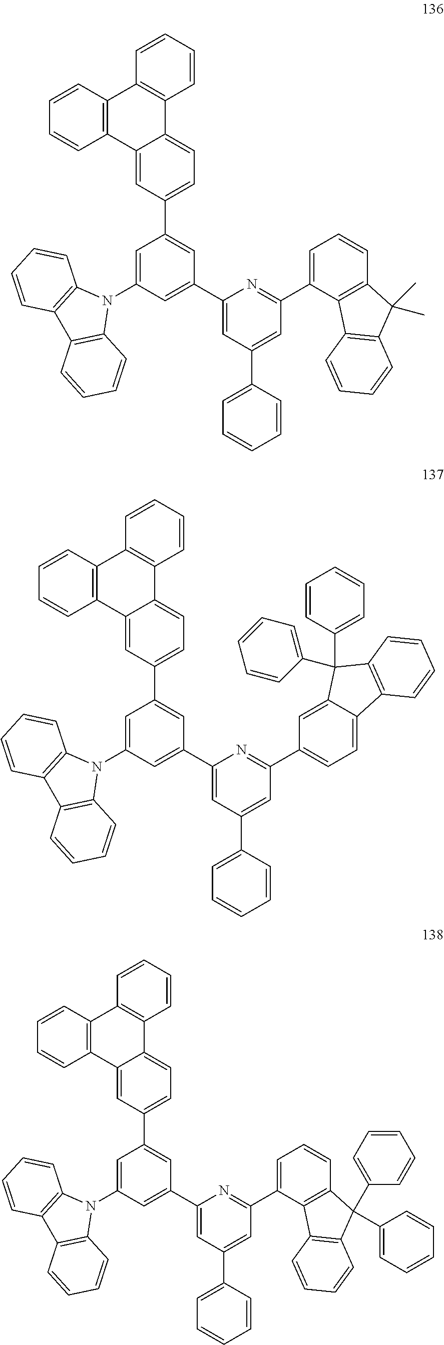 [SMILES: CC1(C)c2ccccc2-c2c(-c3cc(-c4ccccc4)cc(-c4cc(-c5ccc6c7ccccc7c7ccccc7c6c5)cc(-n5c6ccccc6c6ccccc65)c4)n3)cccc21 ; c1ccc(-c2cc(-c3cc(-c4ccc5c6ccccc6c6ccccc6c5c4)cc(-n4c5ccccc5c5ccccc54)c3)nc(-c3ccc4c(c3)C(c3ccccc3)(c3ccccc3)c3ccccc3-4)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3-c3ccccc3C4(c3ccccc3)c3ccccc3)c2)cc1]

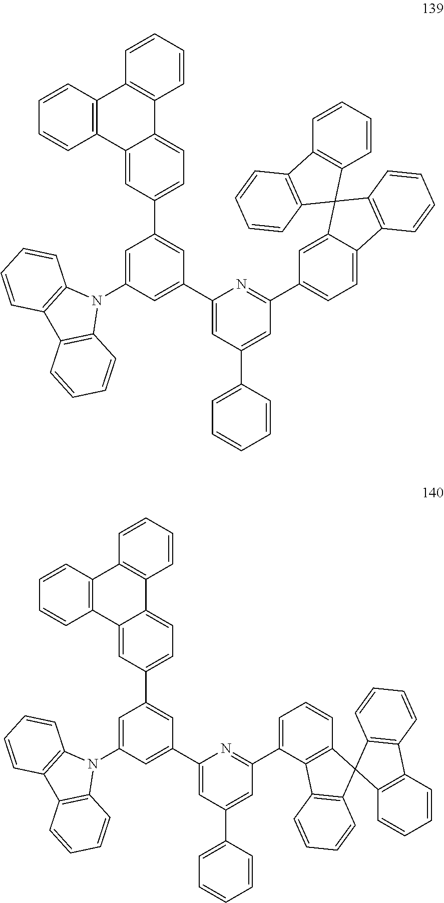 [SMILES: c1ccc(-c2cc(-c3cc(-c4ccc5c6ccccc6c6ccccc6c5c4)cc(-n4c5ccccc5c5ccccc54)c3)nc(-c3ccc4c(c3)C3(c5ccccc5-c5ccccc53)c3ccccc3-4)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3-c3ccccc3C43c4ccccc4-c4ccccc43)c2)cc1]

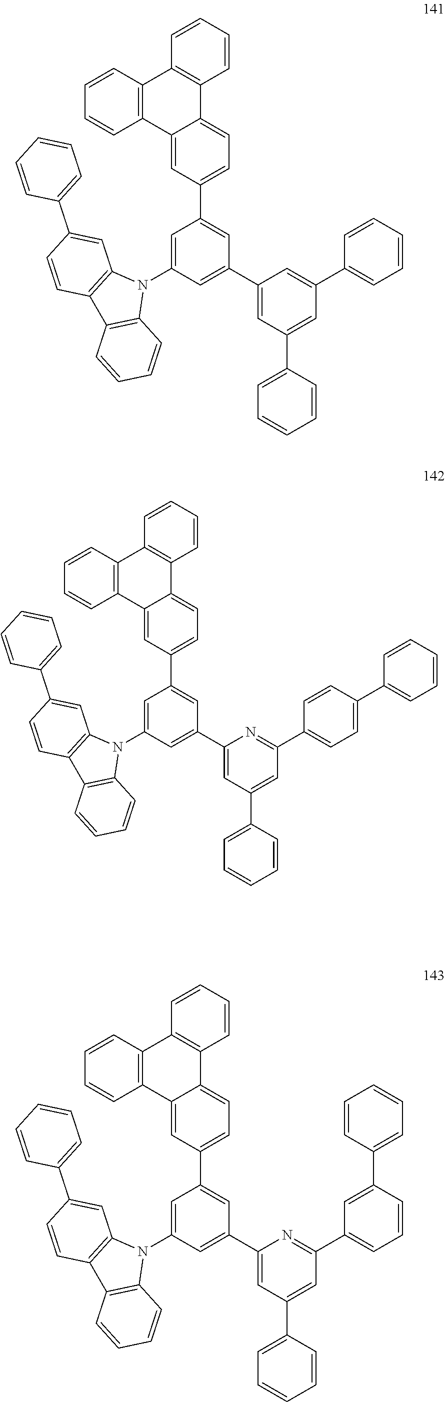 [SMILES: c1ccc(-c2cc(-c3ccccc3)cc(-c3cc(-c4ccc5c6ccccc6c6ccccc6c5c4)cc(-n4c5ccccc5c5ccc(-c6ccccc6)cc54)c3)c2)cc1 ; c1ccc(-c2ccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cc2)cc1 ; c1ccc(-c2cccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)c2)cc1]

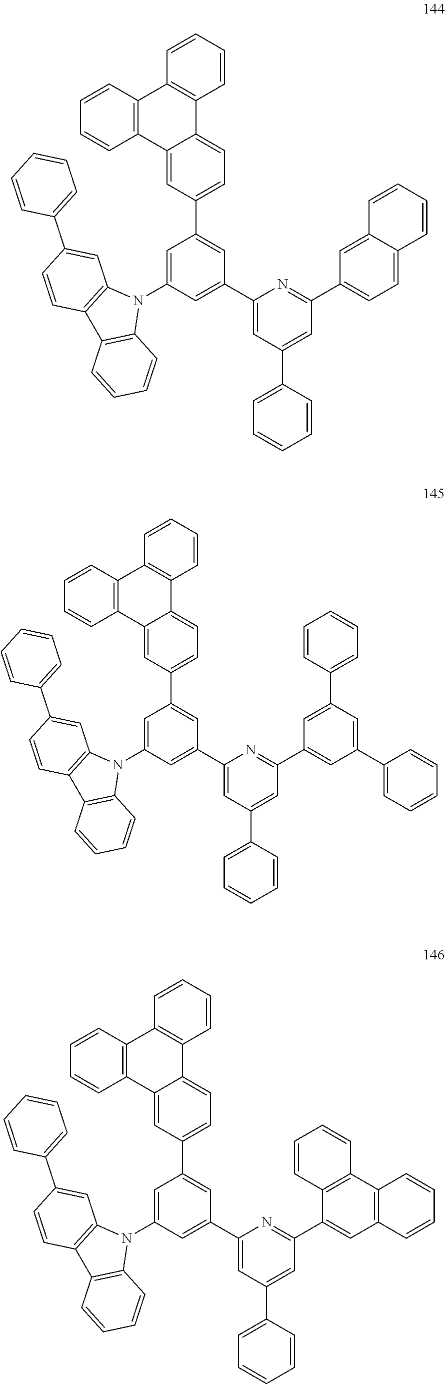 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4ccccc4c3)c2)cc1 ; c1ccc(-c2cc(-c3ccccc3)cc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cc4ccccc4c4ccccc34)c2)cc1]

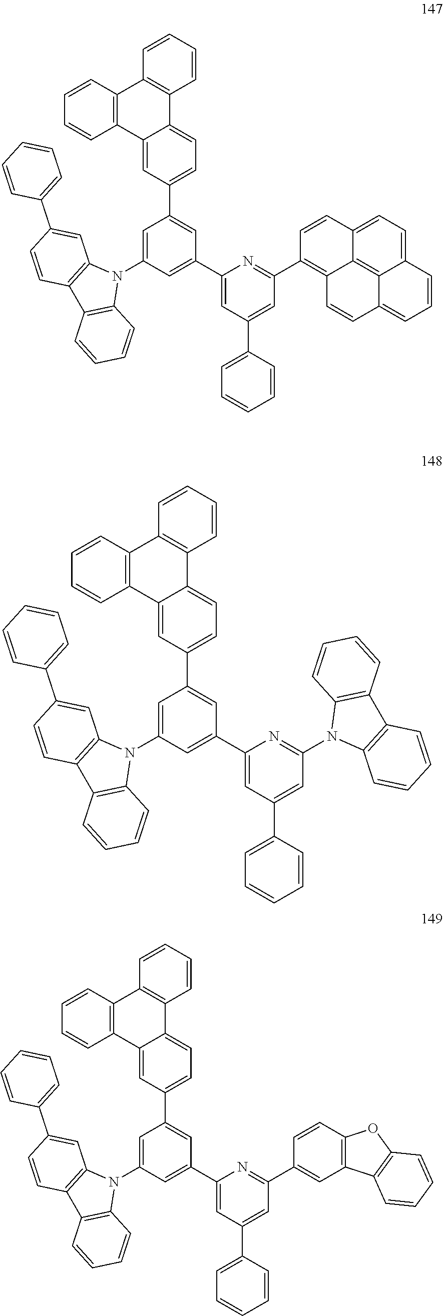 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4ccc5cccc6ccc3c4c56)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-n3c4ccccc4c4ccccc43)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4oc5ccccc5c4c3)c2)cc1]

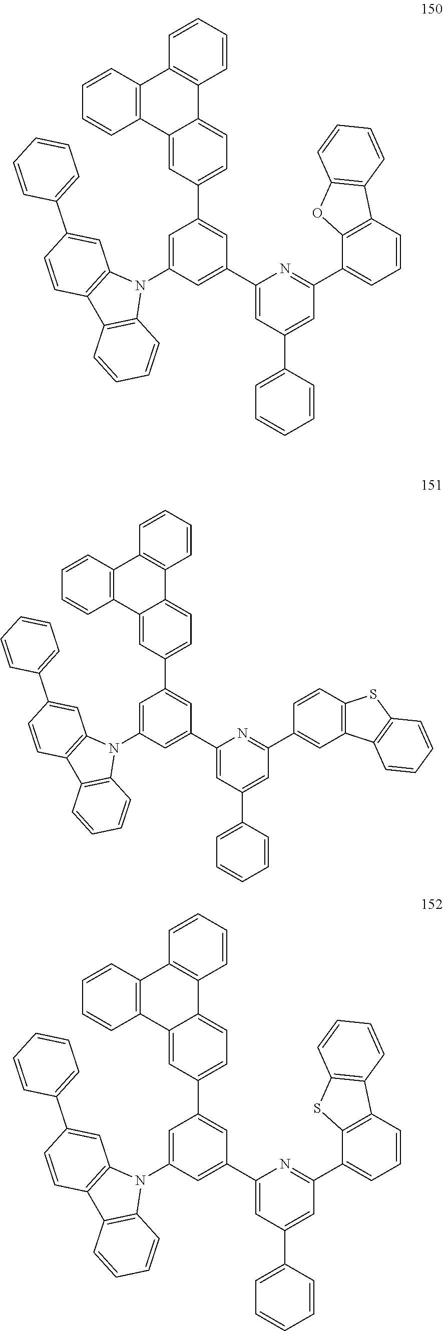 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3oc3ccccc34)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4sc5ccccc5c4c3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3sc3ccccc34)c2)cc1]

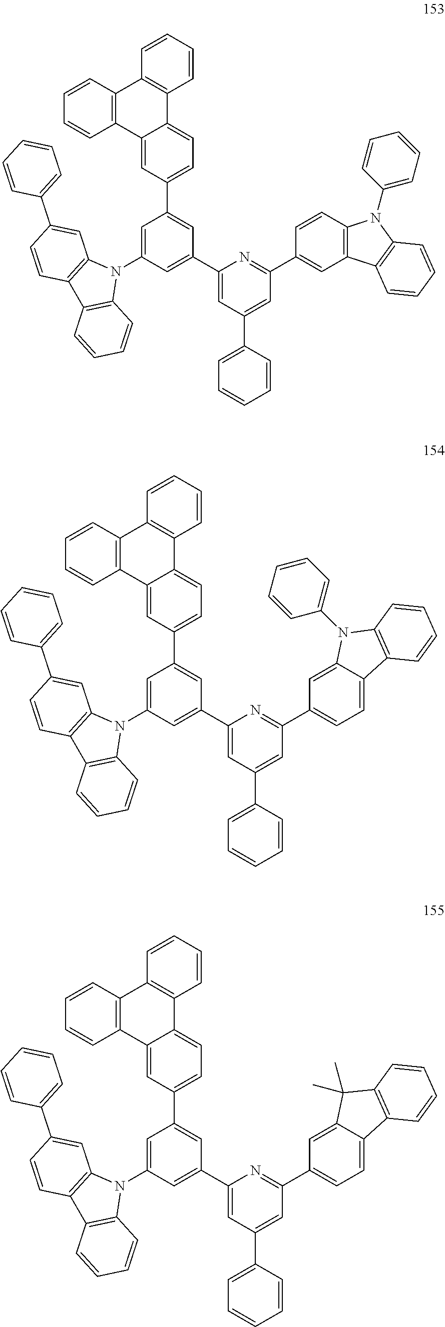 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)c3ccccc3n4-c3ccccc3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cc21]

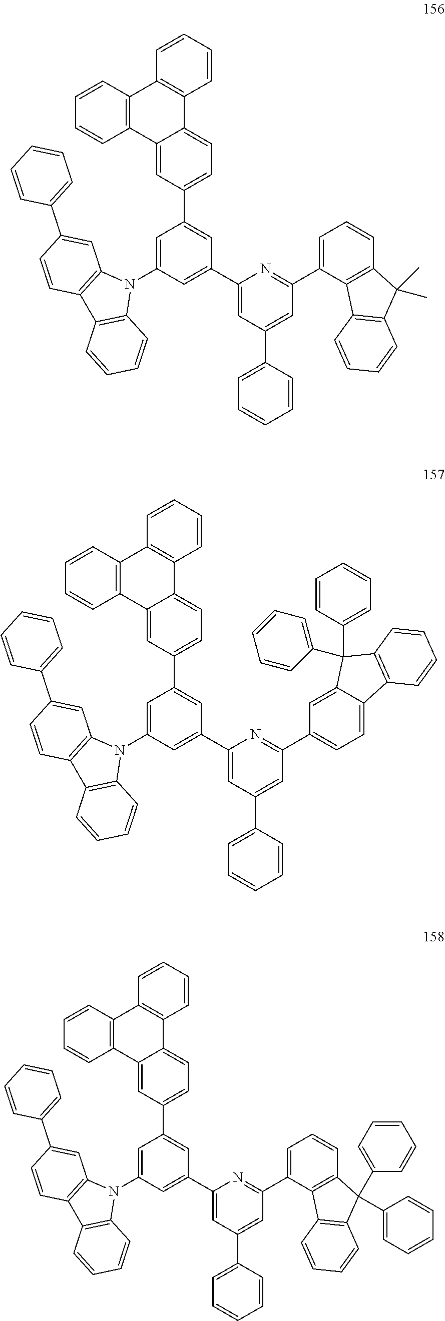 [SMILES: CC1(C)c2ccccc2-c2c(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cccc21 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)C(c3ccccc3)(c3ccccc3)c3ccccc3-4)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3-c3ccccc3C4(c3ccccc3)c3ccccc3)c2)cc1]

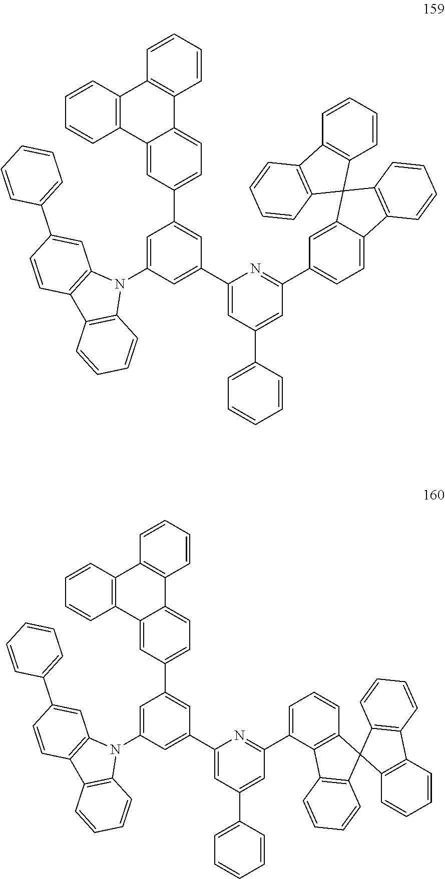 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)C3(c5ccccc5-c5ccccc53)c3ccccc3-4)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3-c3ccccc3C43c4ccccc4-c4ccccc43)c2)cc1]

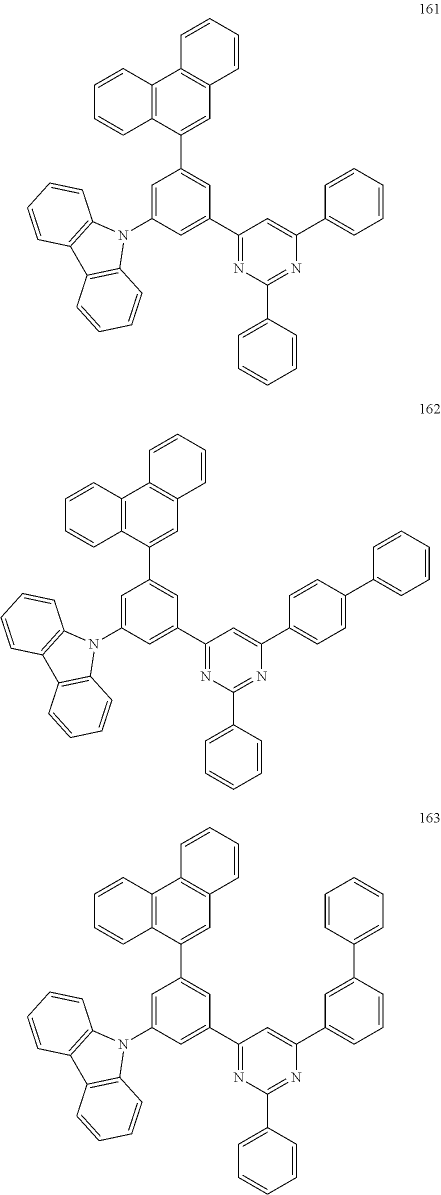

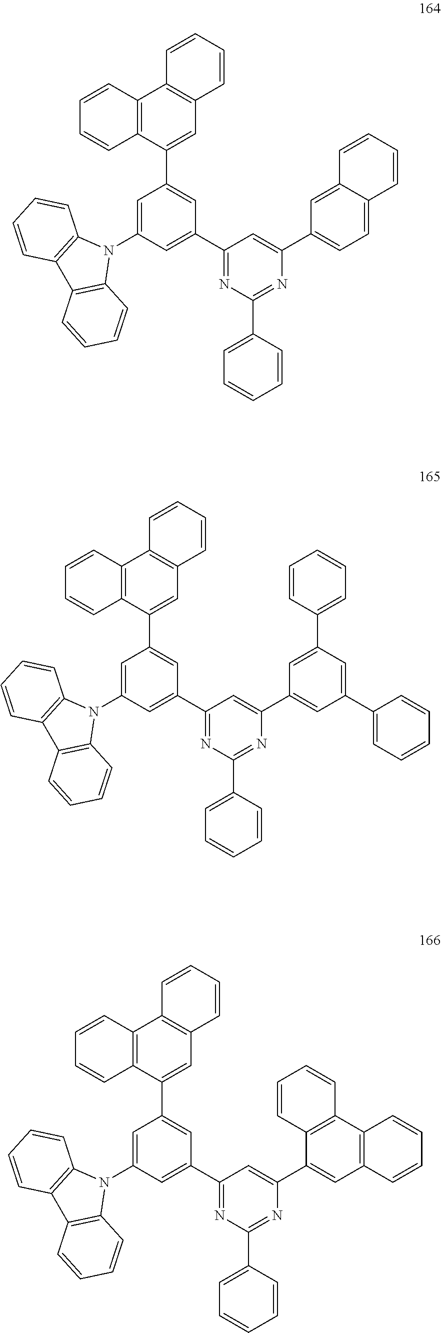 [SMILES: c1ccc(-c2nc(-c3cc(-c4cc5ccccc5c5ccccc45)cc(-n4c5ccccc5c5ccccc54)c3)cc(-c3ccc4ccccc4c3)n2)cc1 ; c1ccc(-c2cc(-c3ccccc3)cc(-c3cc(-c4cc(-c5cc6ccccc6c6ccccc56)cc(-n5c6ccccc6c6ccccc65)c4)nc(-c4ccccc4)n3)c2)cc1 ; c1ccc(-c2nc(-c3cc(-c4cc5ccccc5c5ccccc45)cc(-n4c5ccccc5c5ccccc54)c3)cc(-c3cc4ccccc4c4ccccc34)n2)cc1]

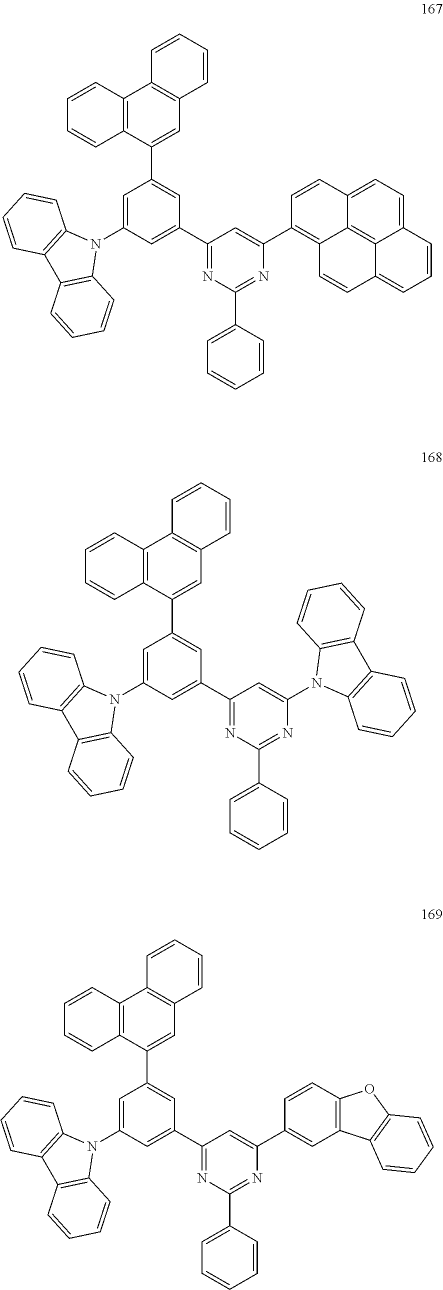 [SMILES: c1ccc(-c2nc(-c3cc(-c4cc5ccccc5c5ccccc45)cc(-n4c5ccccc5c5ccccc54)c3)cc(-c3ccc4ccc5cccc6ccc3c4c56)n2)cc1 ; c1ccc(-c2nc(-c3cc(-c4cc5ccccc5c5ccccc45)cc(-n4c5ccccc5c5ccccc54)c3)cc(-n3c4ccccc4c4ccccc43)n2)cc1 ; c1ccc(-c2nc(-c3cc(-c4cc5ccccc5c5ccccc45)cc(-n4c5ccccc5c5ccccc54)c3)cc(-c3ccc4oc5ccccc5c4c3)n2)cc1]

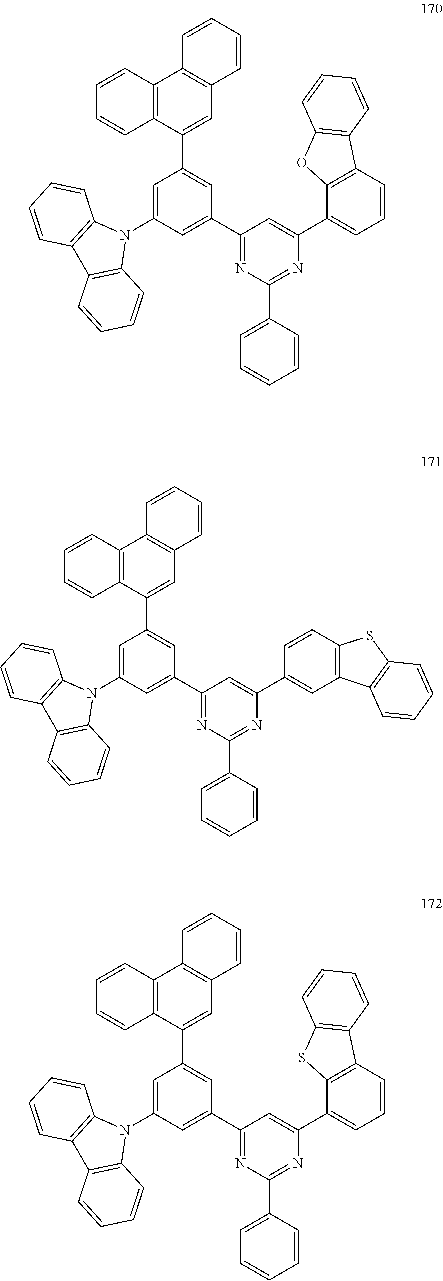 [SMILES: c1ccc(-c2nc(-c3cc(-c4cc5ccccc5c5ccccc45)cc(-n4c5ccccc5c5ccccc54)c3)cc(-c3cccc4c3oc3ccccc34)n2)cc1 ; c1ccc(-c2nc(-c3cc(-c4cc5ccccc5c5ccccc45)cc(-n4c5ccccc5c5ccccc54)c3)cc(-c3ccc4sc5ccccc5c4c3)n2)cc1 ; c1ccc(-c2nc(-c3cc(-c4cc5ccccc5c5ccccc45)cc(-n4c5ccccc5c5ccccc54)c3)cc(-c3cccc4c3sc3ccccc34)n2)cc1]

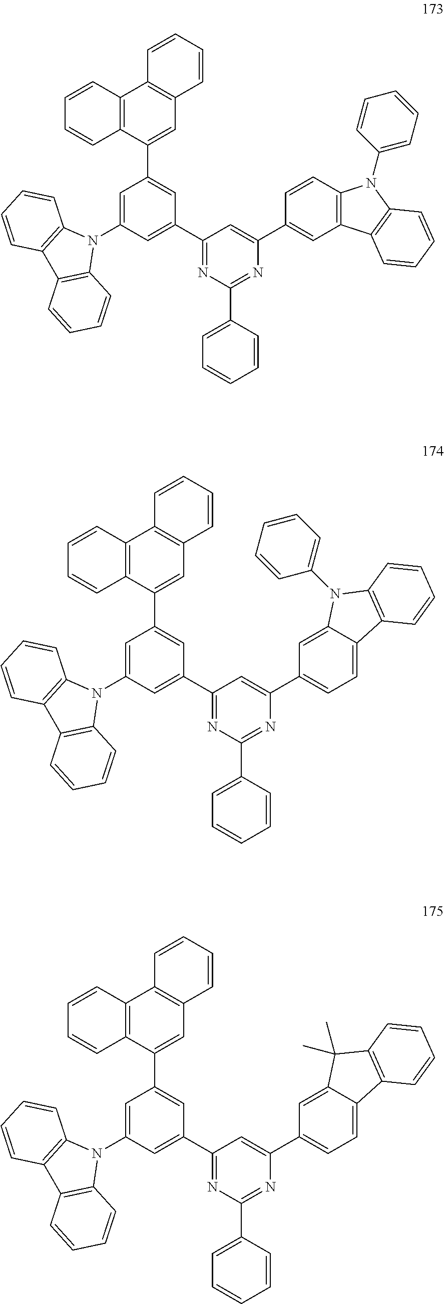 [SMILES: c1ccc(-c2nc(-c3cc(-c4cc5ccccc5c5ccccc45)cc(-n4c5ccccc5c5ccccc54)c3)cc(-c3ccc4c(c3)c3ccccc3n4-c3ccccc3)n2)cc1 ; c1ccc(-c2nc(-c3cc(-c4cc5ccccc5c5ccccc45)cc(-n4c5ccccc5c5ccccc54)c3)cc(-c3ccc4c5ccccc5n(-c5ccccc5)c4c3)n2)cc1 ; CC1(C)c2ccccc2-c2ccc(-c3cc(-c4cc(-c5cc6ccccc6c6ccccc56)cc(-n5c6ccccc6c6ccccc65)c4)nc(-c4ccccc4)n3)cc21]

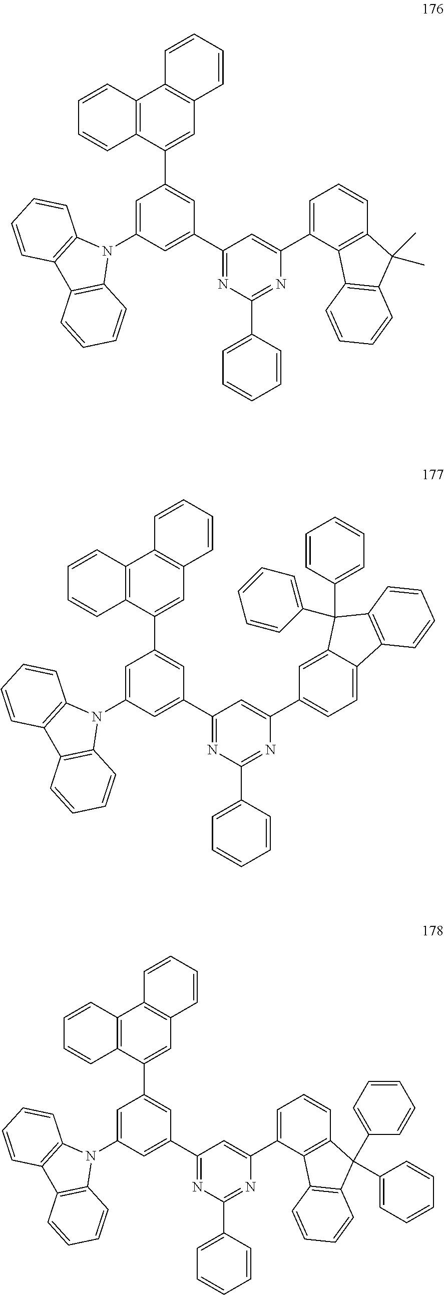 [SMILES: CC1(C)c2ccccc2-c2c(-c3cc(-c4cc(-c5cc6ccccc6c6ccccc56)cc(-n5c6ccccc6c6ccccc65)c4)nc(-c4ccccc4)n3)cccc21 ; c1ccc(-c2nc(-c3cc(-c4cc5ccccc5c5ccccc45)cc(-n4c5ccccc5c5ccccc54)c3)cc(-c3ccc4c(c3)C(c3ccccc3)(c3ccccc3)c3ccccc3-4)n2)cc1 ; c1ccc(-c2nc(-c3cc(-c4cc5ccccc5c5ccccc45)cc(-n4c5ccccc5c5ccccc54)c3)cc(-c3cccc4c3-c3ccccc3C4(c3ccccc3)c3ccccc3)n2)cc1]

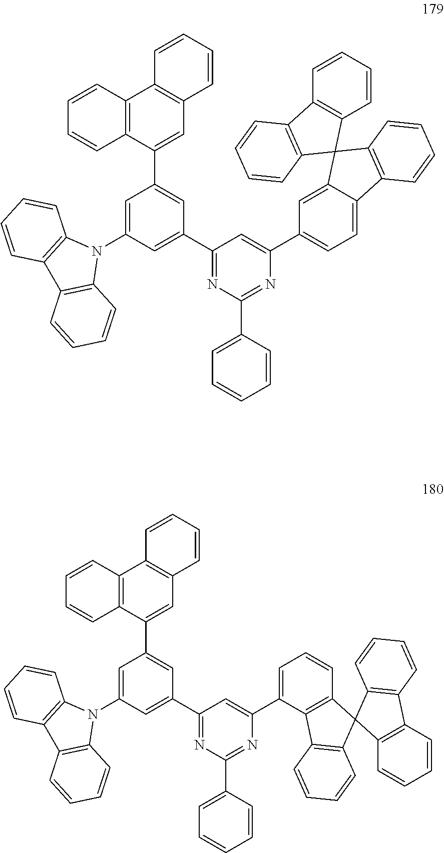 [SMILES: c1ccc(-c2nc(-c3cc(-c4cc5ccccc5c5ccccc45)cc(-n4c5ccccc5c5ccccc54)c3)cc(-c3ccc4c(c3)C3(c5ccccc5-c5ccccc53)c3ccccc3-4)n2)cc1 ; c1ccc(-c2nc(-c3cc(-c4cc5ccccc5c5ccccc45)cc(-n4c5ccccc5c5ccccc54)c3)cc(-c3cccc4c3-c3ccccc3C43c4ccccc4-c4ccccc43)n2)cc1]

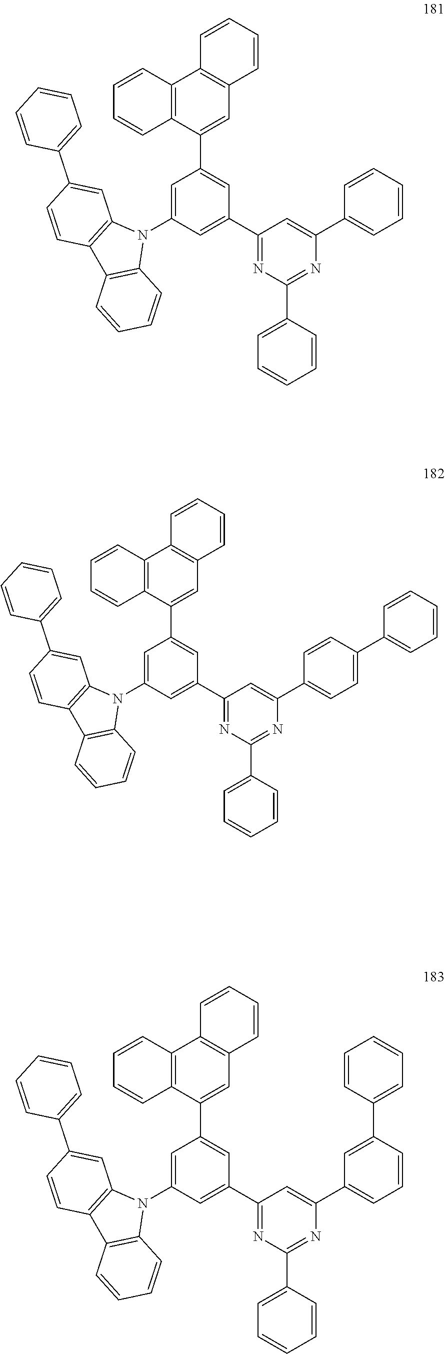 [SMILES: c1ccc(-c2ccc3c4ccccc4n(-c4cc(-c5cc(-c6ccccc6)nc(-c6ccccc6)n5)cc(-c5cc6ccccc6c6ccccc56)c4)c3c2)cc1 ; c1ccc(-c2ccc(-c3cc(-c4cc(-c5cc6ccccc6c6ccccc56)cc(-n5c6ccccc6c6ccc(-c7ccccc7)cc65)c4)nc(-c4ccccc4)n3)cc2)cc1 ; c1ccc(-c2cccc(-c3cc(-c4cc(-c5cc6ccccc6c6ccccc56)cc(-n5c6ccccc6c6ccc(-c7ccccc7)cc65)c4)nc(-c4ccccc4)n3)c2)cc1]

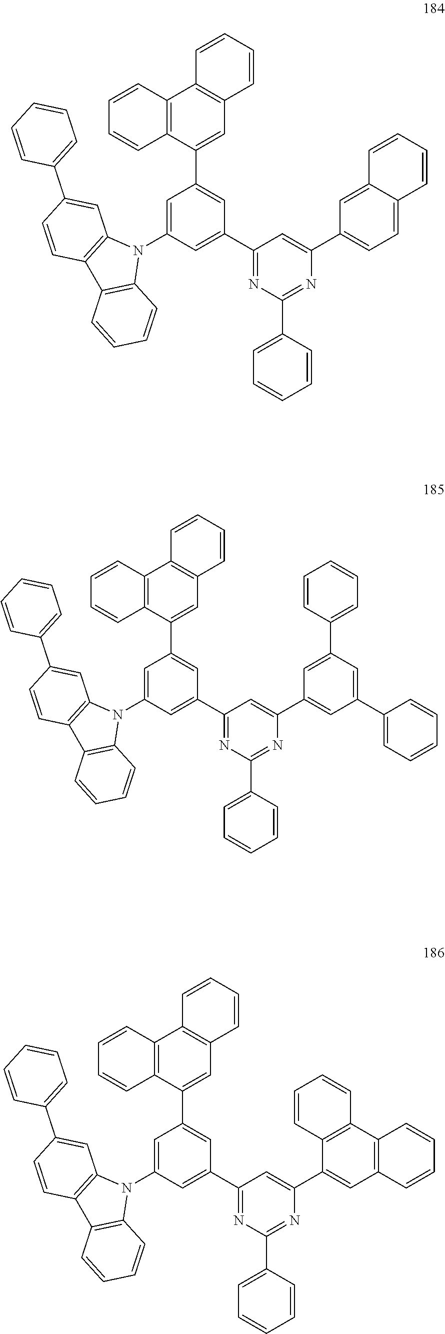 [SMILES: c1ccc(-c2ccc3c4ccccc4n(-c4cc(-c5cc(-c6ccc7ccccc7c6)nc(-c6ccccc6)n5)cc(-c5cc6ccccc6c6ccccc56)c4)c3c2)cc1 ; c1ccc(-c2cc(-c3ccccc3)cc(-c3cc(-c4cc(-c5cc6ccccc6c6ccccc56)cc(-n5c6ccccc6c6ccc(-c7ccccc7)cc65)c4)nc(-c4ccccc4)n3)c2)cc1 ; c1ccc(-c2ccc3c4ccccc4n(-c4cc(-c5cc(-c6cc7ccccc7c7ccccc67)nc(-c6ccccc6)n5)cc(-c5cc6ccccc6c6ccccc56)c4)c3c2)cc1]

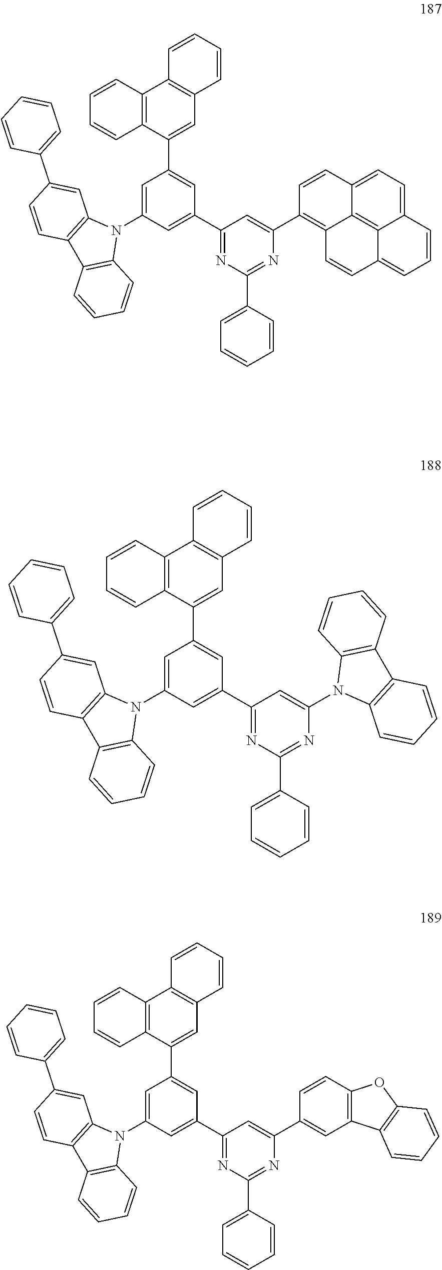 [SMILES: c1ccc(-c2ccc3c4ccccc4n(-c4cc(-c5cc(-c6ccc7ccc8cccc9ccc6c7c89)nc(-c6ccccc6)n5)cc(-c5cc6ccccc6c6ccccc56)c4)c3c2)cc1 ; c1ccc(-c2ccc3c4ccccc4n(-c4cc(-c5cc(-n6c7ccccc7c7ccccc76)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7oc8ccccc8c7c6)nc(-c6ccccc6)n5)cc(-c5cc6ccccc6c6ccccc56)c4)c3c2)cc1]

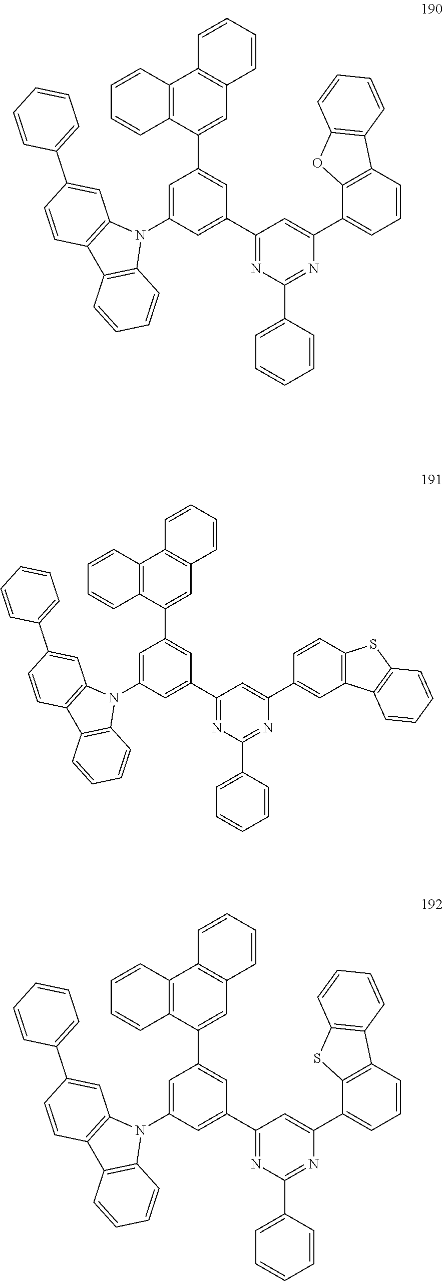 [SMILES: c1ccc(-c2ccc3c4ccccc4n(-c4cc(-c5cc(-c6cccc7c6oc6ccccc67)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7sc8ccccc8c7c6)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6sc6ccccc67)nc(-c6ccccc6)n5)cc(-c5cc6ccccc6c6ccccc56)c4)c3c2)cc1]

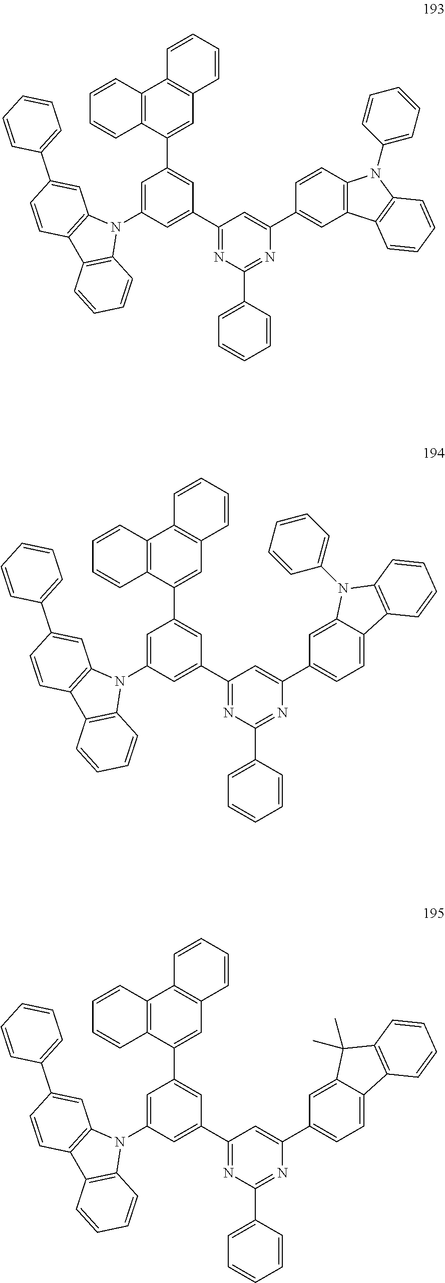 [SMILES: c1ccc(-c2ccc3c4ccccc4n(-c4cc(-c5cc(-c6ccc7c(c6)c6ccccc6n7-c6ccccc6)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7c8ccccc8n(-c8ccccc8)c7c6)nc(-c6ccccc6)n5)cc(-c5cc6ccccc6c6ccccc56)c4)c3c2)cc1 ; CC1(C)c2ccccc2-c2ccc(-c3cc(-c4cc(-c5cc6ccccc6c6ccccc56)cc(-n5c6ccccc6c6ccc(-c7ccccc7)cc65)c4)nc(-c4ccccc4)n3)cc21]

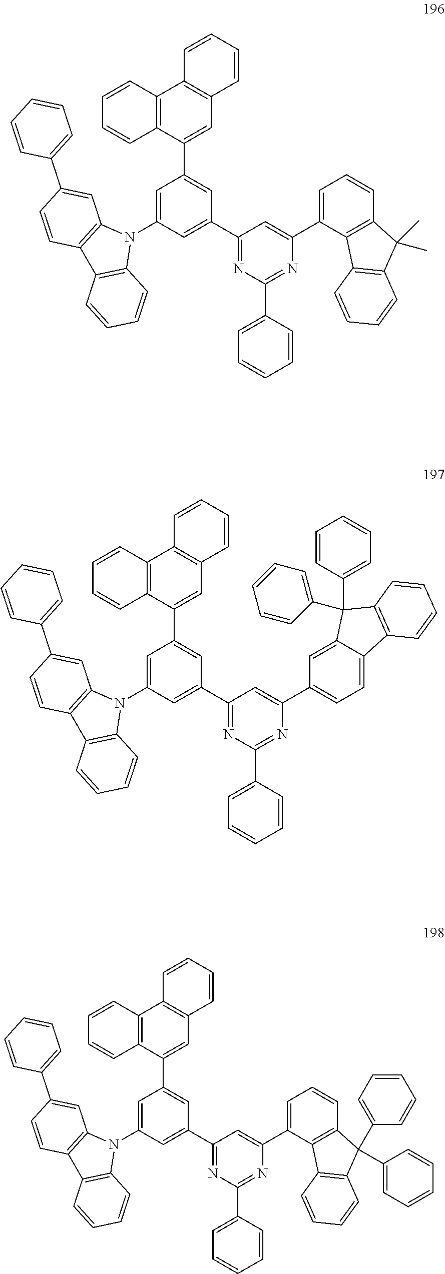 [SMILES: CC1(C)c2ccccc2-c2c(-c3cc(-c4cc(-c5cc6ccccc6c6ccccc56)cc(-n5c6ccccc6c6ccc(-c7ccccc7)cc65)c4)nc(-c4ccccc4)n3)cccc21 ; c1ccc(-c2ccc3c4ccccc4n(-c4cc(-c5cc(-c6ccc7c(c6)C(c6ccccc6)(c6ccccc6)c6ccccc6-7)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6-c6ccccc6C7(c6ccccc6)c6ccccc6)nc(-c6ccccc6)n5)cc(-c5cc6ccccc6c6ccccc56)c4)c3c2)cc1]

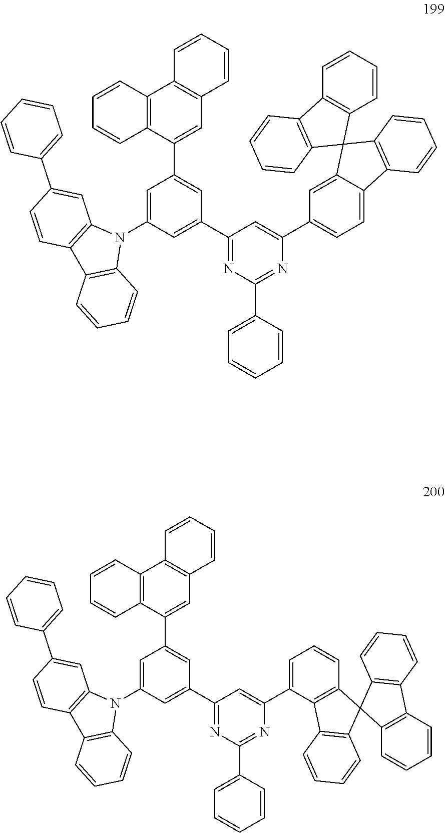 [SMILES: c1ccc(-c2ccc3c4ccccc4n(-c4cc(-c5cc(-c6ccc7c(c6)C6(c8ccccc8-c8ccccc86)c6ccccc6-7)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6-c6ccccc6C76c7ccccc7-c7ccccc76)nc(-c6ccccc6)n5)cc(-c5cc6ccccc6c6ccccc56)c4)c3c2)cc1]

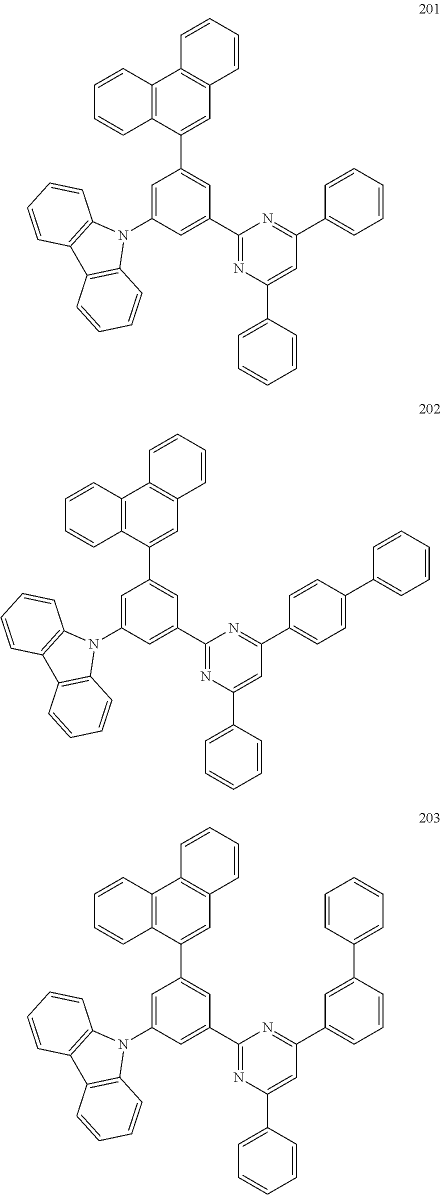

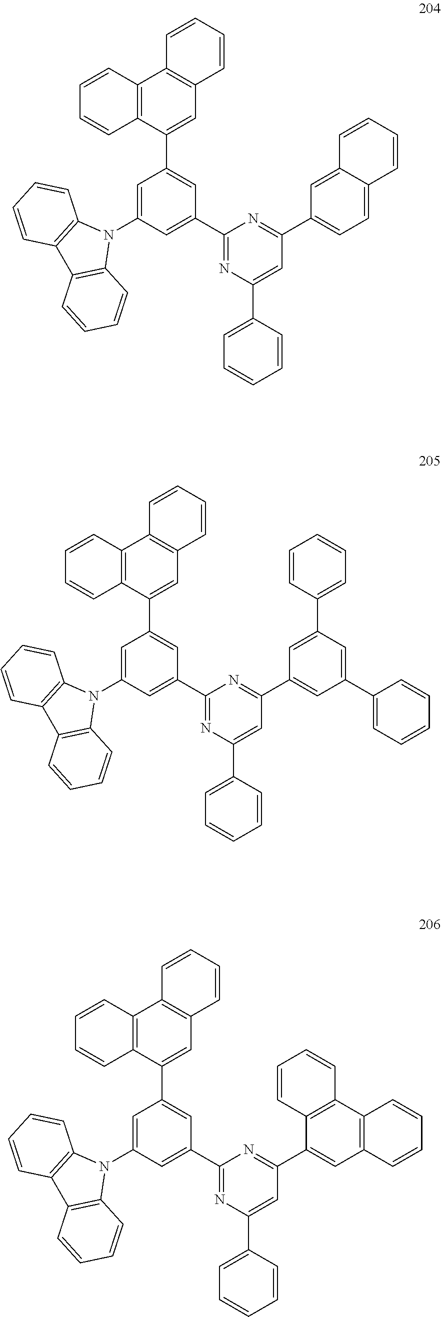 [SMILES: c1ccc(-c2cc(-c3ccc4ccccc4c3)nc(-c3cc(-c4cc5ccccc5c5ccccc45)cc(-n4c5ccccc5c5ccccc54)c3)n2)cc1 ; c1ccc(-c2cc(-c3ccccc3)cc(-c3cc(-c4ccccc4)nc(-c4cc(-c5cc6ccccc6c6ccccc56)cc(-n5c6ccccc6c6ccccc65)c4)n3)c2)cc1 ; c1ccc(-c2cc(-c3cc4ccccc4c4ccccc34)nc(-c3cc(-c4cc5ccccc5c5ccccc45)cc(-n4c5ccccc5c5ccccc54)c3)n2)cc1]

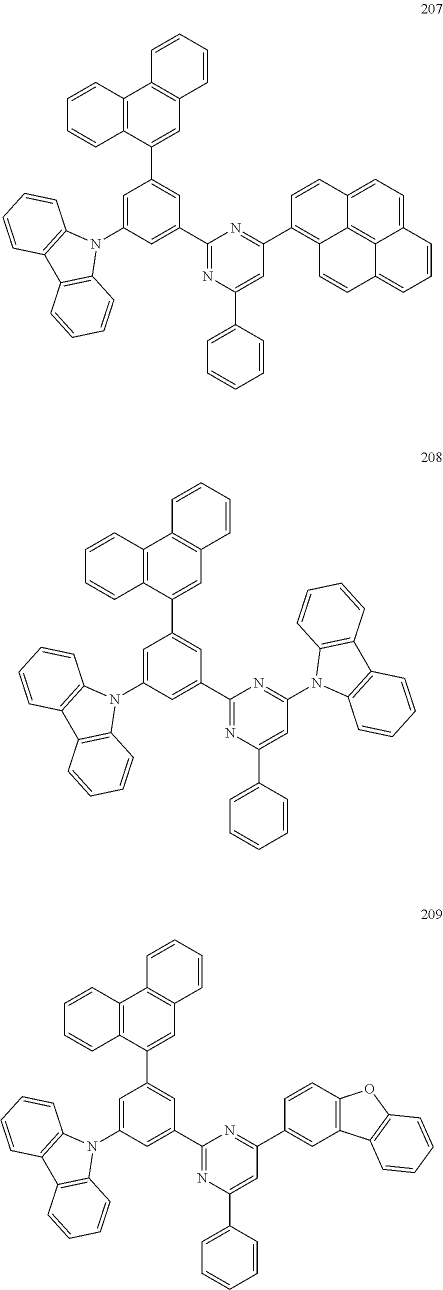 [SMILES: c1ccc(-c2cc(-c3ccc4ccc5cccc6ccc3c4c56)nc(-c3cc(-c4cc5ccccc5c5ccccc45)cc(-n4c5ccccc5c5ccccc54)c3)n2)cc1 ; c1ccc(-c2cc(-n3c4ccccc4c4ccccc43)nc(-c3cc(-c4cc5ccccc5c5ccccc45)cc(-n4c5ccccc5c5ccccc54)c3)n2)cc1 ; c1ccc(-c2cc(-c3ccc4oc5ccccc5c4c3)nc(-c3cc(-c4cc5ccccc5c5ccccc45)cc(-n4c5ccccc5c5ccccc54)c3)n2)cc1]

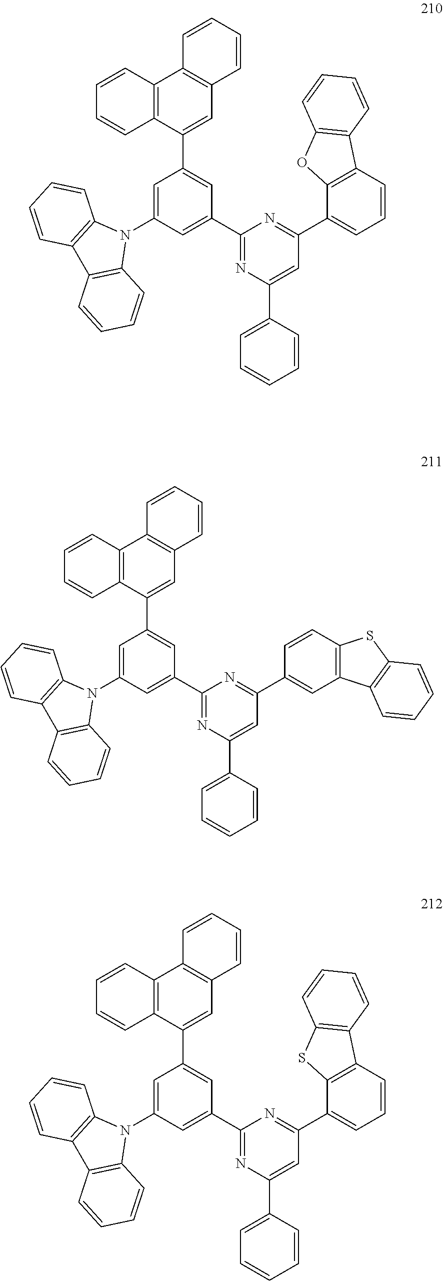 [SMILES: c1ccc(-c2cc(-c3cccc4c3oc3ccccc34)nc(-c3cc(-c4cc5ccccc5c5ccccc45)cc(-n4c5ccccc5c5ccccc54)c3)n2)cc1 ; c1ccc(-c2cc(-c3ccc4sc5ccccc5c4c3)nc(-c3cc(-c4cc5ccccc5c5ccccc45)cc(-n4c5ccccc5c5ccccc54)c3)n2)cc1 ; c1ccc(-c2cc(-c3cccc4c3sc3ccccc34)nc(-c3cc(-c4cc5ccccc5c5ccccc45)cc(-n4c5ccccc5c5ccccc54)c3)n2)cc1]

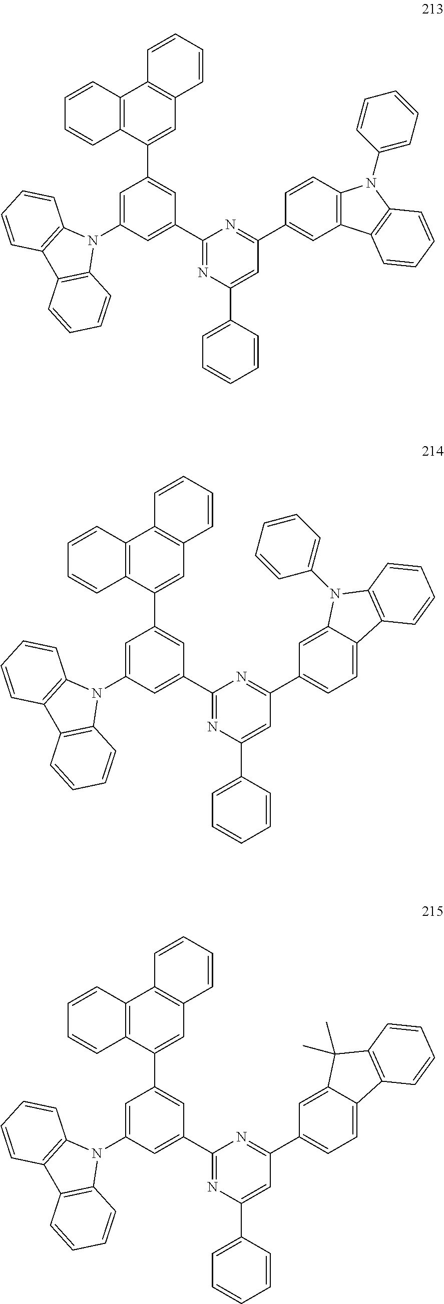 [SMILES: c1ccc(-c2cc(-c3ccc4c(c3)c3ccccc3n4-c3ccccc3)nc(-c3cc(-c4cc5ccccc5c5ccccc45)cc(-n4c5ccccc5c5ccccc54)c3)n2)cc1 ; c1ccc(-c2cc(-c3ccc4c5ccccc5n(-c5ccccc5)c4c3)nc(-c3cc(-c4cc5ccccc5c5ccccc45)cc(-n4c5ccccc5c5ccccc54)c3)n2)cc1 ; CC1(C)c2ccccc2-c2ccc(-c3cc(-c4ccccc4)nc(-c4cc(-c5cc6ccccc6c6ccccc56)cc(-n5c6ccccc6c6ccccc65)c4)n3)cc21]

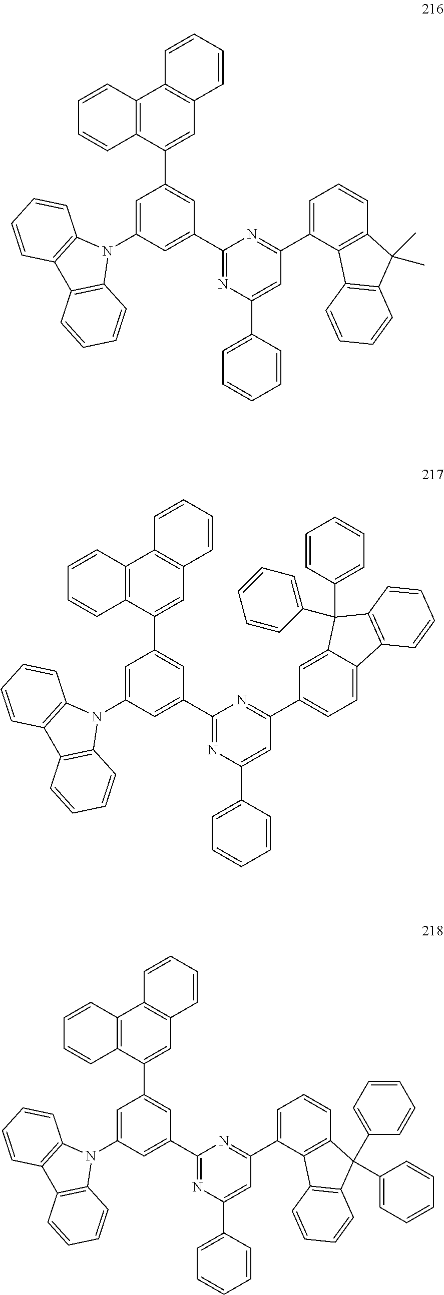 [SMILES: CC1(C)c2ccccc2-c2c(-c3cc(-c4ccccc4)nc(-c4cc(-c5cc6ccccc6c6ccccc56)cc(-n5c6ccccc6c6ccccc65)c4)n3)cccc21 ; c1ccc(-c2cc(-c3ccc4c(c3)C(c3ccccc3)(c3ccccc3)c3ccccc3-4)nc(-c3cc(-c4cc5ccccc5c5ccccc45)cc(-n4c5ccccc5c5ccccc54)c3)n2)cc1 ; c1ccc(-c2cc(-c3cccc4c3-c3ccccc3C4(c3ccccc3)c3ccccc3)nc(-c3cc(-c4cc5ccccc5c5ccccc45)cc(-n4c5ccccc5c5ccccc54)c3)n2)cc1]

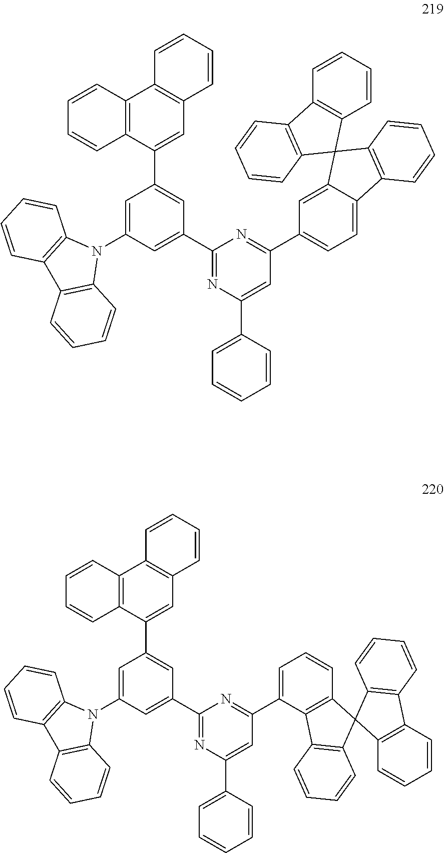 [SMILES: c1ccc(-c2cc(-c3ccc4c(c3)C3(c5ccccc5-c5ccccc53)c3ccccc3-4)nc(-c3cc(-c4cc5ccccc5c5ccccc45)cc(-n4c5ccccc5c5ccccc54)c3)n2)cc1 ; c1ccc(-c2cc(-c3cccc4c3-c3ccccc3C43c4ccccc4-c4ccccc43)nc(-c3cc(-c4cc5ccccc5c5ccccc45)cc(-n4c5ccccc5c5ccccc54)c3)n2)cc1]

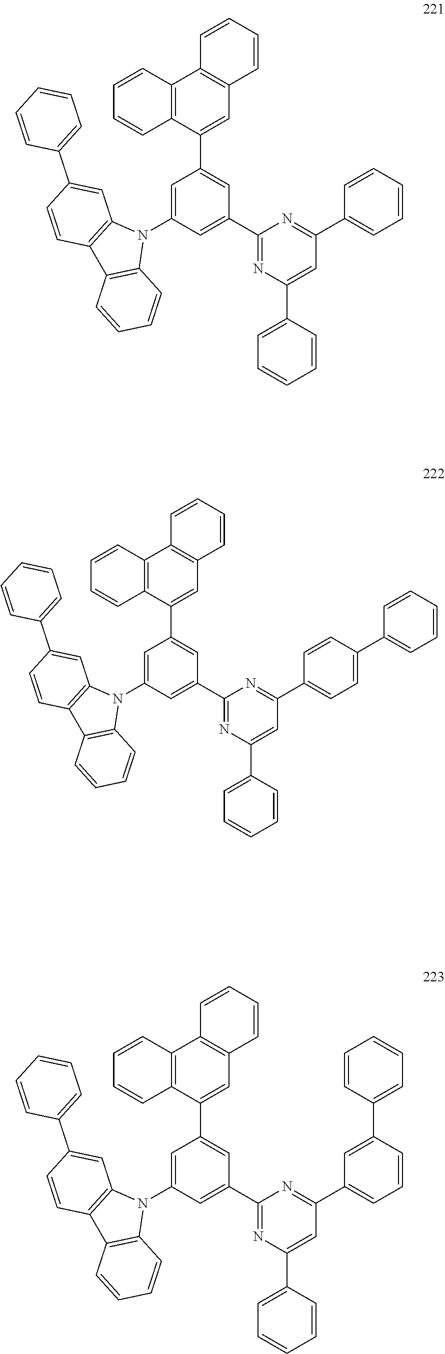 [SMILES: c1ccc(-c2ccc3c4ccccc4n(-c4cc(-c5nc(-c6ccccc6)cc(-c6ccccc6)n5)cc(-c5cc6ccccc6c6ccccc56)c4)c3c2)cc1 ; c1ccc(-c2ccc(-c3cc(-c4ccccc4)nc(-c4cc(-c5cc6ccccc6c6ccccc56)cc(-n5c6ccccc6c6ccc(-c7ccccc7)cc65)c4)n3)cc2)cc1 ; c1ccc(-c2cccc(-c3cc(-c4ccccc4)nc(-c4cc(-c5cc6ccccc6c6ccccc56)cc(-n5c6ccccc6c6ccc(-c7ccccc7)cc65)c4)n3)c2)cc1]

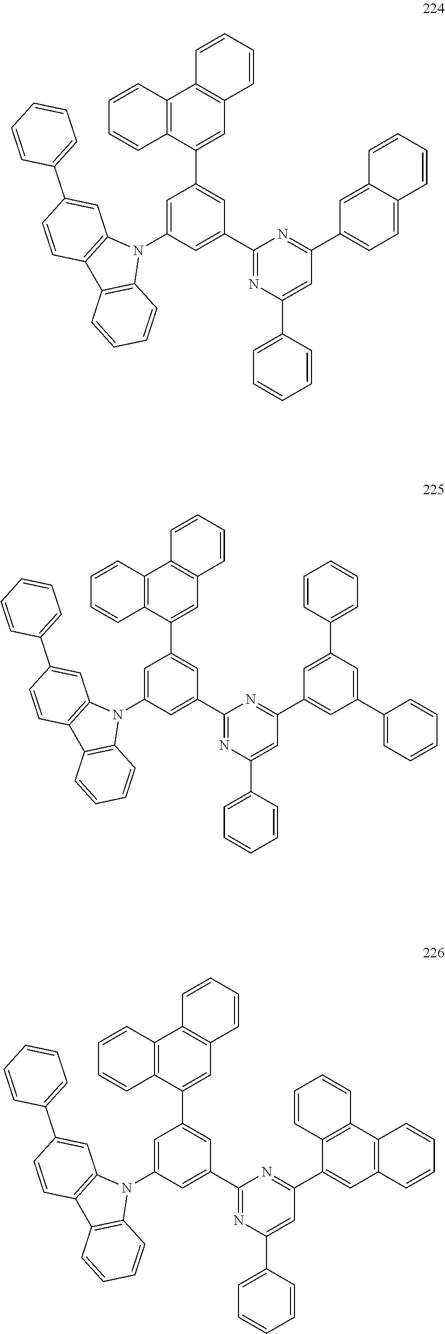 [SMILES: c1ccc(-c2ccc3c4ccccc4n(-c4cc(-c5nc(-c6ccccc6)cc(-c6ccc7ccccc7c6)n5)cc(-c5cc6ccccc6c6ccccc56)c4)c3c2)cc1 ; c1ccc(-c2cc(-c3ccccc3)cc(-c3cc(-c4ccccc4)nc(-c4cc(-c5cc6ccccc6c6ccccc56)cc(-n5c6ccccc6c6ccc(-c7ccccc7)cc65)c4)n3)c2)cc1 ; c1ccc(-c2ccc3c4ccccc4n(-c4cc(-c5nc(-c6ccccc6)cc(-c6cc7ccccc7c7ccccc67)n5)cc(-c5cc6ccccc6c6ccccc56)c4)c3c2)cc1]

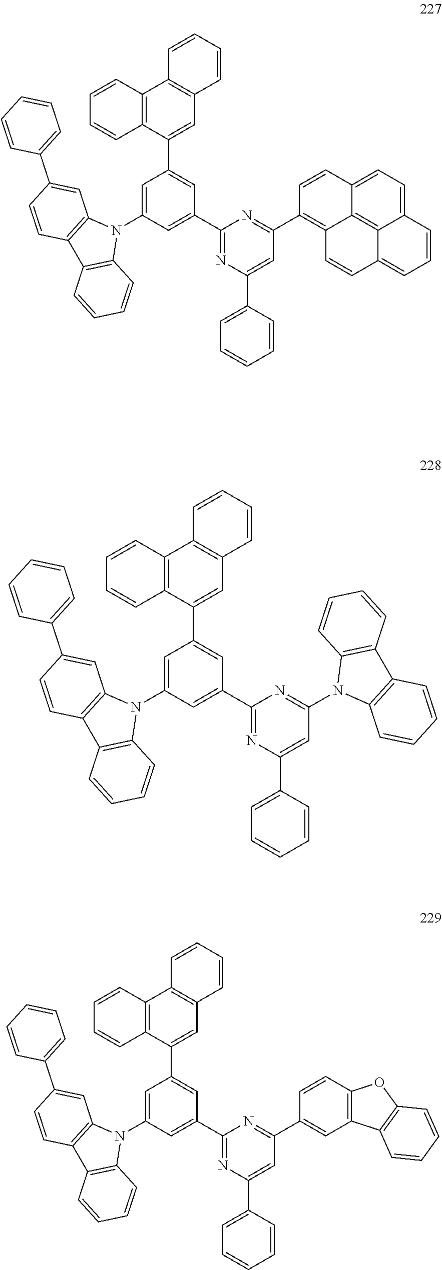 [SMILES: c1ccc(-c2ccc3c4ccccc4n(-c4cc(-c5nc(-c6ccccc6)cc(-c6ccc7ccc8cccc9ccc6c7c89)n5)cc(-c5cc6ccccc6c6ccccc56)c4)c3c2)cc1 ; c1ccc(-c2ccc3c4ccccc4n(-c4cc(-c5nc(-c6ccccc6)cc(-n6c7ccccc7c7ccccc76)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7oc8ccccc8c7c6)n5)cc(-c5cc6ccccc6c6ccccc56)c4)c3c2)cc1]

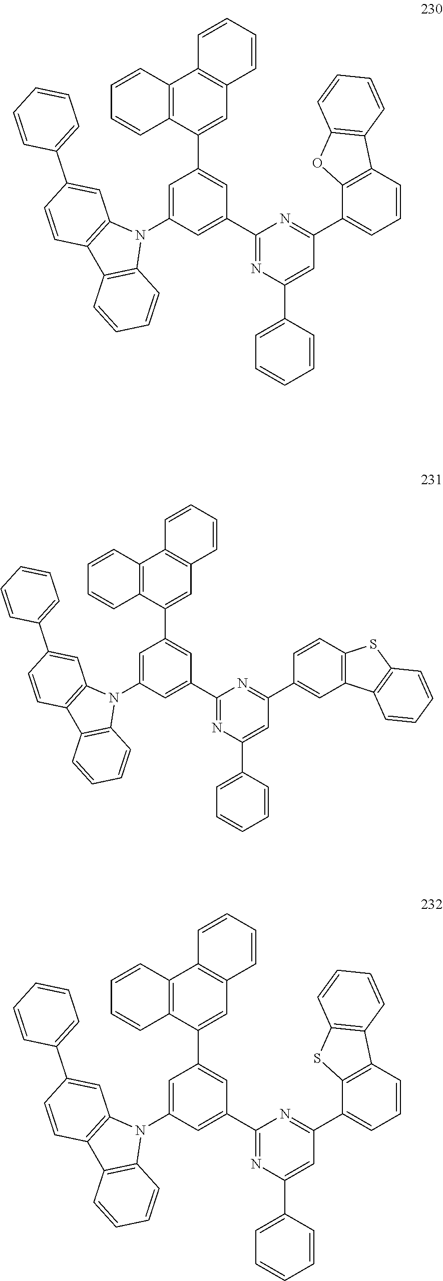 [SMILES: c1ccc(-c2ccc3c4ccccc4n(-c4cc(-c5nc(-c6ccccc6)cc(-c6cccc7c6oc6ccccc67)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7sc8ccccc8c7c6)n5)cc(-c5cc6ccccc6c6ccccc56)c4)c3c2)cc1 ; c1ccc(-c2ccc3c4ccccc4n(-c4cc(-c5nc(-c6ccccc6)cc(-c6cccc7c6sc6ccccc67)n5)cc(-c5cc6ccccc6c6ccccc56)c4)c3c2)cc1]

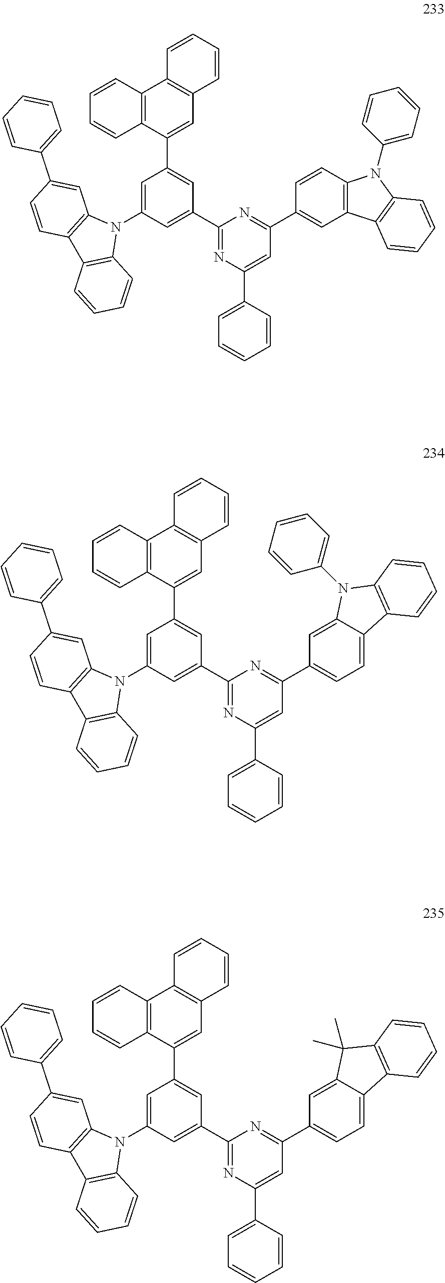 [SMILES: c1ccc(-c2ccc3c4ccccc4n(-c4cc(-c5nc(-c6ccccc6)cc(-c6ccc7c(c6)c6ccccc6n7-c6ccccc6)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7c8ccccc8n(-c8ccccc8)c7c6)n5)cc(-c5cc6ccccc6c6ccccc56)c4)c3c2)cc1 ; CC1(C)c2ccccc2-c2ccc(-c3cc(-c4ccccc4)nc(-c4cc(-c5cc6ccccc6c6ccccc56)cc(-n5c6ccccc6c6ccc(-c7ccccc7)cc65)c4)n3)cc21]

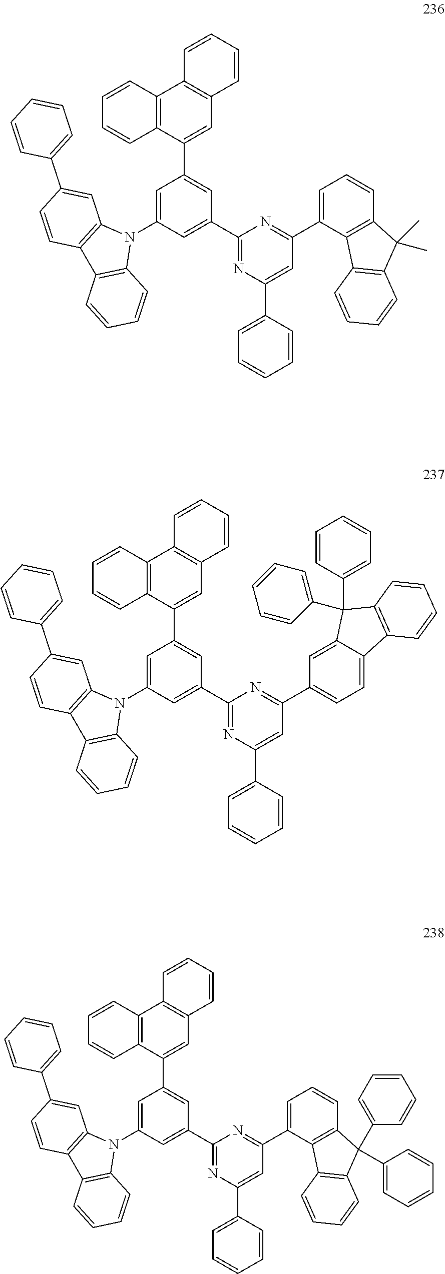 [SMILES: CC1(C)c2ccccc2-c2c(-c3cc(-c4ccccc4)nc(-c4cc(-c5cc6ccccc6c6ccccc56)cc(-n5c6ccccc6c6ccc(-c7ccccc7)cc65)c4)n3)cccc21 ; c1ccc(-c2ccc3c4ccccc4n(-c4cc(-c5nc(-c6ccccc6)cc(-c6ccc7c(c6)C(c6ccccc6)(c6ccccc6)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)cc(-c6cccc7c6-c6ccccc6C7(c6ccccc6)c6ccccc6)n5)cc(-c5cc6ccccc6c6ccccc56)c4)c3c2)cc1]

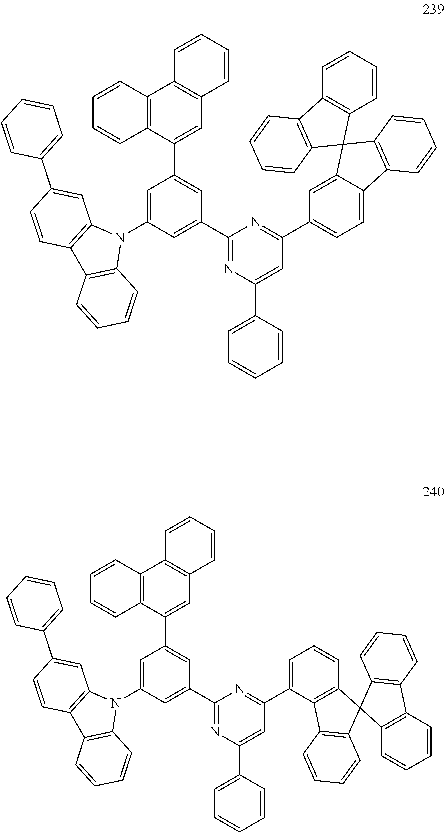 [SMILES: c1ccc(-c2ccc3c4ccccc4n(-c4cc(-c5nc(-c6ccccc6)cc(-c6ccc7c(c6)C6(c8ccccc8-c8ccccc86)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)cc(-c6cccc7c6-c6ccccc6C76c7ccccc7-c7ccccc76)n5)cc(-c5cc6ccccc6c6ccccc56)c4)c3c2)cc1]

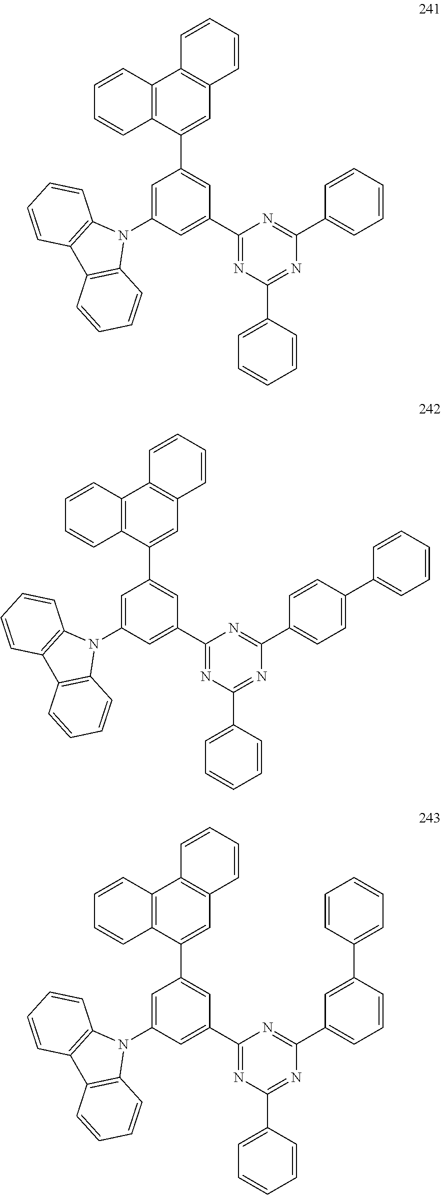

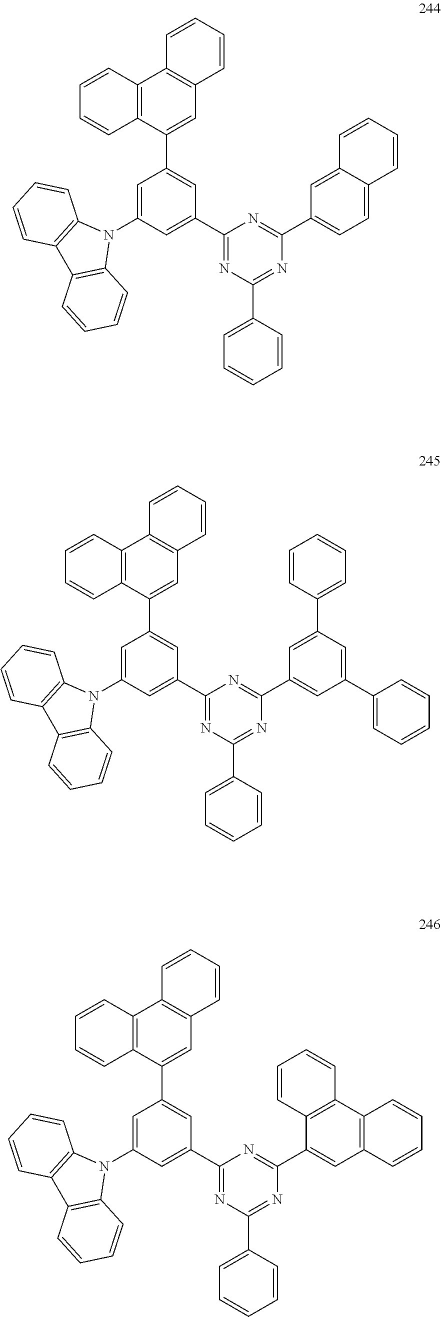 [SMILES: c1ccc(-c2nc(-c3cc(-c4cc5ccccc5c5ccccc45)cc(-n4c5ccccc5c5ccccc54)c3)nc(-c3ccc4ccccc4c3)n2)cc1 ; c1ccc(-c2cc(-c3ccccc3)cc(-c3nc(-c4ccccc4)nc(-c4cc(-c5cc6ccccc6c6ccccc56)cc(-n5c6ccccc6c6ccccc65)c4)n3)c2)cc1 ; c1ccc(-c2nc(-c3cc(-c4cc5ccccc5c5ccccc45)cc(-n4c5ccccc5c5ccccc54)c3)nc(-c3cc4ccccc4c4ccccc34)n2)cc1]

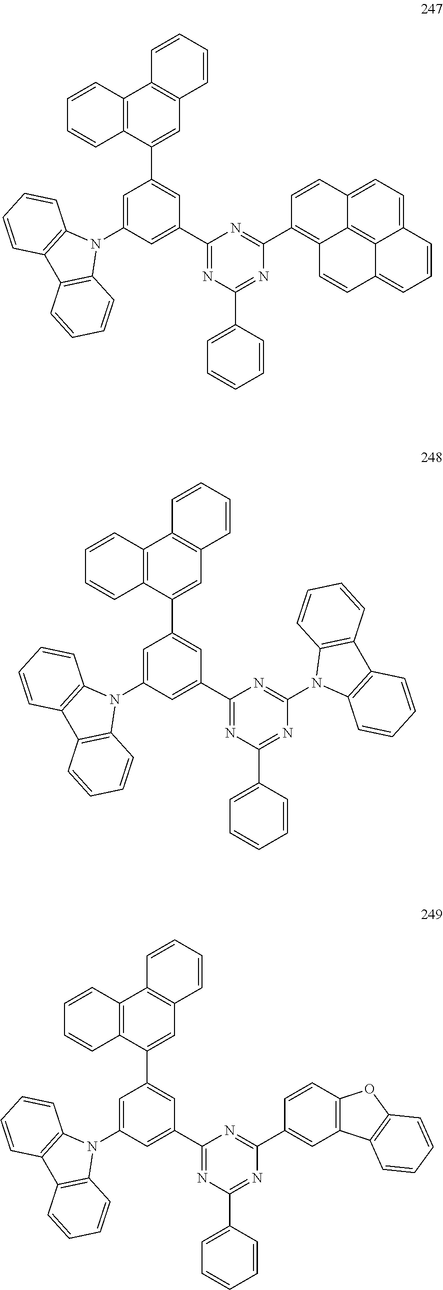 [SMILES: c1ccc(-c2nc(-c3cc(-c4cc5ccccc5c5ccccc45)cc(-n4c5ccccc5c5ccccc54)c3)nc(-c3ccc4ccc5cccc6ccc3c4c56)n2)cc1 ; c1ccc(-c2nc(-c3cc(-c4cc5ccccc5c5ccccc45)cc(-n4c5ccccc5c5ccccc54)c3)nc(-n3c4ccccc4c4ccccc43)n2)cc1 ; c1ccc(-c2nc(-c3cc(-c4cc5ccccc5c5ccccc45)cc(-n4c5ccccc5c5ccccc54)c3)nc(-c3ccc4oc5ccccc5c4c3)n2)cc1]

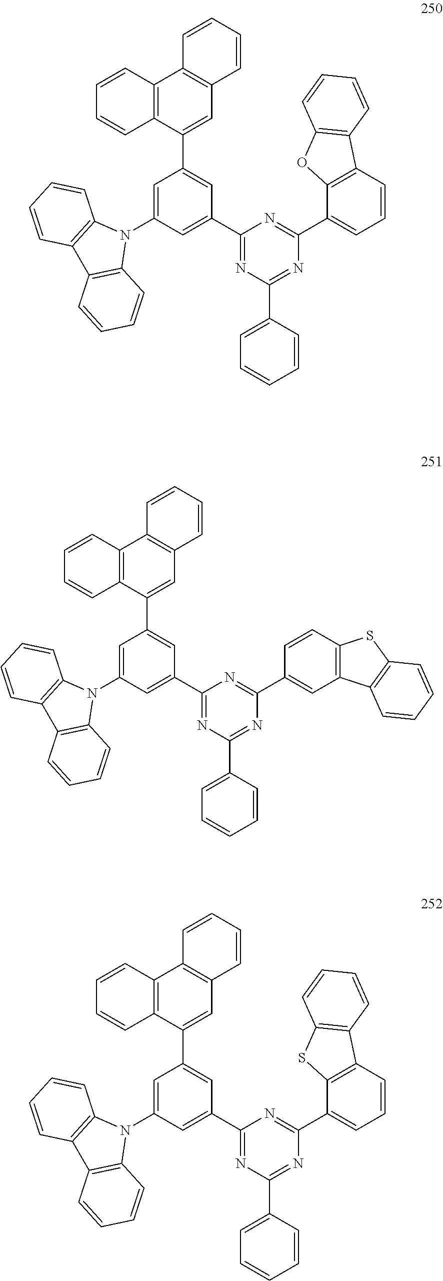 [SMILES: c1ccc(-c2nc(-c3cc(-c4cc5ccccc5c5ccccc45)cc(-n4c5ccccc5c5ccccc54)c3)nc(-c3cccc4c3oc3ccccc34)n2)cc1 ; c1ccc(-c2nc(-c3cc(-c4cc5ccccc5c5ccccc45)cc(-n4c5ccccc5c5ccccc54)c3)nc(-c3ccc4sc5ccccc5c4c3)n2)cc1 ; c1ccc(-c2nc(-c3cc(-c4cc5ccccc5c5ccccc45)cc(-n4c5ccccc5c5ccccc54)c3)nc(-c3cccc4c3sc3ccccc34)n2)cc1]

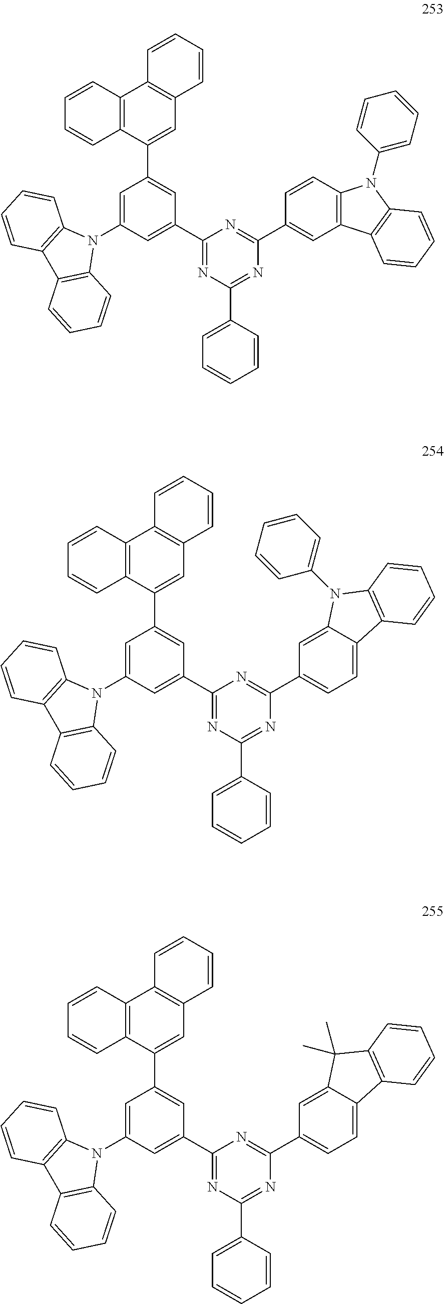 [SMILES: c1ccc(-c2nc(-c3cc(-c4cc5ccccc5c5ccccc45)cc(-n4c5ccccc5c5ccccc54)c3)nc(-c3ccc4c(c3)c3ccccc3n4-c3ccccc3)n2)cc1 ; c1ccc(-c2nc(-c3cc(-c4cc5ccccc5c5ccccc45)cc(-n4c5ccccc5c5ccccc54)c3)nc(-c3ccc4c5ccccc5n(-c5ccccc5)c4c3)n2)cc1 ; CC1(C)c2ccccc2-c2ccc(-c3nc(-c4ccccc4)nc(-c4cc(-c5cc6ccccc6c6ccccc56)cc(-n5c6ccccc6c6ccccc65)c4)n3)cc21]

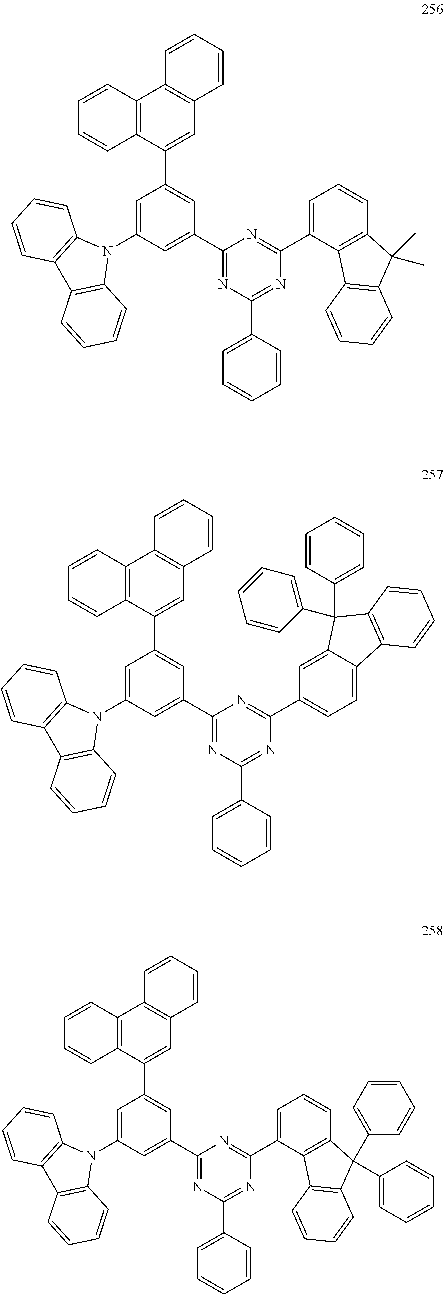 [SMILES: CC1(C)c2ccccc2-c2c(-c3nc(-c4ccccc4)nc(-c4cc(-c5cc6ccccc6c6ccccc56)cc(-n5c6ccccc6c6ccccc65)c4)n3)cccc21 ; c1ccc(-c2nc(-c3cc(-c4cc5ccccc5c5ccccc45)cc(-n4c5ccccc5c5ccccc54)c3)nc(-c3ccc4c(c3)C(c3ccccc3)(c3ccccc3)c3ccccc3-4)n2)cc1 ; c1ccc(-c2nc(-c3cc(-c4cc5ccccc5c5ccccc45)cc(-n4c5ccccc5c5ccccc54)c3)nc(-c3cccc4c3-c3ccccc3C4(c3ccccc3)c3ccccc3)n2)cc1]

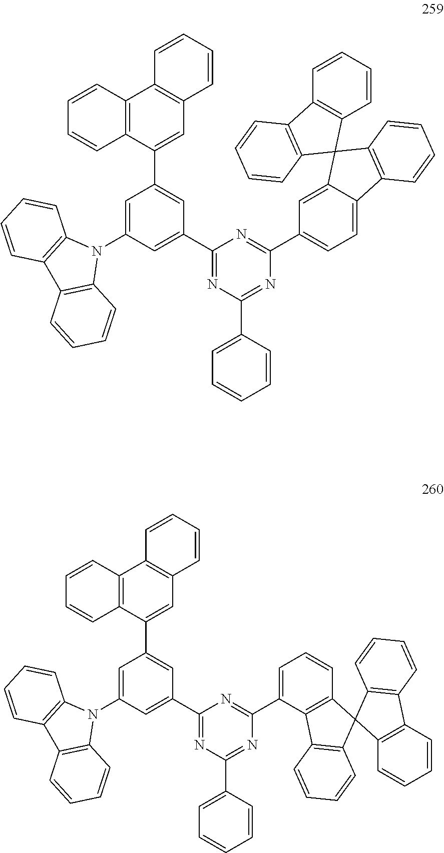 [SMILES: c1ccc(-c2nc(-c3cc(-c4cc5ccccc5c5ccccc45)cc(-n4c5ccccc5c5ccccc54)c3)nc(-c3ccc4c(c3)C3(c5ccccc5-c5ccccc53)c3ccccc3-4)n2)cc1 ; c1ccc(-c2nc(-c3cc(-c4cc5ccccc5c5ccccc45)cc(-n4c5ccccc5c5ccccc54)c3)nc(-c3cccc4c3-c3ccccc3C43c4ccccc4-c4ccccc43)n2)cc1]

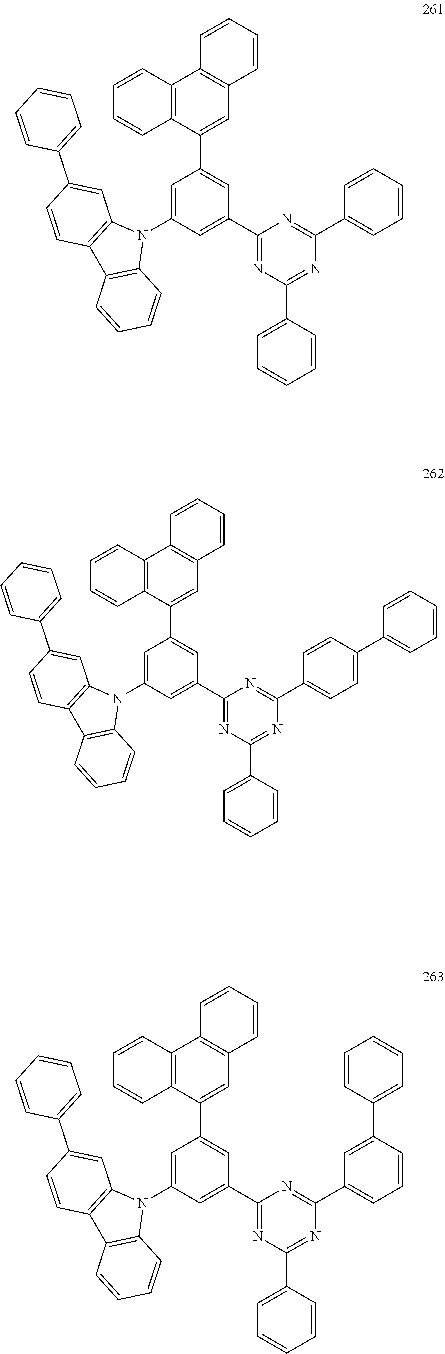 [SMILES: c1ccc(-c2ccc3c4ccccc4n(-c4cc(-c5nc(-c6ccccc6)nc(-c6ccccc6)n5)cc(-c5cc6ccccc6c6ccccc56)c4)c3c2)cc1 ; c1ccc(-c2ccc(-c3nc(-c4ccccc4)nc(-c4cc(-c5cc6ccccc6c6ccccc56)cc(-n5c6ccccc6c6ccc(-c7ccccc7)cc65)c4)n3)cc2)cc1 ; c1ccc(-c2cccc(-c3nc(-c4ccccc4)nc(-c4cc(-c5cc6ccccc6c6ccccc56)cc(-n5c6ccccc6c6ccc(-c7ccccc7)cc65)c4)n3)c2)cc1]

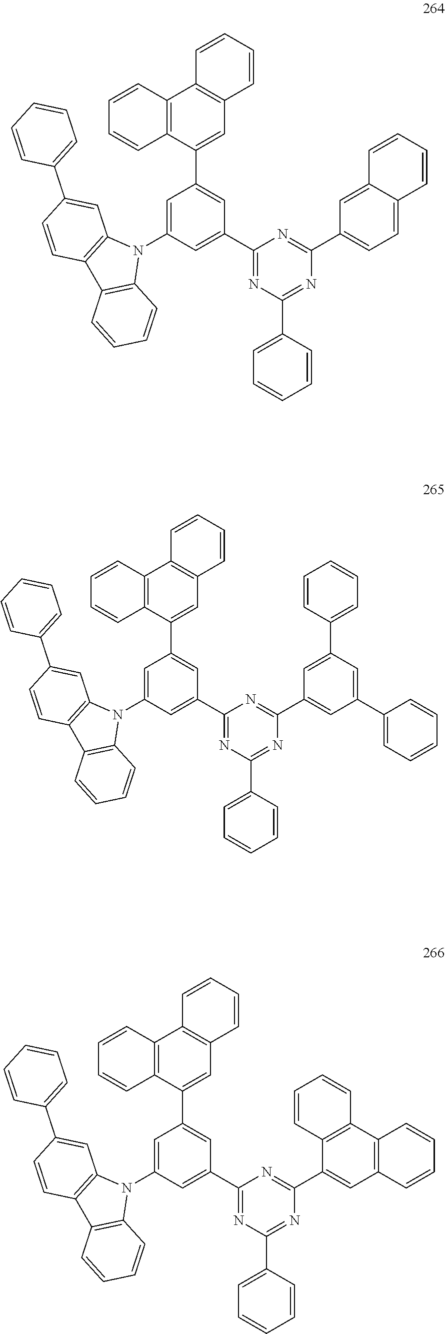 [SMILES: c1ccc(-c2ccc3c4ccccc4n(-c4cc(-c5nc(-c6ccccc6)nc(-c6ccc7ccccc7c6)n5)cc(-c5cc6ccccc6c6ccccc56)c4)c3c2)cc1 ; c1ccc(-c2cc(-c3ccccc3)cc(-c3nc(-c4ccccc4)nc(-c4cc(-c5cc6ccccc6c6ccccc56)cc(-n5c6ccccc6c6ccc(-c7ccccc7)cc65)c4)n3)c2)cc1 ; c1ccc(-c2ccc3c4ccccc4n(-c4cc(-c5nc(-c6ccccc6)nc(-c6cc7ccccc7c7ccccc67)n5)cc(-c5cc6ccccc6c6ccccc56)c4)c3c2)cc1]

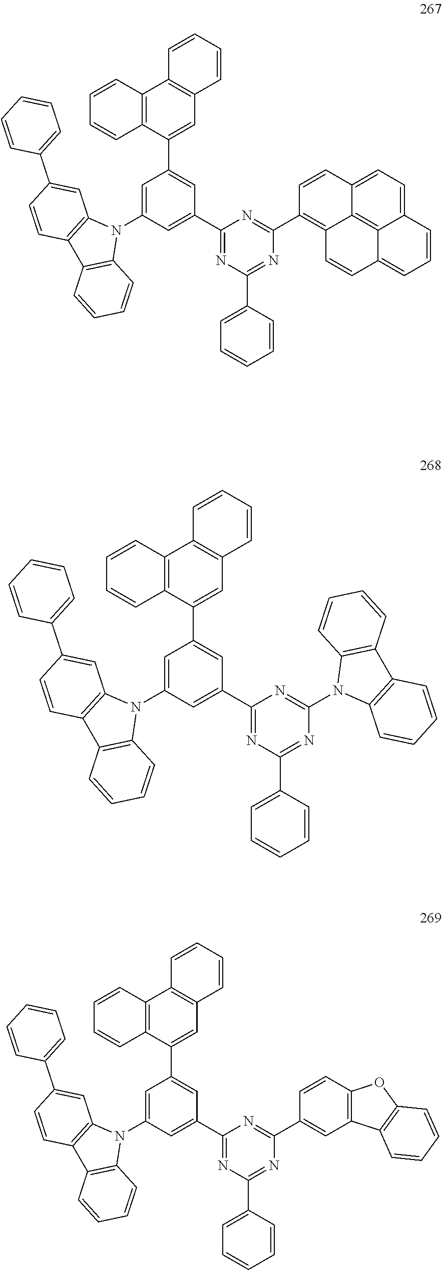 [SMILES: c1ccc(-c2ccc3c4ccccc4n(-c4cc(-c5nc(-c6ccccc6)nc(-c6ccc7ccc8cccc9ccc6c7c89)n5)cc(-c5cc6ccccc6c6ccccc56)c4)c3c2)cc1 ; c1ccc(-c2ccc3c4ccccc4n(-c4cc(-c5nc(-c6ccccc6)nc(-n6c7ccccc7c7ccccc76)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7oc8ccccc8c7c6)n5)cc(-c5cc6ccccc6c6ccccc56)c4)c3c2)cc1]

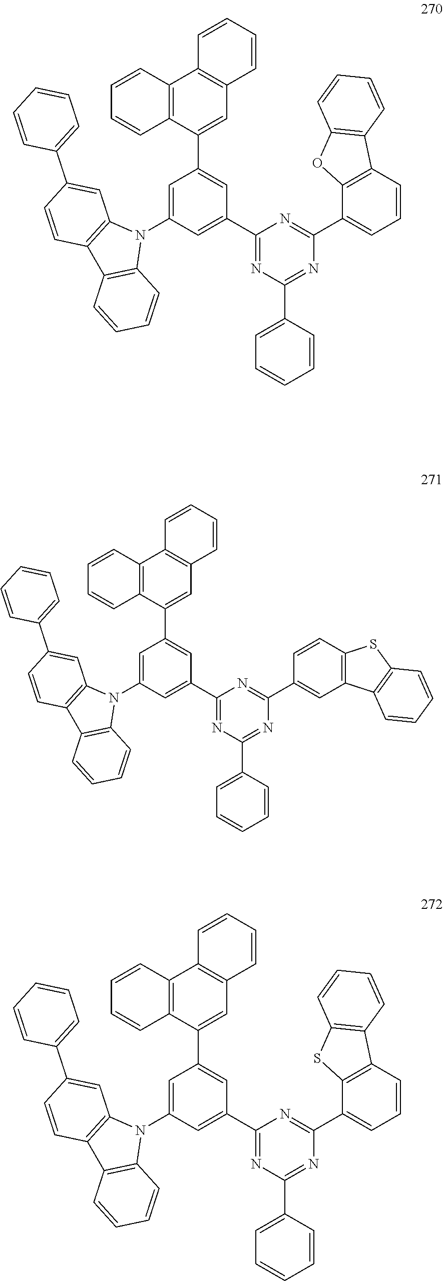 [SMILES: c1ccc(-c2ccc3c4ccccc4n(-c4cc(-c5nc(-c6ccccc6)nc(-c6cccc7c6oc6ccccc67)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7sc8ccccc8c7c6)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6sc6ccccc67)n5)cc(-c5cc6ccccc6c6ccccc56)c4)c3c2)cc1]

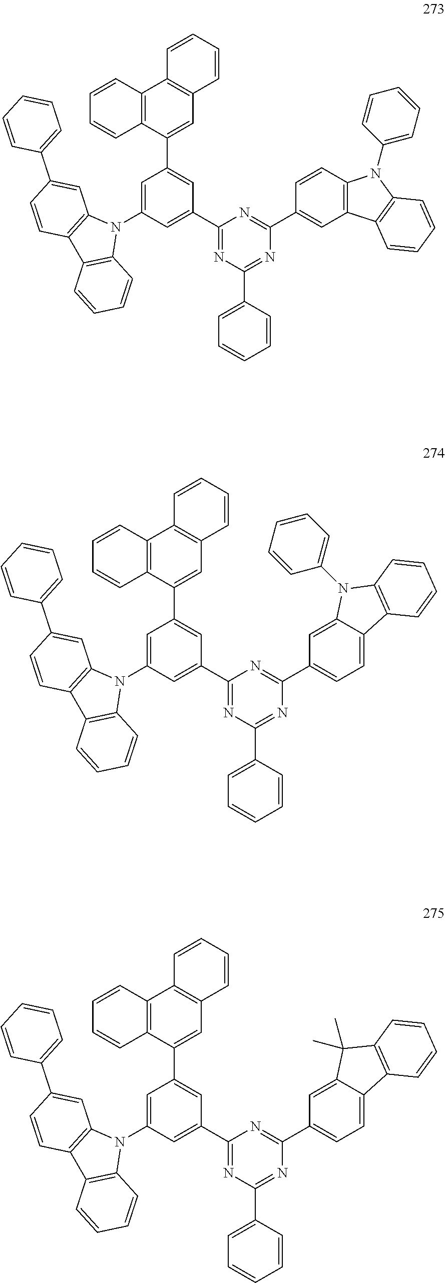 [SMILES: c1ccc(-c2ccc3c4ccccc4n(-c4cc(-c5nc(-c6ccccc6)nc(-c6ccc7c(c6)c6ccccc6n7-c6ccccc6)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7c8ccccc8n(-c8ccccc8)c7c6)n5)cc(-c5cc6ccccc6c6ccccc56)c4)c3c2)cc1 ; CC1(C)c2ccccc2-c2ccc(-c3nc(-c4ccccc4)nc(-c4cc(-c5cc6ccccc6c6ccccc56)cc(-n5c6ccccc6c6ccc(-c7ccccc7)cc65)c4)n3)cc21]

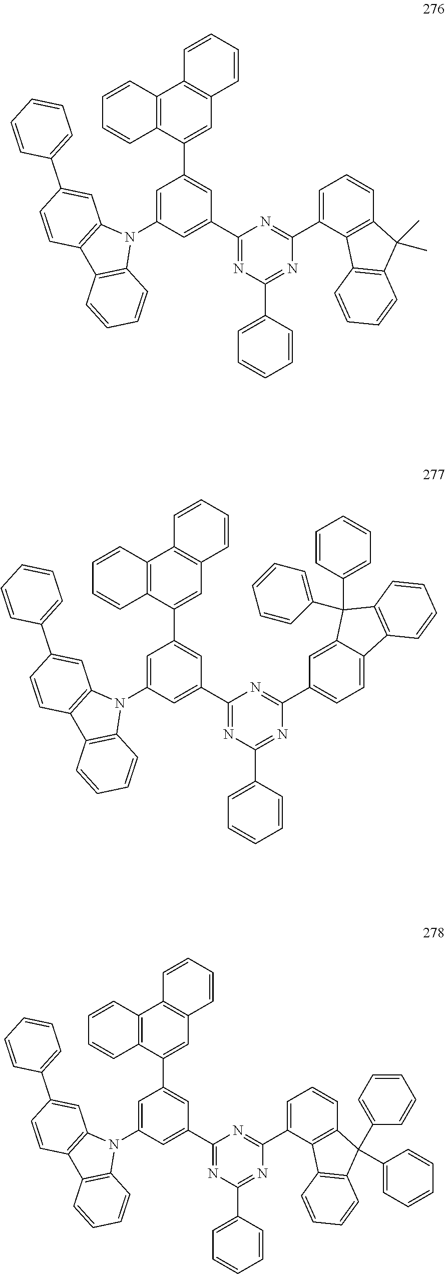 [SMILES: CC1(C)c2ccccc2-c2c(-c3nc(-c4ccccc4)nc(-c4cc(-c5cc6ccccc6c6ccccc56)cc(-n5c6ccccc6c6ccc(-c7ccccc7)cc65)c4)n3)cccc21 ; c1ccc(-c2ccc3c4ccccc4n(-c4cc(-c5nc(-c6ccccc6)nc(-c6ccc7c(c6)C(c6ccccc6)(c6ccccc6)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6-c6ccccc6C7(c6ccccc6)c6ccccc6)n5)cc(-c5cc6ccccc6c6ccccc56)c4)c3c2)cc1]

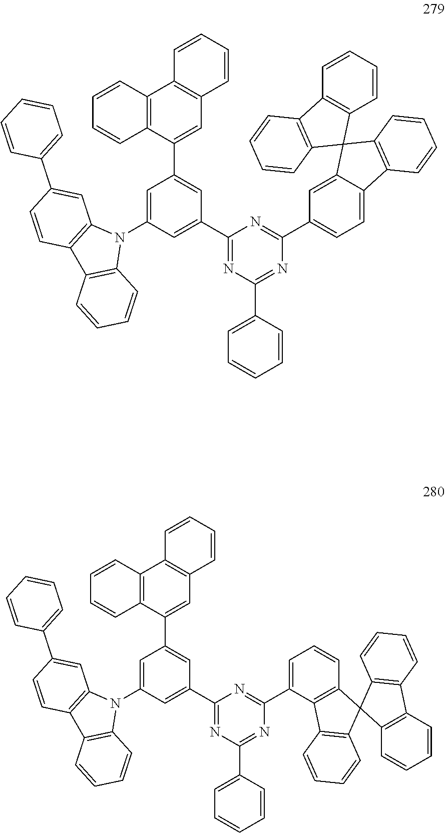 [SMILES: c1ccc(-c2ccc3c4ccccc4n(-c4cc(-c5nc(-c6ccccc6)nc(-c6ccc7c(c6)C6(c8ccccc8-c8ccccc86)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6-c6ccccc6C76c7ccccc7-c7ccccc76)n5)cc(-c5cc6ccccc6c6ccccc56)c4)c3c2)cc1]

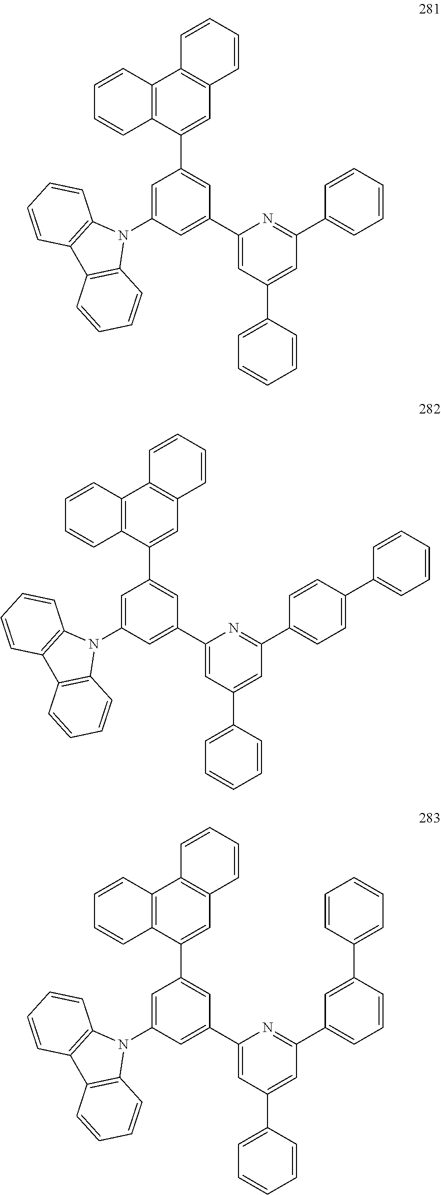

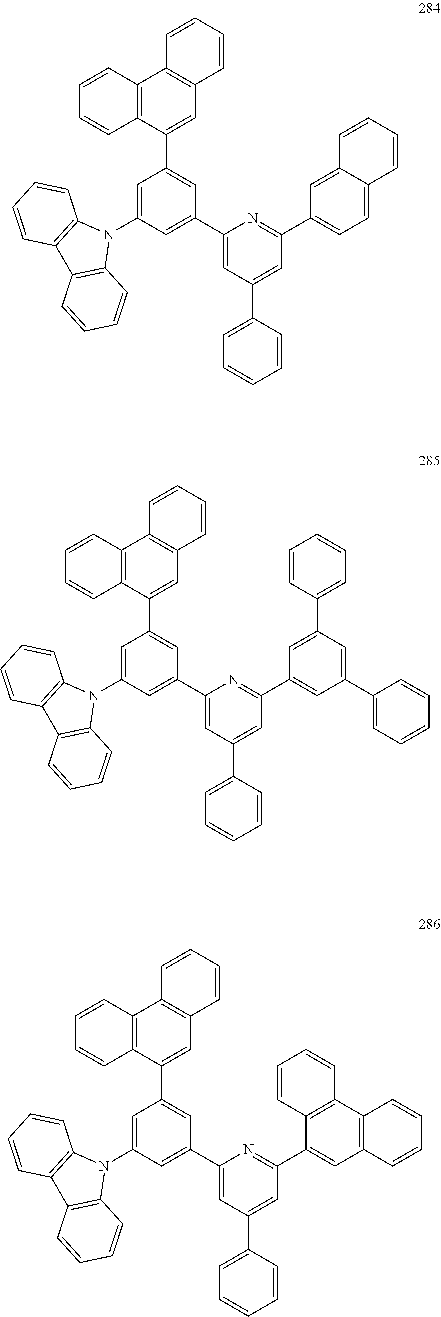 [SMILES: c1ccc(-c2cc(-c3cc(-c4cc5ccccc5c5ccccc45)cc(-n4c5ccccc5c5ccccc54)c3)nc(-c3ccc4ccccc4c3)c2)cc1 ; c1ccc(-c2cc(-c3ccccc3)cc(-c3cc(-c4ccccc4)cc(-c4cc(-c5cc6ccccc6c6ccccc56)cc(-n5c6ccccc6c6ccccc65)c4)n3)c2)cc1 ; c1ccc(-c2cc(-c3cc(-c4cc5ccccc5c5ccccc45)cc(-n4c5ccccc5c5ccccc54)c3)nc(-c3cc4ccccc4c4ccccc34)c2)cc1]

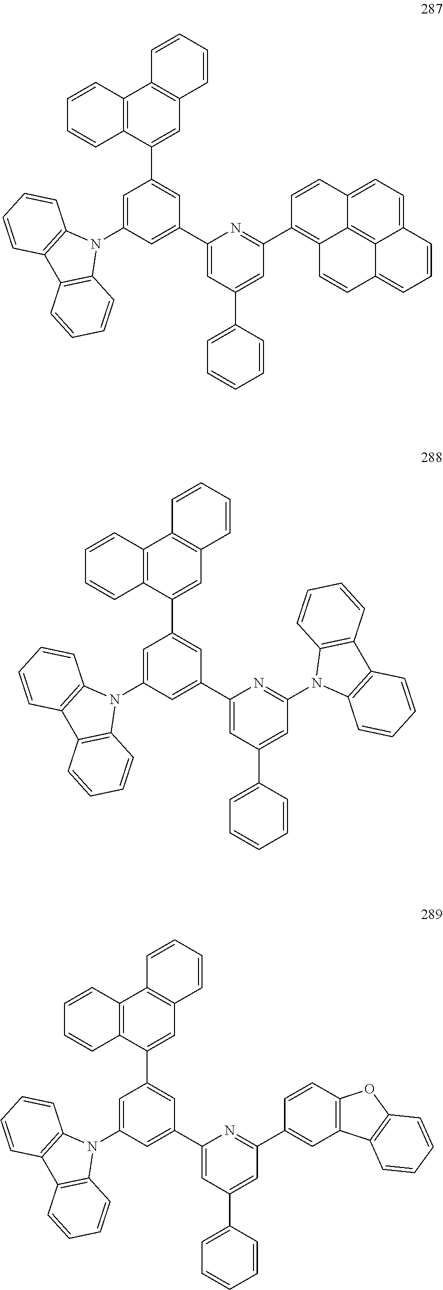 [SMILES: c1ccc(-c2cc(-c3cc(-c4cc5ccccc5c5ccccc45)cc(-n4c5ccccc5c5ccccc54)c3)nc(-c3ccc4ccc5cccc6ccc3c4c56)c2)cc1 ; c1ccc(-c2cc(-c3cc(-c4cc5ccccc5c5ccccc45)cc(-n4c5ccccc5c5ccccc54)c3)nc(-n3c4ccccc4c4ccccc43)c2)cc1 ; c1ccc(-c2cc(-c3cc(-c4cc5ccccc5c5ccccc45)cc(-n4c5ccccc5c5ccccc54)c3)nc(-c3ccc4oc5ccccc5c4c3)c2)cc1]

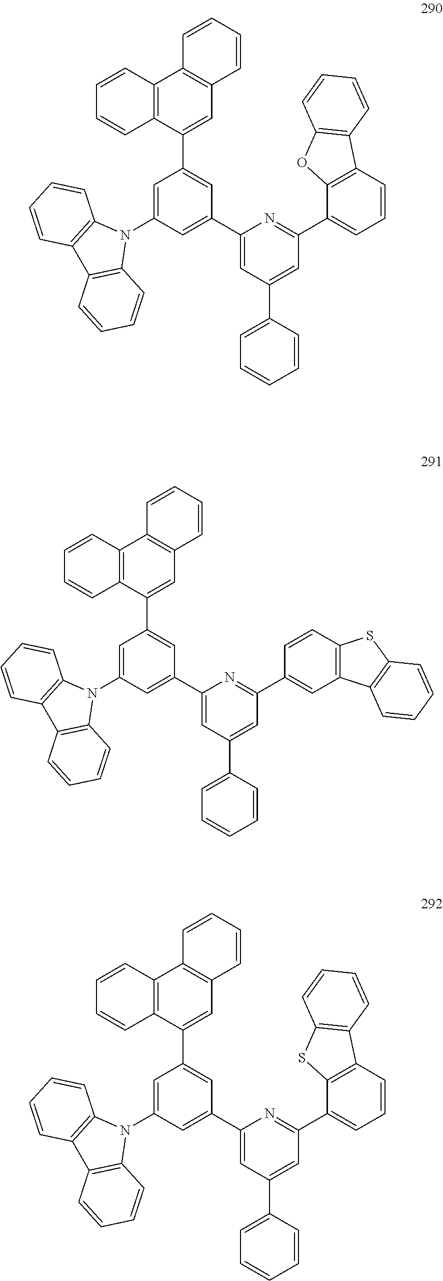 [SMILES: c1ccc(-c2cc(-c3cc(-c4cc5ccccc5c5ccccc45)cc(-n4c5ccccc5c5ccccc54)c3)nc(-c3cccc4c3oc3ccccc34)c2)cc1 ; c1ccc(-c2cc(-c3cc(-c4cc5ccccc5c5ccccc45)cc(-n4c5ccccc5c5ccccc54)c3)nc(-c3ccc4sc5ccccc5c4c3)c2)cc1 ; c1ccc(-c2cc(-c3cc(-c4cc5ccccc5c5ccccc45)cc(-n4c5ccccc5c5ccccc54)c3)nc(-c3cccc4c3sc3ccccc34)c2)cc1]

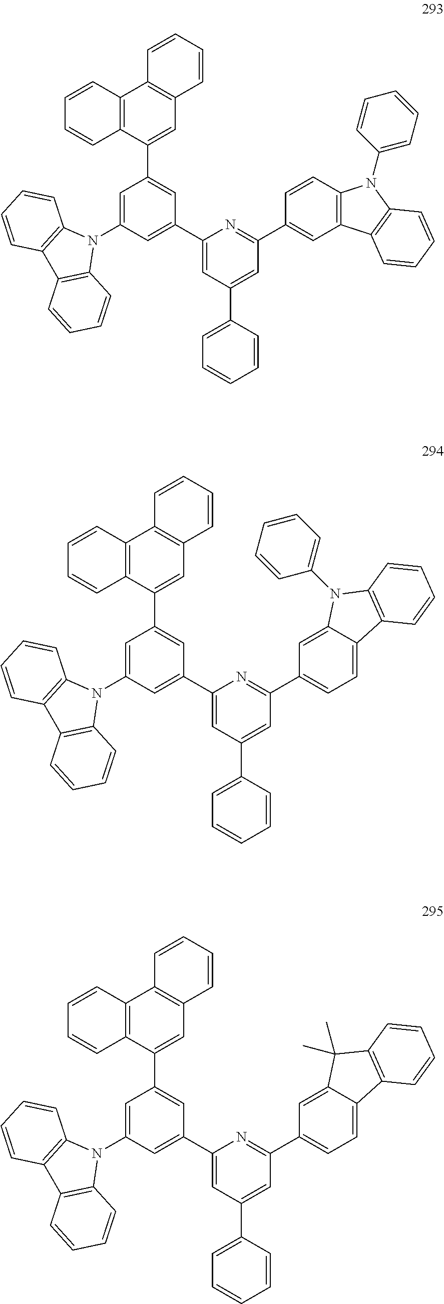 [SMILES: c1ccc(-c2cc(-c3cc(-c4cc5ccccc5c5ccccc45)cc(-n4c5ccccc5c5ccccc54)c3)nc(-c3ccc4c(c3)c3ccccc3n4-c3ccccc3)c2)cc1 ; c1ccc(-c2cc(-c3cc(-c4cc5ccccc5c5ccccc45)cc(-n4c5ccccc5c5ccccc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5cc6ccccc6c6ccccc56)cc(-n5c6ccccc6c6ccccc65)c4)n3)cc21]

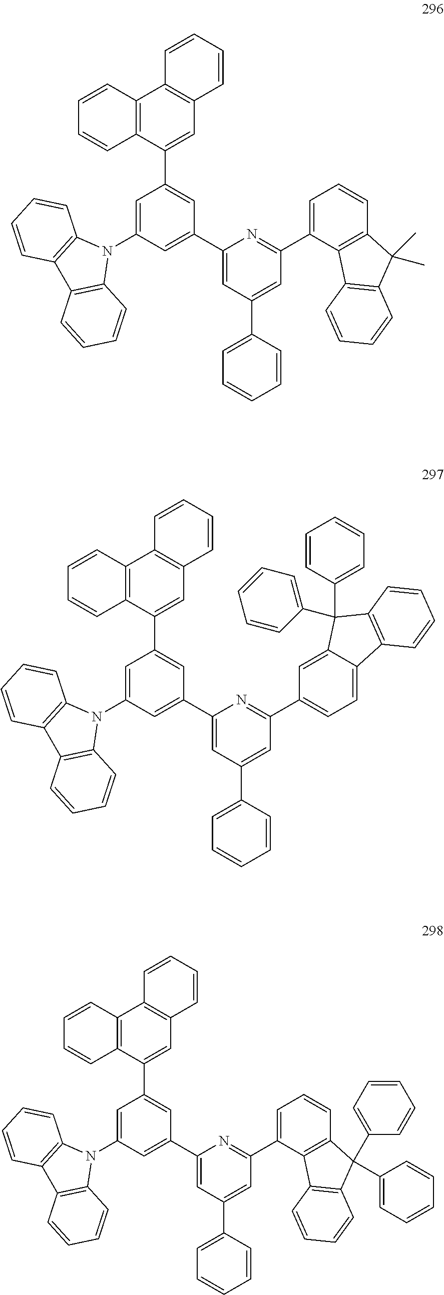 [SMILES: CC1(C)c2ccccc2-c2c(-c3cc(-c4ccccc4)cc(-c4cc(-c5cc6ccccc6c6ccccc56)cc(-n5c6ccccc6c6ccccc65)c4)n3)cccc21 ; c1ccc(-c2cc(-c3cc(-c4cc5ccccc5c5ccccc45)cc(-n4c5ccccc5c5ccccc54)c3)nc(-c3ccc4c(c3)C(c3ccccc3)(c3ccccc3)c3ccccc3-4)c2)cc1 ; c1ccc(-c2cc(-c3cc(-c4cc5ccccc5c5ccccc45)cc(-n4c5ccccc5c5ccccc54)c3)nc(-c3cccc4c3-c3ccccc3C4(c3ccccc3)c3ccccc3)c2)cc1]

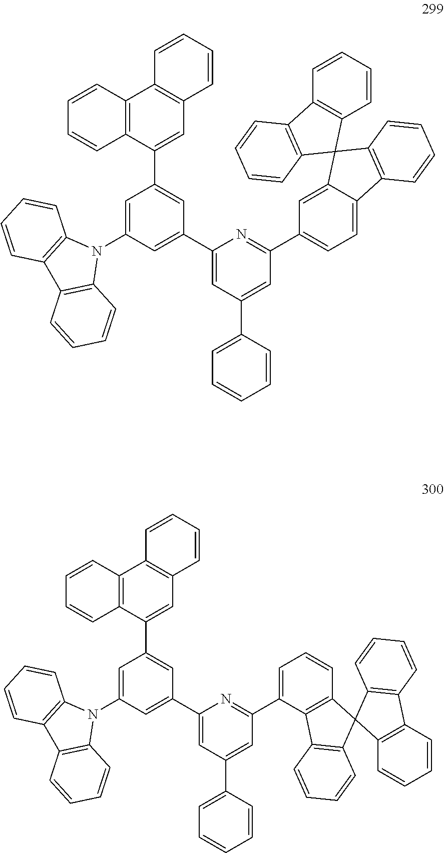 [SMILES: c1ccc(-c2cc(-c3cc(-c4cc5ccccc5c5ccccc45)cc(-n4c5ccccc5c5ccccc54)c3)nc(-c3ccc4c(c3)C3(c5ccccc5-c5ccccc53)c3ccccc3-4)c2)cc1 ; c1ccc(-c2cc(-c3cc(-c4cc5ccccc5c5ccccc45)cc(-n4c5ccccc5c5ccccc54)c3)nc(-c3cccc4c3-c3ccccc3C43c4ccccc4-c4ccccc43)c2)cc1]

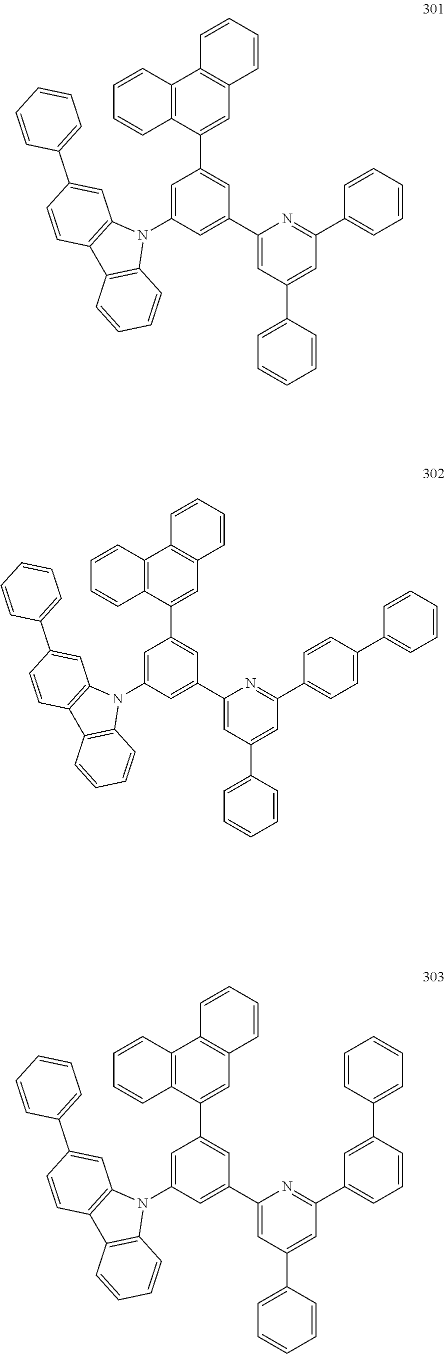 [SMILES: c1ccc(-c2cc(-c3ccccc3)nc(-c3cc(-c4cc5ccccc5c5ccccc45)cc(-n4c5ccccc5c5ccc(-c6ccccc6)cc54)c3)c2)cc1 ; c1ccc(-c2ccc(-c3cc(-c4ccccc4)cc(-c4cc(-c5cc6ccccc6c6ccccc56)cc(-n5c6ccccc6c6ccc(-c7ccccc7)cc65)c4)n3)cc2)cc1 ; c1ccc(-c2cccc(-c3cc(-c4ccccc4)cc(-c4cc(-c5cc6ccccc6c6ccccc56)cc(-n5c6ccccc6c6ccc(-c7ccccc7)cc65)c4)n3)c2)cc1]

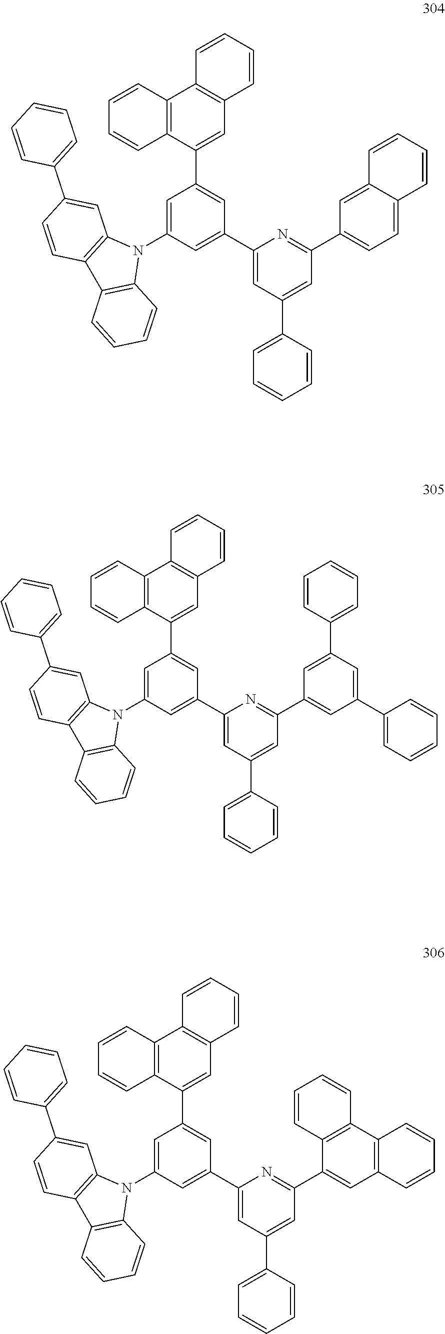 [SMILES: c1ccc(-c2cc(-c3cc(-c4cc5ccccc5c5ccccc45)cc(-n4c5ccccc5c5ccc(-c6ccccc6)cc54)c3)nc(-c3ccc4ccccc4c3)c2)cc1 ; c1ccc(-c2cc(-c3ccccc3)cc(-c3cc(-c4ccccc4)cc(-c4cc(-c5cc6ccccc6c6ccccc56)cc(-n5c6ccccc6c6ccc(-c7ccccc7)cc65)c4)n3)c2)cc1 ; c1ccc(-c2cc(-c3cc(-c4cc5ccccc5c5ccccc45)cc(-n4c5ccccc5c5ccc(-c6ccccc6)cc54)c3)nc(-c3cc4ccccc4c4ccccc34)c2)cc1]

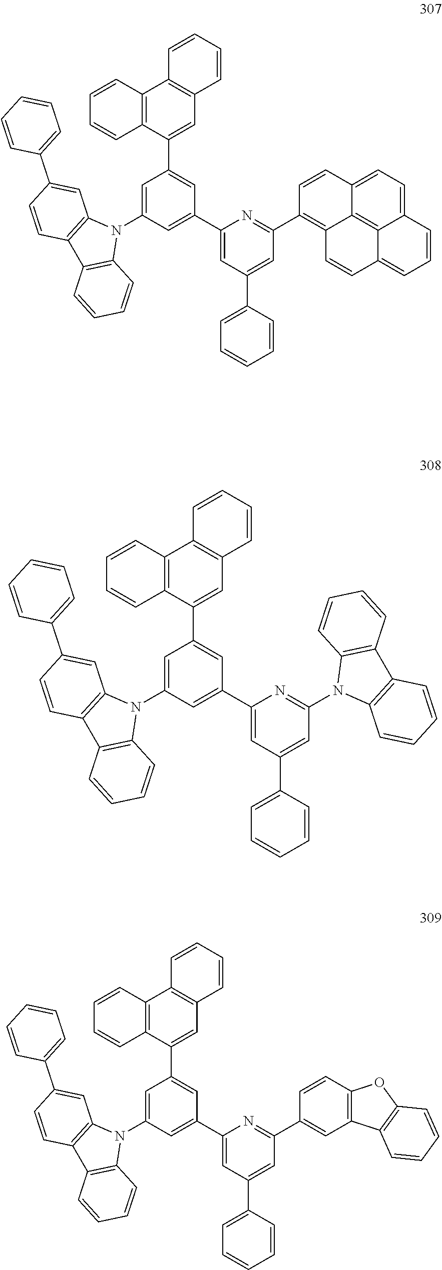 [SMILES: c1ccc(-c2cc(-c3cc(-c4cc5ccccc5c5ccccc45)cc(-n4c5ccccc5c5ccc(-c6ccccc6)cc54)c3)nc(-c3ccc4ccc5cccc6ccc3c4c56)c2)cc1 ; c1ccc(-c2cc(-c3cc(-c4cc5ccccc5c5ccccc45)cc(-n4c5ccccc5c5ccc(-c6ccccc6)cc54)c3)nc(-n3c4ccccc4c4ccccc43)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4oc5ccccc5c4c3)c2)cc1]

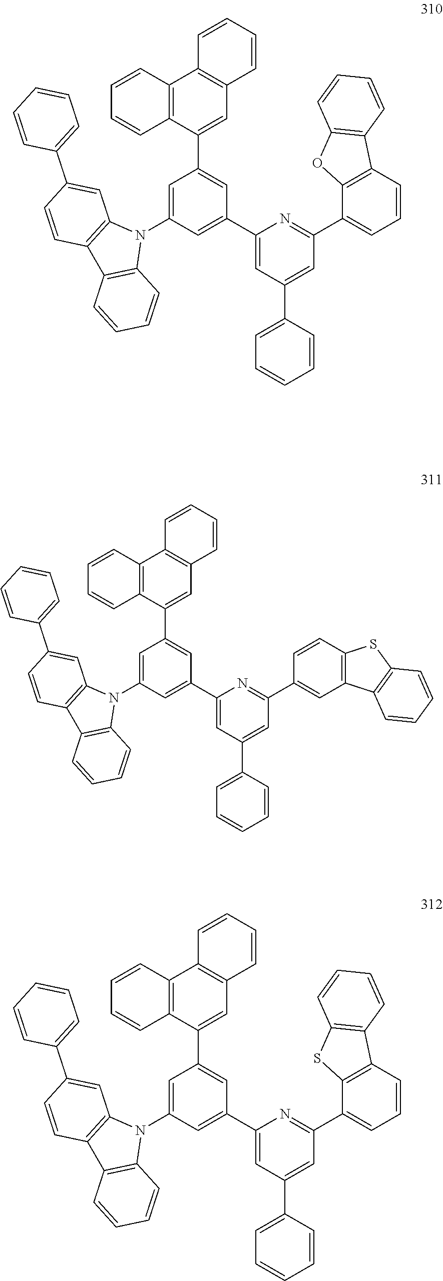 [SMILES: c1ccc(-c2cc(-c3cc(-c4cc5ccccc5c5ccccc45)cc(-n4c5ccccc5c5ccc(-c6ccccc6)cc54)c3)nc(-c3cccc4c3oc3ccccc34)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4sc5ccccc5c4c3)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3sc3ccccc34)c2)cc1]

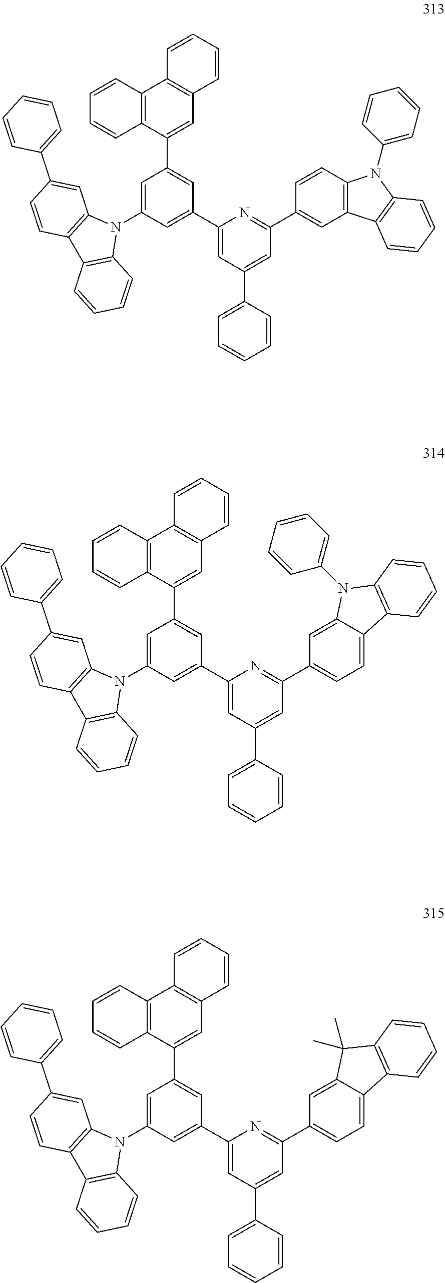 [SMILES: c1ccc(-c2cc(-c3cc(-c4cc5ccccc5c5ccccc45)cc(-n4c5ccccc5c5ccc(-c6ccccc6)cc54)c3)nc(-c3ccc4c(c3)c3ccccc3n4-c3ccccc3)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5cc6ccccc6c6ccccc56)cc(-n5c6ccccc6c6ccc(-c7ccccc7)cc65)c4)n3)cc21]

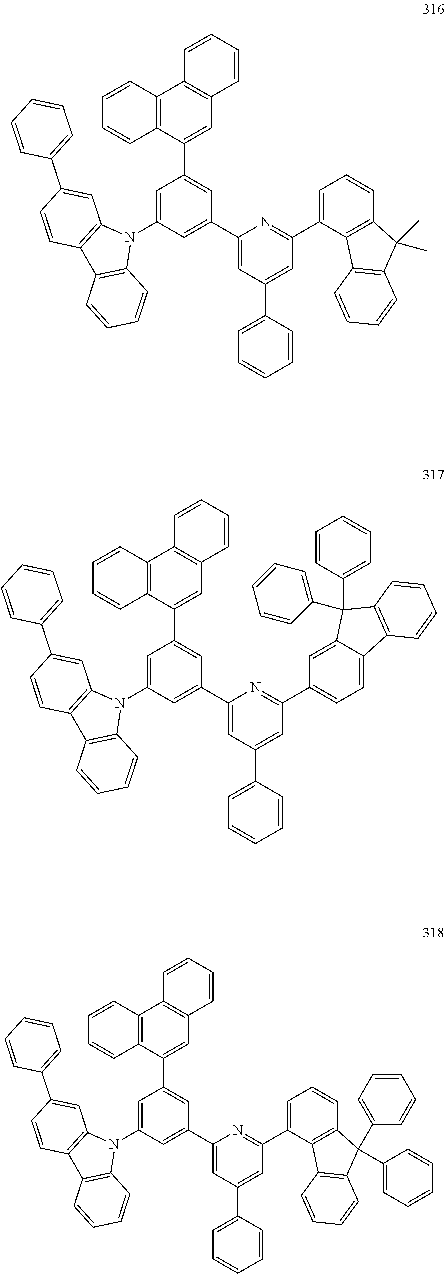 [SMILES: CC1(C)c2ccccc2-c2c(-c3cc(-c4ccccc4)cc(-c4cc(-c5cc6ccccc6c6ccccc56)cc(-n5c6ccccc6c6ccc(-c7ccccc7)cc65)c4)n3)cccc21 ; c1ccc(-c2cc(-c3cc(-c4cc5ccccc5c5ccccc45)cc(-n4c5ccccc5c5ccc(-c6ccccc6)cc54)c3)nc(-c3ccc4c(c3)C(c3ccccc3)(c3ccccc3)c3ccccc3-4)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3-c3ccccc3C4(c3ccccc3)c3ccccc3)c2)cc1]

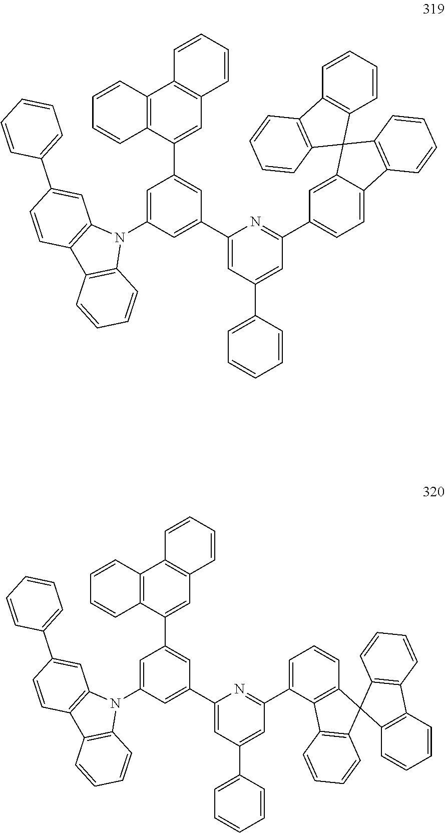 [SMILES: c1ccc(-c2cc(-c3cc(-c4cc5ccccc5c5ccccc45)cc(-n4c5ccccc5c5ccc(-c6ccccc6)cc54)c3)nc(-c3ccc4c(c3)C3(c5ccccc5-c5ccccc53)c3ccccc3-4)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3-c3ccccc3C43c4ccccc4-c4ccccc43)c2)cc1]

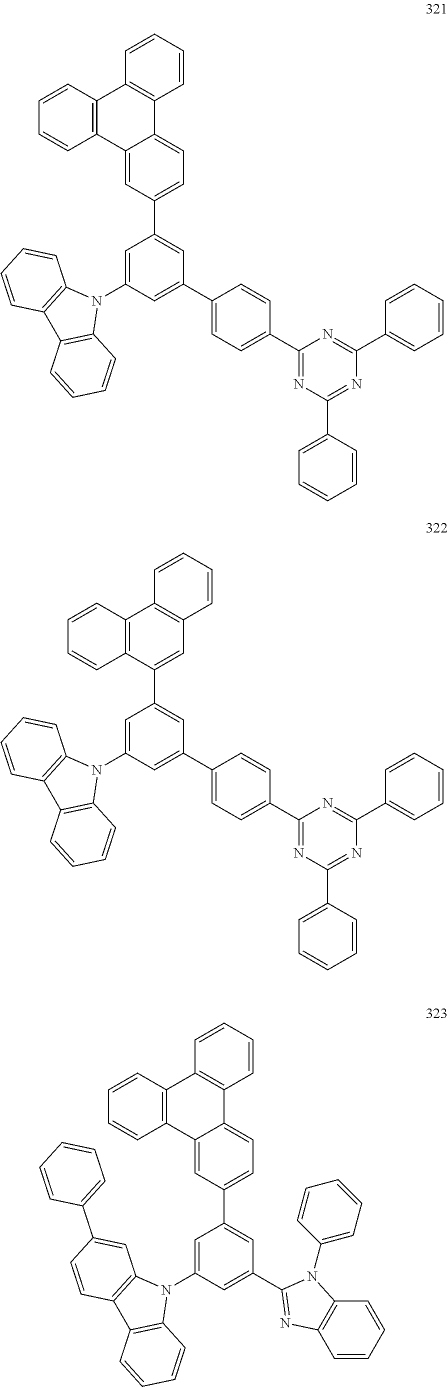 [SMILES: c1ccc(-c2nc(-c3ccccc3)nc(-c3ccc(-c4cc(-c5ccc6c7ccccc7c7ccccc7c6c5)cc(-n5c6ccccc6c6ccccc65)c4)cc3)n2)cc1 ; c1ccc(-c2nc(-c3ccccc3)nc(-c3ccc(-c4cc(-c5cc6ccccc6c6ccccc56)cc(-n5c6ccccc6c6ccccc65)c4)cc3)n2)cc1 ; c1ccc(-c2ccc3c4ccccc4n(-c4cc(-c5ccc6c7ccccc7c7ccccc7c6c5)cc(-c5nc6ccccc6n5-c5ccccc5)c4)c3c2)cc1]

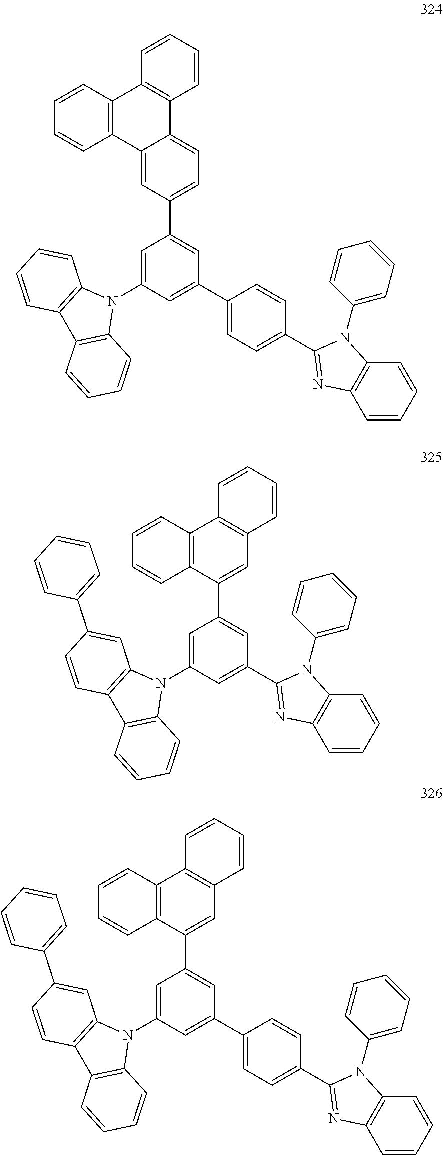 [SMILES: c1ccc(-n2c(-c3ccc(-c4cc(-c5ccc6c7ccccc7c7ccccc7c6c5)cc(-n5c6ccccc6c6ccccc65)c4)cc3)nc3ccccc32)cc1 ; c1ccc(-c2ccc3c4ccccc4n(-c4cc(-c5cc6ccccc6c6ccccc56)cc(-c5nc6ccccc6n5-c5ccccc5)c4)c3c2)cc1 ; c1ccc(-c2ccc3c4ccccc4n(-c4cc(-c5ccc(-c6nc7ccccc7n6-c6ccccc6)cc5)cc(-c5cc6ccccc6c6ccccc56)c4)c3c2)cc1]

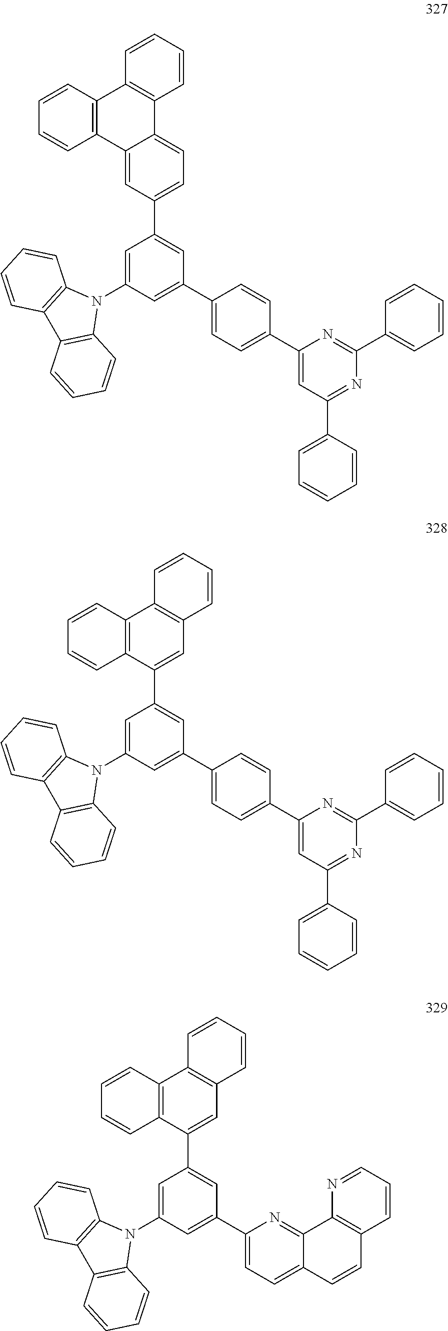 [SMILES: c1ccc(-c2cc(-c3ccc(-c4cc(-c5ccc6c7ccccc7c7ccccc7c6c5)cc(-n5c6ccccc6c6ccccc65)c4)cc3)nc(-c3ccccc3)n2)cc1 ; c1ccc(-c2cc(-c3ccc(-c4cc(-c5cc6ccccc6c6ccccc56)cc(-n5c6ccccc6c6ccccc65)c4)cc3)nc(-c3ccccc3)n2)cc1 ; c1ccc2c(c1)cc(-c1cc(-c3ccc4ccc5cccnc5c4n3)cc(-n3c4ccccc4c4ccccc43)c1)c1ccccc12]

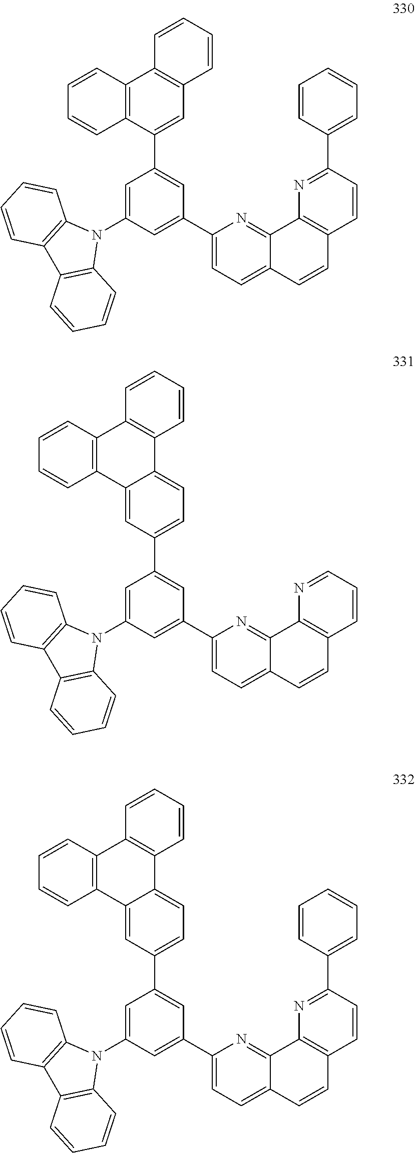 [SMILES: c1ccc(-c2ccc3ccc4ccc(-c5cc(-c6cc7ccccc7c7ccccc67)cc(-n6c7ccccc7c7ccccc76)c5)nc4c3n2)cc1 ; c1cnc2c(c1)ccc1ccc(-c3cc(-c4ccc5c6ccccc6c6ccccc6c5c4)cc(-n4c5ccccc5c5ccccc54)c3)nc12 ; c1ccc(-c2ccc3ccc4ccc(-c5cc(-c6ccc7c8ccccc8c8ccccc8c7c6)cc(-n6c7ccccc7c7ccccc76)c5)nc4c3n2)cc1]

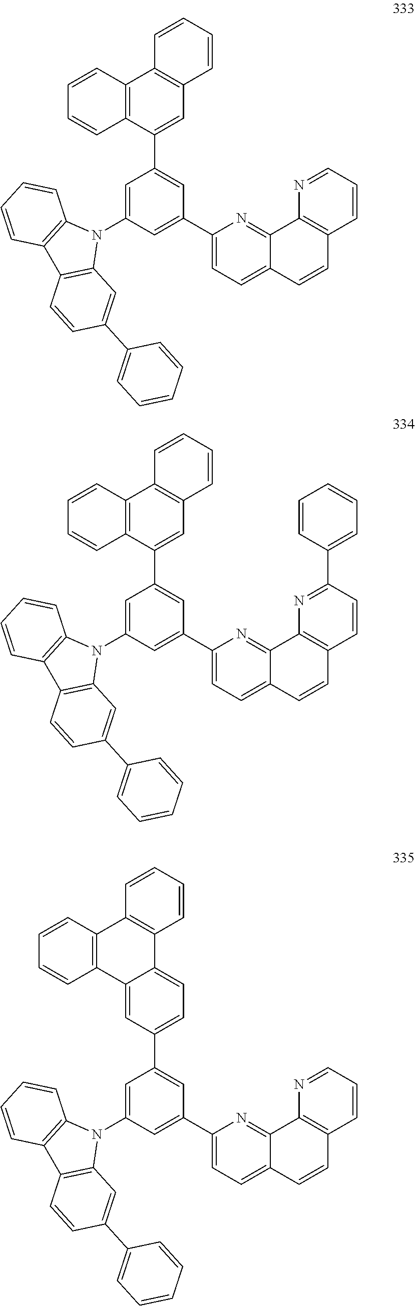

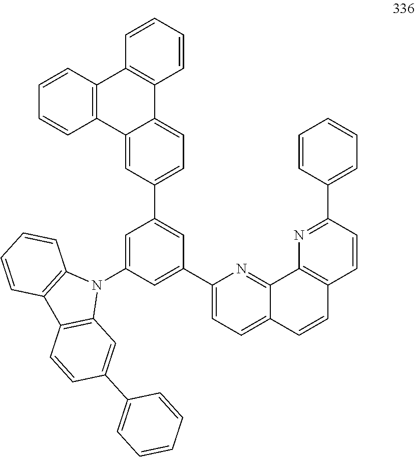 [SMILES: c1ccc(-c2ccc3c4ccccc4n(-c4cc(-c5ccc6c7ccccc7c7ccccc7c6c5)cc(-c5ccc6ccc7ccc(-c8ccccc8)nc7c6n5)c4)c3c2)cc1]

In addition, by introducing various substituents into the structure of the Chemical Formula 1, it is possible to synthesize a compound having intrinsic characteristics of the introduced substituents. For example, by introducing into the core structure a substituent which is mainly used as a hole injecting layer material, a hole transport layer material, a light emitting layer material, a hole blocking layer material, an electron transport layer material or an electron injecting layer material at the time of manufacturing the organic light emitting device, it is possible to synthesize a material that satisfies the conditions required in each organic material layer.

In addition, by introducing various substituents into the structure of Chemical Formula 1, it is possible to finely control the energy band gap, and further, the characteristics at interfaces between the organic materials can be improved, and the use of the materials can be diversified.

Meanwhile, the compound represented by Chemical Formula 1 has a high glass transition temperature (Tg), allowing it to have excellent thermal stability. This increase in thermal stability is an important factor in providing driving stability to the device.

The compound according to an embodiment of the present invention may be prepared by a multistep chemical reaction. Some intermediate compounds may be prepared first, and the compound represented by Chemical Formula 1 may be prepared from intermediate compounds thereof. More specifically, a method for preparing a compound according to an embodiment of the present invention may be performed as in the following Examples.

Another embodiment of the present invention provides an organic light emitting device comprising the compound represented by Chemical Formula 1.

The organic light emitting device according to an embodiment of the present invention may be manufactured by conventional manufacturing methods and using the materials of organic light emitting devices, except that one or more of the layered organic material layers are formed using the above-described compound.

The compound represented by Chemical Formula 1 may be formed as an organic material layer by performing a solution coating method as well as a vacuum deposition method at the time of manufacturing the organic light emitting device. Here, the solution coating method refers to spin coating, dip coating, inkjet printing, screen printing, spraying, roll coating, and the like, but is not limited thereto. For example, even when the compound represented by Chemical Formula 1 is used as a material for the light emitting layer, the hole blocking layer, the electron transport layer, or the electron injecting layer, an organic material layer can be formed by the solution coating method.

As another example, when the compound represented by Chemical Formula 1 is used to form an organic material layer, the organic material layer below may be formed by the solution coating method, and the organic material layer comprising the compound represented by Chemical Formula 1 may be formed by the vacuum deposition method. Specifically, when the compound represented by Chemical Formula 1 is used as the material for the hole blocking layer, the electron transport layer, or the electron injecting layer, the solution coating method may be used when the light emitting layer is formed on the anode, or when the hole injecting layer and/or the hole transport layer and the light emitting layer is formed on the anode, and the organic material layer comprising the compound represented by Chemical Formula 1 may be formed thereon by using the vacuum deposition method. In this case, even though the organic material layer comprising the compound represented by Chemical Formula 1 is manufactured by the vacuum deposition method, the organic material layer comprising the compound represented by Chemical Formula 1 is well matched with the organic material layer below, formed by the solution coating method.

Specifically, the organic light emitting device according to an embodiment of the present invention includes an anode, a cathode, and one or more layered organic material layers provided between the anode and the cathode, wherein one or more of the organic material layers include the compound represented by Chemical Formula 1.

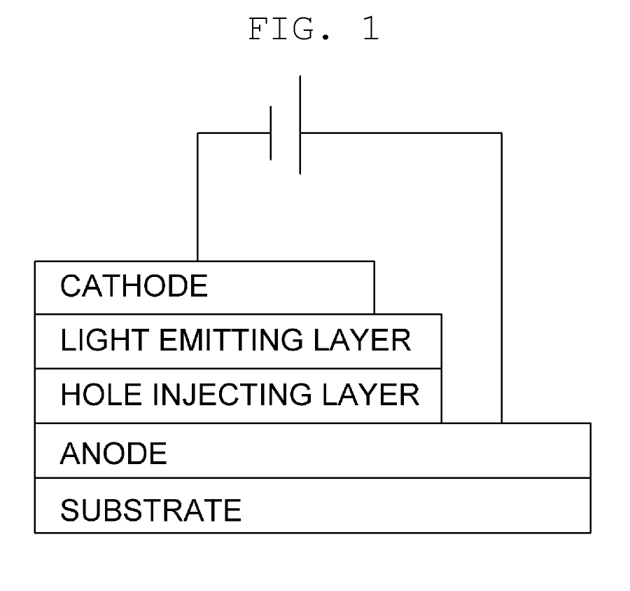

FIG. 1 illustrates a stacking order of an electrode and organic material layers of an organic light emitting device, according to an embodiment of the present invention. However, the above-described drawing is not intended to limit the scope of the present invention, and any structure of an organic light emitting device known in the art can be applied to the present invention.

FIG. 1 illustrates an organic light emitting device in which the anode, the hole injecting layer, the light emitting layer, and the cathode are sequentially stacked on the substrate. However, the present invention is not limited to this structure. Specifically, the compound represented by Chemical Formula 1 may be included in the light emitting layer of the structure illustrated in FIG. 1. In particular, the compound represented by Chemical Formula 1 may be included in the electron transport layer or the hole blocking layer.

Specifically, the organic light emitting device may have a structure of: substrate/anode/light emitting layer/cathode; substrate/anode/hole injecting layer/light emitting layer/cathode; substrate/anode/hole transport layer/light emitting layer/cathode; substrate/anode/hole injecting layer/hole transport layer/light emitting layer/cathode; substrate/anode/light emitting layer/electron transport layer/cathode; substrate/anode/light emitting layer/electron injecting layer/cathode; substrate/anode/light emitting layer/hole blocking layer/cathode; substrate/anode/light emitting layer/electron transport layer/electron injecting layer/cathode; substrate/anode/light emitting layer/hole blocking layer/electron transport layer/cathode; substrate/anode/light emitting layer/hole blocking layer/electron transport layer/electron injecting layer/cathode; or the like, wherein one or more of the layered organic material layers between the anode and the cathode, for example, the hole injecting layer, the hole transport layer, the light emitting layer, the hole blocking layer, the electron transport layer or the electron injecting layer, may include the compound represented by Chemical Formula 1. More specifically, the compound represented by Chemical Formula 1 may be used as the material for the light emitting layer, the hole blocking layer, the electron transport layer, or the electron injecting layer in a device having the above structure.

In another embodiment, the organic light emitting device may include a charge generating layer comprising the compound represented by Chemical Formula 1. For example, the organic light emitting device may comprise two or more light emitting units including the light emitting layer, and the charge generating layer may be provided between two adjacent light emitting units. As another example, the organic light emitting device may include one or more light emitting units, and the charge generating layer may be provided between the light emitting unit and the anode, or between the light emitting unit and the cathode.

Here, since the charge generating layer comprising the compound represented by Chemical Formula 1 can serve as an n-type charge generating layer, the charge generating layer comprising the compound represented by Chemical Formula 1 may be provided in contact with a p-type organic material layer. Specific examples of the p-type organic material layer are HAT-CN, F4-TCNQ, and a transition metal oxide.

The light emitting unit may be composed of only a light emitting layer, and may further include one or more organic material layers such as a hole injecting layer, a hole transport layer, a hole blocking layer, an electron transport layer, an electron injecting layer, and the like, if necessary.

For example, the organic light emitting device may have a structure of: substrate/anode/light emitting unit/charge generating layer (n-type)/charge generating layer (p-type)/light emitting unit/cathode; substrate/anode/charge generating layer (n-type)/charge generating layer (p-type)/light emitting unit/cathode; substrate/anode/light emitting unit/charge generating layer (n-type)/charge generating layer (p-type)/cathode; or the like, wherein the number of light emitting units may be two, three, or more, if necessary. The light emitting unit comprises a light emitting layer, and may further include one or more layers of the hole injecting layer, the hole transport layer, the hole blocking layer, the electron transport layer, and the electron injecting layer, if necessary.

When the compound represented by Chemical Formula 1 is used as a light emitting layer material, the compound represented by Chemical Formula 1 may serve as a light emitting host, and in this case, the light emitting layer further includes a dopant. As an example, the compound represented by Chemical Formula 1 may be used as a p-type or n-type phosphorescent host.

Any dopant known in the art may be used as a dopant capable of being used together with the compound represented by Chemical Formula 1. For example, the dopant used together when the compound represented by Chemical Formula 1 is used as a phosphorescent host may be Ir(ppy)₃, and the like.

The organic light emitting device according to the present invention may be manufactured using materials and methods known in the art, except that the compound represented by Chemical Formula 1 is included in one or more of the organic material layers.

The compound represented by Chemical Formula 1 may be used alone to constitute one or more of the organic material layers of the organic light emitting device. However, if necessary, the organic material layer may be constituted by mixing the compound represented by Chemical Formula 1 with other materials.

In the organic light emitting device according to an embodiment of the present invention, examples of materials other than the compound represented by Chemical Formula 1 which may be used are shown below, but these are provided only by way of example, and are not intended to limit the scope of the present invention, and they may be replaced with other materials known in the art.

As the anode material, materials having a relatively large work function may be used, and a transparent conductive oxide, a metal, a conductive polymer, or the like, may be used.

Specific examples of the anode material include metals such as vanadium, chromium, copper, zinc, gold or their alloys; metal oxides such as zinc oxide, tin oxide, indium tin oxide (ITO), or indium zinc oxide (IZO); combinations of metals and oxides such as Zno: Al or SnO₂: Sb; conductive polymers such as poly(3-methylthiophene), poly[3,4-(ethylene-1,2-dioxy)thiophene] (PEDOT), polypyrrole, polyaniline; and the like, but the anode material is not limited thereto.

As the cathode material, materials having a relatively low work function may be used, and a metal, a metal oxide, a conductive polymer, or the like, may be used.

Specific examples of the cathode material include metals such as magnesium, calcium, sodium, potassium, titanium, yttrium, gadolinium, aluminum, silver, tin, lead, and their alloys; multilayered structured materials such as LiF/Al or LiO₂/Al; and the like, but the cathode material is not limited thereto.

As a hole injection material, a hole injection material known in the related art may also be used, and it is possible to use, for example, a phthalocyanine compound such as copper phthalocyanine, disclosed in U.S. Pat. No. 4,356,429, or starburst-type amine derivatives described in the document [Advanced Material, 6, p. 677 (1994)], for example, tris(4-carbazoyl-9-ylphenyl)amine(TCTA), 4,4′,4″-tri[phenyl(m-tolyl)amino]triphenylamine(m-MTDATA), 1,3,5-tris[4-(3-methylphenylamino)phenyl]benzene(m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid, polyaniline/poly(4-styrene-sulfonate), and the like.

As the hole injecting material, any known hole injecting materials may be used.

As the hole transport material, a pyrazoline derivative, an arylamine derivative, a stilbene derivative, a triphenyldiamine derivative, or the like, may be used, and a low molecular weight material or a high molecular weight material may be used.

As the electron transport material, an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, may be used, and polymer materials as well as low molecular materials may be used.

As the electron injecting material, for example, LiF is generally used in the art, but the present invention is not limited thereto.

As the light emitting material, red, green or blue light emitting material may be used, and if necessary, two or more light emitting materials may be mixed to be used. Further, the light emitting material may be a fluorescent material, but may also be a phosphorescent material. As the light emitting material, a material that emits light by coupling holes and electrons injected from the anode and cathode, respectively, may be used alone, but materials in which both the host material and the dopant material are involved in light emitting may also be used.

The organic light emitting device according to an embodiment of the present invention may be a front emission type, a back emission type, or a double-sided emission type, depending on the material used.

The heterocyclic compound according to an embodiment of the present invention may act on a principle similar to a case applied to organic light emitting devices used among organic electronic devices, including organic solar cells, organic photoconductors, organic transistors, and the like.

Hereinafter, although the present disclosure has been described in detail with reference to Examples, it should be understood that these Examples are provided for illustrative purposes and do not limit the scope of the present disclosure.

### Preparation Example 1

1) Preparation of Compound 1-1

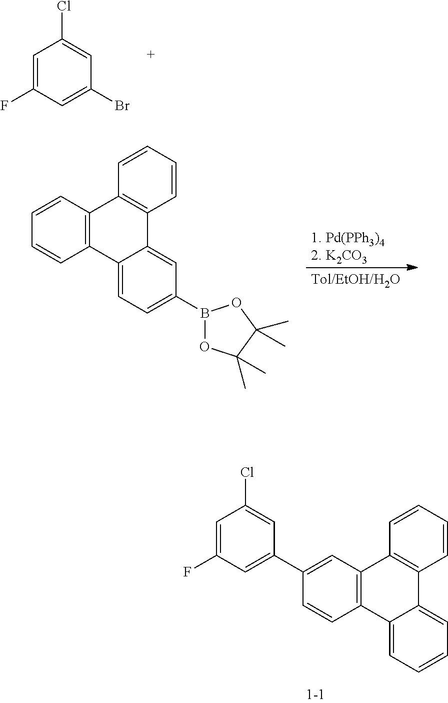 [SMILES: Fc1cc(Cl)cc(Br)c1 ; CC1(C)OB(c2ccc3c4ccccc4c4ccccc4c3c2)OC1(C)C ; Fc1cc(Cl)cc(-c2ccc3c4ccccc4c4ccccc4c3c2)c1]

To a 2 L round bottom flask, benzene, 50 g (238.73 mmol, 1 eq.) of 1-bromo-3-chloro-5-fluorobenzene, 84.6 g (238.73 mmol, 1 eq.) of 2-triphenylenylpinacolborane, 13.8 g (11.94 mmol, 0.05 eq.) of Pd(pph₃)₄, and 99.0 g (716.20 mmol, 3 eq.) of K₂CO₃ were added, after which toluene/EtOH/H₂O (600 ml/120 ml/120 ml) was added, and the obtained mixture was stirred under reflux.

After completion of the reaction, the mixture was extracted with MC/H₂O and the MC layer was dried over MgSO₄. Purification was performed using a silica-gel column to obtain 62.0 g of Compound 1-1 at a yield of 73%.

2) Preparation of Compound 1-2

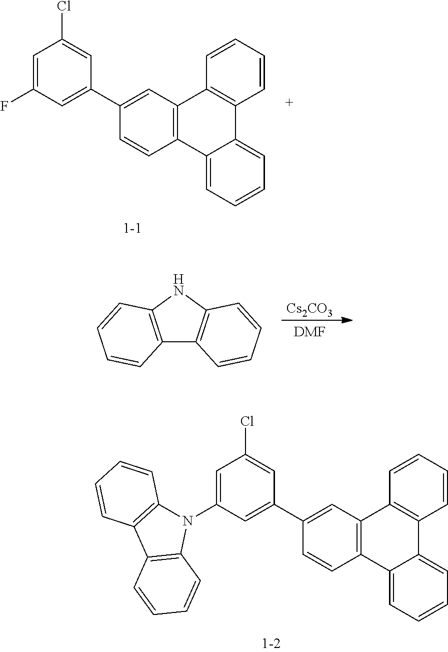

To a 2 L round bottom flask, 62 g (173.76 mmol, 1.1 eq.) of Compound 1-1, 26.4 g (157.96 mmol, 1 eq.) of 9H-carbazole, and 205.9 g (631.84 mmol, 4 eq.) of CsCO₃ were added, after which Dimethyl Formamide (DMF) (800 ml) was added, and the obtained mixture was stirred under reflux.

After completion of the reaction, the mixture was precipitated by adding H₂O and then filtered. The precipitate was extracted by dissolution in MC, and the MC layer was dried over MgSO₄. Purification was performed using a silica-gel column to obtain 69.2 g of Compound 1-2 at a yield of 87%.

3) Preparation of Compound 1-3

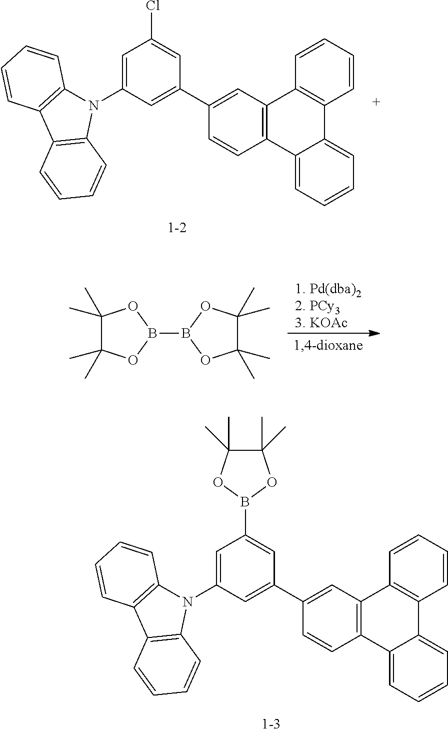

To a 1 L round bottom flask, 69.2 g (137.29 mmol, 1 eq.) of compound 1-2, 52.3 g (205.94 mmol, 1.5 eq.) of bis(pinacolato)diboron, 4.0 g (6.87 mmol, 0.05 eq.) of Pd(dba)₂, 3.9 g (13.73 mmol, 0.1 eq.) of PCy3, and 53.9 g (549.17 mmol, 4 eq.) of Potassium acetate (KOAc) were added, after which 600 ml of 1,4-dioxane was added, and the obtained mixture was stirred under reflux.

After completion of the reaction, the mixture was extracted with MC/H₂O and the MC layer was dried over MgSO₄. Purification was performed using a silica-gel column to obtain 70.3 g of Compound 1-3 at a yield of 86%.

4) Preparation of Target Compound

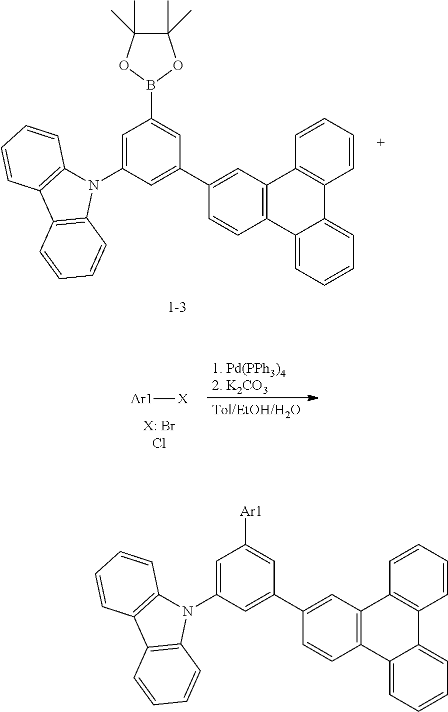

To a 500 ml round bottom flask, Compound 1-3 (1 eq.), Ar1-X (1 eq.) as described in Table 1 below, Pd(pph₃)₄ (0.05 eq.) and K₂CO₃ (3 eq.) were added, after which toluene/EtOH was added, and the obtained mixtures were stirred under reflux.

After completion of the reaction, the mixtures were extracted with MC/H₂O and the MC layers were dried over MgSO₄. Purification was performed using silica-gel columns to obtain the compounds of Table 1 below at the yields listed therein.

TABLE 1

| Com- | | Yield | |
|---|---|---|---|
| pound | Ar1-X | (%) | Structure |
| 2 | 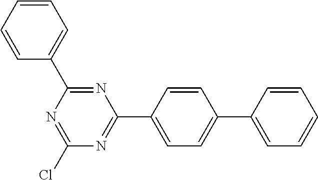 | 81 | 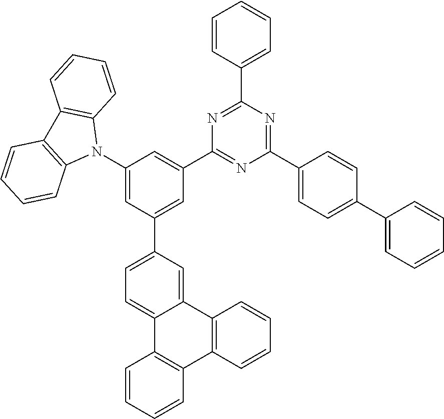 [SMILES: c1ccc(-c2ccc(-c3nc(-c4ccccc4)nc(-c4cc(-c5ccc6c7ccccc7c7ccccc7c6c5)cc(-n5c6ccccc6c6ccccc65)c4)n3)cc2)cc1] |
| 4 | 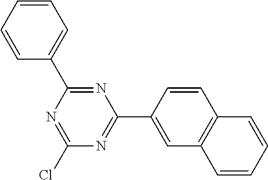 | 79 | 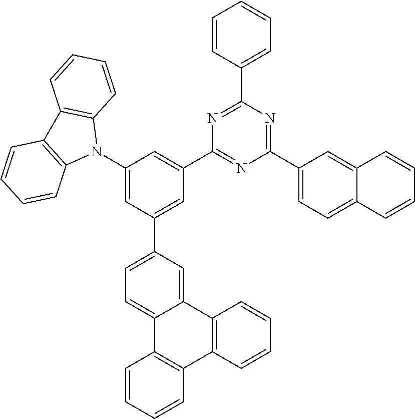 [SMILES: c1ccc(-c2nc(-c3cc(-c4ccc5c6ccccc6c6ccccc6c5c4)cc(-n4c5ccccc5c5ccccc54)c3)nc(-c3ccc4ccccc4c3)n2)cc1] |
| 321 | 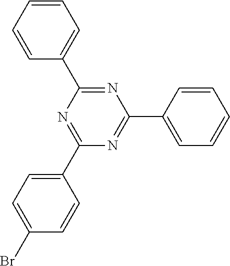 | 89 | 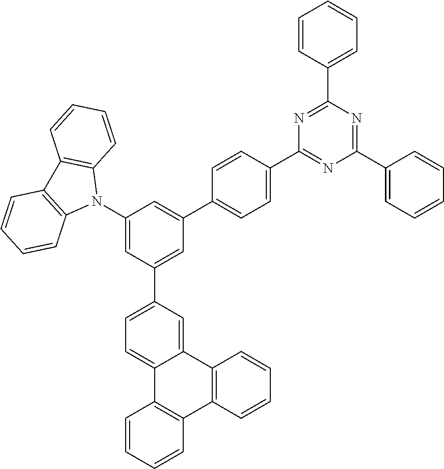 [SMILES: c1ccc(-c2nc(-c3ccccc3)nc(-c3ccc(-c4cc(-c5ccc6c7ccccc7c7ccccc7c6c5)cc(-n5c6ccccc6c6ccccc65)c4)cc3)n2)cc1] |
| 324 | 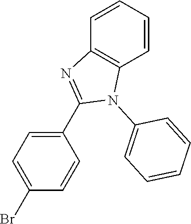 | 86 | 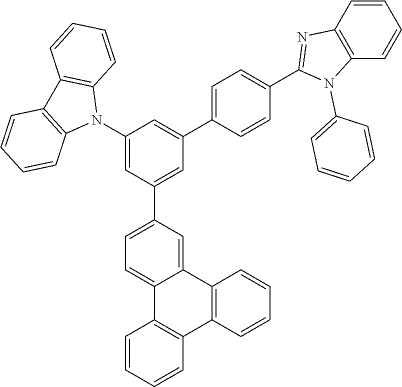 [SMILES: c1ccc(-n2c(-c3ccc(-c4cc(-c5ccc6c7ccccc7c7ccccc7c6c5)cc(-n5c6ccccc6c6ccccc65)c4)cc3)nc3ccccc32)cc1] |
| 327 | 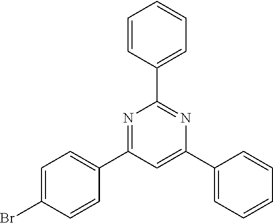 | 79 | 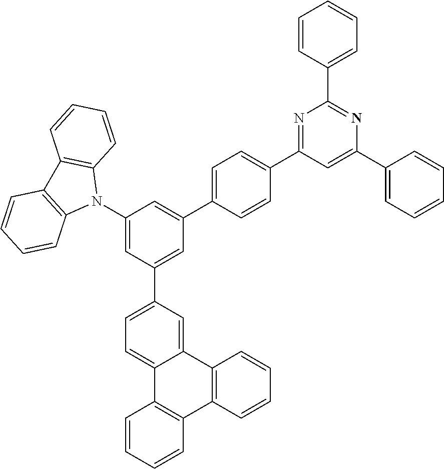 [SMILES: c1ccc(-c2cc(-c3ccc(-c4cc(-c5ccc6c7ccccc7c7ccccc7c6c5)cc(-n5c6ccccc6c6ccccc65)c4)cc3)nc(-c3ccccc3)n2)cc1] |

### Preparation Example 2

1) Preparation of Compound 2-1

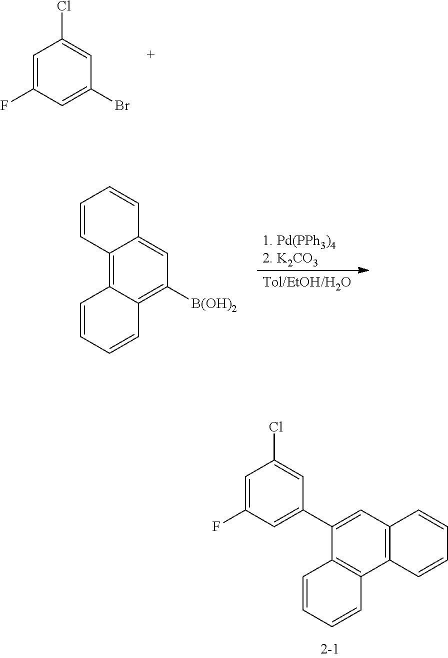 [SMILES: Fc1cc(Cl)cc(Br)c1 ; OB(O)c1cc2ccccc2c2ccccc12 ; Fc1cc(Cl)cc(-c2cc3ccccc3c3ccccc23)c1]

To a 5 L round bottom flask, benzene, 94.3 g (450.35 mmol, 1 eq.) of 1-bromo-3-chloro-5-fluorobenzene, 100 g (450.35 mmol, 1 eq.) of 9-phenanthrenyl boronic acid, 26.0 g (22.52 mmol, 0.05 eq.) of Pd(pph₃)₄, and 186.7 g (1351.05 mmol, 3 eq.) of K₂CO₃ were added, after which toluene/EtOH/H₂O (2 L/400 ml/400 ml) was added, and the obtained mixture was stirred under reflux.

After completion of the reaction, the mixture was extracted with MC/H₂O and the MC layer was dried over MgSO₄. Purification was performed using a silica-gel column to obtain 103.1 g of Compound 2-1 at a yield of 75%.

2) Preparation of Compound 2-2

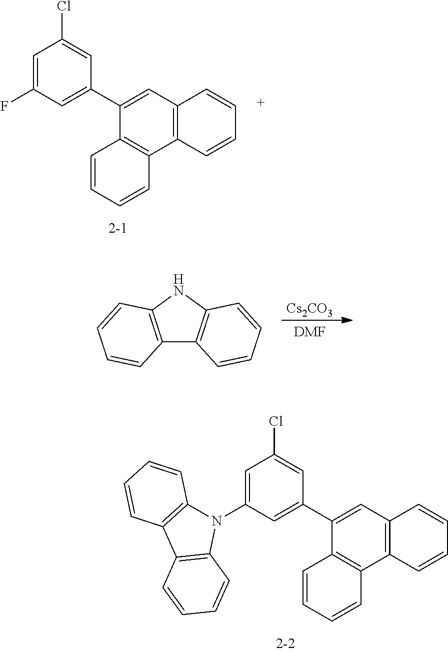

To a 3 L round bottom flask, 103.1 g (336.09 mmol, 1.1 eq.) of Compound 2-1, 51.1 g (305.54 mmol, 1 eq.) of 9-H-carbazole, and 398.2 g (1222.16 mmol, 4 eq.) of CsCO₃ were added, after which DMF (1.5 L) was added, and the obtained mixture was stirred under reflux.

After completion of the reaction, the mixture was precipitated by adding H₂O and then filtered. The precipitate was extracted by dissolution in MC, and the MC layer was dried over MgSO₄. Purification was performed using a silica-gel column to obtain 108.2 g of Compound 2-2 at a yield of 78%.

3) Preparation of Compound 2-3

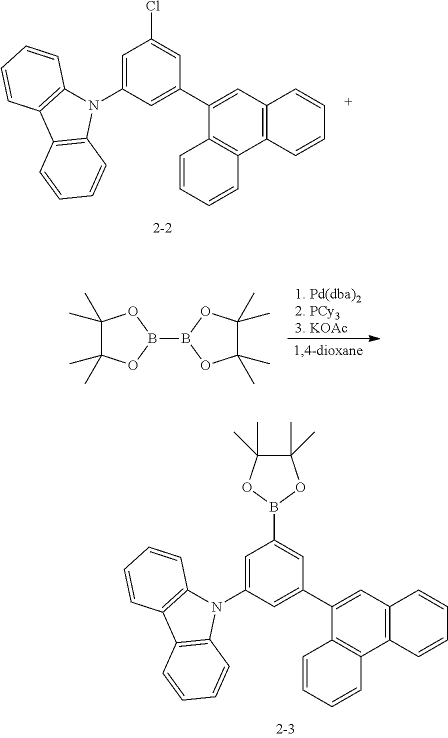

To a 2 L round bottom flask, 108.2 g (238.34 mmol, 2 eq.) of Compound 2-2, 90.8 g (357.51 mmol, 1.5 eq.) of bis(pinacolato)diboron, 6.9 g (11.92 mmol, 0.05 eq.) of Pd(dba)₂, 6.7 g (23.83 mmol, 0.1 eq.) of PCy3, and 93.6 g (953.36 mmol, 4 eq.) of KOAc were added, after which 1.2 L of 1,4-dioxane was added, and the obtained mixture was stirred under reflux.

After completion of the reaction, the mixture was extracted with MC/H₂O, and the MC layer was dried over MgSO₄. Purification was performed using a silica-gel column to obtain 117.1 g of Compound 2-3 at a yield of 90%.

4) Preparation of Target Compound

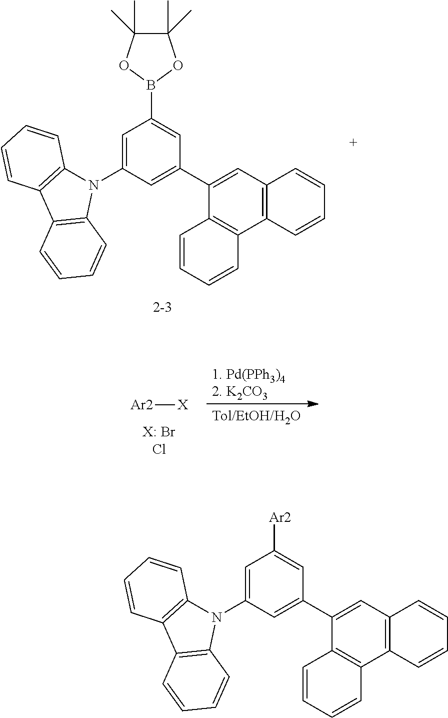

To a 500 ml round bottom flask, Compound 2-3 (1 eq.), Ar2-X (2 eq.) as described in Table 2 below, Pd(pph₃)₄ (0.05 eq.) and K₂CO₃ (3 eq.) were added, after which toluene/EtOH was added, and the obtained mixtures were stirred under reflux.

After completion of the reaction, the mixtures were extracted with MC/H₂O, and the MC layers were dried over MgSO₄. Purification was performed using silica-gel columns to obtain the compounds of Table 2 below at the yields listed therein.

TABLE 2

| Com- | | | |
|---|---|---|---|
| pound | Ar2-X | Yield(%) | Structure |
| 242 | 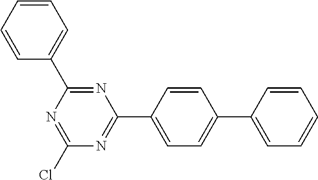 | 83 | 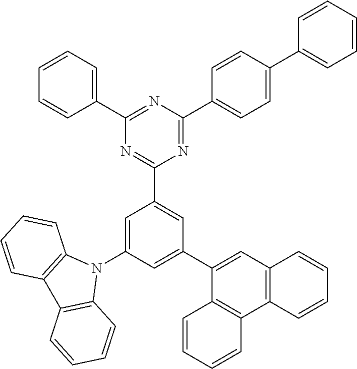 [SMILES: c1ccc(-c2ccc(-c3nc(-c4ccccc4)nc(-c4cc(-c5cc6ccccc6c6ccccc56)cc(-n5c6ccccc6c6ccccc65)c4)n3)cc2)cc1] |
| 244 | 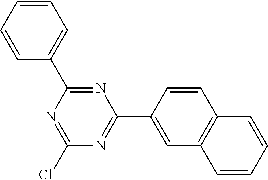 | 89 | 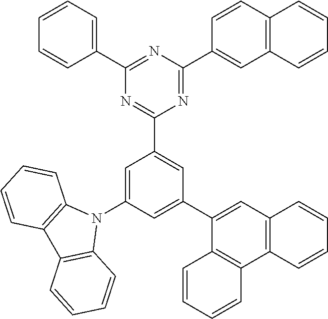 [SMILES: c1ccc(-c2nc(-c3cc(-c4cc5ccccc5c5ccccc45)cc(-n4c5ccccc5c5ccccc54)c3)nc(-c3ccc4ccccc4c3)n2)cc1] |
| 322 | 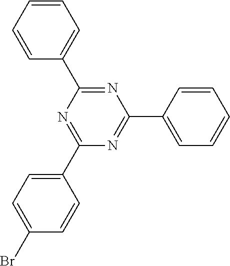 | 81 | 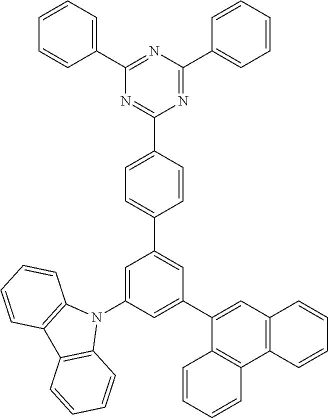 [SMILES: c1ccc(-c2nc(-c3ccccc3)nc(-c3ccc(-c4cc(-c5cc6ccccc6c6ccccc56)cc(-n5c6ccccc6c6ccccc65)c4)cc3)n2)cc1] |
| 328 | 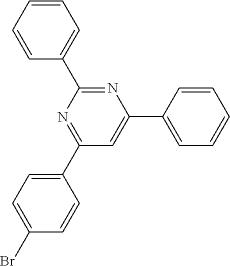 | 86 | 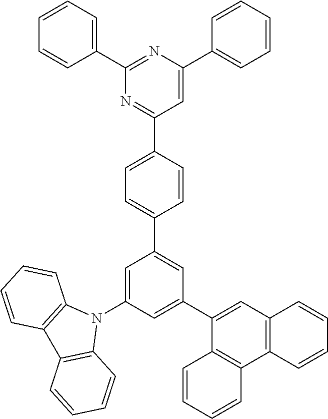 [SMILES: c1ccc(-c2cc(-c3ccc(-c4cc(-c5cc6ccccc6c6ccccc56)cc(-n5c6ccccc6c6ccccc65)c4)cc3)nc(-c3ccccc3)n2)cc1] |

### Preparation Example 3

1) Preparation of Compound 3-1

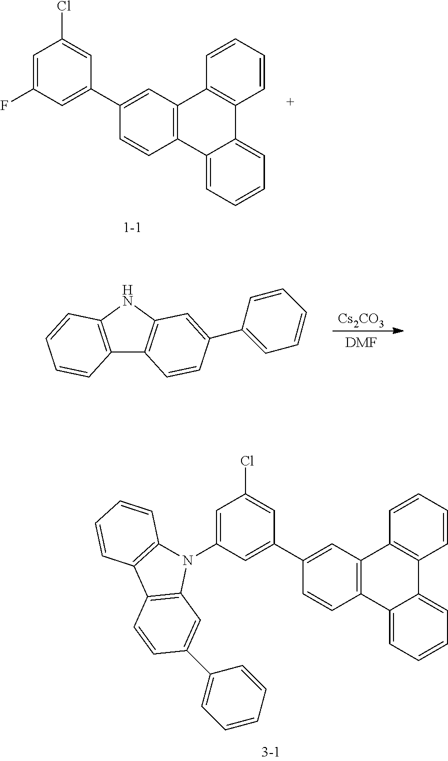

To a 2 L round bottom flask, 50 g (140.13 mmol, 1.1 eq.) of Compound 1-1, 31.0 g (127.39 mmol, 1 eq.) of 2-phenyl 9H-carbazole, and 166.0 g (509.56 mmol, 4 eq.) of CsCO₃ were added, after which DMF (600 ml) was added, and the obtained mixture was stirred under reflux.

After completion of the reaction, the mixture was precipitated by adding H₂O and then filtered. The precipitate was extracted by dissolution in MC, and the MC layer was dried over MgSO₄. Purification was performed using a silica-gel column to obtain 61.3 g of Compound 3-1 at a yield of 83%.

2) Preparation of Compound 3-2

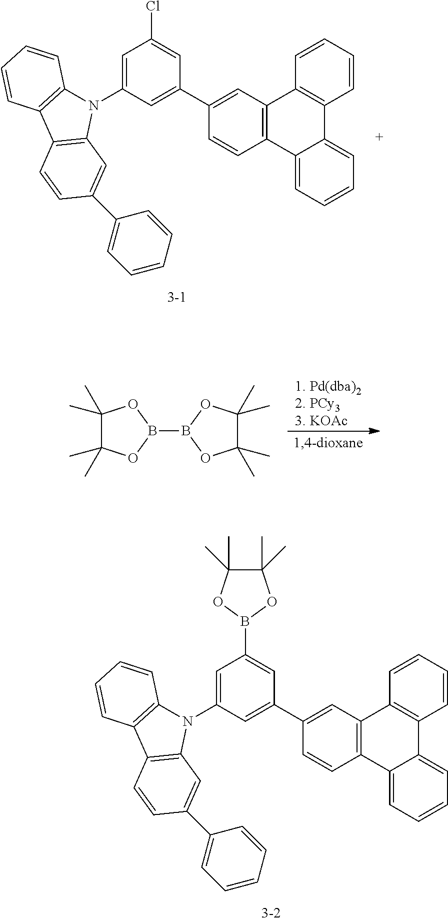

To a 1 L round bottom flask, 61.3 g (105.67 mmol, 1 eq.) of Compound 3-1, 40.3 g (158.50 mmol, 1.5 eq.) of bis(pinacolato)diboron, 3.0 g (5.28 mmol, 0.05 eq.) of Pd(dba)₂, 3.0 g (10.57 mmol, 0.1 eq.) of PCy3, and 41.5 g (442.68 mmol, 4 eq.) of KOAc were added, after which 500 ml of 1,4-dioxane was added, and the obtained mixture was stirred under reflux.

After completion of the reaction, the mixture was extracted with MC/H₂O, and the MC layer was dried over MgSO₄. Purification was performed using a silica-gel column to obtain 51.6 g of Compound 3-2 at a yield of 82%.

3) Preparation of Target Compound

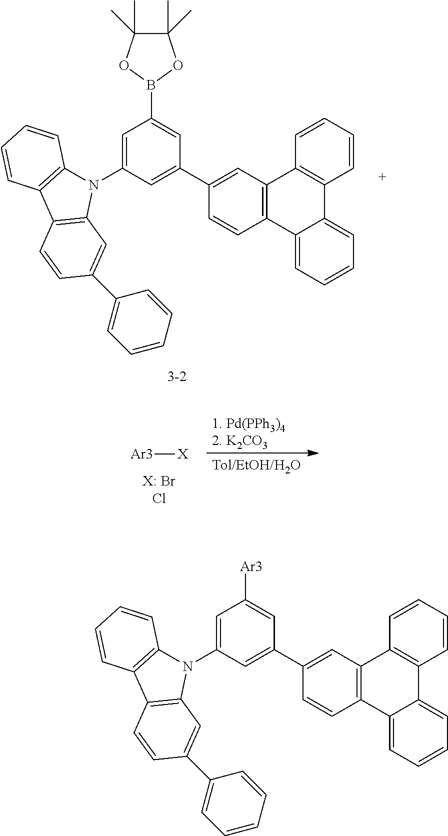

To a 500 ml round bottom flask, Compound 3-2 (1 eq.), Ar1-X (1 eq.) as described in Table 1 below, Pd(pph₃)₄ (0.05 eq.) and K₂CO₃ (3 eq.) were added, after which toluene/EtOH was added, and the obtained mixtures were stirred under reflux.

After completion of the reaction, the mixtures were extracted with MC/H₂O, and the MC layers were dried over MgSO₄. Purification was performed using silica-gel columns to obtain the compounds of Table 3 below at the yields listed therein.

TABLE 3

| Com- | | | |
|---|---|---|---|
| pound | Ar1-X | Yield(%) | Structure |
| 21 | 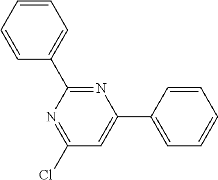 | 77 | 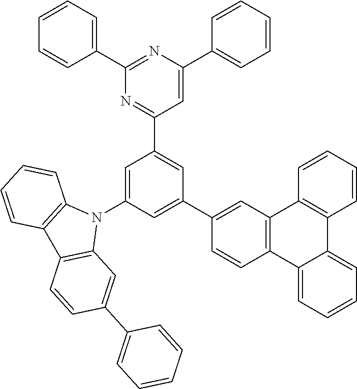 [SMILES: c1ccc(-c2ccc3c4ccccc4n(-c4cc(-c5ccc6c7ccccc7c7ccccc7c6c5)cc(-c5cc(-c6ccccc6)nc(-c6ccccc6)n5)c4)c3c2)cc1] |
| 101 | 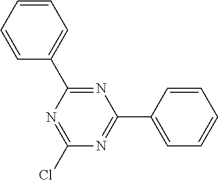 | 87 | 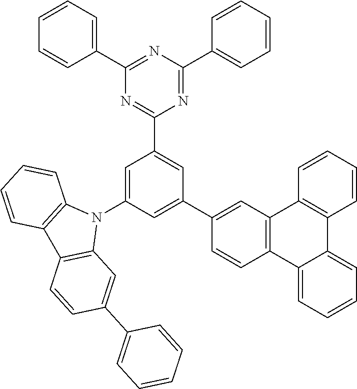 [SMILES: c1ccc(-c2ccc3c4ccccc4n(-c4cc(-c5ccc6c7ccccc7c7ccccc7c6c5)cc(-c5nc(-c6ccccc6)nc(-c6ccccc6)n5)c4)c3c2)cc1] |
| 323 | 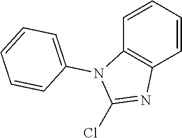 | 83 | 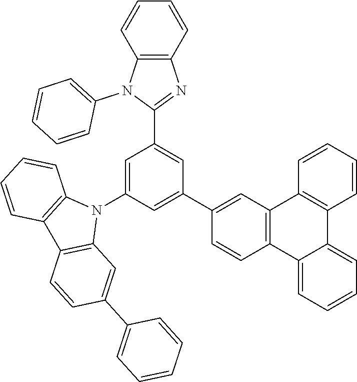 [SMILES: c1ccc(-c2ccc3c4ccccc4n(-c4cc(-c5ccc6c7ccccc7c7ccccc7c6c5)cc(-c5nc6ccccc6n5-c5ccccc5)c4)c3c2)cc1] |

### Preparation Example 4

1) Preparation of Compound 4-1

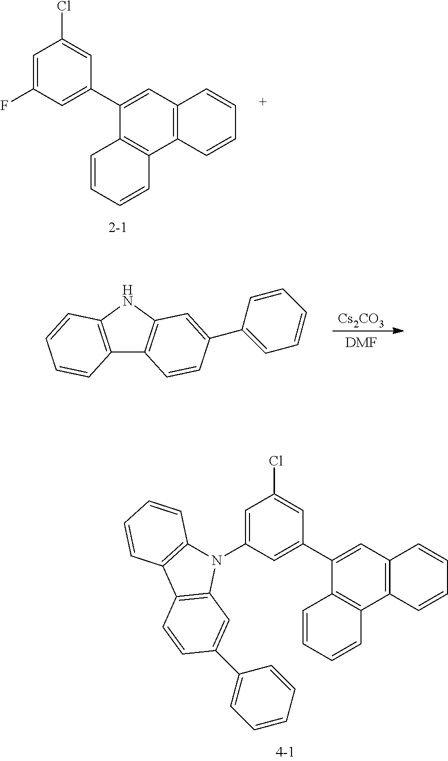

To a 3 L round bottom flask, 50 g (162.99 mmol, 1.1 eq.) of Compound 2-1, 36.1 g (148.18 mmol, 1 eq.) of 2-phenyl 9H-carbazole, and 193.1 g (592.72 mmol, 4 eq.) of CsCO₃ were added, after which DMF (800 ml) was added, and the obtained mixture was stirred under reflux.

After completion of the reaction, the mixture was precipitated by adding H₂O and then filtered. The precipitate was extracted by dissolution in MC, and the MC layer was dried over MgSO₄. Purification was performed using a silica-gel column to obtain 62.0 g of Compound 4-1 at a yield of 79%.

2) Preparation of Compound 4-2

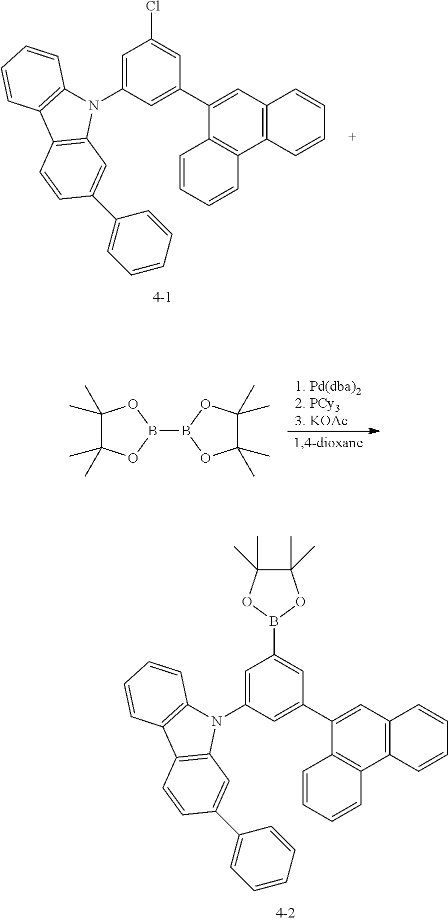

To a 2 L round bottom flask, 62.0 g (116.97 mmol, 1 eq.) of Compound 4-1, 44.6 g (175.45 mmol, 1.5 eq.) of bis(pinacolato)diboron, 3.4 g (5.85 mmol, 0.05 eq.) of Pd(dba)₂, 3.3 g (11.70 mmol, 0.1 eq.) of PCy₃, and 45.9 g (467.88 mmol, 4 eq.) of KOAc were added, after which 500 ml of 1,4-dioxane was added, and the obtained mixture was stirred under reflux.

After completion of the reaction, the mixture was extracted with MC/H₂O, and the MC layer was dried over MgSO₄. Purification was performed using a silica-gel column to obtain 61.3 g of Compound 4-2 at a yield of 84%.

3) Preparation of Target Compound

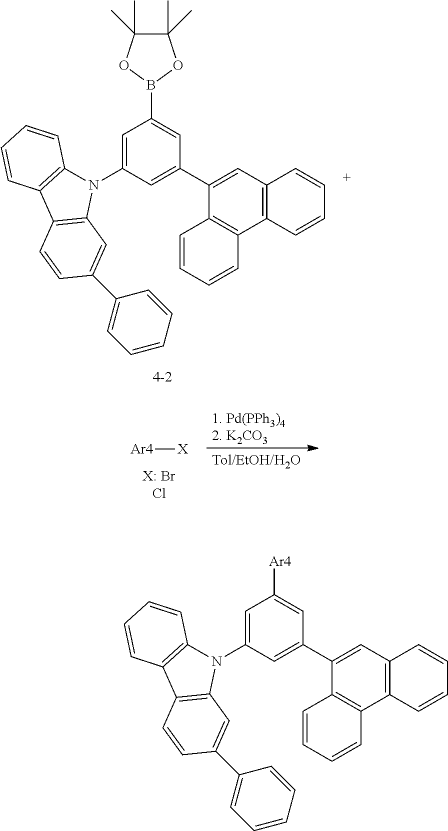

To a 500 ml round bottom flask, Compound 4-2 (1 eq.), Ar1-X (4 eq.) as described in Table 4 below, Pd(pph₃)₄ (0.05 eq.) and K₂CO₃ (3 eq.) were added, after which toluene/EtOH was added, and the obtained mixtures were stirred under reflux.

After completion of the reaction, the mixtures were extracted with MC/H₂O, and the MC layers were dried over MgSO₄. Purification was performed using silica-gel columns to obtain the compounds of Table 4 below at the yields listed therein.

| Com- | | | |
|---|---|---|---|
| pound | Ar4-X | Yield(%) | Structure |
| 261 | 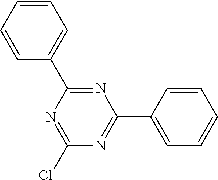 | 81 | 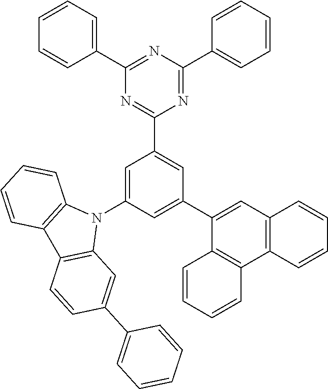 [SMILES: c1ccc(-c2ccc3c4ccccc4n(-c4cc(-c5nc(-c6ccccc6)nc(-c6ccccc6)n5)cc(-c5cc6ccccc6c6ccccc56)c4)c3c2)cc1] |
| 325 | 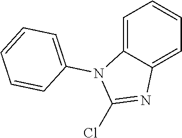 | 73 | 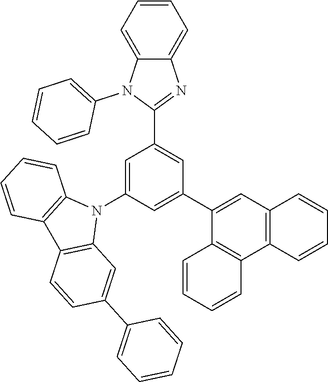 [SMILES: c1ccc(-c2ccc3c4ccccc4n(-c4cc(-c5cc6ccccc6c6ccccc56)cc(-c5nc6ccccc6n5-c5ccccc5)c4)c3c2)cc1] |
| 326 | 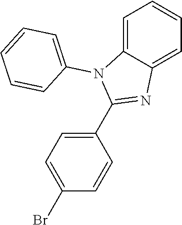 | 76 | 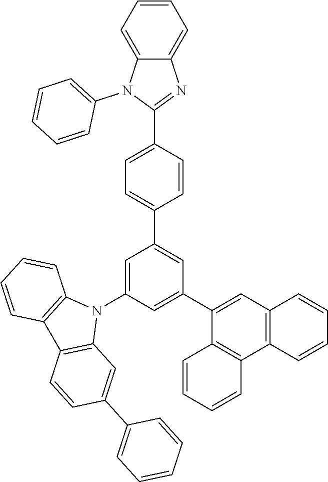 [SMILES: c1ccc(-c2ccc3c4ccccc4n(-c4cc(-c5ccc(-c6nc7ccccc7n6-c6ccccc6)cc5)cc(-c5cc6ccccc6c6ccccc56)c4)c3c2)cc1] |

The compounds were prepared in the same manner as described in the above Preparation Examples, and results obtained by confirming the synthesis are shown in Tables 5 and 6 below.

TABLE 5

| Com- | |
|---|---|
| pound | ¹H NMR (CDCl₃), 300 MHz) |
| 2 | 9.27 (1H, s), 8.79 (1H, d), 8.55 (1H, d), 8.37~8.19 (12H, m), |
| | 7.94~7.85 (4H, m), 7.75~7.35 (16H, m), 7.20~7.16 (2H, m) |
| 4 | 9.27 (1H, s), 8.79 (1H, d), 8.55 (1H, s), 8.37~8.31 (8H, m), |
| | 8.23~8.19 (2H, m), 8.06~7.90 (6H, m), 7.70~7.50 (12H, m), |
| | 7.35 (1H, m), 7.20~7.16 (2H, m) |
| 21 | 9.27 (1H, s), 8.79 (1H, d), 8.55 (1H, d), 8.37~8.30 (9H, m), |
| | 7.94~7.90 (5H, m), 7.75~7.35 (18H, m), 7.16 (1H, t) |
| 82 | 9.27 (1H, s), 8.79 (1H, d), 8.55 (1H, d), 8.37~8.30 (8H, m), |
| | 8.19 (1H, d), 7.96~7.90 (4H, m), 7.75~7.35 (16H, m), |
| | 7.25~7.16 (4H, m) |
| 84 | 9.27 (1H, s), 9.09 (1H, s), 8.79 (1H, d), 8.55~8.49 (2H, m), |
| | 8.37~8.30 (8H, m), 8.19~7.90 (6H, m), 7.70~7.50 (12H, m), |
| | 7.35 (1H, m), 7.20~7.16 (2H, m) |
| 101 | 9.27 (1H, s), 8.79 (1H, d), 8.55 (1H, d), 8.37~8.30 (11H, m), |
| | 7.94~7.90 (3H, m), 7.75~7.64 (7H, m), 7.52~7.41 (11H, m), |
| | 7.16 (1H, t) |
| 181 | 9.08 (1H, d), 8.84 (1H, d), 8.55 (1H, d), 8.37~8.23 (7H, m), |
| | 8.05 (1H, s), 7.94~7.90 (6H, m), 7.70~7.41 (17H, m), 7.16 (1H, |
| | t) |
| 242 | 9.08 (1H, d), 8.84 (1H, d), 8.55 (1H, d), 8.36~8.27 (5H, m), |
| | 8.19 (1H, d), 8.05 (1H, s), 7.96~7.90 (5H, m), 7.75~7.35 (15H, |
| | m), 7.25~7.16 (4H, m) |
| 244 | 9.09~9.08 (2H, m), 8.84 (1H, d), 8.55~8.49 (2H, m), |
| | 8.36~7.90 (13H, m), 7.70~7.50 (11H, m), 7.35 (1H, t), |
| | 7.20~7.16 (2H, m) |
| 248 | 9.08 (1H, d), 8.84 (1H, d), 8.55 (2H, m), 8.36~8.19 (7H, m), |
| | 8.05 (1H, s), 7.94~7.90 (4H, m), 7.70~7.50 (11H, m), 7.35 (2H, |
| | m), 7.20~7.16 (4H, m) |
| 261 | 9.08 (1H, d), 8.84 (1H, d), 8.55 (1H, d), 8.36~8.27 (8H, m), |
| | 8.05 (1H, s), 7.94~7.90 (4H, m), 7.75~7.63 (7H, m), |
| | 7.50~7.35 (10H, m), 7.16 (1H, t) |
| 321 | 9.27 (1H, s), 8.79 (1H, d), 8.55 (1H, d), 8.37~8.30 (10H, m), |
| | 8.19 (1H, d), 7.96~7.90 (4H, m), 7.70~7.50 (13H, m), 7.35 (1H, |
| | t), 7.25~7.16 (4H, m) |
| 322 | 9.08 (1H, d), 8.84 (1H, d), 8.55 (1H, d), 8.36~8.27 (7H, m), |
| | 8.19 (1H, d), 8.05 (1H, s), 7.96~7.90 (5H, m), 7.70~7.50 (12H, |
| | m), 7.35 (1H, t), 7.25~7.16 (4H, m) |
| 323 | 9.27 (1H, s), 8.79 (1H, d), 8.56~8.55 (2H, m), 8.33~8.30 (7H, |
| | m), 7.94~7.90 (3H, m), 7.81~7.30 (20H, m), 7.16 (1H, t) |
| 324 | 9.27 (1H, s), 8.79 (1H, d), 8.56~8.55 (2H, m), 8.37~8.30 (6H, |
| | m), 8.19 (1H, d), 7.96~7.90 (4H, m), 7.81 (1H, d), |
| | 7.70~7.48 (11H, m), 7.38~7.16 (8H, m) |
| 325 | 9.08 (1H, d), 8.84 (1H, d), 8.56~8.55 (2H, m), 8.31~8.27 (4H, |
| | m), 8.05 (1H, s), 7.94~7.35 (23H, m), 7.16 (1H, t) |
| 326 | 9.08 (1H, d), 8.84 (1H, d), 8.56~8.55 (2H, m), 8.31~8.27 (4H, |
| | m), 8.05 (1H, s), 7.90~7.16 (28H, m) |
| 327 | 9.27 (1H, s), 8.79 (1H, d), 8.55 (1H, d), 8.37~8.19 (12H, m), |
| | 7.94~7.85 (6H, m), 7.70~7.49 (13H, m), 7.35 (1H, t), |
| | 7.20~7.16 (2H, m) |
| 328 | 9.08 (1H, d), 8.84 (1H, d), 8.55 (1H, d), 8.35~8.19 (9H, m), |
| | 8.05 (1H, s), 7.94~7.85 (7H, m), 7.70~7.49 (12H, m), 7.35 (1H, |
| | t), 7.20~7.16 (2H, m) |

TABLE 6

| Com- | | Com- | |
|---|---|---|---|
| pound | FD-MS | pound | FD-MS |
| 1 | m/z = 699.27 | 2 | m/z = 775.30 |
| | C52H33N3 = 699.86 | | C58H37N3 = 775.96 |
| 3 | m/z = 775.30 | 4 | m/z = 749.28 |
| | C58H37N3 = 775.96 | | C56H35N3 = 749.92 |
| 5 | m/z = 851.33 | 6 | m/z = 799.30 |
| | C64H41N3 = 852.05 | | C60H37N3 = 799.98 |
| 7 | m/z = 823.30 | 8 | m/z = 788.29 |
| | C62H37N3 = 824.00 | | C58H36N4 = 788.95 |
| 9 | m/z = 789.28 | 10 | m/z = 789.28 |
| | C58H35N3O = 789.94 | | C58H35N3O = 789.94 |
| 11 | m/z = 805.26 | 12 | m/z = 805.26 |
| | C58H35N3S = 806.00 | | C58H35N3S = 806.00 |
| 13 | m/z = 864.33 | 14 | m/z = 864.33 |
| | C64H40N4 = 865.05 | | C64H40N4 = 865.05 |
| 15 | m/z = 815.33 | 16 | m/z = 815.33 |
| | C61H41N3 = 816.02 | | C61H41N3 = 816.02 |
| 17 | m/z = 939.36 | 18 | m/z = 939.36 |
| | C71H45N3 = 940.16 | | C71H45N3 = 940.16 |
| 19 | m/z = 937.35 | 20 | m/z = 937.35 |
| | C71H43N3 = 938.15 | | C71H43N3 = 938.15 |
| 21 | m/z = 775.30 | 22 | m/z = 851.33 |
| | C58H37N3 = 775.96 | | C64H41N3 = 852.05 |
| 23 | m/z = 851.33 | 24 | m/z = 825.31 |
| | C64H41N3 = 852.05 | | C62H39N3 = 826.02 |
| 25 | m/z = 927.36 | 26 | m/z = 875.33 |
| | C70H45N3 = 928.15 | | C66H41N3 = 876.08 |
| 27 | m/z = 899.33 | 28 | m/z = 864.33 |
| | C68H41N3 = 900.10 | | C64H40N4 = 865.05 |
| 29 | m/z = 865.31 | 30 | m/z = 865.31 |
| | C64H39N3O = 866.04 | | C64H39N3O = 866.04 |
| 31 | m/z = 881.29 | 32 | m/z = 881.29 |
| | C64H39N3S = 882.10 | | C64H39N3S = 882.10 |
| 33 | m/z = 940.36 | 34 | m/z = 940.36 |
| | C70H44N4 = 941.15 | | C70H44N4 = 941.15 |
| 35 | m/z = 891.36 | 36 | m/z = 891.36 |
| | C67H45N3 = 892.12 | | C67H45N3 = 892.12 |
| 37 | m/z = 1015.39 | 38 | m/z = 1015.39 |
| | C77H49N3 = 1016.26 | | C77H49N3 = 1016.26 |
| 39 | m/z = 1013.38 | 40 | m/z = 1013.38 |
| | C77H47N3 = 1014.24 | | C77H47N3 = 1014.24 |
| 41 | m/z = 699.27 | 42 | m/z = 775.30 |
| | C52H33N3 = 699.86 | | C58H37N3 = 775.96 |
| 43 | m/z = 775.30 | 44 | m/z = 749.28 |
| | C58H37N3 = 775.96 | | C56H35N3 = 749.92 |
| 45 | m/z = 851.33 | 46 | m/z = 799.30 |
| | C64H41N3 = 852.05 | | C60H37N3 = 799.98 |
| 47 | m/z = 823.30 | 48 | m/z = 788.29 |
| | C62H37N3 = 824.00 | | C58H36N4 = 788.95 |
| 49 | m/z = 789.28 | 50 | m/z = 789.28 |
| | C58H35N3O = 789.94 | | C58H35N3O = 789.94 |
| 51 | m/z = 805.26 | 52 | m/z = 805.26 |
| | C58H35N3S = 806.00 | | C58H35N3S = 806.00 |
| 53 | m/z = 864.33 | 54 | m/z = 864.33 |
| | C64H40N4 = 865.05 | | C64H40N4 = 865.05 |
| 55 | m/z = 815.33 | 56 | m/z = 815.33 |
| | C61H41N3 = 816.02 | | C61H41N3 = 816.02 |
| 57 | m/z = 939.36 | 58 | m/z = 939.36 |
| | C71H45N3 = 940.16 | | C71H45N3 = 940.16 |
| 59 | m/z = 937.35 | 60 | m/z = 937.35 |
| | C71H43N3 = 938.15 | | C71H43N3 = 938.15 |
| 61 | m/z = 775.30 | 62 | m/z = 851.33 |
| | C58H37N3 = 775.96 | | C64H41N3 = 852.05 |
| 63 | m/z = 851.33 | 64 | m/z = 825.31 |
| | C64H41N3 = 852.05 | | C62H39N3 = 826.02 |
| 65 | m/z = 927.36 | 66 | m/z = 875.33 |
| | C70H45N3 = 928.15 | | C66H41N3 = 876.08 |
| 67 | m/z = 899.33 | 68 | m/z = 864.33 |
| | C68H41N3 = 900.10 | | C64H40N4 = 865.05 |
| 69 | m/z = 865.31 | 70 | m/z = 865.31 |
| | C64H39N3O = 866.04 | | C64H39N3O = 866.04 |
| 71 | m/z = 881.29 | 72 | m/z = 881.29 |
| | C64H39N3S = 882.10 | | C64H39N3S = 882.10 |
| 73 | m/z = 940.36 | 74 | m/z = 940.36 |
| | C70H44N4 = 941.15 | | C70H44N4 = 941.15 |
| 75 | m/z = 891.36 | 76 | m/z = 891.36 |
| | C67H45N3 = 892.12 | | C67H45N3 = 892.12 |
| 77 | m/z = 1015.39 | 78 | m/z = 1015.39 |
| | C77H49N3 = 1016.26 | | C77H49N3 = 1016.26 |
| 79 | m/z = 1013.38 | 80 | m/z = 1013.38 |
| | C77H47N3 = 1014.24 | | C77H47N3 = 1014.24 |
| 81 | m/z = 700.26 | 82 | m/z = 776.29 |
| | C51H32N4 = 700.85 | | C57H36N4 = 776.94 |
| 83 | m/z = 776.29 | 84 | m/z = 750.28 |
| | C57H36N4 = 776.94 | | C55H34N4 = 750.91 |
| 85 | m/z = 852.33 | 86 | m/z = 800.29 |
| | C63H40N4 = 853.04 | | C59H36N4 = 800.97 |
| 87 | m/z = 824.29 | 88 | m/z = 789.29 |
| | C61H36N4 = 824.99 | | C57H35N5 = 789.94 |
| 89 | m/z = 790.27 | 90 | m/z = 790.27 |
| | C57H34N4O = 790.93 | | C57H34N4O = 790.93 |
| 91 | m/z = 806.25 | 92 | m/z = 806.25 |
| | C57H34N4S = 806.99 | | C57H34N4S = 806.99 |
| 93 | m/z = 865.32 | 94 | m/z = 865.32 |
| | C63H39N5 = 866.04 | | C63H39N5 = 866.04 |
| 95 | m/z = 816.33 | 96 | m/z = 816.33 |
| | C60H40N4 = 817.01 | | C60H40N4 = 817.01 |
| 97 | m/z = 940.36 | 98 | m/z = 940.36 |
| | C70H44N4 = 941.15 | | C70H44N4 = 941.15 |
| 99 | m/z = 938.34 | 100 | m/z = 938.34 |
| | C70H42N4 = 939.13 | | C70H42N4 = 939.13 |
| 101 | m/z = 776.29 | 102 | m/z = 852.33 |
| | C57H36N4 = 776.94 | | C63H40N4 = 853.04 |
| 103 | m/z = 852.33 | 104 | m/z = 826.31 |
| | C63H40N4 = 853.04 | | C61H38N4 = 827.00 |
| 105 | m/z = 928.36 | 106 | m/z = 876.33 |
| | C69H44N4 = 929.14 | | C65H40N4 = 877.06 |
| 107 | m/z = 900.33 | 108 | m/z = 865.32 |
| | C67H40N4 = 901.09 | | C63H39N5 = 866.04 |
| 109 | m/z = 866.30 | 110 | m/z = 866.30 |
| | C63H38N4O = 867.02 | | C63H38N4O = 867.02 |
| 111 | m/z = 882.28 | 112 | m/z = 882.28 |
| | C63H38N4S = 883.09 | | C63H38N4S = 883.09 |
| 113 | m/z = 941.35 | 114 | m/z = 941.35 |
| | C69H43N5 = 942.14 | | C69H43N5 = 942.14 |
| 115 | m/z = 892.36 | 116 | m/z = 892.36 |
| | C66H44N4 = 893.11 | | C66H44N4 = 893.11 |
| 117 | m/z = 1016.39 | 118 | m/z = 1016.39 |
| | C76H48N4 = 1017.25 | | C76H48N4 = 1017.25 |
| 119 | m/z = 1014.37 | 120 | m/z = 1014.37 |
| | C76H46N4 = 1015.23 | | C76H46N4 = 1015.23 |
| 121 | m/z = 698.27 | 122 | m/z = 774.30 |
| | C53H34N2 = 698.87 | | C59H38N2 = 774.97 |
| 123 | m/z = 774.30 | 124 | m/z = 748.29 |
| | C59H38N2 = 774.97 | | C57H36N2 = 748.93 |
| 125 | m/z = 850.33 | 126 | m/z = 798.30 |
| | C65H42N2 = 851.07 | | C61H38N2 = 798.99 |
| 127 | m/z = 822.30 | 128 | m/z = 787.30 |
| | C63H38N2 = 823.01 | | C59H37N3 = 787.97 |
| 129 | m/z = 788.28 | 130 | m/z = 788.28 |
| | C59H36N2O = 788.95 | | C59H36N2O = 788.95 |
| 131 | m/z = 804.26 | 132 | m/z = 804.26 |
| | C59H36N2S = 805.01 | | C59H36N2S = 805.01 |
| 133 | m/z = 863.33 | 134 | m/z = 863.33 |
| | C65H41N3 = 864.06 | | C65H41N3 = 864.06 |
| 135 | m/z = 814.33 | 136 | m/z = 814.33 |
| | C62H42N2 = 815.03 | | C62H42N2 = 815.03 |
| 137 | m/z = 938.37 | 138 | m/z = 938.37 |
| | C72H46N2 = 939.17 | | C72H46N2 = 939.17 |
| 139 | m/z = 936.35 | 140 | m/z = 936.35 |
| | C72H44N2 = 937.16 | | C72H44N2 = 937.16 |
| 141 | m/z = 774.30 | 142 | m/z = 850.33 |
| | C59H38N2 = 774.97 | | C65H42N2 = 851.07 |
| 143 | m/z = 850.33 | 144 | m/z = 824.32 |
| | C65H42N2 = 851.07 | | C63H40N2 = 825.03 |
| 145 | m/z = 926.37 | 146 | m/z = 874.33 |
| | C71H46N2 = 927.16 | | C67H42N2 = 875.09 |
| 147 | m/z = 898.33 | 148 | m/z = 863.33 |
| | C69H42N2 = 899.11 | | C65H41N3 = 864.06 |
| 149 | m/z = 864.31 | 150 | m/z = 864.31 |
| | C65H40N2O = 865.05 | | C65H40N2O = 865.05 |
| 151 | m/z = 880.29 | 152 | m/z = 880.29 |
| | C65H40N2S = 881.11 | | C65H40N2S = 881.11 |
| 153 | m/z = 939.36 | 154 | m/z = 939.36 |
| | C71H45N3 = 940.16 | | C71H45N3 = 940.16 |
| 155 | m/z = 890.37 | 156 | m/z = 890.37 |
| | C68H46N2 = 891.13 | | C68H46N2 = 891.13 |
| 157 | m/z = 1014.40 | 158 | m/z = 1014.40 |
| | C78H50N2 = 1015.27 | | C78H50N2 = 1015.27 |
| 159 | m/z = 1012.38 | 160 | m/z = 1012.38 |
| | C78H48N2 = 1013.26 | | C78H48N2 = 1013.26 |
| 161 | m/z = 649.25 | 162 | m/z = 725.28 |
| | C48H31N3 = 649.80 | | C54H35N3 = 725.90 |
| 163 | m/z = 725.28 | 164 | m/z = 699.27 |
| | C54H35N3 = 725.90 | | C52H33N3 = 699.86 |
| 165 | m/z = 801.31 | 166 | m/z = 749.28 |
| | C60H39N3 = 801.99 | | C56H35N3 = 749.92 |
| 167 | m/z = 773.28 | 168 | m/z = 738.28 |
| | C58H35N3 = 773.94 | | C54H34N4 = 738.89 |
| 169 | m/z = 739.26 | 170 | m/z = 739.26 |
| | C54H33N3O = 739.88 | | C54H33N3O = 739.88 |
| 171 | m/z = 755.24 | 172 | m/z = 755.24 |
| | C54H33N3S = 755.94 | | C54H33N3S = 755.94 |
| 173 | m/z = 814.31 | 174 | m/z = 814.31 |
| | C60H38N4 = 814.99 | | C60H38N4 = 814.99 |
| 175 | m/z = 765.31 | 176 | m/z = 765.31 |
| | C57H39N3 = 765.96 | | C57H39N3 = 765.96 |
| 177 | m/z = 889.35 | 178 | m/z = 889.35 |
| | C67H43N3 = 890.10 | | C67H43N3 = 890.10 |
| 179 | m/z = 887.33 | 180 | m/z = 887.33 |
| | C67H41N3 = 888.09 | | C67H41N3 = 888.09 |
| 181 | m/z = 725.28 | 182 | m/z = 801.31 |
| | C54H35N3 = 725.90 | | C60H39N3 = 801.99 |
| 183 | m/z = 801.31 | 184 | m/z = 775.30 |
| | C60H39N3 = 801.99 | | C58H37N3 = 775.96 |
| 185 | m/z = 877.35 | 186 | m/z = 825.31 |
| | C66H43N3 = 878.09 | | C62H39N3 = 826.02 |
| 187 | m/z = 849.31 | 188 | m/z = 814.31 |
| | C64H39N3 = 850.04 | | C60H38N4 = 814.99 |
| 189 | m/z = 815.29 | 190 | m/z = 815.29 |
| | C60H37N3O = 815.98 | | C60H37N3O = 815.98 |
| 191 | m/z = 831.27 | 192 | m/z = 831.27 |
| | C60H37N3S = 832.04 | | C60H37N3S = 832.04 |
| 193 | m/z = 890.34 | 194 | m/z = 890.34 |
| | C66H42N4 = 891.09 | | C66H42N4 = 891.09 |
| 195 | m/z = 841.35 | 196 | m/z = 841.35 |
| | C63H43N3 = 842.06 | | C63H43N3 = 842.06 |
| 197 | m/z = 965.38 | 198 | m/z = 965.38 |
| | C73H47N3 = 966.20 | | C73H47N3 = 966.20 |
| 199 | m/z = 963.36 | 200 | m/z = 963.36 |
| | C73H45N3 = 964.18 | | C73H45N3 = 964.18 |
| 201 | m/z = 649.25 | 202 | m/z = 725.28 |
| | C48H31N3 = 649.80 | | C54H35N3 = 725.90 |
| 203 | m/z = 725.28 | 204 | m/z = 699.27 |
| | C54H35N3 = 725.90 | | C52H33N3 = 699.86 |
| 205 | m/z = 801.31 | 206 | m/z = 749.28 |
| | C60H39N3 = 801.99 | | C56H35N3 = 749.92 |
| 207 | m/z = 773.28 | 208 | m/z = 738.28 |
| | C58H35N3 = 773.94 | | C54H34N4 = 738.89 |
| 209 | m/z = 739.26 | 210 | m/z = 739.26 |
| | C54H33N3O = 739.88 | | C54H33N3O = 739.88 |
| 211 | m/z = 755.24 | 212 | m/z = 755.24 |
| | C54H33N3S = 755.94 | | C54H33N3S = 755.94 |
| 213 | m/z = 814.31 | 214 | m/z = 814.31 |
| | C60H38N4 = 814.99 | | C60H38N4 = 814.99 |
| 215 | m/z = 765.31 | 216 | m/z = 765.31 |
| | C57H39N3 = 765.96 | | C57H39N3 = 765.96 |
| 217 | m/z = 889.35 | 218 | m/z = 889.35 |
| | C67H43N3 = 890.10 | | C67H43N3 = 890.10 |
| 219 | m/z = 887.33 | 220 | m/z = 887.33 |
| | C67H41N3 = 888.09 | | C67H41N3 = 888.09 |
| 221 | m/z = 725.28 | 222 | m/z = 801.31 |
| | C54H35N3 = 725.90 | | C60H39N3 = 801.99 |
| 223 | m/z = 801.31 | 224 | m/z = 775.30 |
| | C60H39N3 = 801.99 | | C58H37N3 = 775.96 |
| 225 | m/z = 877.35 | 226 | m/z = 825.31 |
| | C66H43N3 = 878.09 | | C62H39N3 = 826.02 |
| 227 | m/z = 849.31 | 228 | m/z = 814.31 |
| | C64H39N3 = 850.04 | | C60H38N4 = 814.99 |
| 229 | m/z = 815.29 | 230 | m/z = 815.29 |
| | C60H37N3O = 815.98 | | C60H37N3O = 815.98 |
| 231 | m/z = 831.27 | 232 | m/z = 831.27 |
| | C60H37N3S = 832.04 | | C60H37N3S = 832.04 |
| 233 | m/z = 890.34 | 234 | m/z = 890.34 |
| | C66H42N4 = 891.09 | | C66H42N4 = 891.09 |
| 235 | m/z = 841.35 | 236 | m/z = 841.35 |
| | C63H43N3 = 842.06 | | C63H43N3 = 842.06 |
| 237 | m/z = 965.38 | 238 | m/z = 965.38 |
| | C73H47N3 = 966.20 | | C73H47N3 = 966.20 |
| 239 | m/z = 963.36 | 240 | m/z = 963.36 |
| | C73H45N3 = 964.18 | | C73H45N3 = 964.18 |
| 241 | m/z = 650.25 | 242 | m/z = 726.28 |
| | C47H30N4 = 650.79 | | C53H34N4 = 726.88 |
| 243 | m/z = 726.28 | 244 | m/z = 700.26 |
| | C53H34N4 = 726.88 | | C51H32N4 = 700.85 |
| 245 | m/z = 802.31 | 246 | m/z = 750.28 |
| | C59H38N4 = 802.98 | | C55H34N4 = 750.91 |
| 247 | m/z = 774.28 | 248 | m/z = 739.27 |
| | C57H34N4 = 774.93 | | C53H33N5 = 739.88 |
| 249 | m/z = 740.26 | 250 | m/z = 740.26 |
| | C53H32N4O = 740.87 | | C53H32N4O = 740.87 |
| 251 | m/z = 756.23 | 252 | m/z = 756.23 |
| | C53H32N4S = 756.93 | | C53H32N4S = 756.93 |
| 253 | m/z = 815.30 | 254 | m/z = 815.30 |
| | C59H37N5 = 815.98 | | C59H37N5 = 815.98 |
| 255 | m/z = 766.31 | 256 | m/z = 766.31 |
| | C56H38N4 = 766.95 | | C56H38N4 = 766.95 |
| 257 | m/z = 890.34 | 258 | m/z = 890.34 |
| | C66H42N4 = 891.09 | | C66H42N4 = 891.09 |
| 259 | m/z = 888.33 | 260 | m/z = 888.33 |
| | C66H40N4 = 889.07 | | C66H40N4 = 889.07 |
| 261 | m/z = 726.28 | 262 | m/z = 802.31 |
| | C53H34N4 = 726.88 | | C59H38N4 = 802.98 |
| 263 | m/z = 802.31 | 264 | m/z = 776.29 |
| | C59H38N4 = 802.98 | | C57H36N4 = 776.94 |
| 265 | m/z = 878.34 | 266 | m/z = 826.31 |
| | C65H42N4 = 879.08 | | C61H38N4 = 827.00 |
| 267 | m/z = 850.31 | 268 | m/z = 815.30 |
| | C63H38N4 = 851.03 | | C59H37N5 = 815.98 |
| 269 | m/z = 816.29 | 270 | m/z = 816.29 |
| | C59H36N4O = 816.96 | | C59H36N4O = 816.96 |
| 271 | m/z = 832.27 | 272 | m/z = 832.27 |
| | C59H36N4S = 833.03 | | C59H36N4S = 833.03 |
| 273 | m/z = 891.34 | 274 | m/z = 891.34 |
| | C65H41N5 = 892.08 | | C65H41N5 = 892.08 |
| 275 | m/z = 842.34 | 276 | m/z = 842.34 |
| | C62H42N4 = 843.05 | | C62H42N4 = 843.05 |
| 277 | m/z = 966.37 | 278 | m/z = 966.37 |
| | C72H46N4 = 967.19 | | C72H46N4 = 967.19 |
| 279 | m/z = 964.36 | 280 | m/z = 964.36 |
| | C72H44N4 = 965.17 | | C72H44N4 = 965.17 |
| 281 | m/z = 648.26 | 282 | m/z = 724.29 |
| | C49H32N2 = 648.81 | | C55H36N2 = 724.91 |
| 283 | m/z = 724.29 | 284 | m/z = 698.27 |
| | C55H36N2 = 724.91 | | C53H34N2 = 698.87 |
| 285 | m/z = 800.32 | 286 | m/z = 748.29 |
| | C61H40N2 = 801.01 | | C57H36N2 = 748.93 |
| 287 | m/z = 772.29 | 288 | m/z = 737.28 |
| | C59H36N2 = 772.95 | | C55H35N3 = 737.91 |
| 289 | m/z = 738.27 | 290 | m/z = 738.27 |
| | C55H34N2O = 738.89 | | C55H34N2O = 738.89 |
| 291 | m/z = 754.24 | 292 | m/z = 754.24 |
| | C55H34N2S = 754.95 | | C55H34N2S = 754.95 |
| 293 | m/z = 813.31 | 294 | m/z = 813.31 |
| | C61H39N3 = 814.00 | | C61H39N3 = 814.00 |
| 295 | m/z = 764.32 | 296 | m/z = 764.32 |
| | C58H40N2 = 764.97 | | C58H40N2 = 764.97 |
| 297 | m/z = 888.35 | 298 | m/z = 888.35 |
| | C68H44N2 = 889.11 | | C68H44N2 = 889.11 |
| 299 | m/z = 886.33 | 300 | m/z = 886.33 |
| | C68H42N2 = 887.10 | | C68H42N2 = 887.10 |
| 301 | m/z = 724.29 | 302 | m/z = 800.32 |
| | C55H36N2 = 724.91 | | C61H40N2 = 801.01 |
| 303 | m/z = 800.32 | 304 | m/z = 774.30 |
| | C61H40N2 = 801.01 | | C59H38N2 = 774.97 |
| 305 | m/z = 876.35 | 306 | m/z = 824.32 |
| | C67H44N2 = 877.10 | | C63H40N2 = 825.03 |
| 307 | m/z = 848.32 | 308 | m/z = 813.31 |
| | C65H40N2 = 849.05 | | C61H39N3 = 814.00 |
| 309 | m/z = 814.30 | 310 | m/z = 814.30 |
| | C61H38N2O = 814.99 | | C61H38N2O = 814.99 |
| 311 | m/z = 830.28 | 312 | m/z = 830.28 |
| | C61H38N2S = 831.05 | | C61H38N2S = 831.05 |
| 313 | m/z = 889.35 | 314 | m/z = 889.35 |
| | C67H43N3 = 890.10 | | C67H43N3 = 890.10 |
| 315 | m/z = 840.35 | 316 | m/z = 840.35 |
| | C64H44N2 = 841.07 | | C64H44N2 = 841.07 |
| 317 | m/z = 964.38 | 318 | m/z = 964.38 |
| | C74H48N2 = 965.21 | | C74H48N2 = 965.21 |
| 319 | m/z = 962.37 | 320 | m/z = 962.37 |
| | C74H46N2 = 963.20 | | C74H46N2 = 963.20 |
| 321 | m/z = 776.29 | 322 | m/z = 726.28 |
| | C57H36N4 = 776.94 | | C53H34N4 = 726.88 |
| 323 | m/z = 737.28 | 324 | m/z = 737.28 |
| | C55H35N3 = 737.91 | | C55H35N3 = 737.91 |
| 325 | m/z = 687.27 | 326 | m/z = 763.30 |
| | C51H33N3 = 687.85 | | C57H37N3 = 763.94 |
| 327 | m/z = 775.30 | 328 | m/z = 725.28 |
| | C58H37N3 = 775.96 | | C54H35N3 = 725.90 |
| 329 | m/z = 597.22 | 330 | m/z = 673.25 |
| | C44H27N3 = 597.72 | | C50H31N3 = 673.82 |
| 331 | m/z = 647.24 | 332 | m/z = 723.27 |
| | C48H29N3 = 647.78 | | C54H33N3 = 723.88 |
| 333 | m/z = 673.25 | 334 | m/z = 749.28 |
| | C50H31N3 = 673.82 | | C56H35N3 = 749.92 |
| 335 | m/z = 723.27 | 336 | m/z = 799.30 |
| | C54H33N3 = 723.88 | | C60H37N3 = 799.98 |

### Example

### Example 1

A substrate used for manufacturing a device was ultrasonically cleaned with distilled water for 10 minutes, dried in an oven at 100° C. for 30 minutes, and transferred to a vacuum deposition apparatus chamber.

The substrate used in the present invention was formed in a top emission manner, and an anode electrode was formed as a metal/ITO layer. A metal material used herein may be Ag, Au, Pt, Al, Cu, Ni, Mo, Cr, or an alloy thereof. The indium tin oxide (ITO) may be stacked at a thickness of 7 to 15 nm. On the ITO electrode, a hole injecting layer, a hole transport layer, an electron blocking layer, a light emitting layer, an electron transport layer, and an electron injecting layer are formed sequentially. The hole injecting layer (HIL) was deposited at a thickness of 10 nm and about 3% dopant was added to smoothly perform hole injection. The hole transport layer (HTL) was deposited at a thickness of 120 nm. On the deposited hole transport layer, the electron blocking layer (EBL) was deposited at a thickness of 15 nm. Next, the organic light emitting layer was deposited at a thickness of 20 nm and 5% of dopant was added. Further, on the organic light emitting layer, the compound 4 synthesized in Preparation Example 1 and lithium quinolate (LiQ) were formed as the electron transport layer at a weight ratio of 2:1, and deposited at a thickness of 30 nm. During this process, the deposition rate of the organic material was maintained at 0.5 to 1.0 Å/sec, and the vacuum degree at the time of deposition was maintained at 1 to 4×10⁻⁷ torr. To form a resonance structure, the total thickness of the organic material has a specific thickness according to the luminescent color. Further, in order to maximize the resonance effect, the electrode was constituted as a semi-transparent electrode (cathode). The metal used for this electrode may include Al, Mg, Ag, LiF, or an alloy thereof, and the ratio and specific thickness are applied so that a light reflection characteristic is generated. The thickness of the negative electrode used was 14 nm. Finally, a light efficiency improvement layer (capping layer) was deposited at 63 nm. After vacuum deposition, the substrate was transferred to a glove box, and a sealing process was performed. A sealing member may be a glass cap provided with a moisture absorbent (getter) therein, and a sealing resin material may be applied to perform UV curing and to block permeation of oxygen and moisture into the deposition surface.

### Example 2

Example 2 was prepared in the same manner as in Example 1, except that Compound 21 was used instead of Compound 4 as the electron transport layer.

### Example 3

Example 3 was prepared in the same manner as in Example 1, except that Compound 82 was used instead of Compound 4 as the electron transport layer.

### Example 4

Example 4 was prepared in the same manner as in Example 1, except that Compound 84 was used instead of Compound 4 as the electron transport layer.

### Example 5

Example 5 was prepared in the same manner as in Example 1, except that Compound 101 was used instead of Compound 4 as the electron transport layer.

### Example 6

Example 6 was prepared in the same manner as in Example 1, except that Compound 181 was used instead of Compound 4 as the electron transport layer.

### Example 7

Example 7 was prepared in the same manner as in Example 1, except that Compound 242 was used instead of Compound 4 as the electron transport layer.

### Example 8

Example 8 was prepared in the same manner as in Example 1, except that Compound 244 was used instead of Compound 4 as the electron transport layer.

### Example 9

Example 9 was prepared in the same manner as in Example 1, except that Compound 248 was used instead of Compound 4 as the electron transport layer.

### Example 10

Example 10 was prepared in the same manner as in Example 1, except that Compound 261 was used instead of Compound 4 as the electron transport layer.

### Example 11

Example 11 was prepared in the same manner as in Example 1, except that Compound 321 was used instead of Compound 4 as the electron transport layer.

### Example 12

Example 12 was prepared in the same manner as in Example 1, except that Compound 322 was used instead of Compound 4 as the electron transport layer.

### Example 13

Example 13 was prepared in the same manner as in Example 1, except that Compound 327 was used instead of Compound 4 as the electron transport layer.

### Example 14

Example 14 was prepared in the same manner as in Example 1, except that Compound 328 was used instead of Compound 4 as the electron transport layer.

### Comparative Example 1

Comparative Example 1 was prepared in the same manner as in Example 1, except that Compound ET1 was used instead of Compound 4 as the electron transport layer.

### Comparative Example 2

Comparative Example 2 was prepared in the same manner as in Example 1, except that Compound ET2 was used instead of Compound 4 as the electron transport layer.

### Comparative Example 3

Comparative Example 3 was prepared in the same manner as in Example 1, except that Compound ET3 was used instead of Compound 4 as the electron transport layer.

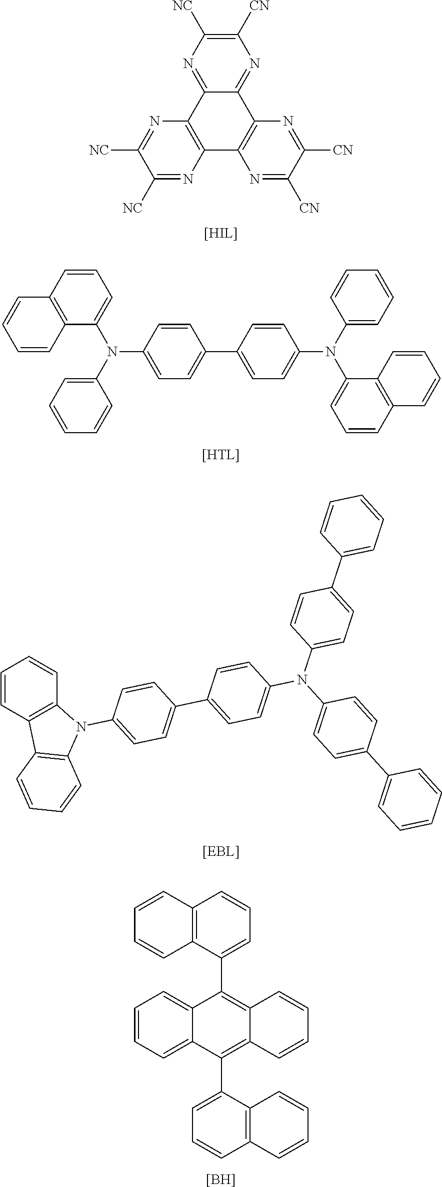

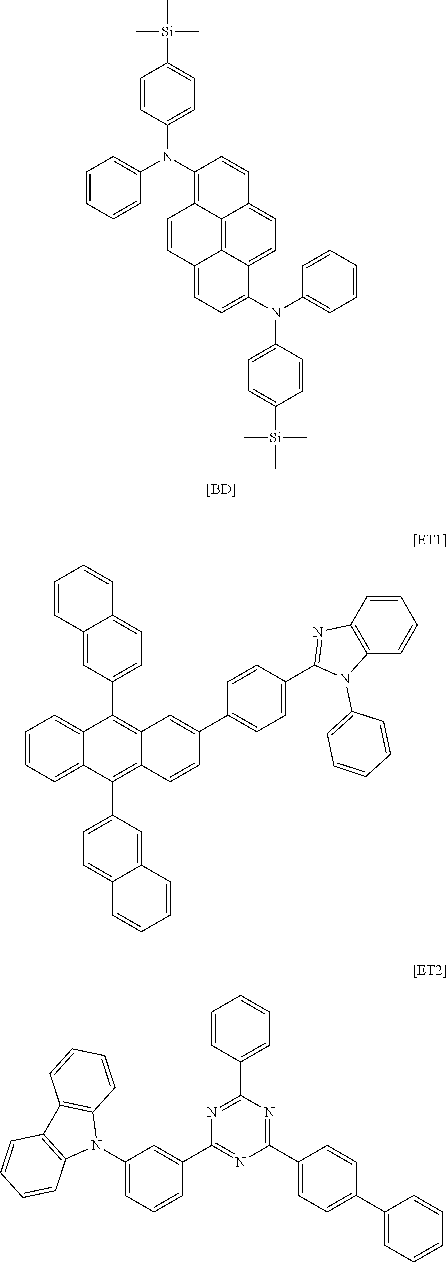

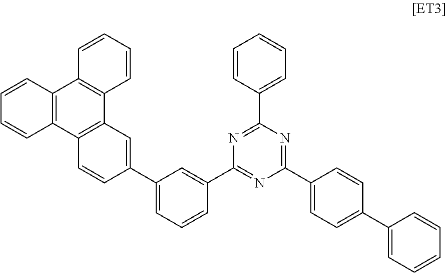 [SMILES: c1ccc(-c2ccc(-c3nc(-c4ccccc4)nc(-c4cccc(-c5ccc6c7ccccc7c7ccccc7c6c5)c4)n3)cc2)cc1]

### <Experimental Example>—Evaluation of Characteristics

Luminance, luminous efficiency, and luminous peak were respectively evaluated for each of the light emitting devices manufactured in the Examples and Comparative Examples above, using a Keithley SourceMeter “2400” and a KONIKA MINOLTA “CS-2000”, with a standard of 10 mA/cm².

The driving voltage and light emitting efficiency of the organic light emitting devices were measured at a current density of 10 mA/cm², and the time (LT95) corresponding to 95% relative to the initial luminance of 1,000 cd/m² was measured using an LTS-1004AC from ENC technology, with a standard of 700 nit. The results of the measurements are listed in Table 7 below.

TABLE 7

| | | | Current | Life Time |
|---|---|---|---|---|
| | | Voltage | Efficiency | 95 at |
| | Compound | (V) | (cd/A) | 1000 cd/m² |
| Example 1 | 4 | 4.03 | 7.43 | 173 |
| Example 2 | 21 | 4.24 | 7.45 | 131 |
| Example 3 | 82 | 4.15 | 7.24 | 150 |
| Example 4 | 84 | 4.19 | 7.19 | 134 |
| Example 5 | 101 | 4.25 | 7.29 | 191 |
| Example 6 | 181 | 4.03 | 7.49 | 144 |
| Example 7 | 242 | 4.12 | 7.40 | 137 |
| Example 8 | 244 | 4.10 | 7.35 | 153 |
| Example 9 | 248 | 4.24 | 7.27 | 248 |
| Example 10 | 261 | 4.19 | 7.37 | 189 |
| Example 11 | 321 | 4.20 | 7.35 | 139 |
| Example 12 | 322 | 4.01 | 7.38 | 141 |
| Example 13 | 327 | 4.18 | 7.58 | 130 |
| Example 14 | 328 | 4.22 | 7.52 | 134 |
| Comparative | ET1 | 4.58 | 6.71 | 105 |
| Example 1 | | | | |
| Comparative | ET2 | 4.68 | 6.63 | 113 |
| Example 2 | | | | |
| Comparative | ET3 | 4.46 | 6.94 | 95 |
| Example 3 | | | | |

Referring to Table 7 above, it was confirmed that the light emitting devices according to Examples 1 to 14 have higher efficiency, longer lifetime, and lower driving voltage than those of Comparative Examples 1 to 3.

The compounds according to the embodiments of the present invention may have excellent electrochemical and thermal stability, resulting in excellent lifetime characteristics and a high light emitting efficiency even at a low driving voltage. In addition, organic light emitting devices having high efficiency, long lifetime, high color purity, and low driving voltage may be manufactured by using the compounds represented by Chemical Formula 1 of the present invention.

In particular, the compound according to an embodiment of the present invention exhibits a high efficiency and long lifetime characteristics when the compound is used for an organic light emitting device as an electron transporting material having a reinforced hole block function.

What is claimed is:

1. A compound represented by Chemical Formula 1 below: 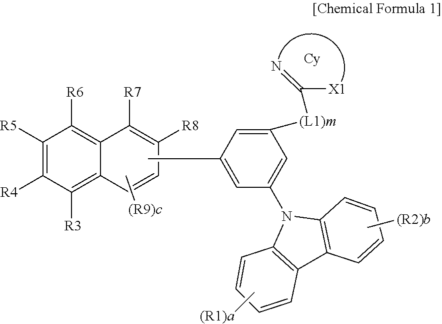
in Chemical Formula 1,
Cy is a substituted or unsubstituted monocyclic or polycyclic heterocycle group including at least one nitrogen atom,
X1 is N or CR11 when it is a member of 6-membered rings,
X1 is NR12 or NR13R14 when it is a member of 5-membered rings,
L1 is a direct bond; or a substituted or unsubstituted arylene group,
R1 to R9, and R11 to R14 are the same as or different from each other, and each independently selected from the group consisting of hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; and an amine group substituted or unsubstituted with an alkyl group, an aryl group, or a heteroaryl group, and adjacent groups may be bonded to each other to form a hydrocarbon ring or a heterocycle,
a and b are each an integer of 0 to 4, each R1 is the same as or different from each other when a is an integer of 2 or more, each R2 is the same as or different from each other when b is an integer of 2 or more, c is an integer of 0 or 1, and m is an integer of 1 or 2.

2. The compound of claim 1, wherein
the Chemical Formula 1 is represented by Chemical Formula 2 or 3 below: 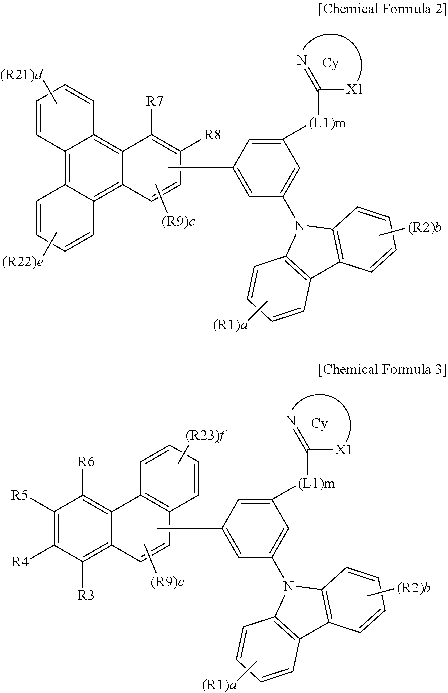
R21 to R23 are the same as or different from each other, and each independently hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; or a substituted or unsubstituted heteroaryl group, and
d, e and f are each independently an integer of 0 to 4, each R21 is the same as or different from each other when d is an integer of 2 or more, each R22 is the same as or different from each other when e is an integer of 2 or more, and each R23 is the same as or different from each other when f is an integer of 2 or more.

3. The compound of claim 1, wherein
the Chemical Formula 1 is represented by any one of Chemical Formulas 4-1 to 4-3 below: 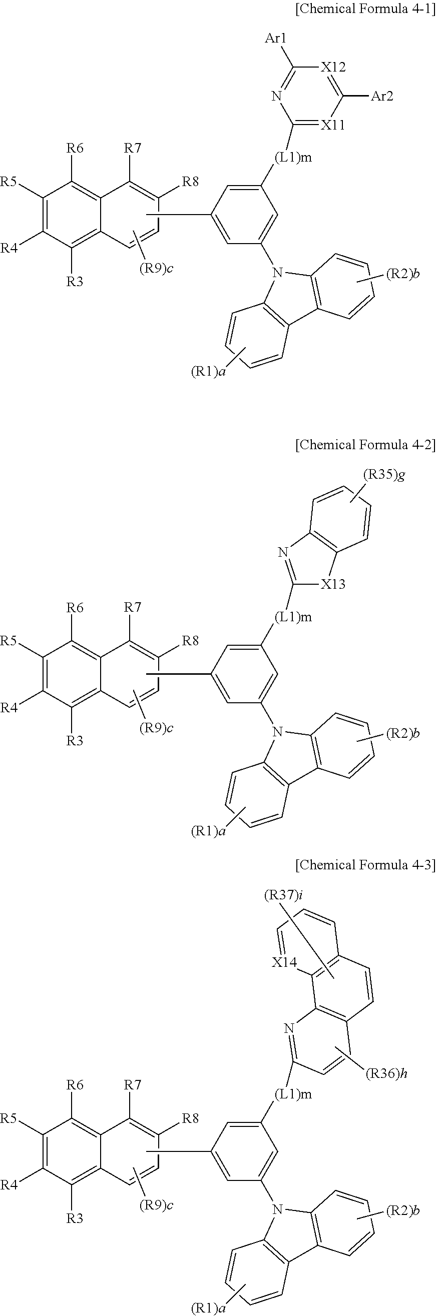
X11, X12, and X14 are the same as or different from each other and each independently N or CR31, and X13 is NR32 or (R36)CR33R34,
R31 to R37, Ar1 and Ar2 are the same as or different from each other, and each independently hydrogen; deuterium; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; or a substituted or unsubstituted heteroaryl group, and R33 and R34 may be bonded to each other to form a hydrocarbon ring, and
g is an integer of 1 to 4, each R35 is the same as or different from each other when g is an integer of 2 or more, h is an integer of 1 or 2, each R36 is the same as or different from each other when h is an integer of 2, i is an integer of 1 to 5, each R37 is the same as or different from each other when g is an integer of 2 or more, and remaining substituents are the same as defined in Chemical Formula 1.

4. The compound of claim 3, wherein
Ar1 and Ar2 are the same as or different from each other, and each independently hydrogen; a substituted or unsubstituted aryl group; or a substituted or unsubstituted heteroaryl group, and adjacent groups may be bonded to each other to form a hydrocarbon ring or a heterocycle.

5. The compound of claim 2, wherein
the Chemical Formula 2 is represented by Chemical Formula 2-2 below: 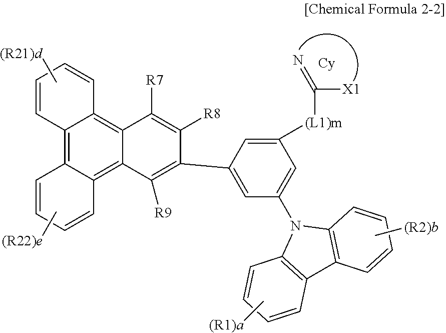
in Chemical Formula 2-2,
the definition of the substituents are the same as for Chemical Formula 2.

6. The compound of claim 1, wherein
the Chemical Formula 1 is selected from the following structural formulas:

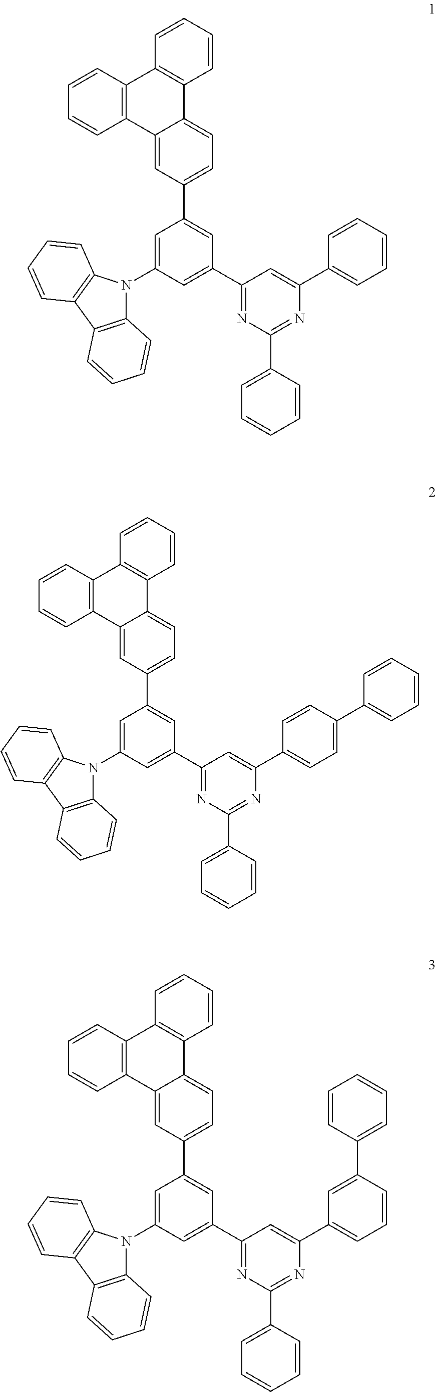 [SMILES: c1ccc(-c2cc(-c3cc(-c4ccc5c6ccccc6c6ccccc6c5c4)cc(-n4c5ccccc5c5ccccc54)c3)nc(-c3ccccc3)n2)cc1 ; c1ccc(-c2ccc(-c3cc(-c4cc(-c5ccc6c7ccccc7c7ccccc7c6c5)cc(-n5c6ccccc6c6ccccc65)c4)nc(-c4ccccc4)n3)cc2)cc1 ; c1ccc(-c2cccc(-c3cc(-c4cc(-c5ccc6c7ccccc7c7ccccc7c6c5)cc(-n5c6ccccc6c6ccccc65)c4)nc(-c4ccccc4)n3)c2)cc1]

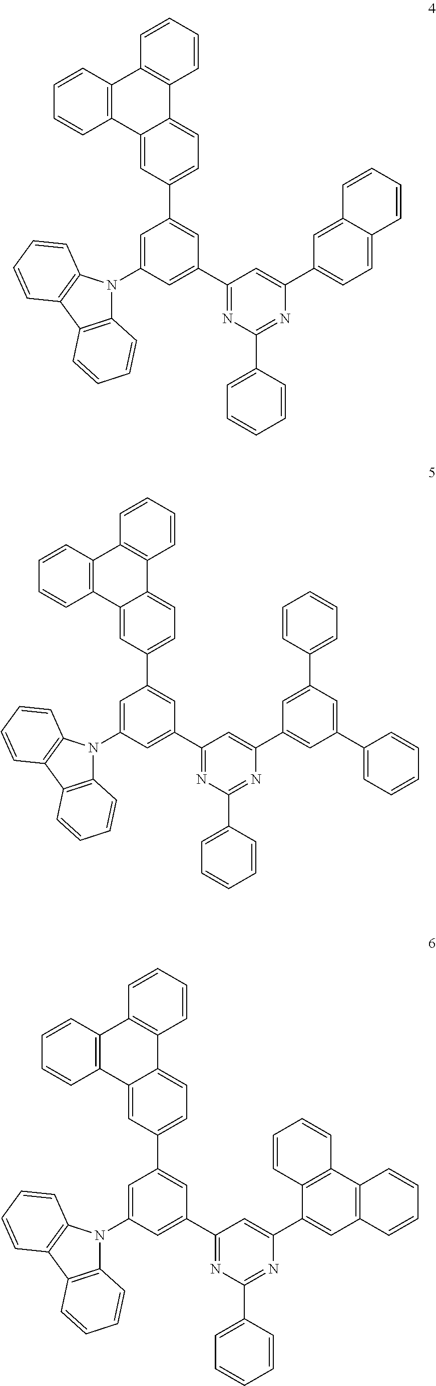 [SMILES: c1ccc(-c2nc(-c3cc(-c4ccc5c6ccccc6c6ccccc6c5c4)cc(-n4c5ccccc5c5ccccc54)c3)cc(-c3ccc4ccccc4c3)n2)cc1 ; c1ccc(-c2cc(-c3ccccc3)cc(-c3cc(-c4cc(-c5ccc6c7ccccc7c7ccccc7c6c5)cc(-n5c6ccccc6c6ccccc65)c4)nc(-c4ccccc4)n3)c2)cc1 ; c1ccc(-c2nc(-c3cc(-c4ccc5c6ccccc6c6ccccc6c5c4)cc(-n4c5ccccc5c5ccccc54)c3)cc(-c3cc4ccccc4c4ccccc34)n2)cc1]

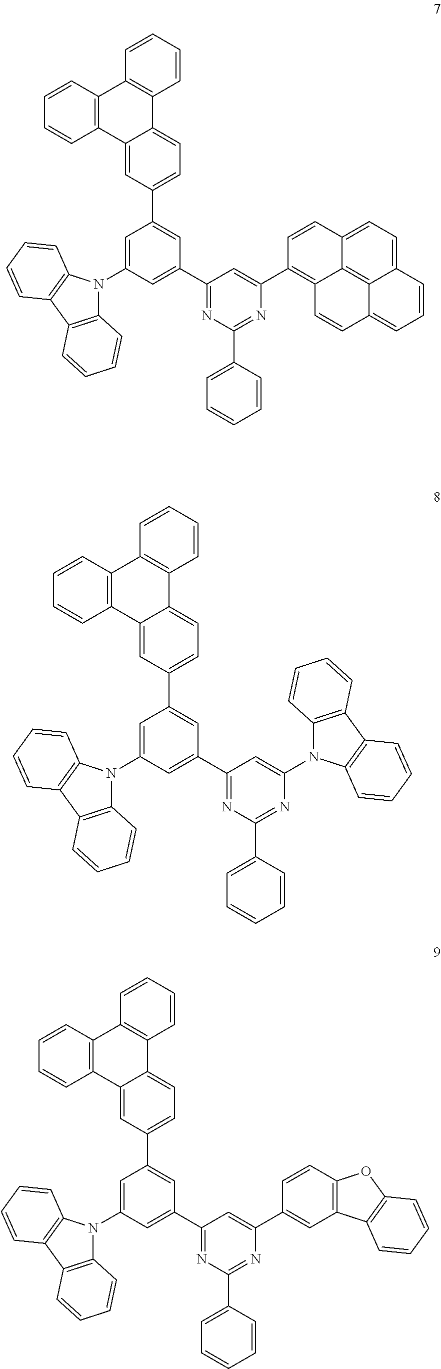 [SMILES: c1ccc(-c2nc(-c3cc(-c4ccc5c6ccccc6c6ccccc6c5c4)cc(-n4c5ccccc5c5ccccc54)c3)cc(-c3ccc4ccc5cccc6ccc3c4c56)n2)cc1 ; c1ccc(-c2nc(-c3cc(-c4ccc5c6ccccc6c6ccccc6c5c4)cc(-n4c5ccccc5c5ccccc54)c3)cc(-n3c4ccccc4c4ccccc43)n2)cc1 ; c1ccc(-c2nc(-c3cc(-c4ccc5c6ccccc6c6ccccc6c5c4)cc(-n4c5ccccc5c5ccccc54)c3)cc(-c3ccc4oc5ccccc5c4c3)n2)cc1]

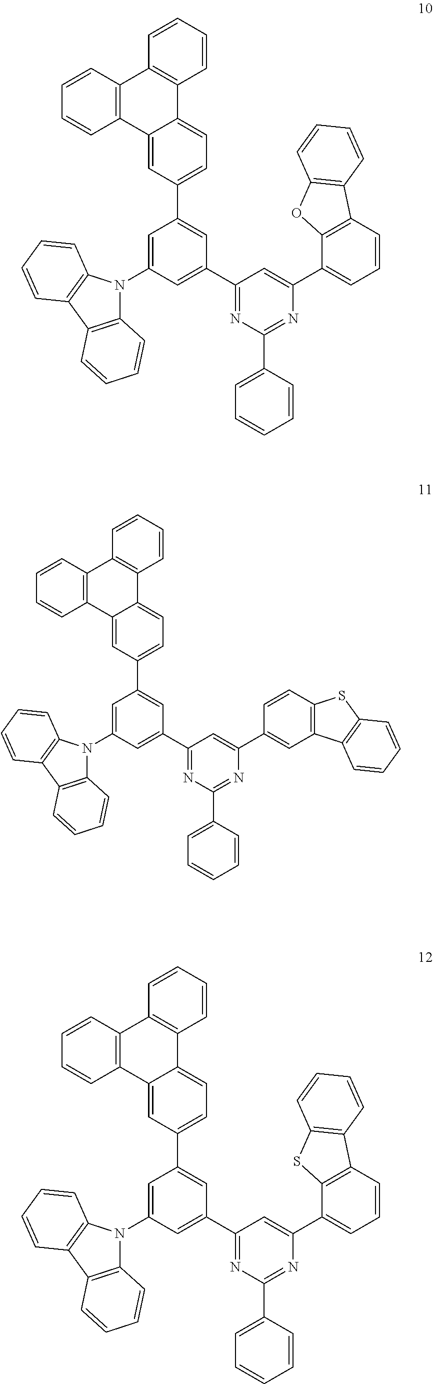 [SMILES: c1ccc(-c2nc(-c3cc(-c4ccc5c6ccccc6c6ccccc6c5c4)cc(-n4c5ccccc5c5ccccc54)c3)cc(-c3cccc4c3oc3ccccc34)n2)cc1 ; c1ccc(-c2nc(-c3cc(-c4ccc5c6ccccc6c6ccccc6c5c4)cc(-n4c5ccccc5c5ccccc54)c3)cc(-c3ccc4sc5ccccc5c4c3)n2)cc1 ; c1ccc(-c2nc(-c3cc(-c4ccc5c6ccccc6c6ccccc6c5c4)cc(-n4c5ccccc5c5ccccc54)c3)cc(-c3cccc4c3sc3ccccc34)n2)cc1]

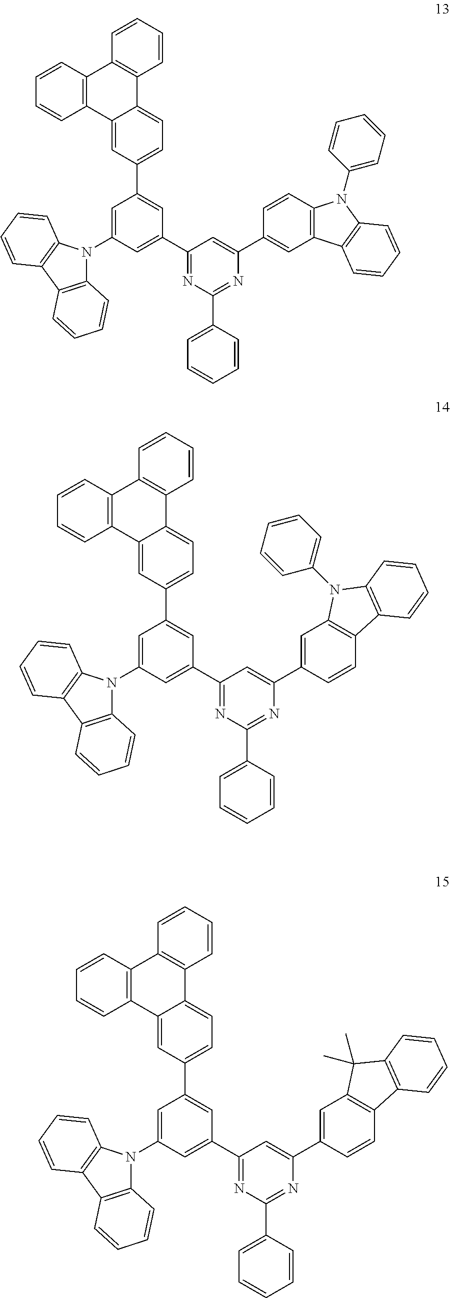 [SMILES: c1ccc(-c2nc(-c3cc(-c4ccc5c6ccccc6c6ccccc6c5c4)cc(-n4c5ccccc5c5ccccc54)c3)cc(-c3ccc4c(c3)c3ccccc3n4-c3ccccc3)n2)cc1 ; c1ccc(-c2nc(-c3cc(-c4ccc5c6ccccc6c6ccccc6c5c4)cc(-n4c5ccccc5c5ccccc54)c3)cc(-c3ccc4c5ccccc5n(-c5ccccc5)c4c3)n2)cc1 ; CC1(C)c2ccccc2-c2ccc(-c3cc(-c4cc(-c5ccc6c7ccccc7c7ccccc7c6c5)cc(-n5c6ccccc6c6ccccc65)c4)nc(-c4ccccc4)n3)cc21]

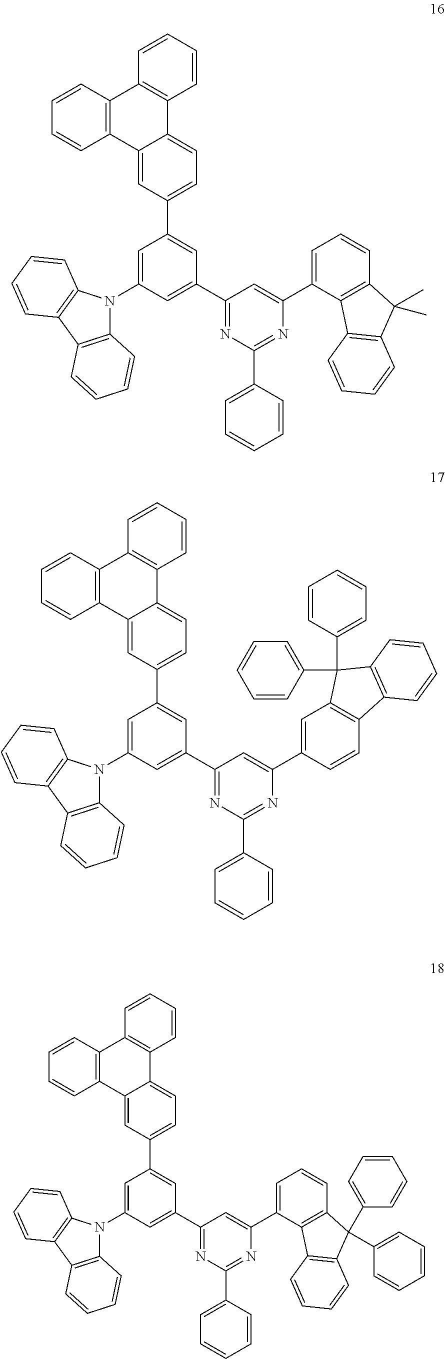 [SMILES: CC1(C)c2ccccc2-c2c(-c3cc(-c4cc(-c5ccc6c7ccccc7c7ccccc7c6c5)cc(-n5c6ccccc6c6ccccc65)c4)nc(-c4ccccc4)n3)cccc21 ; c1ccc(-c2nc(-c3cc(-c4ccc5c6ccccc6c6ccccc6c5c4)cc(-n4c5ccccc5c5ccccc54)c3)cc(-c3ccc4c(c3)C(c3ccccc3)(c3ccccc3)c3ccccc3-4)n2)cc1 ; c1ccc(-c2nc(-c3cc(-c4ccc5c6ccccc6c6ccccc6c5c4)cc(-n4c5ccccc5c5ccccc54)c3)cc(-c3cccc4c3-c3ccccc3C4(c3ccccc3)c3ccccc3)n2)cc1]

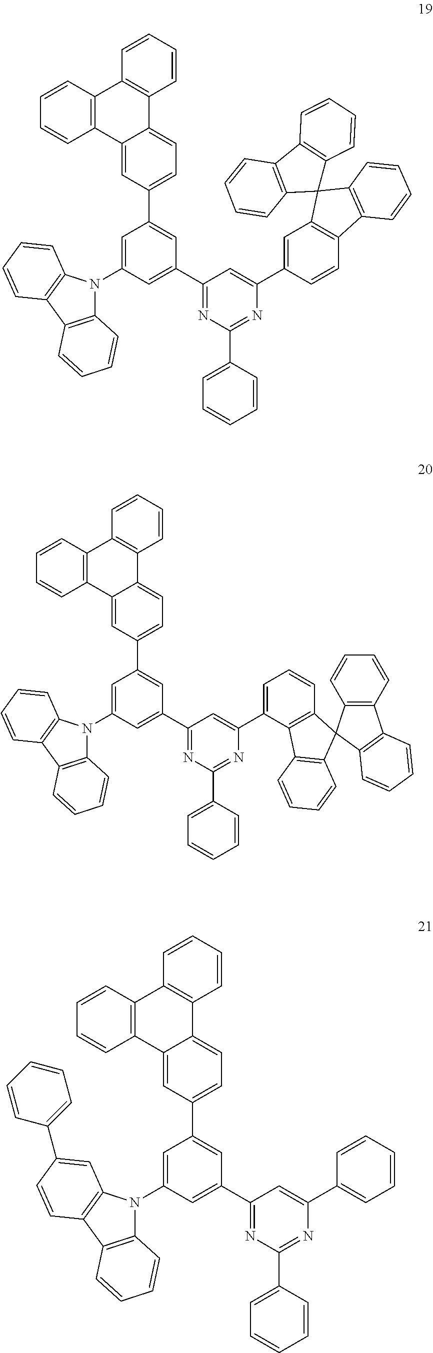 [SMILES: c1ccc(-c2nc(-c3cc(-c4ccc5c6ccccc6c6ccccc6c5c4)cc(-n4c5ccccc5c5ccccc54)c3)cc(-c3ccc4c(c3)C3(c5ccccc5-c5ccccc53)c3ccccc3-4)n2)cc1 ; c1ccc(-c2nc(-c3cc(-c4ccc5c6ccccc6c6ccccc6c5c4)cc(-n4c5ccccc5c5ccccc54)c3)cc(-c3cccc4c3-c3ccccc3C43c4ccccc4-c4ccccc43)n2)cc1 ; c1ccc(-c2ccc3c4ccccc4n(-c4cc(-c5ccc6c7ccccc7c7ccccc7c6c5)cc(-c5cc(-c6ccccc6)nc(-c6ccccc6)n5)c4)c3c2)cc1]

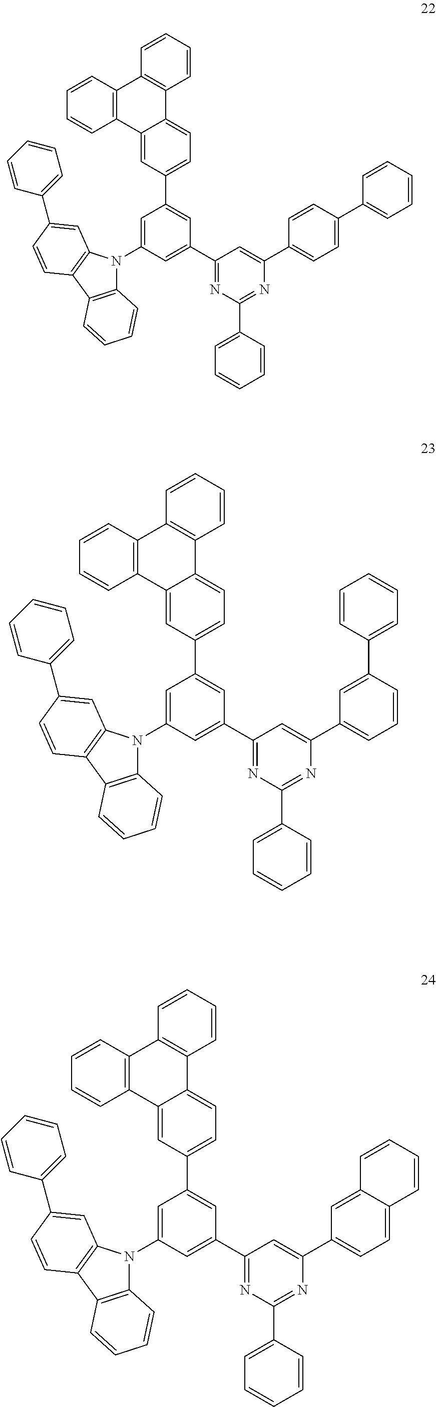 [SMILES: c1ccc(-c2ccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cc2)cc1 ; c1ccc(-c2cccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)c2)cc1 ; c1ccc(-c2ccc3c4ccccc4n(-c4cc(-c5ccc6c7ccccc7c7ccccc7c6c5)cc(-c5cc(-c6ccc7ccccc7c6)nc(-c6ccccc6)n5)c4)c3c2)cc1]

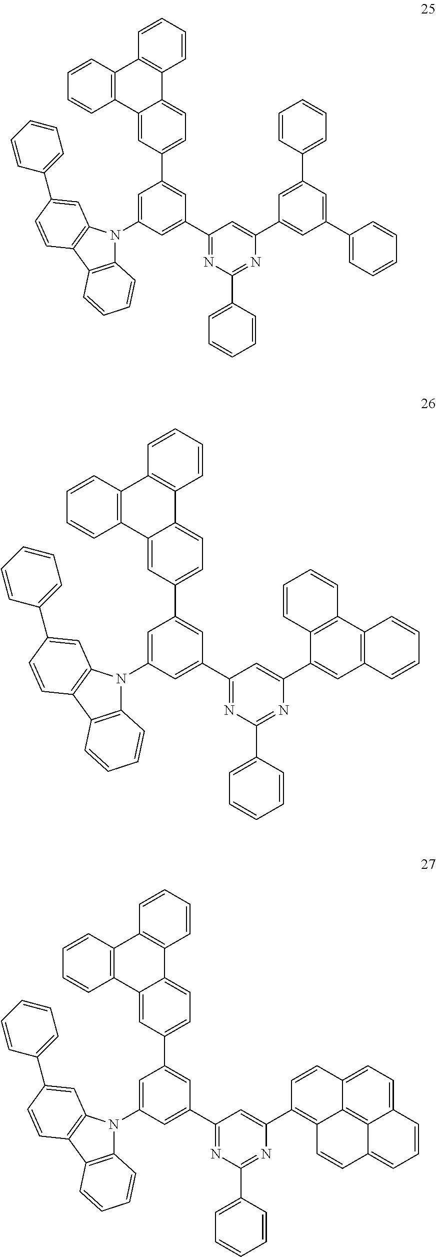 [SMILES: c1ccc(-c2cc(-c3ccccc3)cc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)c2)cc1 ; c1ccc(-c2ccc3c4ccccc4n(-c4cc(-c5ccc6c7ccccc7c7ccccc7c6c5)cc(-c5cc(-c6cc7ccccc7c7ccccc67)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7ccc8cccc9ccc6c7c89)nc(-c6ccccc6)n5)c4)c3c2)cc1]

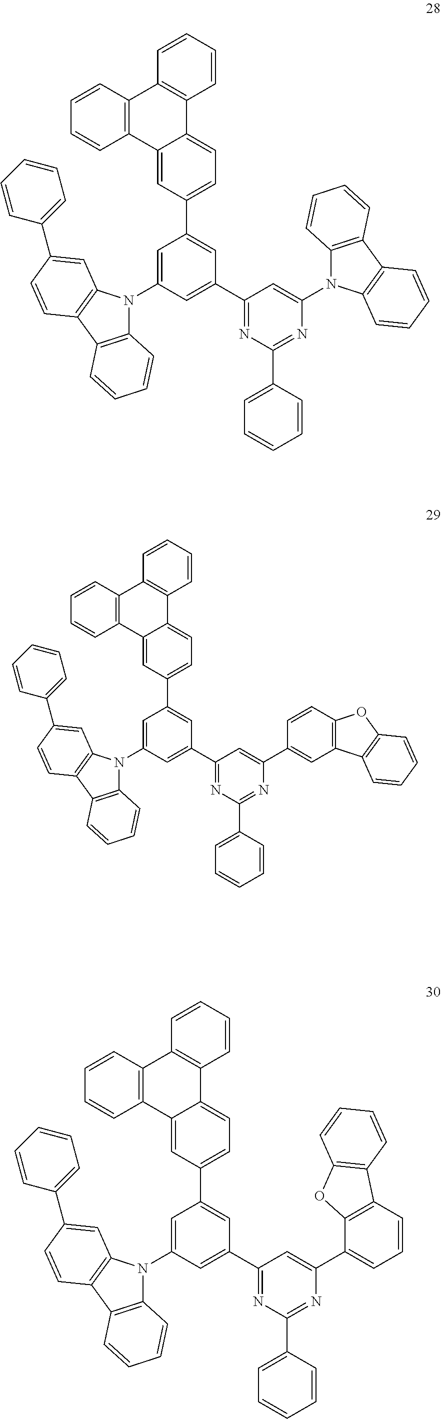 [SMILES: c1ccc(-c2ccc3c4ccccc4n(-c4cc(-c5ccc6c7ccccc7c7ccccc7c6c5)cc(-c5cc(-n6c7ccccc7c7ccccc76)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7oc8ccccc8c7c6)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6oc6ccccc67)nc(-c6ccccc6)n5)c4)c3c2)cc1]

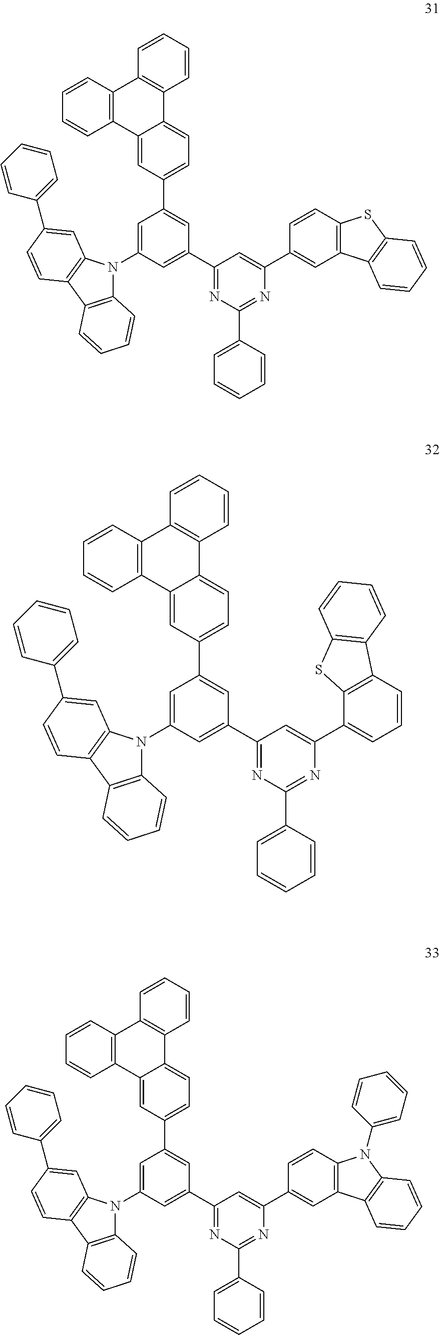 [SMILES: c1ccc(-c2ccc3c4ccccc4n(-c4cc(-c5ccc6c7ccccc7c7ccccc7c6c5)cc(-c5cc(-c6ccc7sc8ccccc8c7c6)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6sc6ccccc67)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7c(c6)c6ccccc6n7-c6ccccc6)nc(-c6ccccc6)n5)c4)c3c2)cc1]

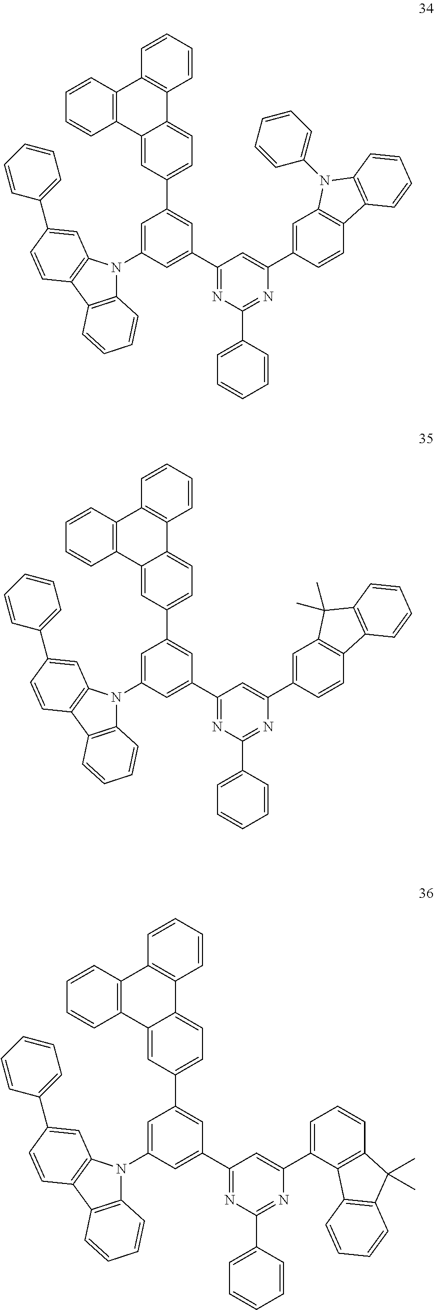 [SMILES: c1ccc(-c2ccc3c4ccccc4n(-c4cc(-c5ccc6c7ccccc7c7ccccc7c6c5)cc(-c5cc(-c6ccc7c8ccccc8n(-c8ccccc8)c7c6)nc(-c6ccccc6)n5)c4)c3c2)cc1 ; CC1(C)c2ccccc2-c2ccc(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cc21 ; CC1(C)c2ccccc2-c2c(-c3cc(-c4cc(-c5ccc6c7ccccc7c7ccccc7c6c5)cc(-n5c6ccccc6c6ccc(-c7ccccc7)cc65)c4)nc(-c4ccccc4)n3)cccc21]

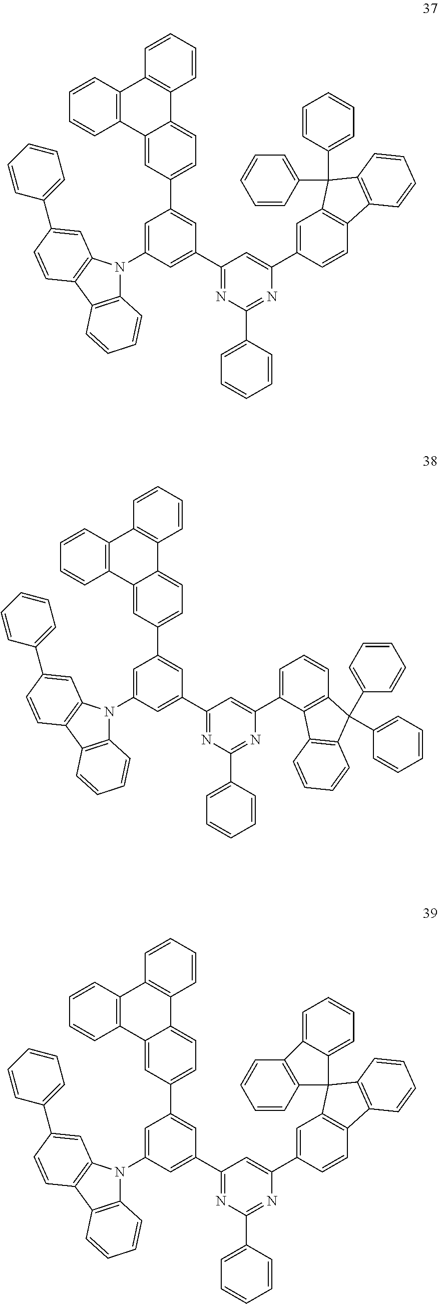 [SMILES: c1ccc(-c2ccc3c4ccccc4n(-c4cc(-c5ccc6c7ccccc7c7ccccc7c6c5)cc(-c5cc(-c6ccc7c(c6)C(c6ccccc6)(c6ccccc6)c6ccccc6-7)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6cccc7c6-c6ccccc6C7(c6ccccc6)c6ccccc6)nc(-c6ccccc6)n5)c4)c3c2)cc1 ; c1ccc(-c2ccc3c4ccccc4n(-c4cc(-c5ccc6c7ccccc7c7ccccc7c6c5)cc(-c5cc(-c6ccc7c(c6)C6(c8ccccc8-c8ccccc86)c6ccccc6-7)nc(-c6ccccc6)n5)c4)c3c2)cc1]

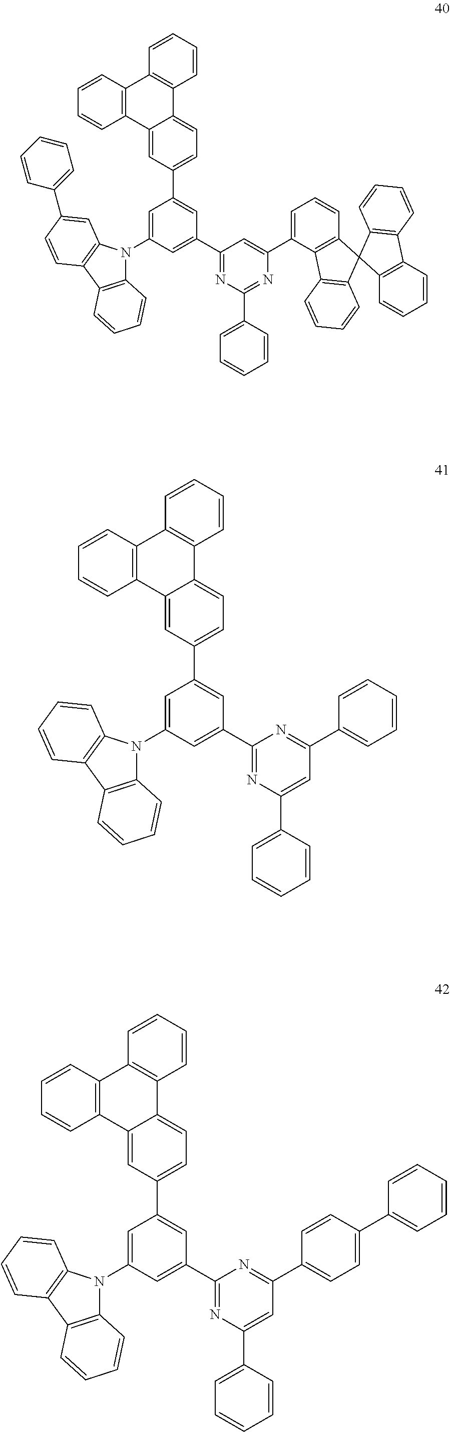 [SMILES: c1ccc(-c2ccc3c4ccccc4n(-c4cc(-c5ccc6c7ccccc7c7ccccc7c6c5)cc(-c5cc(-c6cccc7c6-c6ccccc6C76c7ccccc7-c7ccccc76)nc(-c6ccccc6)n5)c4)c3c2)cc1 ; c1ccc(-c2cc(-c3ccccc3)nc(-c3cc(-c4ccc5c6ccccc6c6ccccc6c5c4)cc(-n4c5ccccc5c5ccccc54)c3)n2)cc1 ; c1ccc(-c2ccc(-c3cc(-c4ccccc4)nc(-c4cc(-c5ccc6c7ccccc7c7ccccc7c6c5)cc(-n5c6ccccc6c6ccccc65)c4)n3)cc2)cc1]

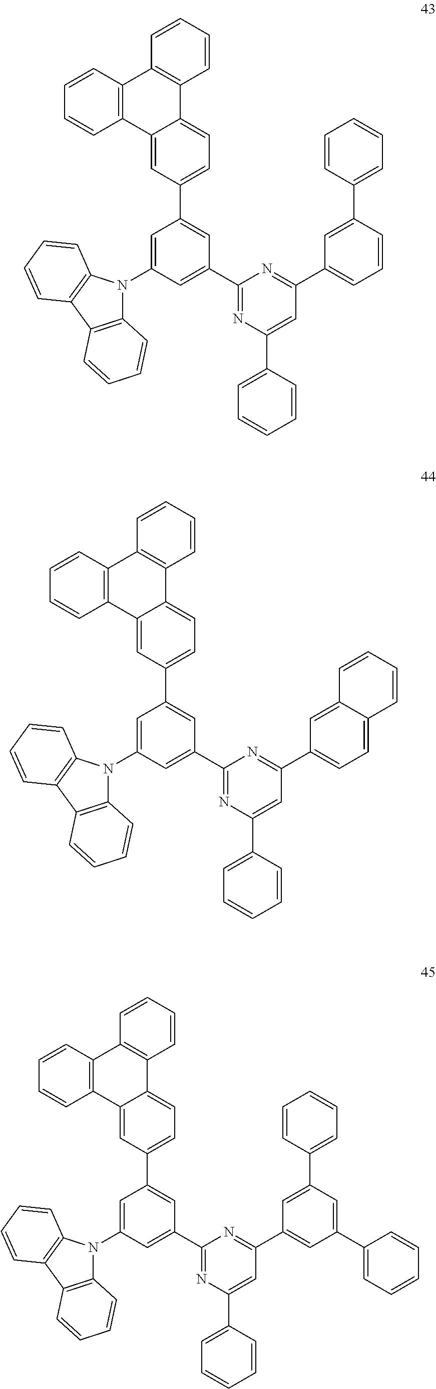 [SMILES: c1ccc(-c2cccc(-c3cc(-c4ccccc4)nc(-c4cc(-c5ccc6c7ccccc7c7ccccc7c6c5)cc(-n5c6ccccc6c6ccccc65)c4)n3)c2)cc1 ; c1ccc(-c2cc(-c3ccc4ccccc4c3)nc(-c3cc(-c4ccc5c6ccccc6c6ccccc6c5c4)cc(-n4c5ccccc5c5ccccc54)c3)n2)cc1 ; c1ccc(-c2cc(-c3ccccc3)cc(-c3cc(-c4ccccc4)nc(-c4cc(-c5ccc6c7ccccc7c7ccccc7c6c5)cc(-n5c6ccccc6c6ccccc65)c4)n3)c2)cc1]

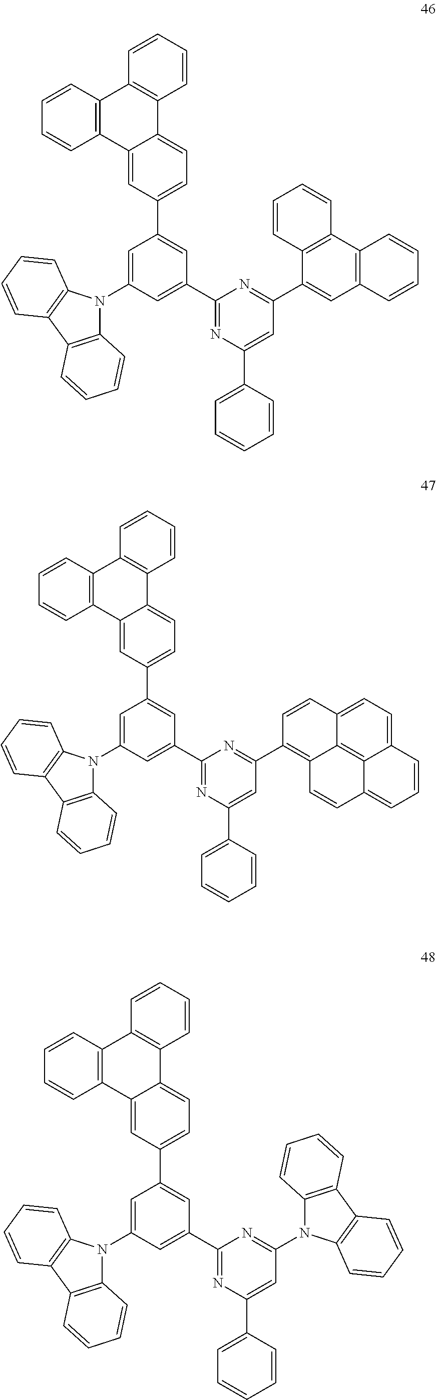 [SMILES: c1ccc(-c2cc(-c3cc4ccccc4c4ccccc34)nc(-c3cc(-c4ccc5c6ccccc6c6ccccc6c5c4)cc(-n4c5ccccc5c5ccccc54)c3)n2)cc1 ; c1ccc(-c2cc(-c3ccc4ccc5cccc6ccc3c4c56)nc(-c3cc(-c4ccc5c6ccccc6c6ccccc6c5c4)cc(-n4c5ccccc5c5ccccc54)c3)n2)cc1 ; c1ccc(-c2cc(-n3c4ccccc4c4ccccc43)nc(-c3cc(-c4ccc5c6ccccc6c6ccccc6c5c4)cc(-n4c5ccccc5c5ccccc54)c3)n2)cc1]

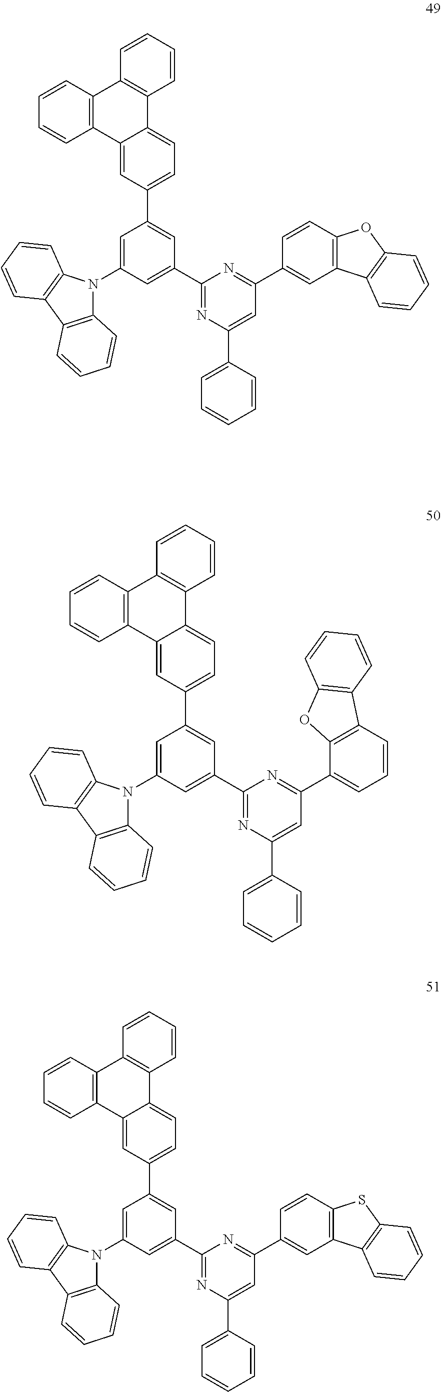 [SMILES: c1ccc(-c2cc(-c3ccc4oc5ccccc5c4c3)nc(-c3cc(-c4ccc5c6ccccc6c6ccccc6c5c4)cc(-n4c5ccccc5c5ccccc54)c3)n2)cc1 ; c1ccc(-c2cc(-c3cccc4c3oc3ccccc34)nc(-c3cc(-c4ccc5c6ccccc6c6ccccc6c5c4)cc(-n4c5ccccc5c5ccccc54)c3)n2)cc1 ; c1ccc(-c2cc(-c3ccc4sc5ccccc5c4c3)nc(-c3cc(-c4ccc5c6ccccc6c6ccccc6c5c4)cc(-n4c5ccccc5c5ccccc54)c3)n2)cc1]

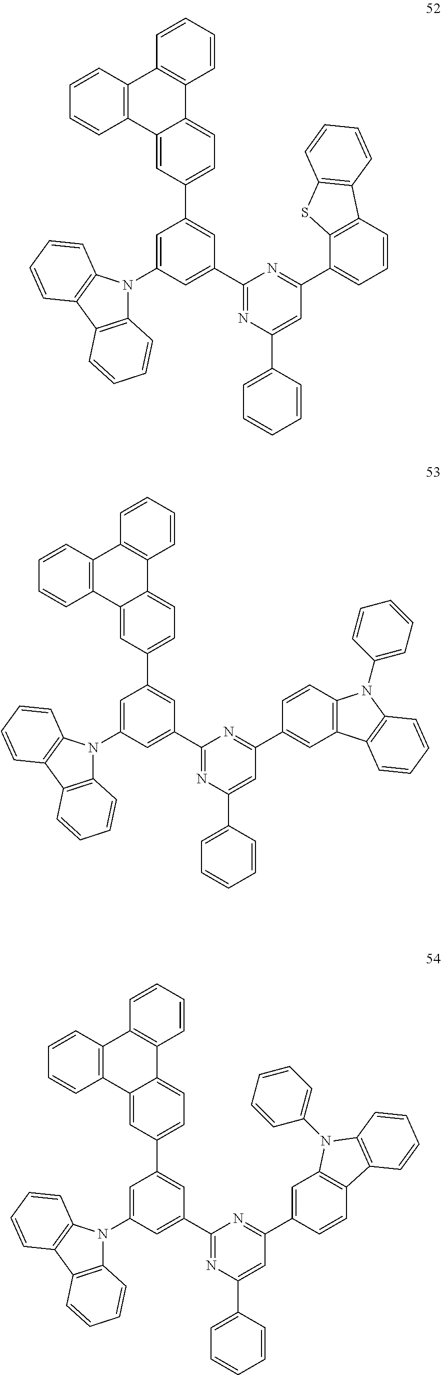 [SMILES: c1ccc(-c2cc(-c3cccc4c3sc3ccccc34)nc(-c3cc(-c4ccc5c6ccccc6c6ccccc6c5c4)cc(-n4c5ccccc5c5ccccc54)c3)n2)cc1 ; c1ccc(-c2cc(-c3ccc4c(c3)c3ccccc3n4-c3ccccc3)nc(-c3cc(-c4ccc5c6ccccc6c6ccccc6c5c4)cc(-n4c5ccccc5c5ccccc54)c3)n2)cc1 ; c1ccc(-c2cc(-c3ccc4c5ccccc5n(-c5ccccc5)c4c3)nc(-c3cc(-c4ccc5c6ccccc6c6ccccc6c5c4)cc(-n4c5ccccc5c5ccccc54)c3)n2)cc1]

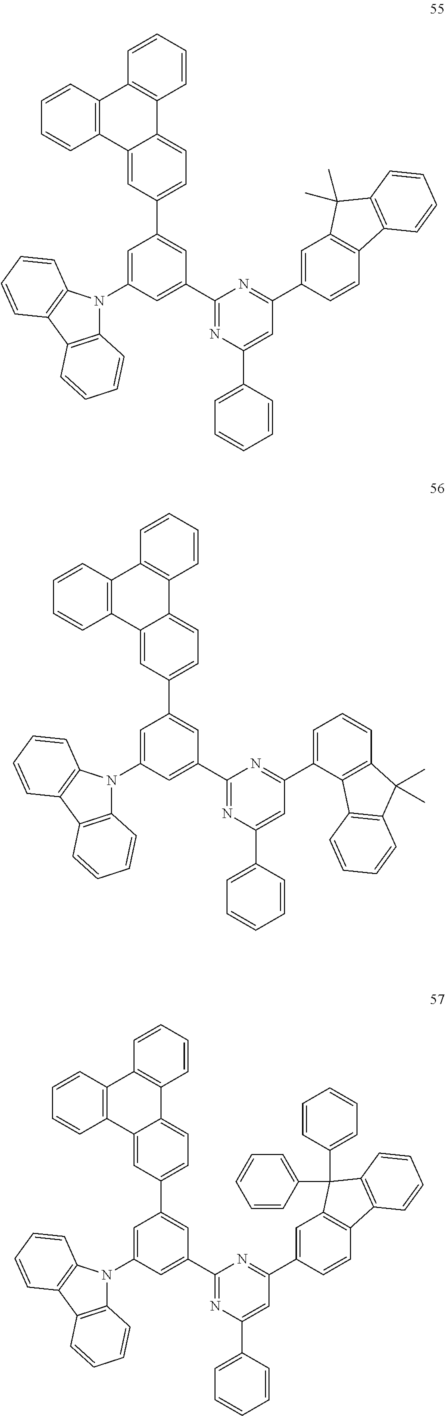 [SMILES: CC1(C)c2ccccc2-c2ccc(-c3cc(-c4ccccc4)nc(-c4cc(-c5ccc6c7ccccc7c7ccccc7c6c5)cc(-n5c6ccccc6c6ccccc65)c4)n3)cc21 ; CC1(C)c2ccccc2-c2c(-c3cc(-c4ccccc4)nc(-c4cc(-c5ccc6c7ccccc7c7ccccc7c6c5)cc(-n5c6ccccc6c6ccccc65)c4)n3)cccc21 ; c1ccc(-c2cc(-c3ccc4c(c3)C(c3ccccc3)(c3ccccc3)c3ccccc3-4)nc(-c3cc(-c4ccc5c6ccccc6c6ccccc6c5c4)cc(-n4c5ccccc5c5ccccc54)c3)n2)cc1]

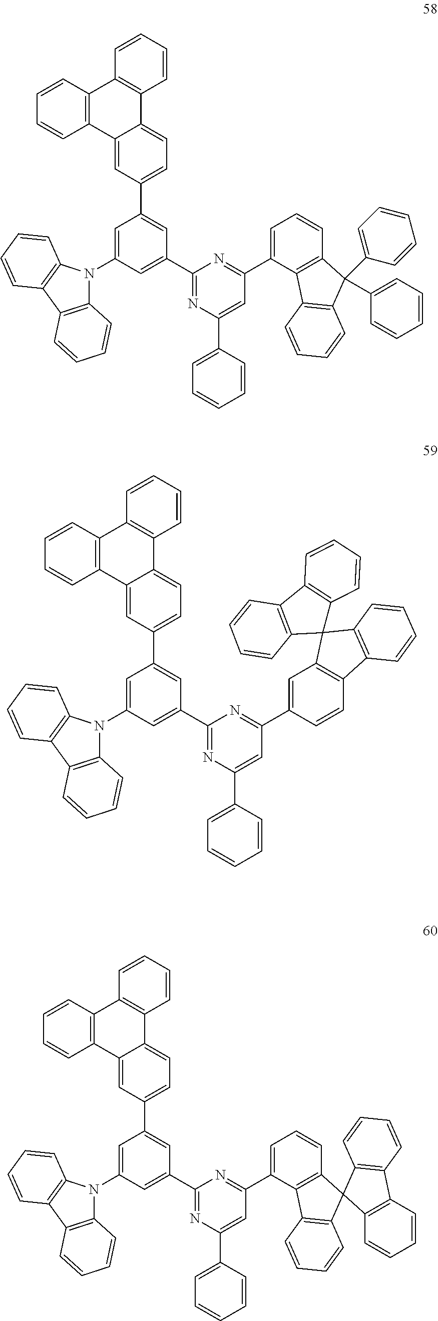 [SMILES: c1ccc(-c2cc(-c3cccc4c3-c3ccccc3C4(c3ccccc3)c3ccccc3)nc(-c3cc(-c4ccc5c6ccccc6c6ccccc6c5c4)cc(-n4c5ccccc5c5ccccc54)c3)n2)cc1 ; c1ccc(-c2cc(-c3ccc4c(c3)C3(c5ccccc5-c5ccccc53)c3ccccc3-4)nc(-c3cc(-c4ccc5c6ccccc6c6ccccc6c5c4)cc(-n4c5ccccc5c5ccccc54)c3)n2)cc1 ; c1ccc(-c2cc(-c3cccc4c3-c3ccccc3C43c4ccccc4-c4ccccc43)nc(-c3cc(-c4ccc5c6ccccc6c6ccccc6c5c4)cc(-n4c5ccccc5c5ccccc54)c3)n2)cc1]

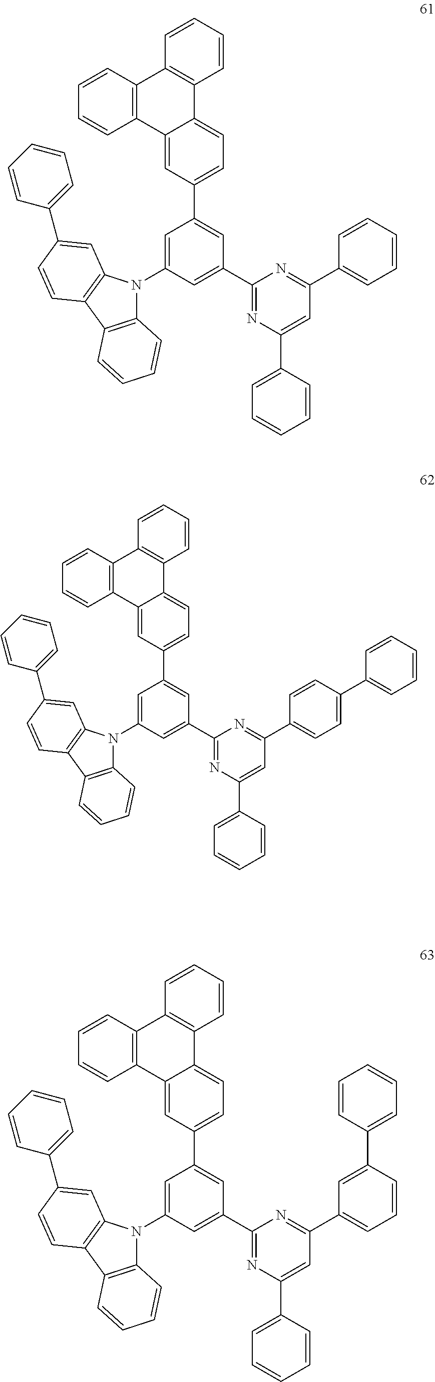 [SMILES: c1ccc(-c2ccc3c4ccccc4n(-c4cc(-c5ccc6c7ccccc7c7ccccc7c6c5)cc(-c5nc(-c6ccccc6)cc(-c6ccccc6)n5)c4)c3c2)cc1 ; c1ccc(-c2ccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cc2)cc1 ; c1ccc(-c2cccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1]

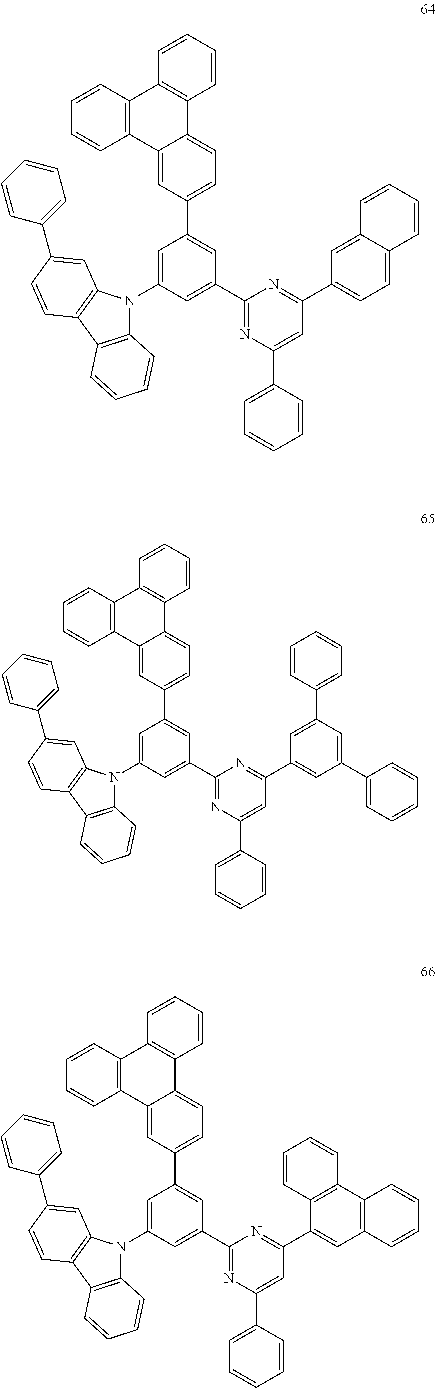 [SMILES: c1ccc(-c2ccc3c4ccccc4n(-c4cc(-c5ccc6c7ccccc7c7ccccc7c6c5)cc(-c5nc(-c6ccccc6)cc(-c6ccc7ccccc7c6)n5)c4)c3c2)cc1 ; c1ccc(-c2cc(-c3ccccc3)cc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2ccc3c4ccccc4n(-c4cc(-c5ccc6c7ccccc7c7ccccc7c6c5)cc(-c5nc(-c6ccccc6)cc(-c6cc7ccccc7c7ccccc67)n5)c4)c3c2)cc1]

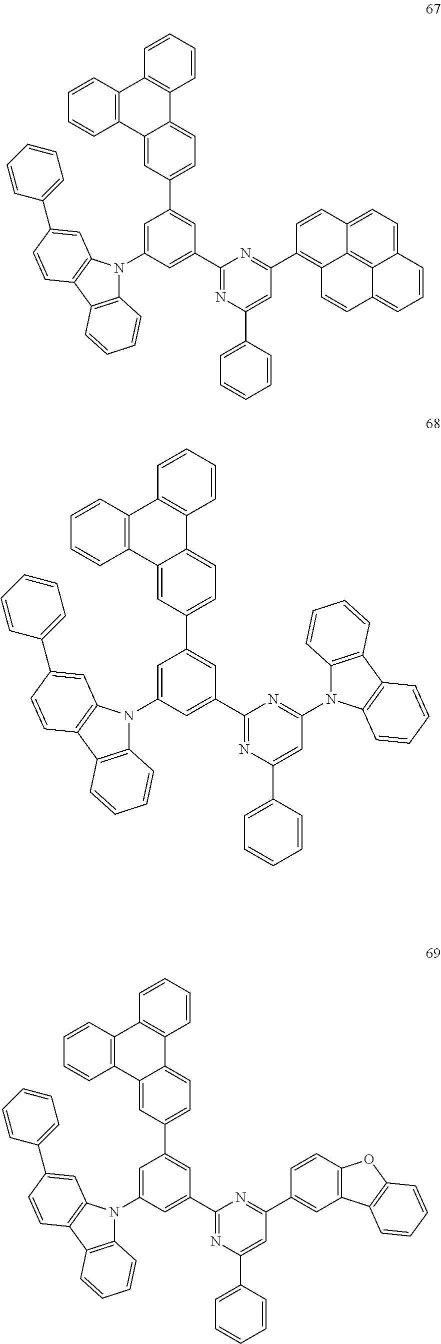 [SMILES: c1ccc(-c2ccc3c4ccccc4n(-c4cc(-c5ccc6c7ccccc7c7ccccc7c6c5)cc(-c5nc(-c6ccccc6)cc(-c6ccc7ccc8cccc9ccc6c7c89)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-n6c7ccccc7c7ccccc76)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7oc8ccccc8c7c6)n5)c4)c3c2)cc1]

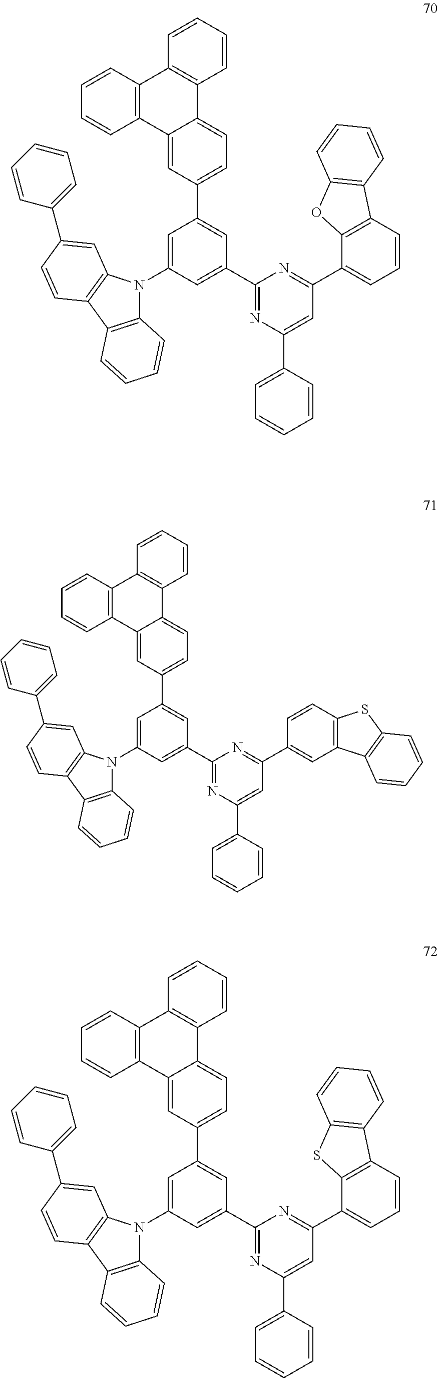 [SMILES: c1ccc(-c2ccc3c4ccccc4n(-c4cc(-c5ccc6c7ccccc7c7ccccc7c6c5)cc(-c5nc(-c6ccccc6)cc(-c6cccc7c6oc6ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7sc8ccccc8c7c6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6sc6ccccc67)n5)c4)c3c2)cc1]

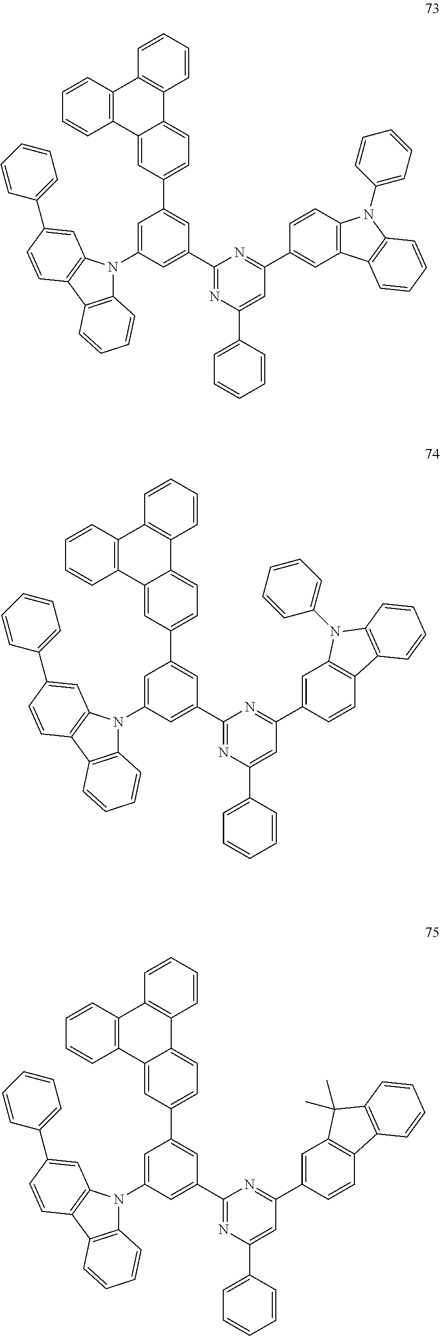 [SMILES: c1ccc(-c2ccc3c4ccccc4n(-c4cc(-c5ccc6c7ccccc7c7ccccc7c6c5)cc(-c5nc(-c6ccccc6)cc(-c6ccc7c(c6)c6ccccc6n7-c6ccccc6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6ccc7c8ccccc8n(-c8ccccc8)c7c6)n5)c4)c3c2)cc1 ; CC1(C)c2ccccc2-c2ccc(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cc21]

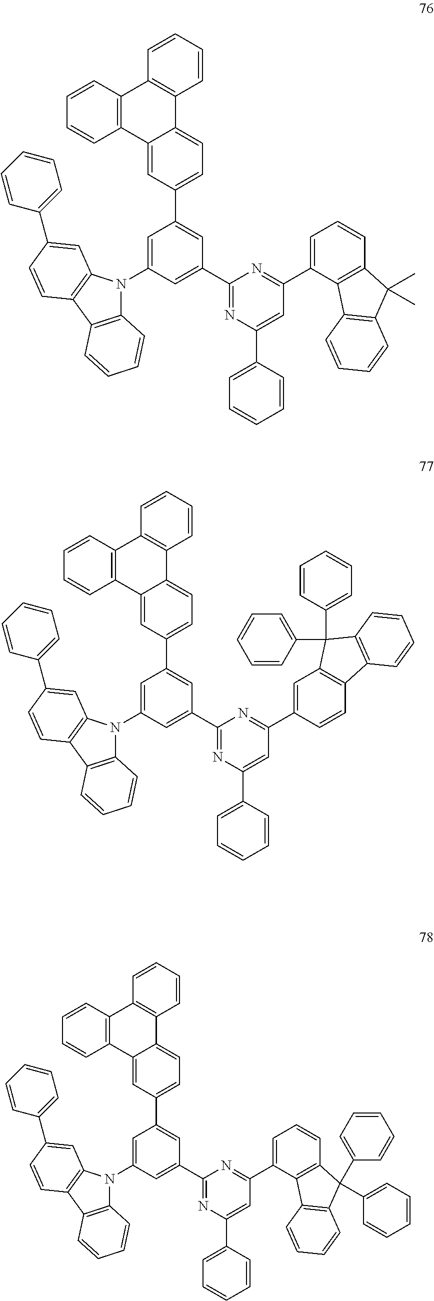 [SMILES: CC1(C)c2ccccc2-c2c(-c3cc(-c4ccccc4)nc(-c4cc(-c5ccc6c7ccccc7c7ccccc7c6c5)cc(-n5c6ccccc6c6ccc(-c7ccccc7)cc65)c4)n3)cccc21 ; c1ccc(-c2ccc3c4ccccc4n(-c4cc(-c5ccc6c7ccccc7c7ccccc7c6c5)cc(-c5nc(-c6ccccc6)cc(-c6ccc7c(c6)C(c6ccccc6)(c6ccccc6)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6-c6ccccc6C7(c6ccccc6)c6ccccc6)n5)c4)c3c2)cc1]

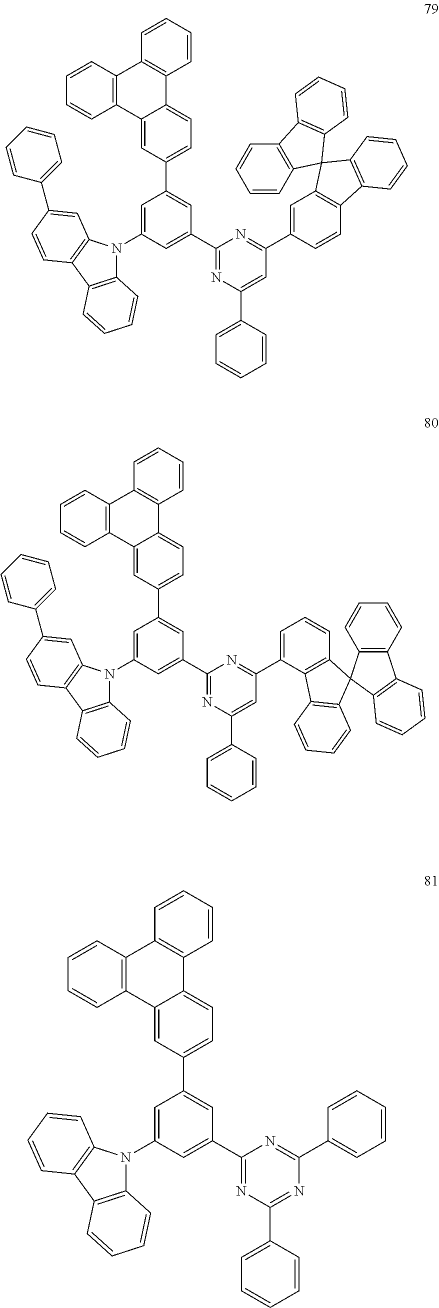 [SMILES: c1ccc(-c2ccc3c4ccccc4n(-c4cc(-c5ccc6c7ccccc7c7ccccc7c6c5)cc(-c5nc(-c6ccccc6)cc(-c6ccc7c(c6)C6(c8ccccc8-c8ccccc86)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)cc(-c6cccc7c6-c6ccccc6C76c7ccccc7-c7ccccc76)n5)c4)c3c2)cc1 ; c1ccc(-c2nc(-c3ccccc3)nc(-c3cc(-c4ccc5c6ccccc6c6ccccc6c5c4)cc(-n4c5ccccc5c5ccccc54)c3)n2)cc1]

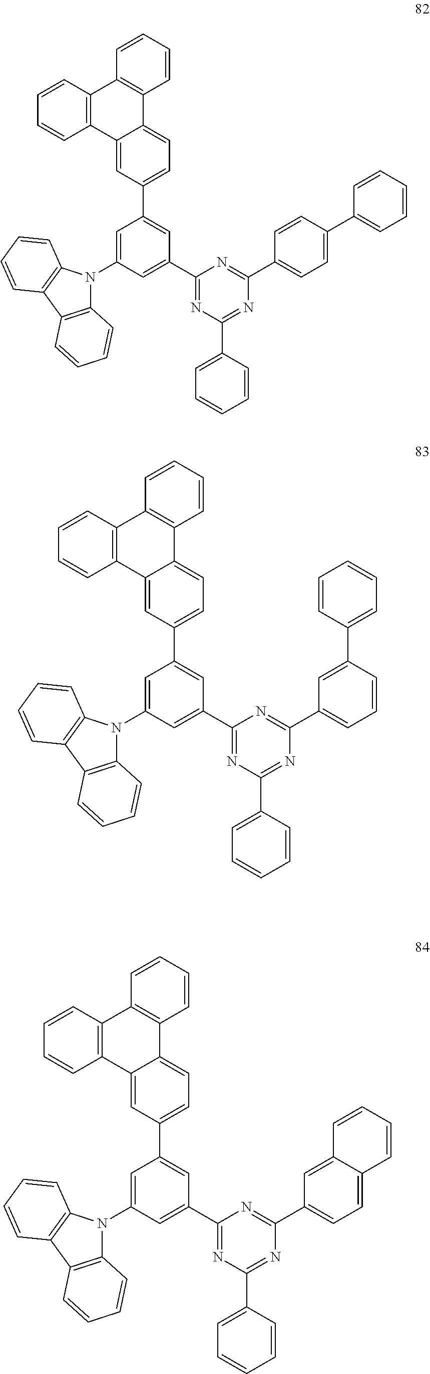 [SMILES: c1ccc(-c2ccc(-c3nc(-c4ccccc4)nc(-c4cc(-c5ccc6c7ccccc7c7ccccc7c6c5)cc(-n5c6ccccc6c6ccccc65)c4)n3)cc2)cc1 ; c1ccc(-c2cccc(-c3nc(-c4ccccc4)nc(-c4cc(-c5ccc6c7ccccc7c7ccccc7c6c5)cc(-n5c6ccccc6c6ccccc65)c4)n3)c2)cc1 ; c1ccc(-c2nc(-c3cc(-c4ccc5c6ccccc6c6ccccc6c5c4)cc(-n4c5ccccc5c5ccccc54)c3)nc(-c3ccc4ccccc4c3)n2)cc1]

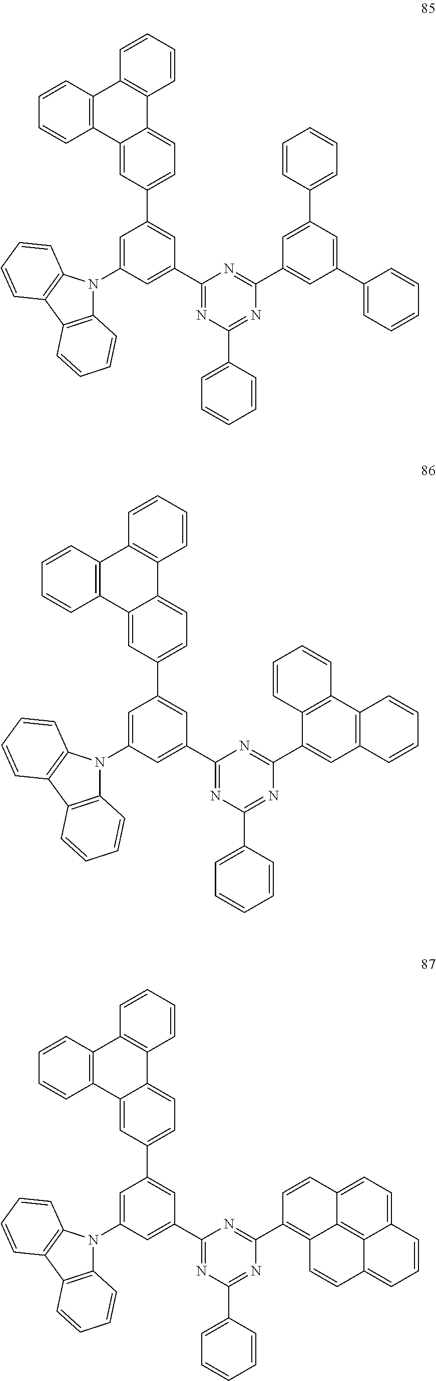 [SMILES: c1ccc(-c2cc(-c3ccccc3)cc(-c3nc(-c4ccccc4)nc(-c4cc(-c5ccc6c7ccccc7c7ccccc7c6c5)cc(-n5c6ccccc6c6ccccc65)c4)n3)c2)cc1 ; c1ccc(-c2nc(-c3cc(-c4ccc5c6ccccc6c6ccccc6c5c4)cc(-n4c5ccccc5c5ccccc54)c3)nc(-c3cc4ccccc4c4ccccc34)n2)cc1 ; c1ccc(-c2nc(-c3cc(-c4ccc5c6ccccc6c6ccccc6c5c4)cc(-n4c5ccccc5c5ccccc54)c3)nc(-c3ccc4ccc5cccc6ccc3c4c56)n2)cc1]

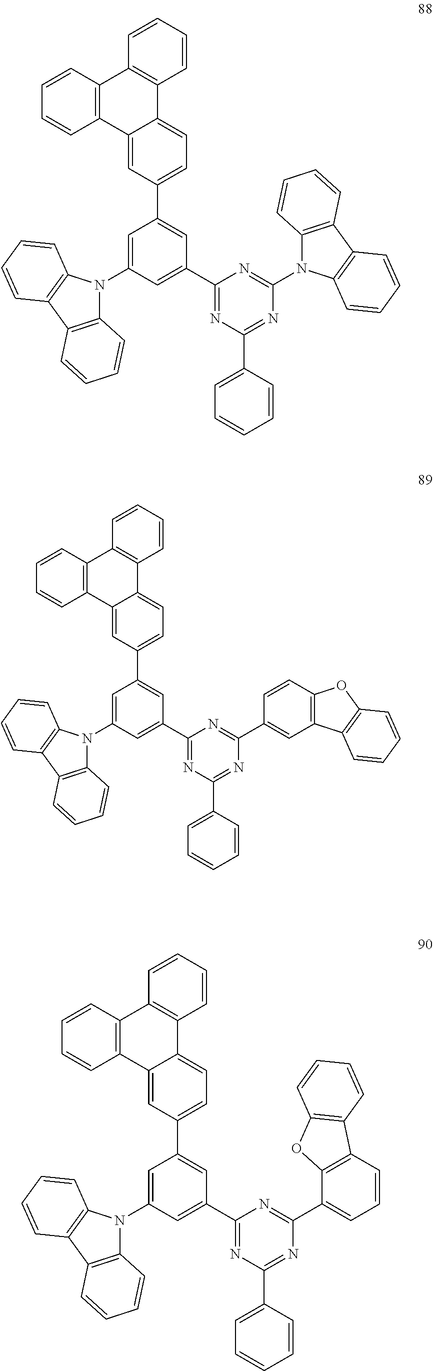 [SMILES: c1ccc(-c2nc(-c3cc(-c4ccc5c6ccccc6c6ccccc6c5c4)cc(-n4c5ccccc5c5ccccc54)c3)nc(-n3c4ccccc4c4ccccc43)n2)cc1 ; c1ccc(-c2nc(-c3cc(-c4ccc5c6ccccc6c6ccccc6c5c4)cc(-n4c5ccccc5c5ccccc54)c3)nc(-c3ccc4oc5ccccc5c4c3)n2)cc1 ; c1ccc(-c2nc(-c3cc(-c4ccc5c6ccccc6c6ccccc6c5c4)cc(-n4c5ccccc5c5ccccc54)c3)nc(-c3cccc4c3oc3ccccc34)n2)cc1]

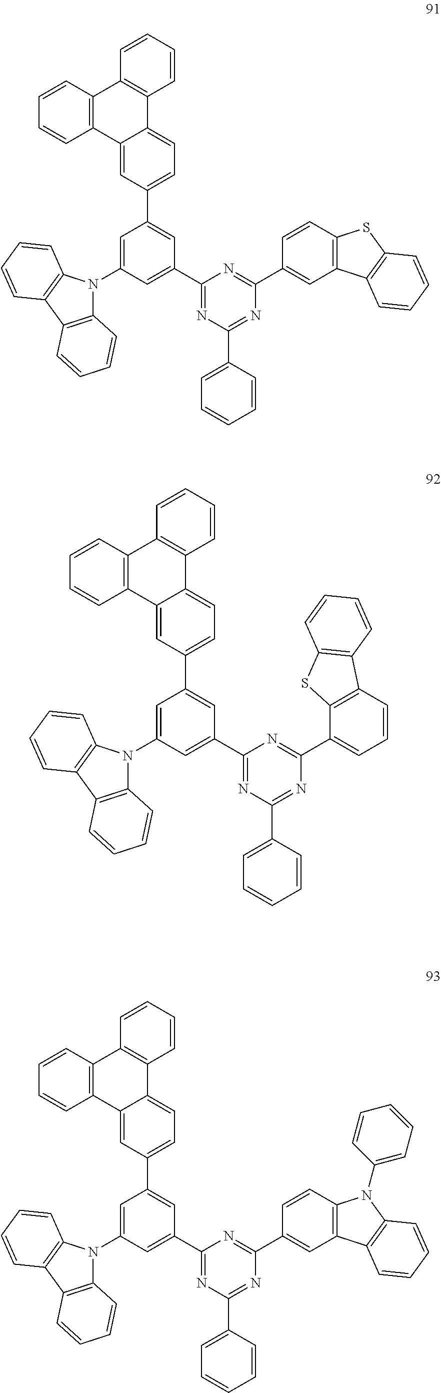 [SMILES: c1ccc(-c2nc(-c3cc(-c4ccc5c6ccccc6c6ccccc6c5c4)cc(-n4c5ccccc5c5ccccc54)c3)nc(-c3ccc4sc5ccccc5c4c3)n2)cc1 ; c1ccc(-c2nc(-c3cc(-c4ccc5c6ccccc6c6ccccc6c5c4)cc(-n4c5ccccc5c5ccccc54)c3)nc(-c3cccc4c3sc3ccccc34)n2)cc1 ; c1ccc(-c2nc(-c3cc(-c4ccc5c6ccccc6c6ccccc6c5c4)cc(-n4c5ccccc5c5ccccc54)c3)nc(-c3ccc4c(c3)c3ccccc3n4-c3ccccc3)n2)cc1]

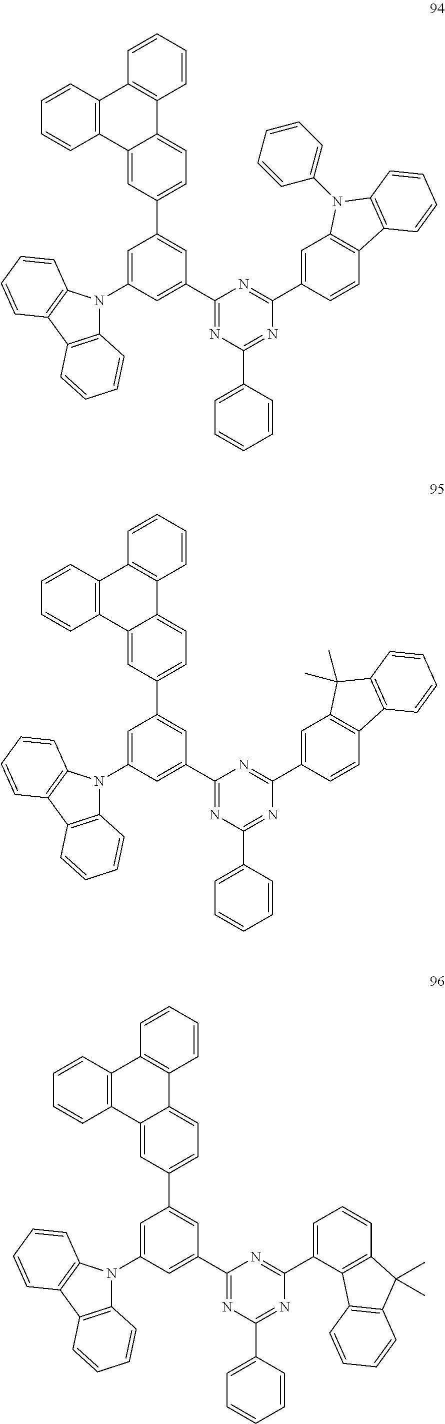 [SMILES: c1ccc(-c2nc(-c3cc(-c4ccc5c6ccccc6c6ccccc6c5c4)cc(-n4c5ccccc5c5ccccc54)c3)nc(-c3ccc4c5ccccc5n(-c5ccccc5)c4c3)n2)cc1 ; CC1(C)c2ccccc2-c2ccc(-c3nc(-c4ccccc4)nc(-c4cc(-c5ccc6c7ccccc7c7ccccc7c6c5)cc(-n5c6ccccc6c6ccccc65)c4)n3)cc21 ; CC1(C)c2ccccc2-c2c(-c3nc(-c4ccccc4)nc(-c4cc(-c5ccc6c7ccccc7c7ccccc7c6c5)cc(-n5c6ccccc6c6ccccc65)c4)n3)cccc21]

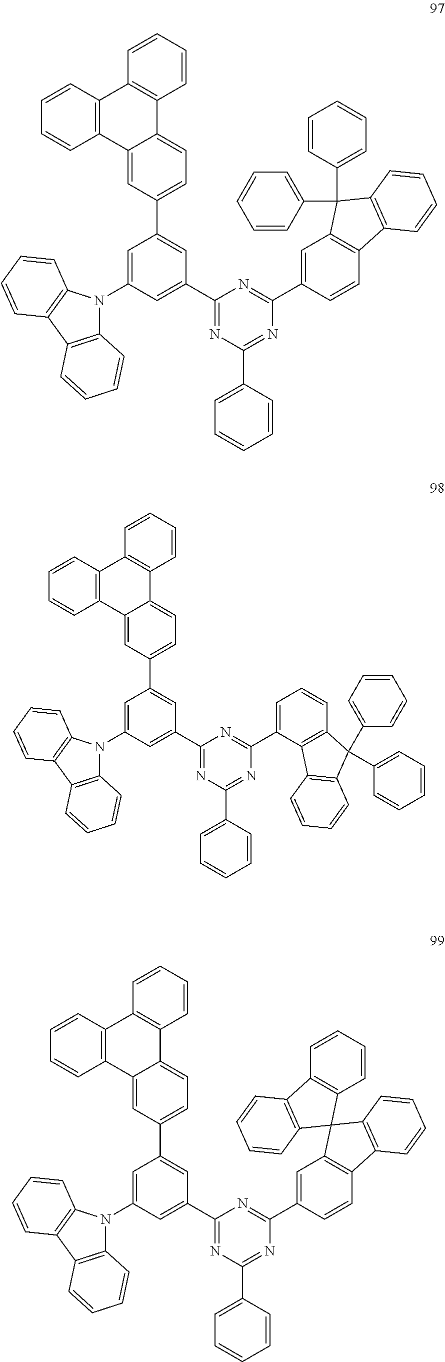 [SMILES: c1ccc(-c2nc(-c3cc(-c4ccc5c6ccccc6c6ccccc6c5c4)cc(-n4c5ccccc5c5ccccc54)c3)nc(-c3ccc4c(c3)C(c3ccccc3)(c3ccccc3)c3ccccc3-4)n2)cc1 ; c1ccc(-c2nc(-c3cc(-c4ccc5c6ccccc6c6ccccc6c5c4)cc(-n4c5ccccc5c5ccccc54)c3)nc(-c3cccc4c3-c3ccccc3C4(c3ccccc3)c3ccccc3)n2)cc1 ; c1ccc(-c2nc(-c3cc(-c4ccc5c6ccccc6c6ccccc6c5c4)cc(-n4c5ccccc5c5ccccc54)c3)nc(-c3ccc4c(c3)C3(c5ccccc5-c5ccccc53)c3ccccc3-4)n2)cc1]

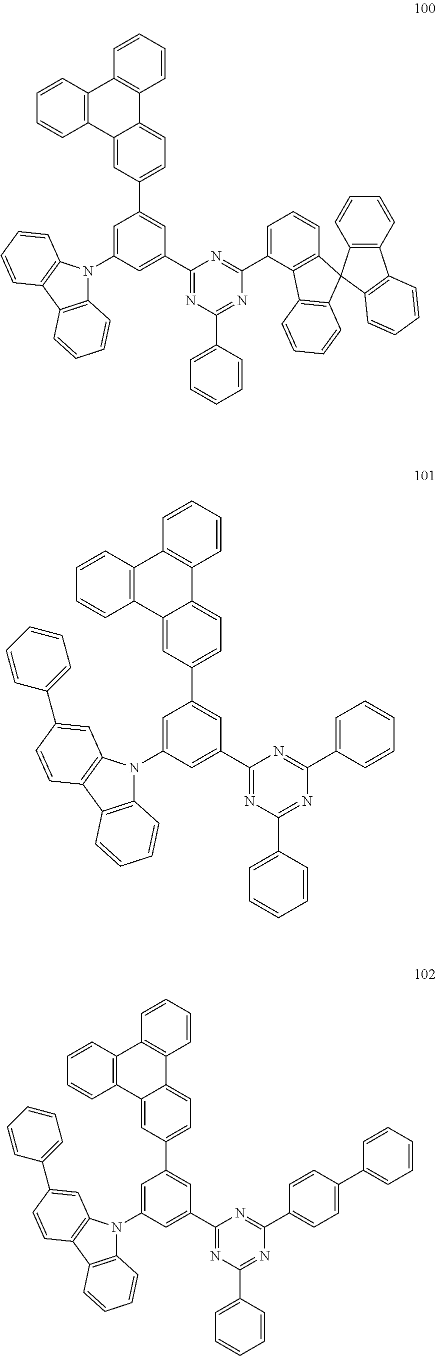 [SMILES: c1ccc(-c2nc(-c3cc(-c4ccc5c6ccccc6c6ccccc6c5c4)cc(-n4c5ccccc5c5ccccc54)c3)nc(-c3cccc4c3-c3ccccc3C43c4ccccc4-c4ccccc43)n2)cc1 ; c1ccc(-c2ccc3c4ccccc4n(-c4cc(-c5ccc6c7ccccc7c7ccccc7c6c5)cc(-c5nc(-c6ccccc6)nc(-c6ccccc6)n5)c4)c3c2)cc1 ; c1ccc(-c2ccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cc2)cc1]

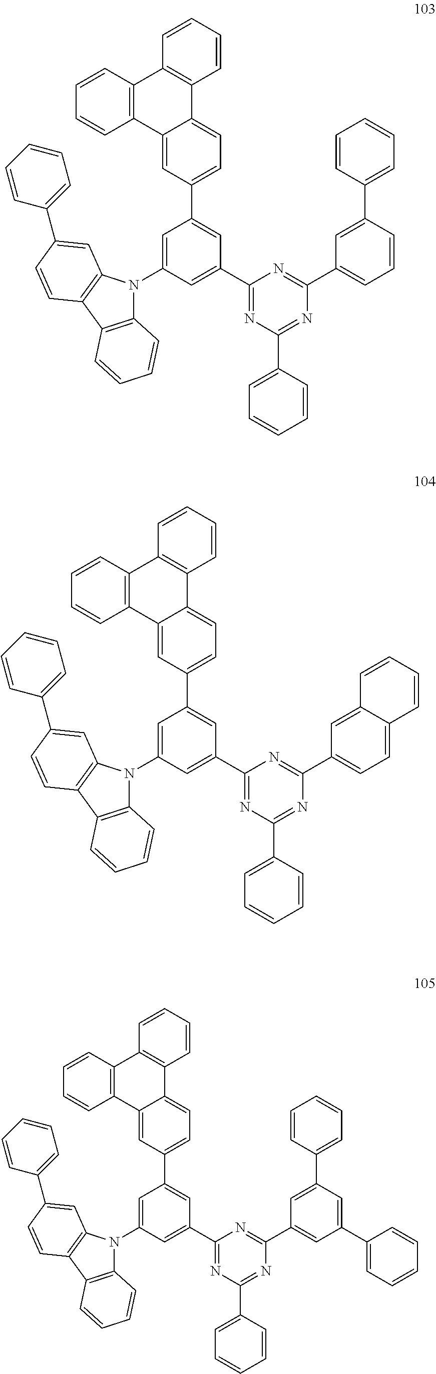 [SMILES: c1ccc(-c2cccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7ccccc7c6)n5)c4)c3c2)cc1 ; c1ccc(-c2cc(-c3ccccc3)cc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)c2)cc1]

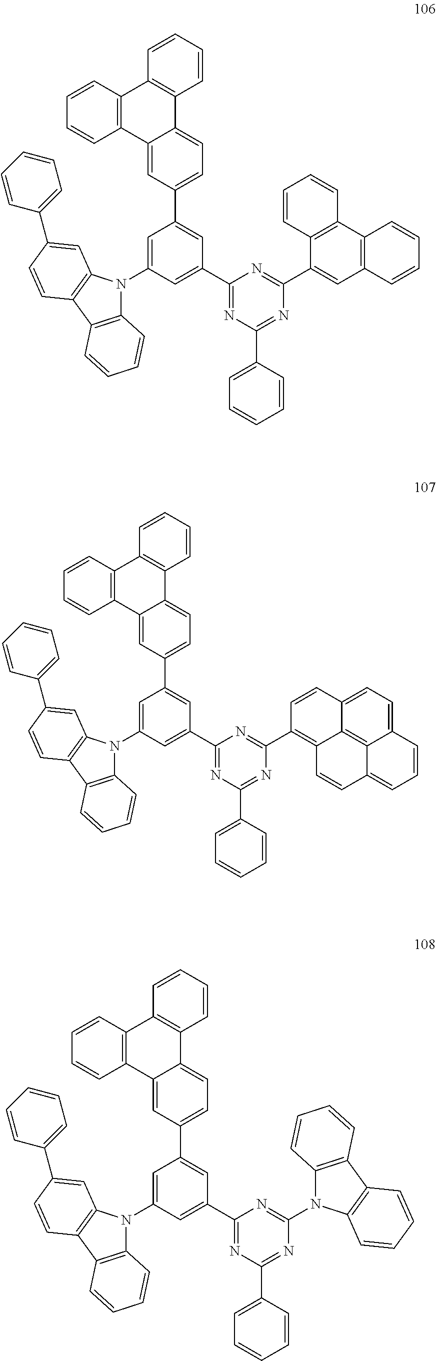 [SMILES: c1ccc(-c2ccc3c4ccccc4n(-c4cc(-c5ccc6c7ccccc7c7ccccc7c6c5)cc(-c5nc(-c6ccccc6)nc(-c6cc7ccccc7c7ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7ccc8cccc9ccc6c7c89)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-n6c7ccccc7c7ccccc76)n5)c4)c3c2)cc1]

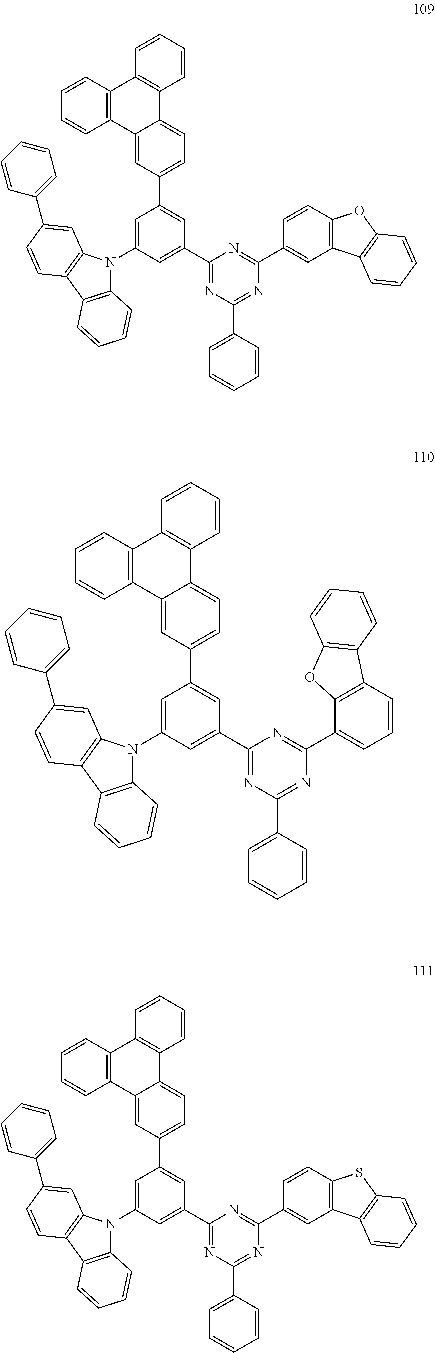 [SMILES: c1ccc(-c2ccc3c4ccccc4n(-c4cc(-c5ccc6c7ccccc7c7ccccc7c6c5)cc(-c5nc(-c6ccccc6)nc(-c6ccc7oc8ccccc8c7c6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6cccc7c6oc6ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7sc8ccccc8c7c6)n5)c4)c3c2)cc1]

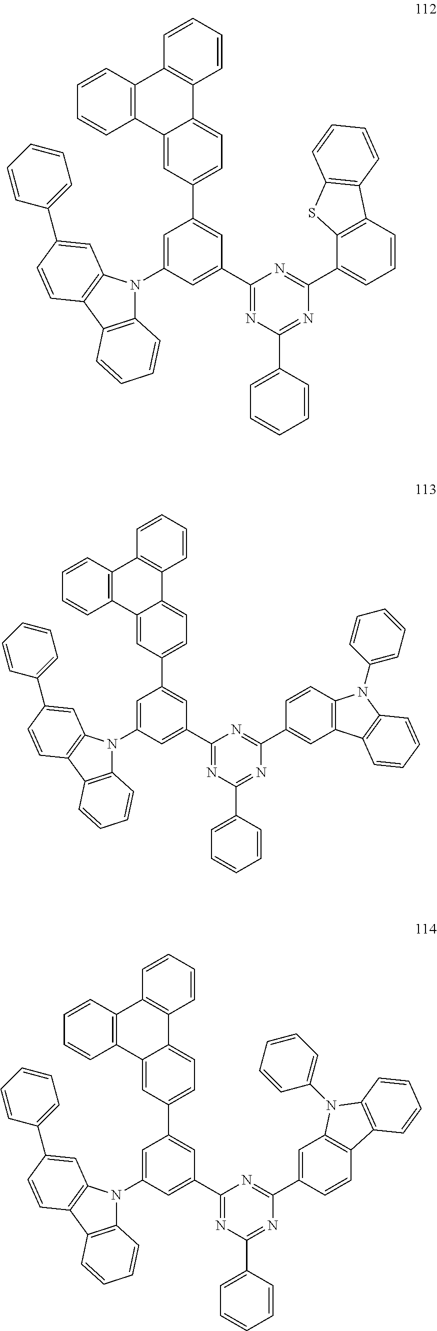 [SMILES: c1ccc(-c2ccc3c4ccccc4n(-c4cc(-c5ccc6c7ccccc7c7ccccc7c6c5)cc(-c5nc(-c6ccccc6)nc(-c6cccc7c6sc6ccccc67)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7c(c6)c6ccccc6n7-c6ccccc6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7c8ccccc8n(-c8ccccc8)c7c6)n5)c4)c3c2)cc1]

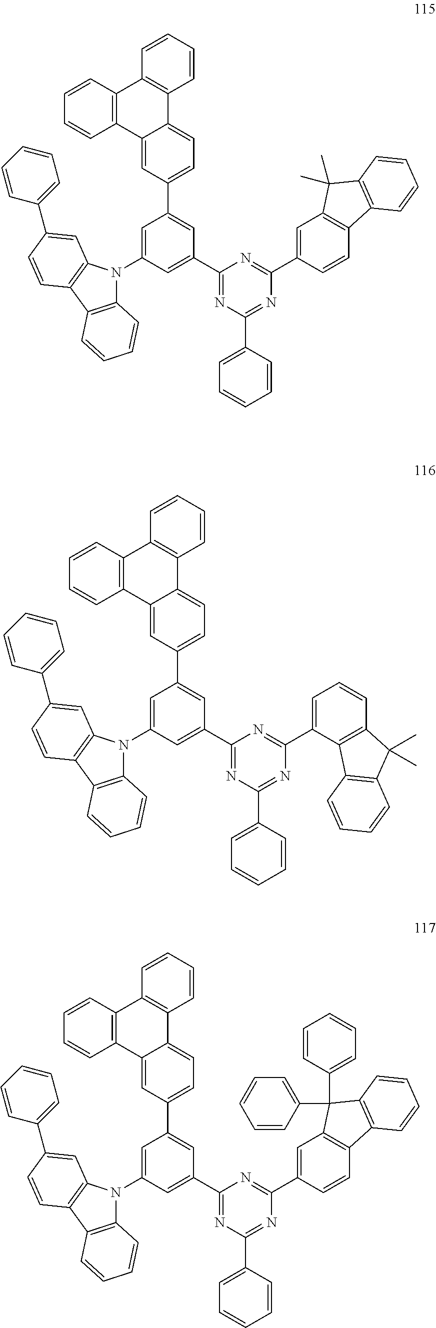 [SMILES: CC1(C)c2ccccc2-c2ccc(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cc21 ; CC1(C)c2ccccc2-c2c(-c3nc(-c4ccccc4)nc(-c4cc(-c5ccc6c7ccccc7c7ccccc7c6c5)cc(-n5c6ccccc6c6ccc(-c7ccccc7)cc65)c4)n3)cccc21 ; c1ccc(-c2ccc3c4ccccc4n(-c4cc(-c5ccc6c7ccccc7c7ccccc7c6c5)cc(-c5nc(-c6ccccc6)nc(-c6ccc7c(c6)C(c6ccccc6)(c6ccccc6)c6ccccc6-7)n5)c4)c3c2)cc1]

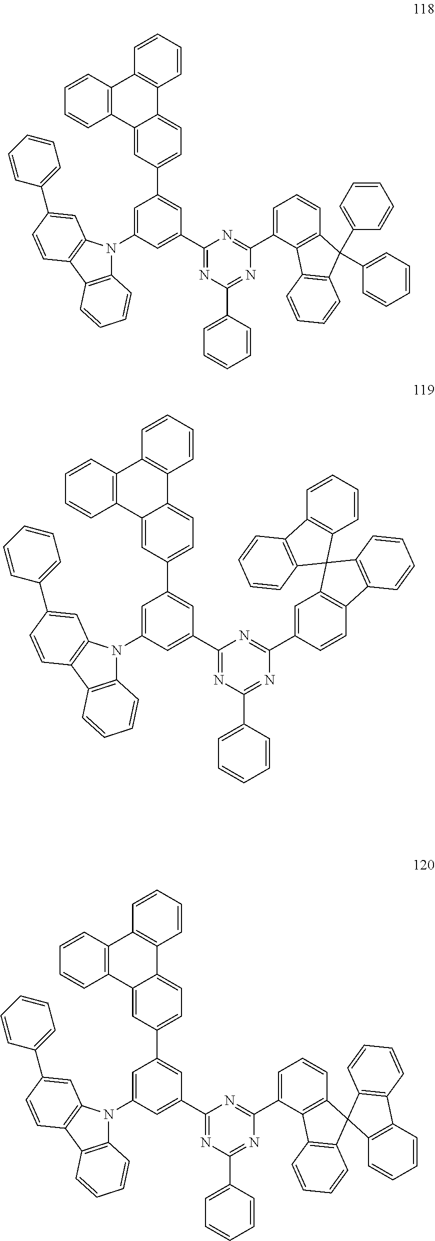 [SMILES: c1ccc(-c2ccc3c4ccccc4n(-c4cc(-c5ccc6c7ccccc7c7ccccc7c6c5)cc(-c5nc(-c6ccccc6)nc(-c6cccc7c6-c6ccccc6C7(c6ccccc6)c6ccccc6)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6ccc7c(c6)C6(c8ccccc8-c8ccccc86)c6ccccc6-7)n5)c4)c3c2)cc1 ; c1ccc(-c2ccc3c4ccccc4n(-c4cc(-c5ccc6c7ccccc7c7ccccc7c6c5)cc(-c5nc(-c6ccccc6)nc(-c6cccc7c6-c6ccccc6C76c7ccccc7-c7ccccc76)n5)c4)c3c2)cc1]

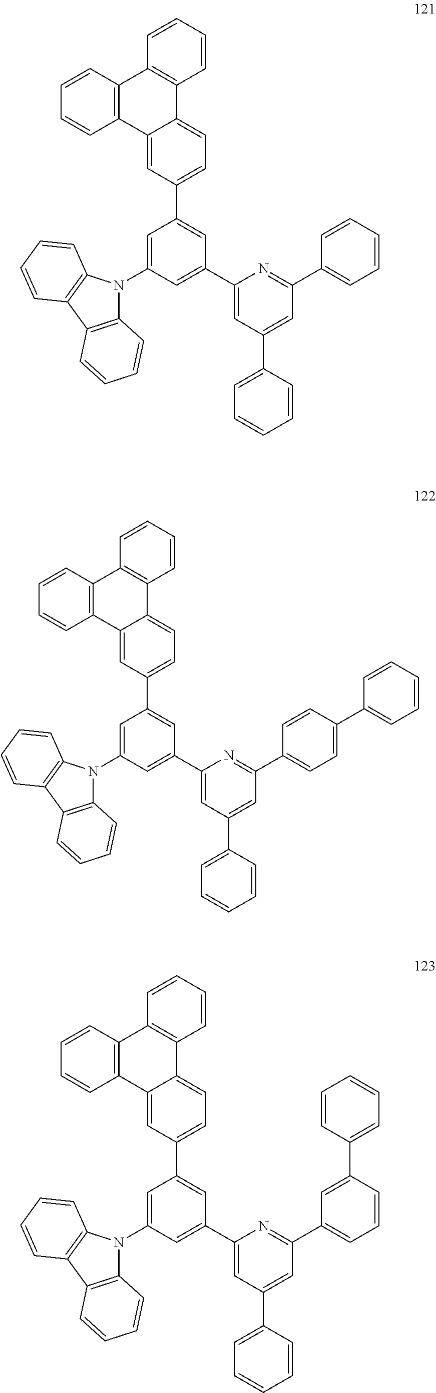 [SMILES: c1ccc(-c2cc(-c3ccccc3)nc(-c3cc(-c4ccc5c6ccccc6c6ccccc6c5c4)cc(-n4c5ccccc5c5ccccc54)c3)c2)cc1 ; c1ccc(-c2ccc(-c3cc(-c4ccccc4)cc(-c4cc(-c5ccc6c7ccccc7c7ccccc7c6c5)cc(-n5c6ccccc6c6ccccc65)c4)n3)cc2)cc1 ; c1ccc(-c2cccc(-c3cc(-c4ccccc4)cc(-c4cc(-c5ccc6c7ccccc7c7ccccc7c6c5)cc(-n5c6ccccc6c6ccccc65)c4)n3)c2)cc1]

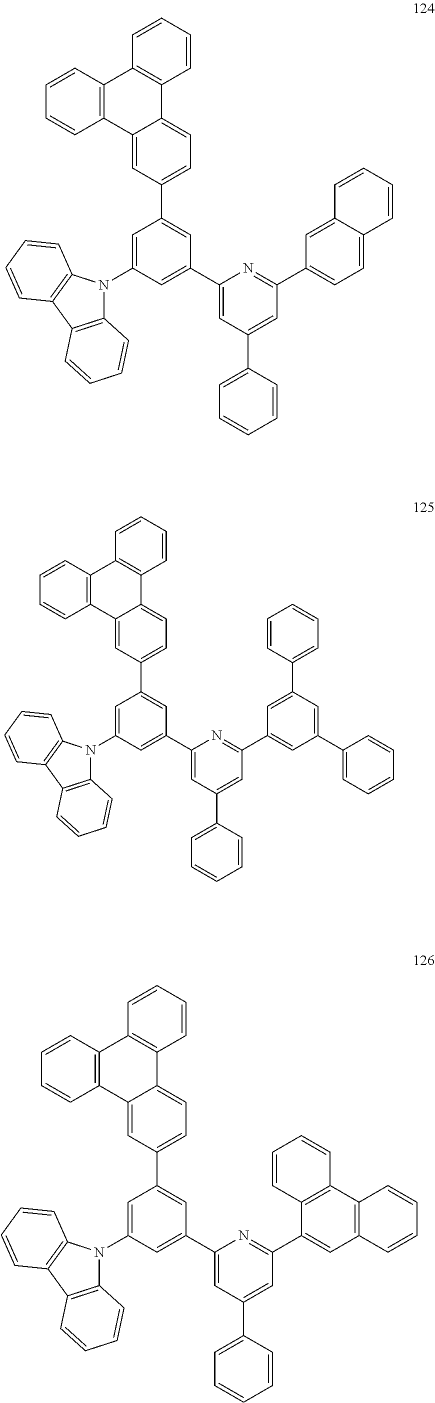 [SMILES: c1ccc(-c2cc(-c3cc(-c4ccc5c6ccccc6c6ccccc6c5c4)cc(-n4c5ccccc5c5ccccc54)c3)nc(-c3ccc4ccccc4c3)c2)cc1 ; c1ccc(-c2cc(-c3ccccc3)cc(-c3cc(-c4ccccc4)cc(-c4cc(-c5ccc6c7ccccc7c7ccccc7c6c5)cc(-n5c6ccccc6c6ccccc65)c4)n3)c2)cc1 ; c1ccc(-c2cc(-c3cc(-c4ccc5c6ccccc6c6ccccc6c5c4)cc(-n4c5ccccc5c5ccccc54)c3)nc(-c3cc4ccccc4c4ccccc34)c2)cc1]

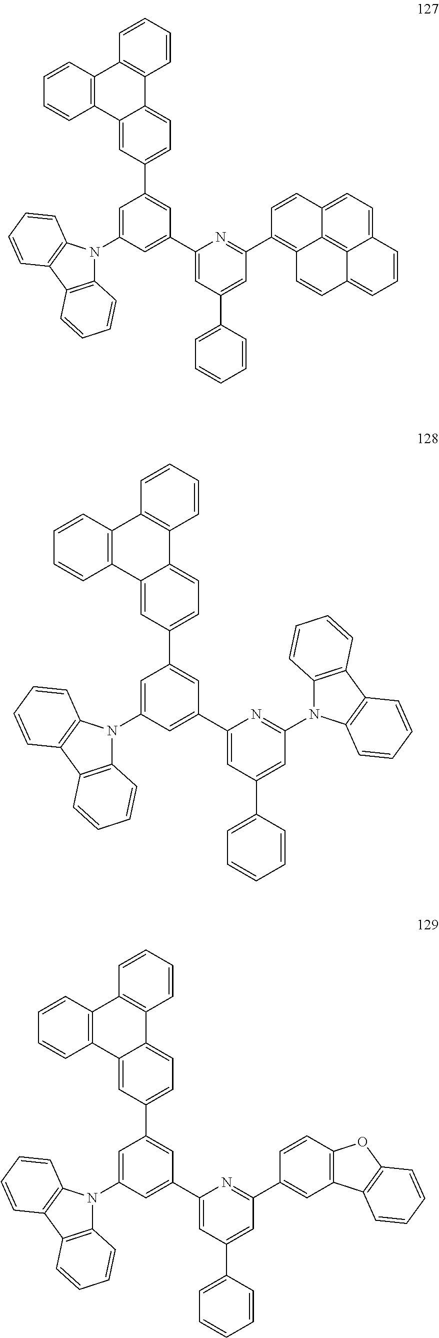 [SMILES: c1ccc(-c2cc(-c3cc(-c4ccc5c6ccccc6c6ccccc6c5c4)cc(-n4c5ccccc5c5ccccc54)c3)nc(-c3ccc4ccc5cccc6ccc3c4c56)c2)cc1 ; c1ccc(-c2cc(-c3cc(-c4ccc5c6ccccc6c6ccccc6c5c4)cc(-n4c5ccccc5c5ccccc54)c3)nc(-n3c4ccccc4c4ccccc43)c2)cc1 ; c1ccc(-c2cc(-c3cc(-c4ccc5c6ccccc6c6ccccc6c5c4)cc(-n4c5ccccc5c5ccccc54)c3)nc(-c3ccc4oc5ccccc5c4c3)c2)cc1]

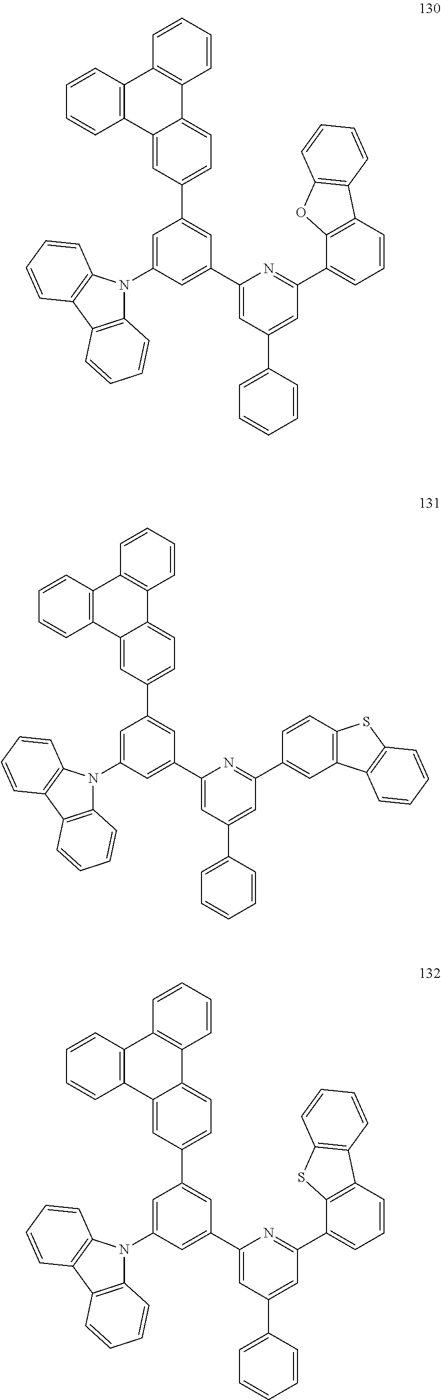 [SMILES: c1ccc(-c2cc(-c3cc(-c4ccc5c6ccccc6c6ccccc6c5c4)cc(-n4c5ccccc5c5ccccc54)c3)nc(-c3cccc4c3oc3ccccc34)c2)cc1 ; c1ccc(-c2cc(-c3cc(-c4ccc5c6ccccc6c6ccccc6c5c4)cc(-n4c5ccccc5c5ccccc54)c3)nc(-c3ccc4sc5ccccc5c4c3)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3sc3ccccc34)c2)cc1]

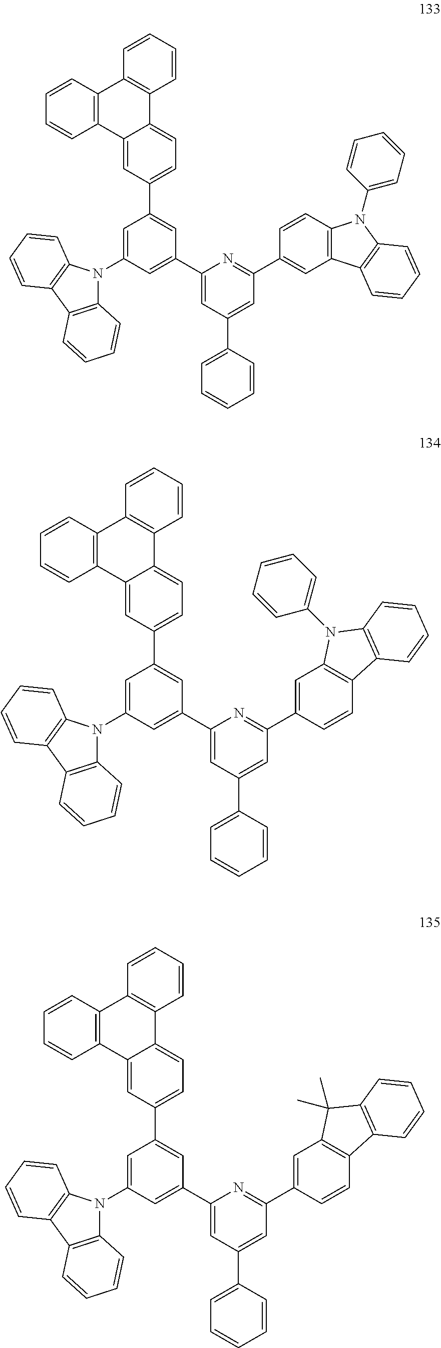 [SMILES: c1ccc(-c2cc(-c3cc(-c4ccc5c6ccccc6c6ccccc6c5c4)cc(-n4c5ccccc5c5ccccc54)c3)nc(-c3ccc4c(c3)c3ccccc3n4-c3ccccc3)c2)cc1 ; c1ccc(-c2cc(-c3cc(-c4ccc5c6ccccc6c6ccccc6c5c4)cc(-n4c5ccccc5c5ccccc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5ccc6c7ccccc7c7ccccc7c6c5)cc(-n5c6ccccc6c6ccccc65)c4)n3)cc21]

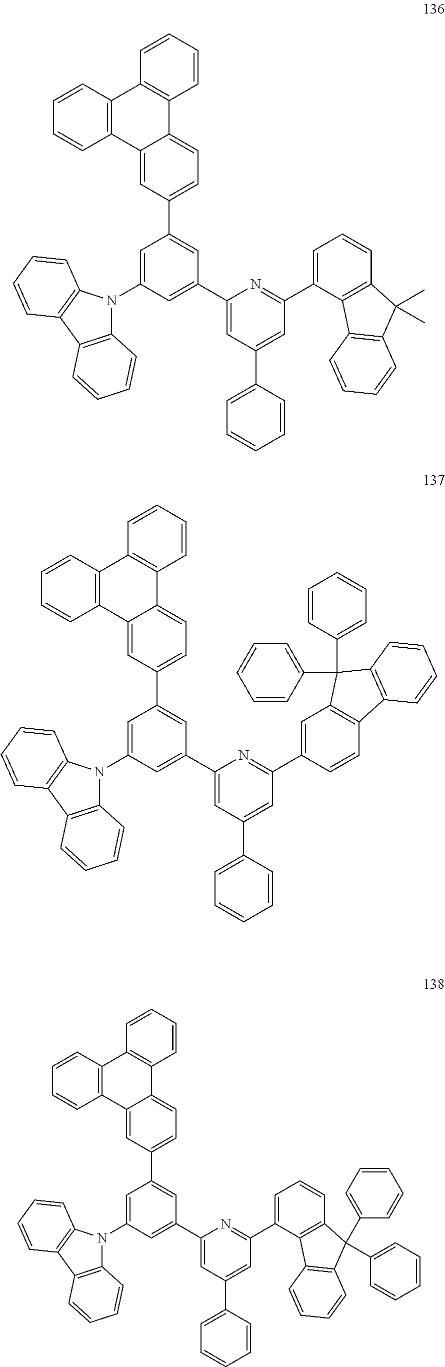 [SMILES: CC1(C)c2ccccc2-c2c(-c3cc(-c4ccccc4)cc(-c4cc(-c5ccc6c7ccccc7c7ccccc7c6c5)cc(-n5c6ccccc6c6ccccc65)c4)n3)cccc21 ; c1ccc(-c2cc(-c3cc(-c4ccc5c6ccccc6c6ccccc6c5c4)cc(-n4c5ccccc5c5ccccc54)c3)nc(-c3ccc4c(c3)C(c3ccccc3)(c3ccccc3)c3ccccc3-4)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3-c3ccccc3C4(c3ccccc3)c3ccccc3)c2)cc1]

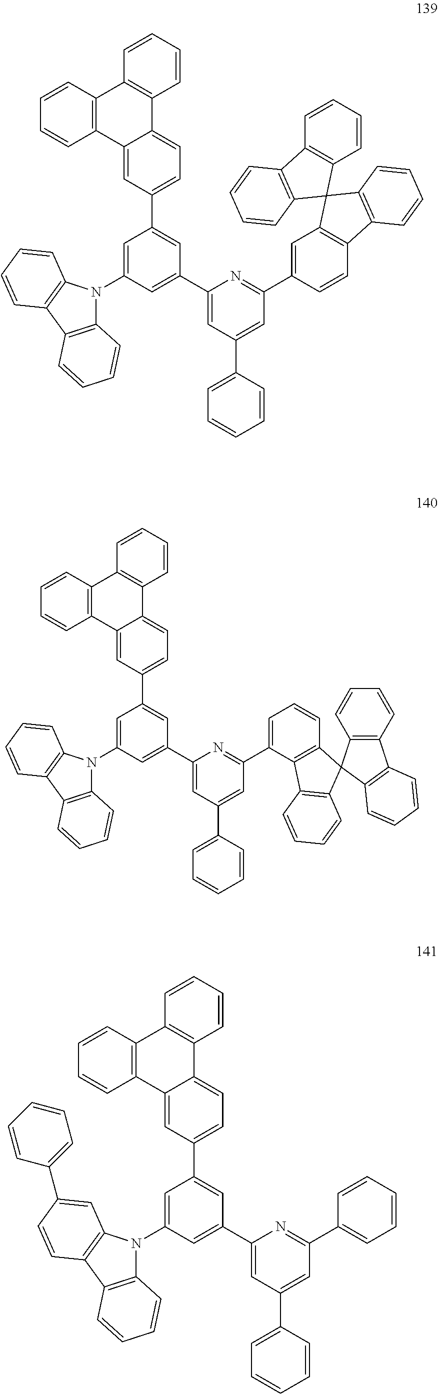 [SMILES: c1ccc(-c2cc(-c3cc(-c4ccc5c6ccccc6c6ccccc6c5c4)cc(-n4c5ccccc5c5ccccc54)c3)nc(-c3ccc4c(c3)C3(c5ccccc5-c5ccccc53)c3ccccc3-4)c2)cc1 ; c1ccc(-c2cc(-c3cc(-c4ccc5c6ccccc6c6ccccc6c5c4)cc(-n4c5ccccc5c5ccccc54)c3)nc(-c3cccc4c3-c3ccccc3C43c4ccccc4-c4ccccc43)c2)cc1 ; c1ccc(-c2cc(-c3ccccc3)nc(-c3cc(-c4ccc5c6ccccc6c6ccccc6c5c4)cc(-n4c5ccccc5c5ccc(-c6ccccc6)cc54)c3)c2)cc1]

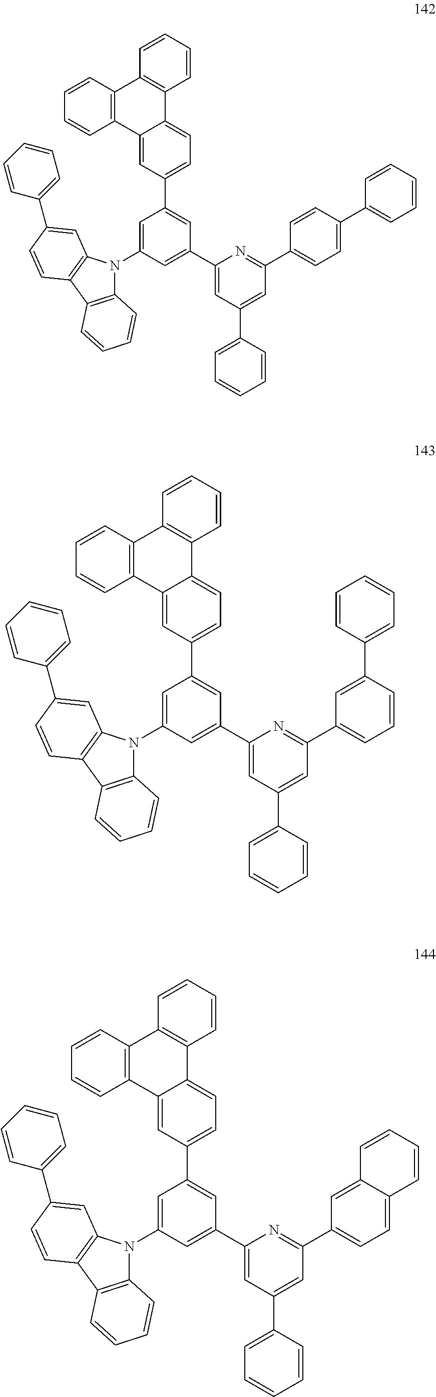 [SMILES: c1ccc(-c2ccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cc2)cc1 ; c1ccc(-c2cccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4ccccc4c3)c2)cc1]

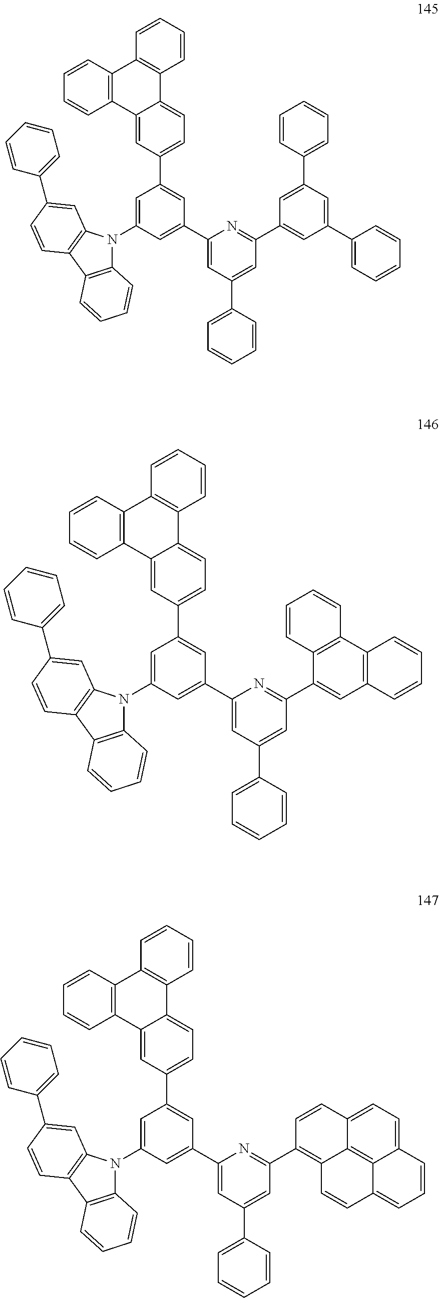 [SMILES: c1ccc(-c2cc(-c3ccccc3)cc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cc4ccccc4c4ccccc34)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4ccc5cccc6ccc3c4c56)c2)cc1]

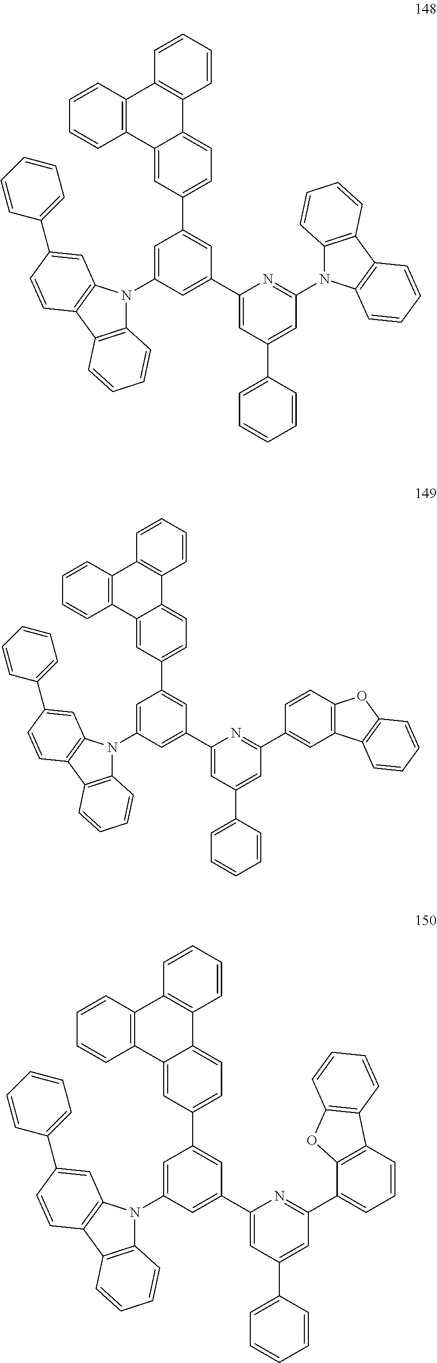 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-n3c4ccccc4c4ccccc43)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4oc5ccccc5c4c3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3oc3ccccc34)c2)cc1]

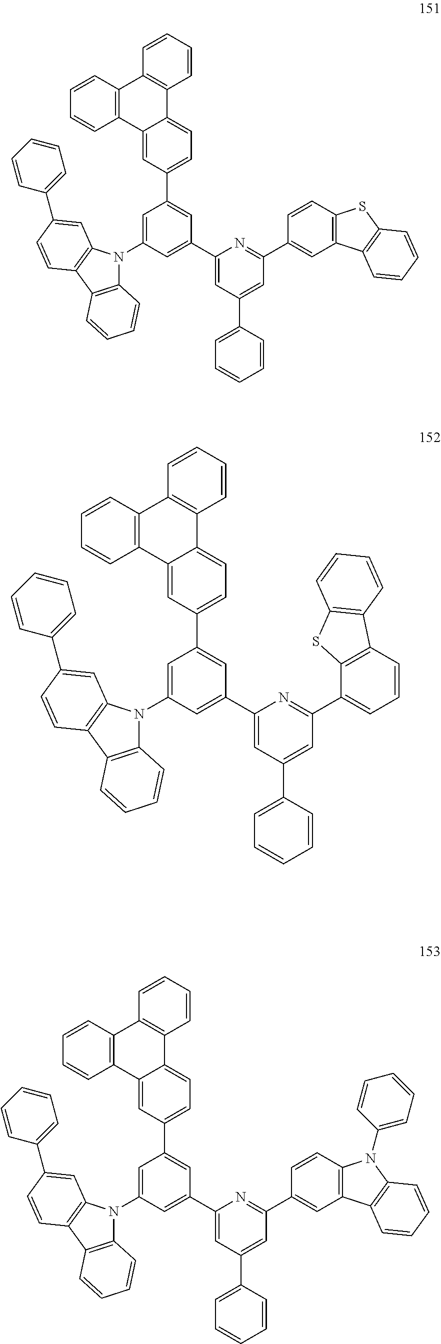 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4sc5ccccc5c4c3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3sc3ccccc34)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)c3ccccc3n4-c3ccccc3)c2)cc1]

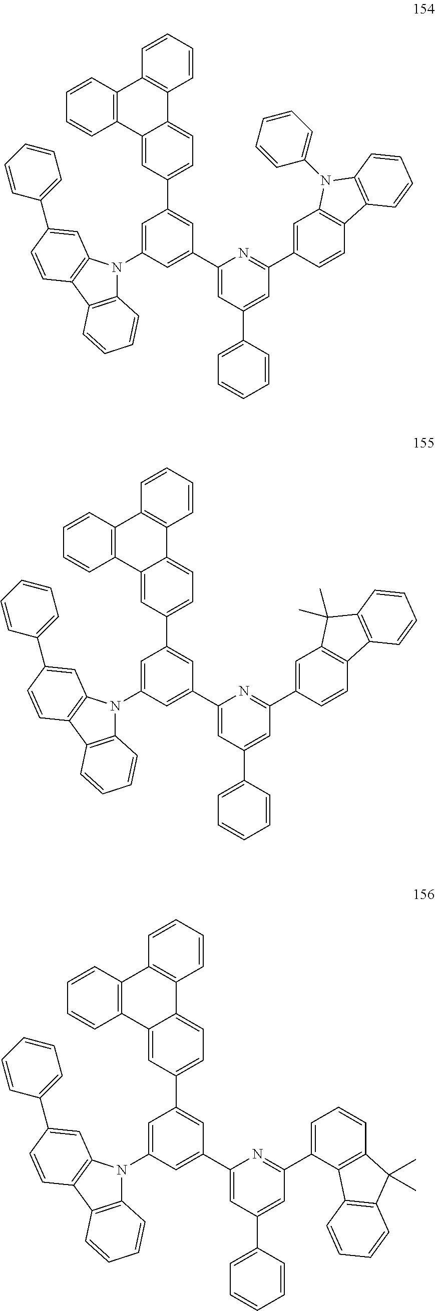 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cc21 ; CC1(C)c2ccccc2-c2c(-c3cc(-c4ccccc4)cc(-c4cc(-c5ccc6c7ccccc7c7ccccc7c6c5)cc(-n5c6ccccc6c6ccc(-c7ccccc7)cc65)c4)n3)cccc21]

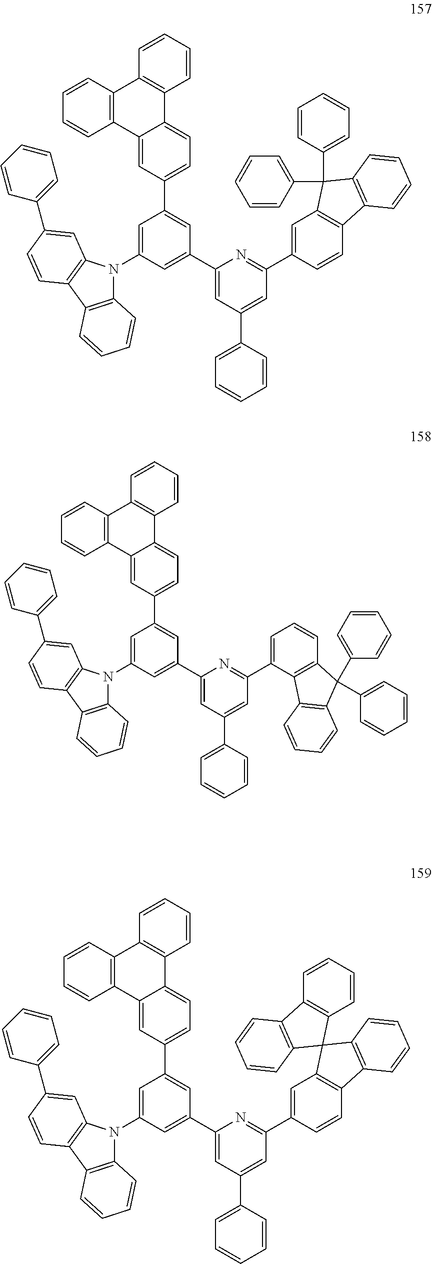 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)C(c3ccccc3)(c3ccccc3)c3ccccc3-4)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3-c3ccccc3C4(c3ccccc3)c3ccccc3)c2)cc1 ; c1ccc(-c2cc(-c3cc(-c4ccc5c6ccccc6c6ccccc6c5c4)cc(-n4c5ccccc5c5ccc(-c6ccccc6)cc54)c3)nc(-c3ccc4c(c3)C3(c5ccccc5-c5ccccc53)c3ccccc3-4)c2)cc1]

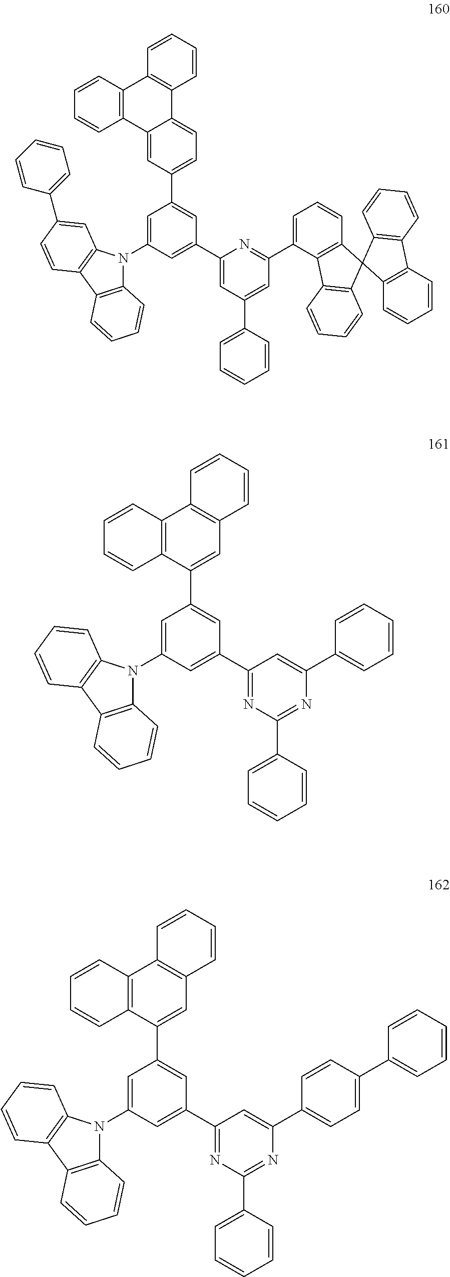 [SMILES: c1ccc(-c2cc(-c3cc(-c4ccc5c6ccccc6c6ccccc6c5c4)cc(-n4c5ccccc5c5ccc(-c6ccccc6)cc54)c3)nc(-c3cccc4c3-c3ccccc3C43c4ccccc4-c4ccccc43)c2)cc1 ; c1ccc(-c2cc(-c3cc(-c4cc5ccccc5c5ccccc45)cc(-n4c5ccccc5c5ccccc54)c3)nc(-c3ccccc3)n2)cc1 ; c1ccc(-c2ccc(-c3cc(-c4cc(-c5cc6ccccc6c6ccccc56)cc(-n5c6ccccc6c6ccccc65)c4)nc(-c4ccccc4)n3)cc2)cc1]

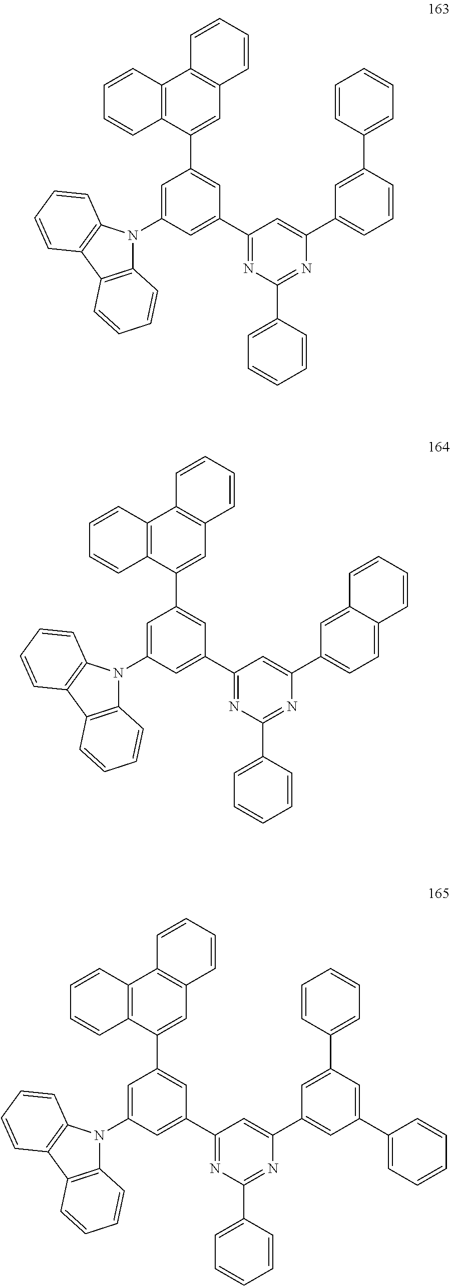 [SMILES: c1ccc(-c2cccc(-c3cc(-c4cc(-c5cc6ccccc6c6ccccc56)cc(-n5c6ccccc6c6ccccc65)c4)nc(-c4ccccc4)n3)c2)cc1 ; c1ccc(-c2nc(-c3cc(-c4cc5ccccc5c5ccccc45)cc(-n4c5ccccc5c5ccccc54)c3)cc(-c3ccc4ccccc4c3)n2)cc1 ; c1ccc(-c2cc(-c3ccccc3)cc(-c3cc(-c4cc(-c5cc6ccccc6c6ccccc56)cc(-n5c6ccccc6c6ccccc65)c4)nc(-c4ccccc4)n3)c2)cc1]

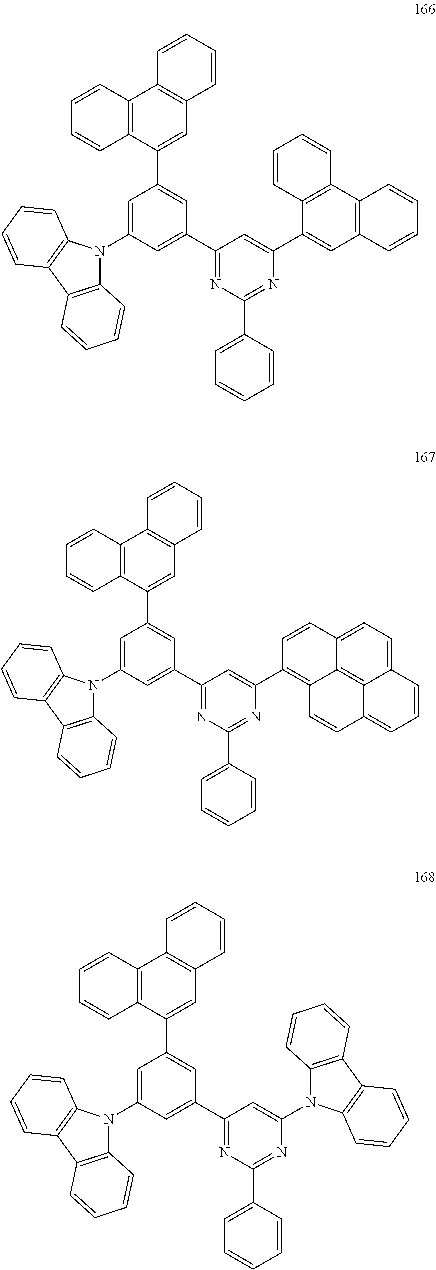 [SMILES: c1ccc(-c2nc(-c3cc(-c4cc5ccccc5c5ccccc45)cc(-n4c5ccccc5c5ccccc54)c3)cc(-c3cc4ccccc4c4ccccc34)n2)cc1 ; c1ccc(-c2nc(-c3cc(-c4cc5ccccc5c5ccccc45)cc(-n4c5ccccc5c5ccccc54)c3)cc(-c3ccc4ccc5cccc6ccc3c4c56)n2)cc1 ; c1ccc(-c2nc(-c3cc(-c4cc5ccccc5c5ccccc45)cc(-n4c5ccccc5c5ccccc54)c3)cc(-n3c4ccccc4c4ccccc43)n2)cc1]

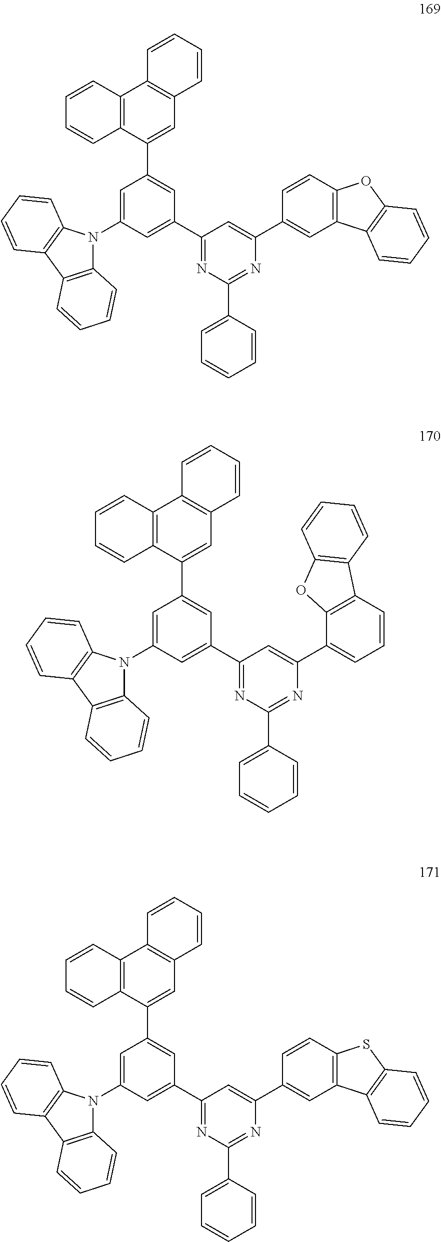 [SMILES: c1ccc(-c2nc(-c3cc(-c4cc5ccccc5c5ccccc45)cc(-n4c5ccccc5c5ccccc54)c3)cc(-c3ccc4oc5ccccc5c4c3)n2)cc1 ; c1ccc(-c2nc(-c3cc(-c4cc5ccccc5c5ccccc45)cc(-n4c5ccccc5c5ccccc54)c3)cc(-c3cccc4c3oc3ccccc34)n2)cc1 ; c1ccc(-c2nc(-c3cc(-c4cc5ccccc5c5ccccc45)cc(-n4c5ccccc5c5ccccc54)c3)cc(-c3ccc4sc5ccccc5c4c3)n2)cc1]

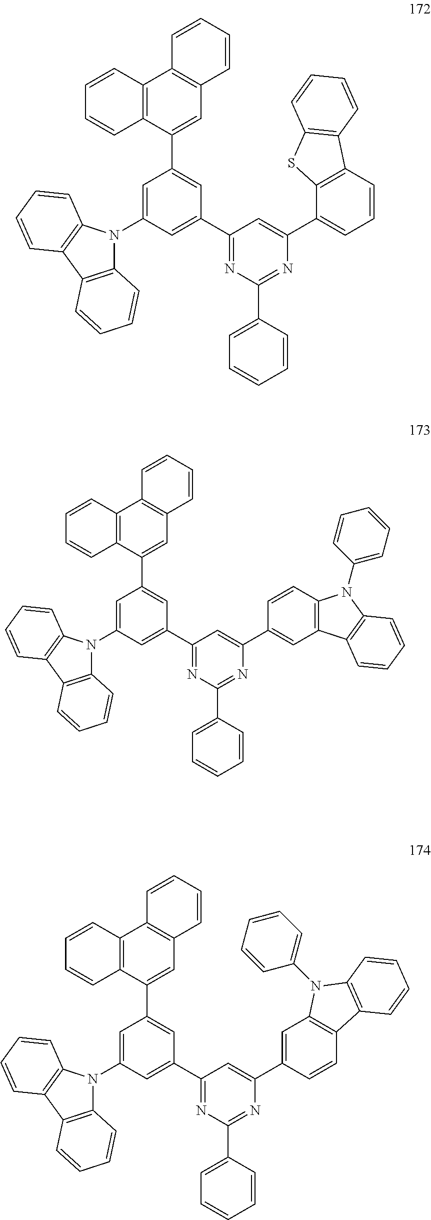 [SMILES: c1ccc(-c2nc(-c3cc(-c4cc5ccccc5c5ccccc45)cc(-n4c5ccccc5c5ccccc54)c3)cc(-c3cccc4c3sc3ccccc34)n2)cc1 ; c1ccc(-c2nc(-c3cc(-c4cc5ccccc5c5ccccc45)cc(-n4c5ccccc5c5ccccc54)c3)cc(-c3ccc4c(c3)c3ccccc3n4-c3ccccc3)n2)cc1 ; c1ccc(-c2nc(-c3cc(-c4cc5ccccc5c5ccccc45)cc(-n4c5ccccc5c5ccccc54)c3)cc(-c3ccc4c5ccccc5n(-c5ccccc5)c4c3)n2)cc1]

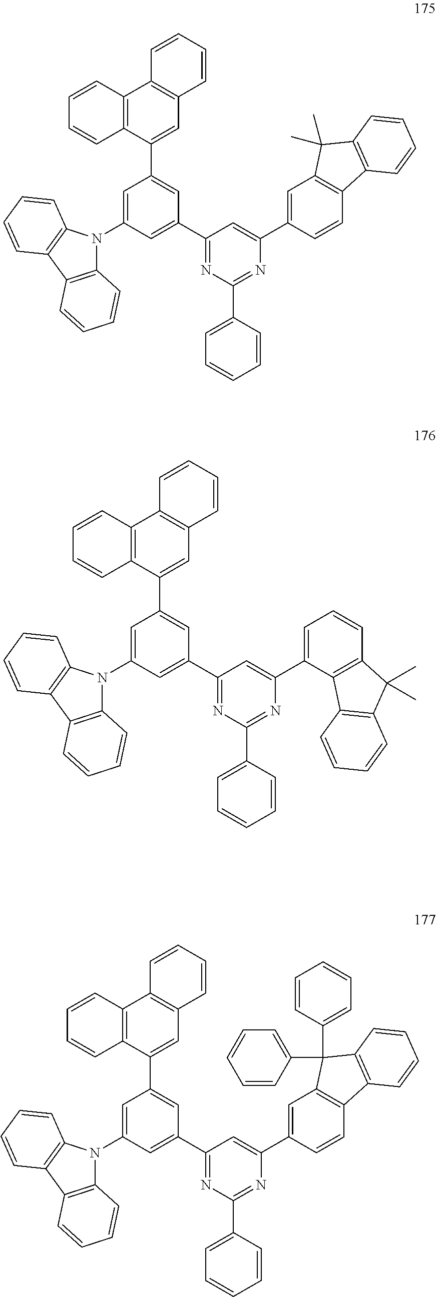 [SMILES: CC1(C)c2ccccc2-c2ccc(-c3cc(-c4cc(-c5cc6ccccc6c6ccccc56)cc(-n5c6ccccc6c6ccccc65)c4)nc(-c4ccccc4)n3)cc21 ; CC1(C)c2ccccc2-c2c(-c3cc(-c4cc(-c5cc6ccccc6c6ccccc56)cc(-n5c6ccccc6c6ccccc65)c4)nc(-c4ccccc4)n3)cccc21 ; c1ccc(-c2nc(-c3cc(-c4cc5ccccc5c5ccccc45)cc(-n4c5ccccc5c5ccccc54)c3)cc(-c3ccc4c(c3)C(c3ccccc3)(c3ccccc3)c3ccccc3-4)n2)cc1]

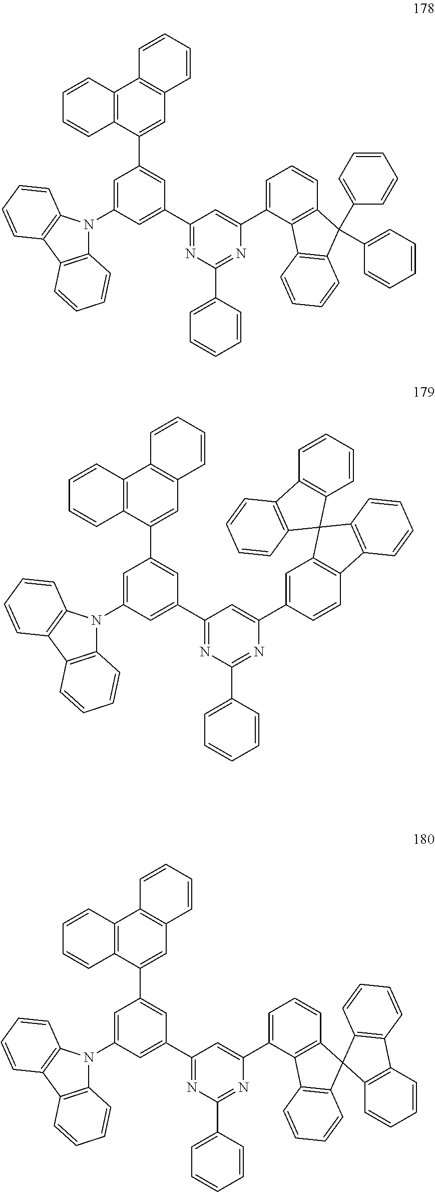 [SMILES: c1ccc(-c2nc(-c3cc(-c4cc5ccccc5c5ccccc45)cc(-n4c5ccccc5c5ccccc54)c3)cc(-c3cccc4c3-c3ccccc3C4(c3ccccc3)c3ccccc3)n2)cc1 ; c1ccc(-c2nc(-c3cc(-c4cc5ccccc5c5ccccc45)cc(-n4c5ccccc5c5ccccc54)c3)cc(-c3ccc4c(c3)C3(c5ccccc5-c5ccccc53)c3ccccc3-4)n2)cc1 ; c1ccc(-c2nc(-c3cc(-c4cc5ccccc5c5ccccc45)cc(-n4c5ccccc5c5ccccc54)c3)cc(-c3cccc4c3-c3ccccc3C43c4ccccc4-c4ccccc43)n2)cc1]

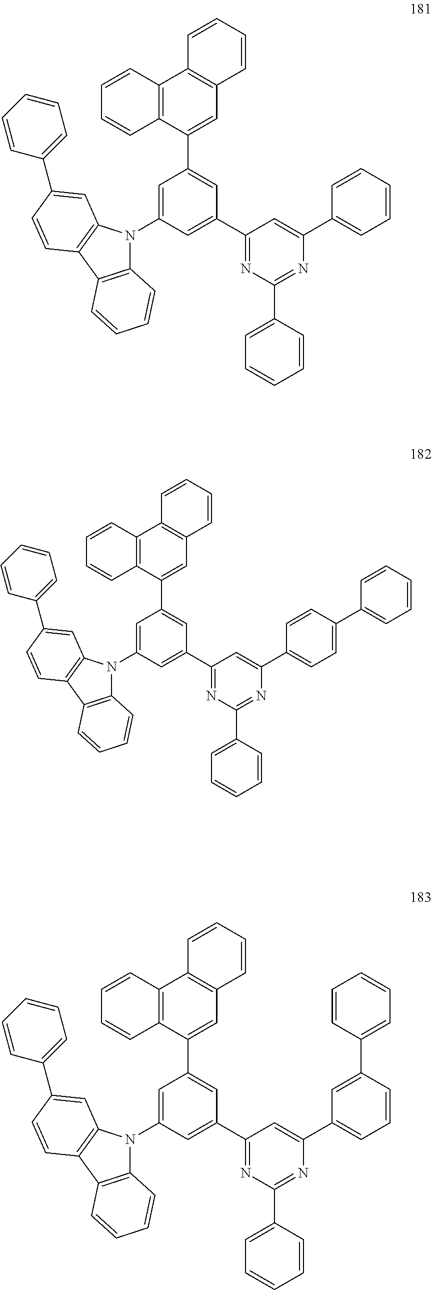 [SMILES: c1ccc(-c2ccc3c4ccccc4n(-c4cc(-c5cc(-c6ccccc6)nc(-c6ccccc6)n5)cc(-c5cc6ccccc6c6ccccc56)c4)c3c2)cc1 ; c1ccc(-c2ccc(-c3cc(-c4cc(-c5cc6ccccc6c6ccccc56)cc(-n5c6ccccc6c6ccc(-c7ccccc7)cc65)c4)nc(-c4ccccc4)n3)cc2)cc1 ; c1ccc(-c2cccc(-c3cc(-c4cc(-c5cc6ccccc6c6ccccc56)cc(-n5c6ccccc6c6ccc(-c7ccccc7)cc65)c4)nc(-c4ccccc4)n3)c2)cc1]

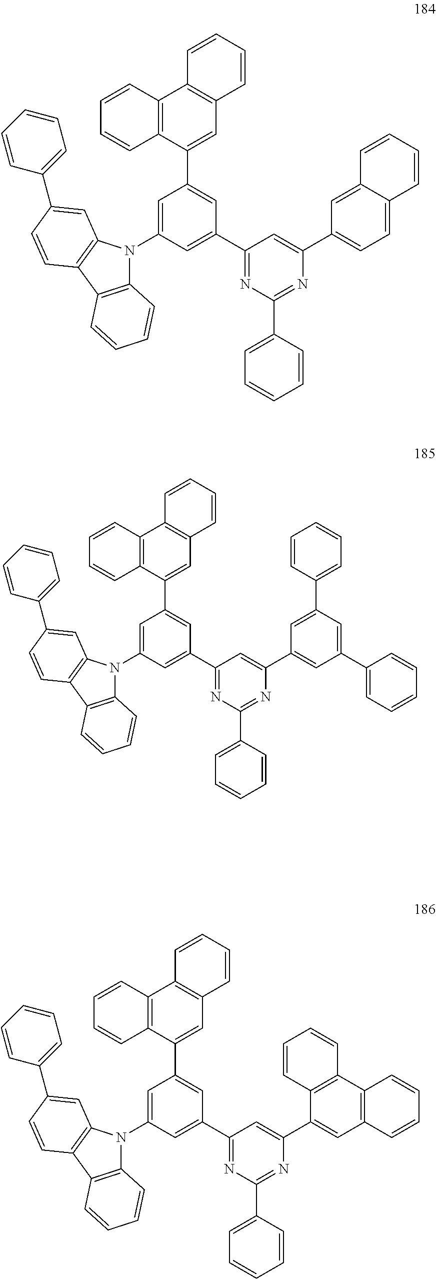 [SMILES: c1ccc(-c2ccc3c4ccccc4n(-c4cc(-c5cc(-c6ccc7ccccc7c6)nc(-c6ccccc6)n5)cc(-c5cc6ccccc6c6ccccc56)c4)c3c2)cc1 ; c1ccc(-c2cc(-c3ccccc3)cc(-c3cc(-c4cc(-c5cc6ccccc6c6ccccc56)cc(-n5c6ccccc6c6ccc(-c7ccccc7)cc65)c4)nc(-c4ccccc4)n3)c2)cc1 ; c1ccc(-c2ccc3c4ccccc4n(-c4cc(-c5cc(-c6cc7ccccc7c7ccccc67)nc(-c6ccccc6)n5)cc(-c5cc6ccccc6c6ccccc56)c4)c3c2)cc1]

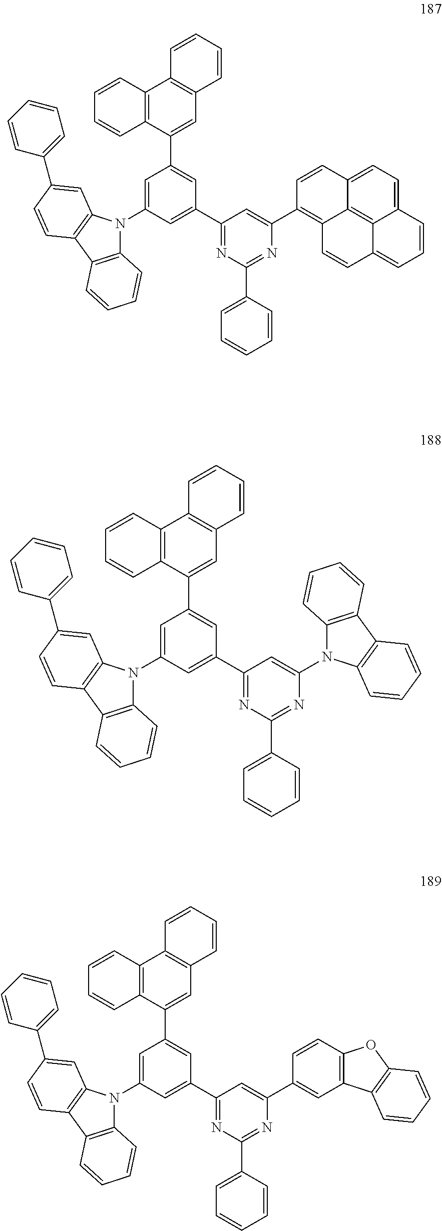 [SMILES: c1ccc(-c2ccc3c4ccccc4n(-c4cc(-c5cc(-c6ccc7ccc8cccc9ccc6c7c89)nc(-c6ccccc6)n5)cc(-c5cc6ccccc6c6ccccc56)c4)c3c2)cc1 ; c1ccc(-c2ccc3c4ccccc4n(-c4cc(-c5cc(-n6c7ccccc7c7ccccc76)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7oc8ccccc8c7c6)nc(-c6ccccc6)n5)cc(-c5cc6ccccc6c6ccccc56)c4)c3c2)cc1]

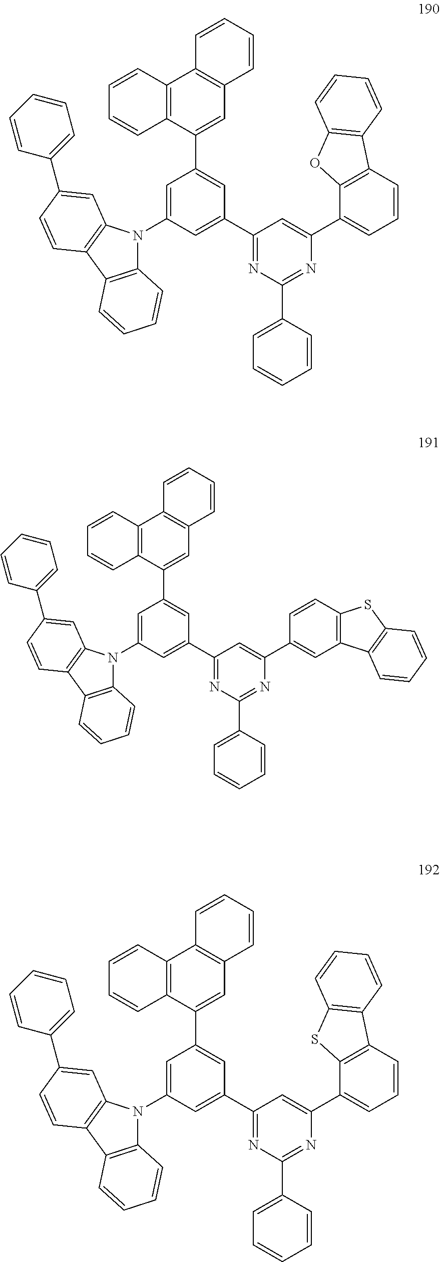 [SMILES: c1ccc(-c2ccc3c4ccccc4n(-c4cc(-c5cc(-c6cccc7c6oc6ccccc67)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7sc8ccccc8c7c6)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6sc6ccccc67)nc(-c6ccccc6)n5)cc(-c5cc6ccccc6c6ccccc56)c4)c3c2)cc1]

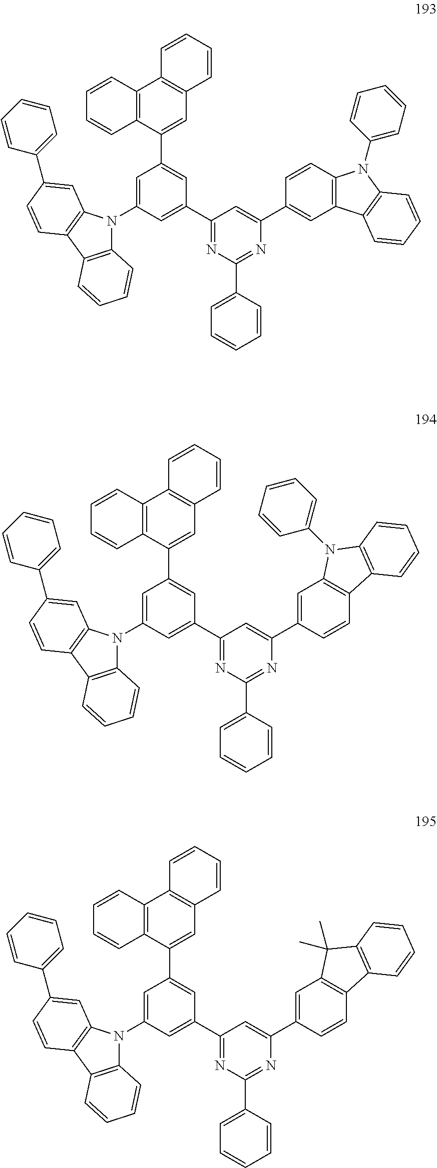 [SMILES: c1ccc(-c2ccc3c4ccccc4n(-c4cc(-c5cc(-c6ccc7c(c6)c6ccccc6n7-c6ccccc6)nc(-c6ccccc6)n5)cc(-c5cc6ccccc6c6ccccc56)c4)c3c2)cc1 ; c1ccc(-c2ccc3c4ccccc4n(-c4cc(-c5cc(-c6ccc7c8ccccc8n(-c8ccccc8)c7c6)nc(-c6ccccc6)n5)cc(-c5cc6ccccc6c6ccccc56)c4)c3c2)cc1 ; CC1(C)c2ccccc2-c2ccc(-c3cc(-c4cc(-c5cc6ccccc6c6ccccc56)cc(-n5c6ccccc6c6ccc(-c7ccccc7)cc65)c4)nc(-c4ccccc4)n3)cc21]

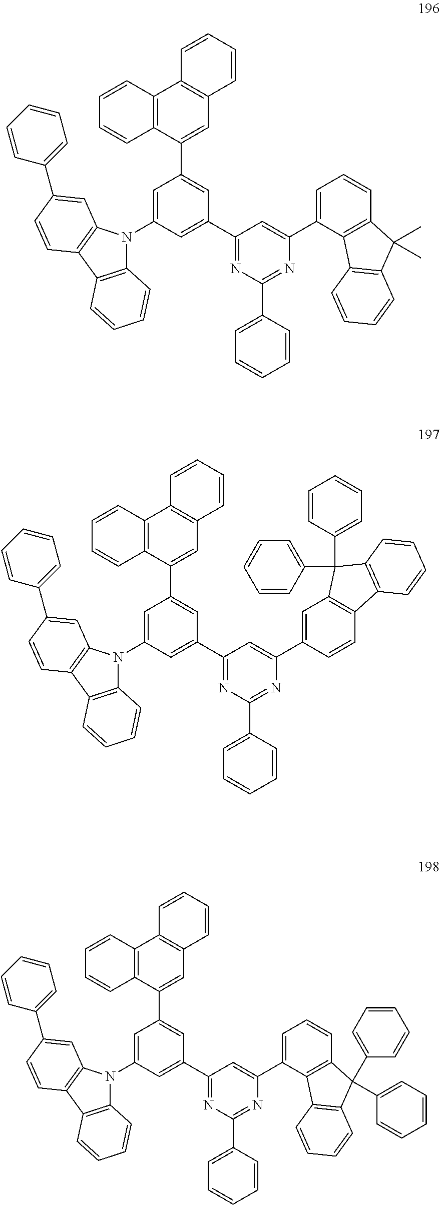 [SMILES: CC1(C)c2ccccc2-c2c(-c3cc(-c4cc(-c5cc6ccccc6c6ccccc56)cc(-n5c6ccccc6c6ccc(-c7ccccc7)cc65)c4)nc(-c4ccccc4)n3)cccc21 ; c1ccc(-c2ccc3c4ccccc4n(-c4cc(-c5cc(-c6ccc7c(c6)C(c6ccccc6)(c6ccccc6)c6ccccc6-7)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6-c6ccccc6C7(c6ccccc6)c6ccccc6)nc(-c6ccccc6)n5)cc(-c5cc6ccccc6c6ccccc56)c4)c3c2)cc1]

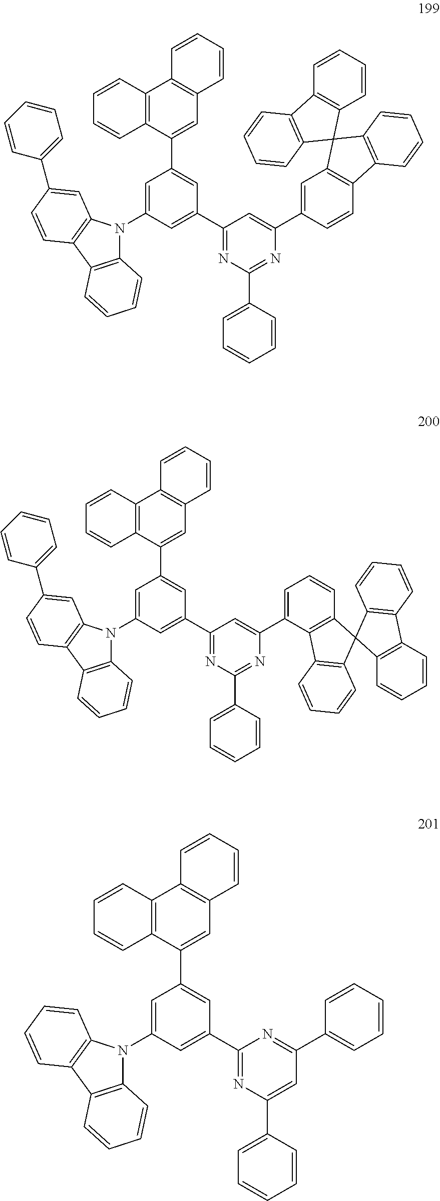 [SMILES: c1ccc(-c2ccc3c4ccccc4n(-c4cc(-c5cc(-c6ccc7c(c6)C6(c8ccccc8-c8ccccc86)c6ccccc6-7)nc(-c6ccccc6)n5)cc(-c5cc6ccccc6c6ccccc56)c4)c3c2)cc1 ; c1ccc(-c2ccc3c4ccccc4n(-c4cc(-c5cc(-c6cccc7c6-c6ccccc6C76c7ccccc7-c7ccccc76)nc(-c6ccccc6)n5)cc(-c5cc6ccccc6c6ccccc56)c4)c3c2)cc1 ; c1ccc(-c2cc(-c3ccccc3)nc(-c3cc(-c4cc5ccccc5c5ccccc45)cc(-n4c5ccccc5c5ccccc54)c3)n2)cc1]

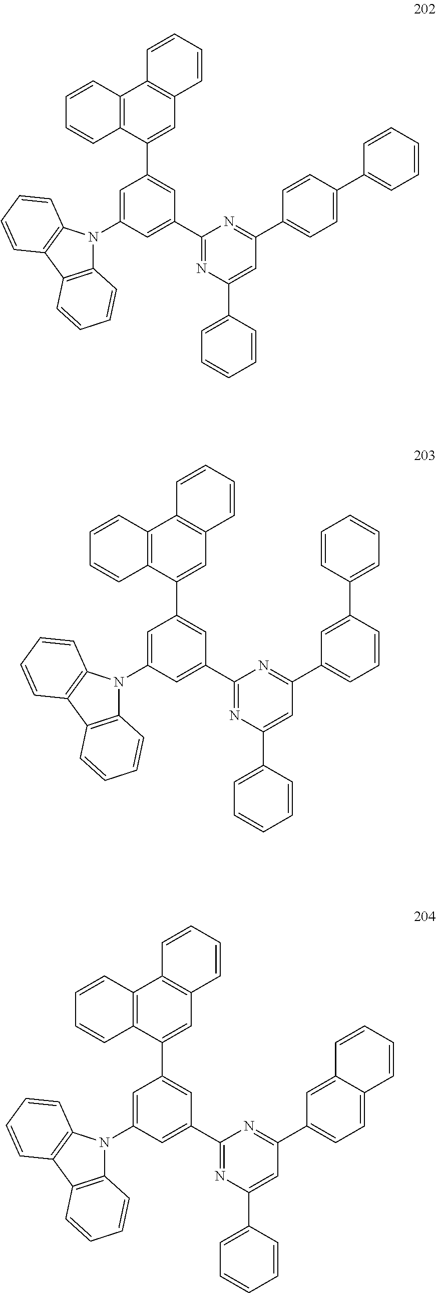 [SMILES: c1ccc(-c2ccc(-c3cc(-c4ccccc4)nc(-c4cc(-c5cc6ccccc6c6ccccc56)cc(-n5c6ccccc6c6ccccc65)c4)n3)cc2)cc1 ; c1ccc(-c2cccc(-c3cc(-c4ccccc4)nc(-c4cc(-c5cc6ccccc6c6ccccc56)cc(-n5c6ccccc6c6ccccc65)c4)n3)c2)cc1 ; c1ccc(-c2cc(-c3ccc4ccccc4c3)nc(-c3cc(-c4cc5ccccc5c5ccccc45)cc(-n4c5ccccc5c5ccccc54)c3)n2)cc1]

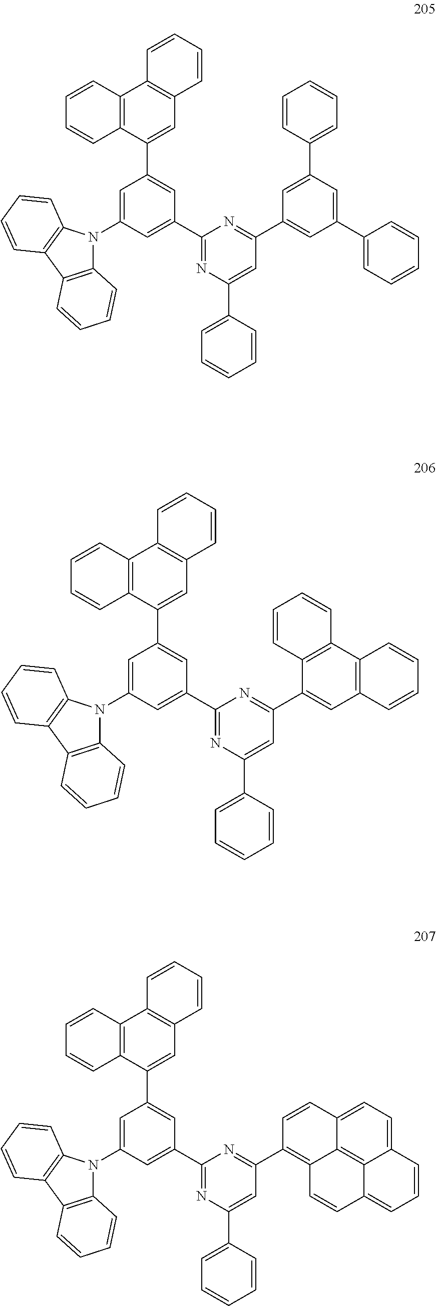 [SMILES: c1ccc(-c2cc(-c3ccccc3)cc(-c3cc(-c4ccccc4)nc(-c4cc(-c5cc6ccccc6c6ccccc56)cc(-n5c6ccccc6c6ccccc65)c4)n3)c2)cc1 ; c1ccc(-c2cc(-c3cc4ccccc4c4ccccc34)nc(-c3cc(-c4cc5ccccc5c5ccccc45)cc(-n4c5ccccc5c5ccccc54)c3)n2)cc1 ; c1ccc(-c2cc(-c3ccc4ccc5cccc6ccc3c4c56)nc(-c3cc(-c4cc5ccccc5c5ccccc45)cc(-n4c5ccccc5c5ccccc54)c3)n2)cc1]

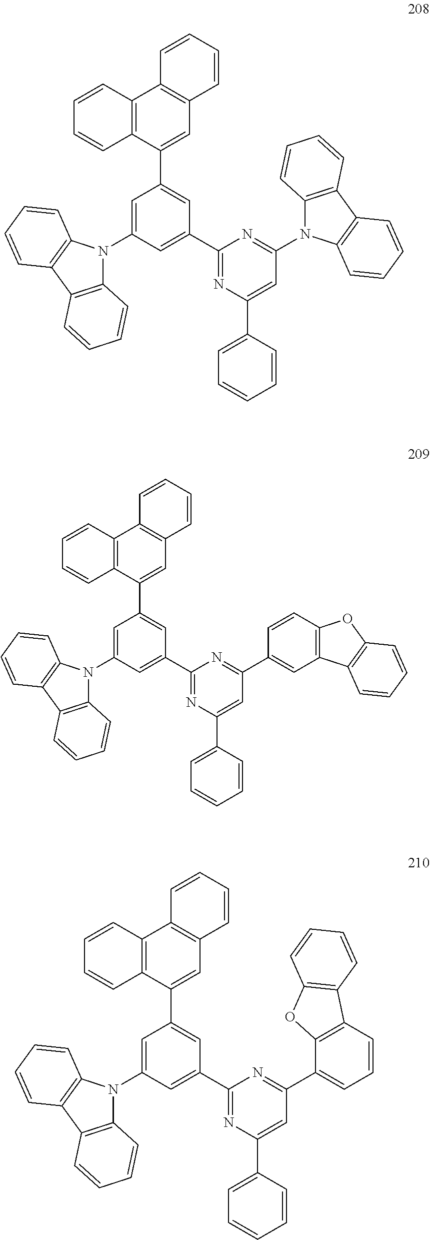 [SMILES: c1ccc(-c2cc(-n3c4ccccc4c4ccccc43)nc(-c3cc(-c4cc5ccccc5c5ccccc45)cc(-n4c5ccccc5c5ccccc54)c3)n2)cc1 ; c1ccc(-c2cc(-c3ccc4oc5ccccc5c4c3)nc(-c3cc(-c4cc5ccccc5c5ccccc45)cc(-n4c5ccccc5c5ccccc54)c3)n2)cc1 ; c1ccc(-c2cc(-c3cccc4c3oc3ccccc34)nc(-c3cc(-c4cc5ccccc5c5ccccc45)cc(-n4c5ccccc5c5ccccc54)c3)n2)cc1]

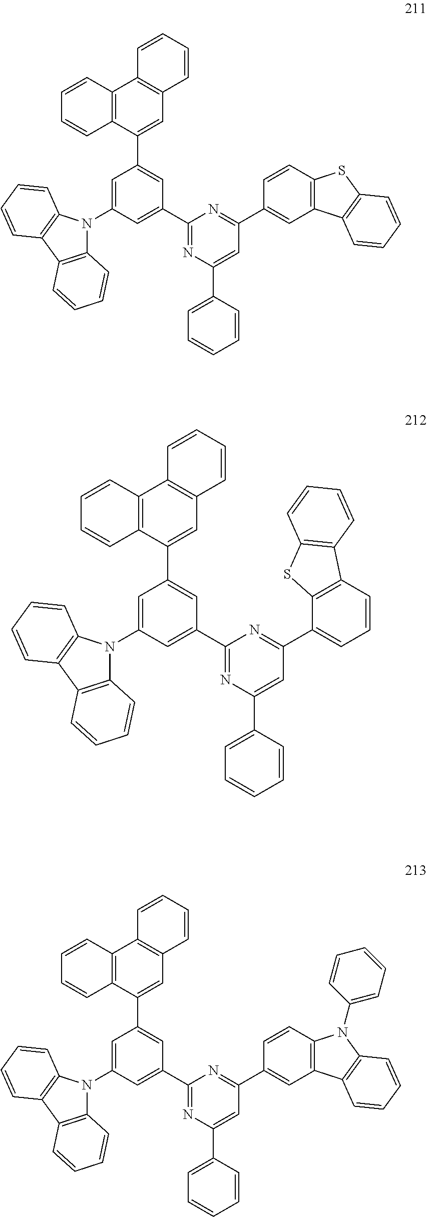 [SMILES: c1ccc(-c2cc(-c3ccc4sc5ccccc5c4c3)nc(-c3cc(-c4cc5ccccc5c5ccccc45)cc(-n4c5ccccc5c5ccccc54)c3)n2)cc1 ; c1ccc(-c2cc(-c3cccc4c3sc3ccccc34)nc(-c3cc(-c4cc5ccccc5c5ccccc45)cc(-n4c5ccccc5c5ccccc54)c3)n2)cc1 ; c1ccc(-c2cc(-c3ccc4c(c3)c3ccccc3n4-c3ccccc3)nc(-c3cc(-c4cc5ccccc5c5ccccc45)cc(-n4c5ccccc5c5ccccc54)c3)n2)cc1]

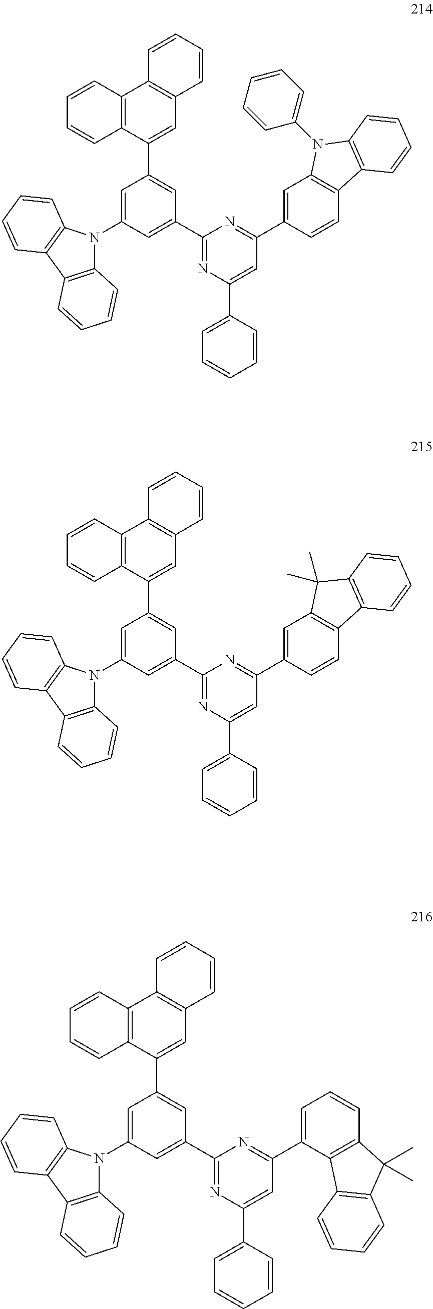 [SMILES: c1ccc(-c2cc(-c3ccc4c5ccccc5n(-c5ccccc5)c4c3)nc(-c3cc(-c4cc5ccccc5c5ccccc45)cc(-n4c5ccccc5c5ccccc54)c3)n2)cc1 ; CC1(C)c2ccccc2-c2ccc(-c3cc(-c4ccccc4)nc(-c4cc(-c5cc6ccccc6c6ccccc56)cc(-n5c6ccccc6c6ccccc65)c4)n3)cc21 ; CC1(C)c2ccccc2-c2c(-c3cc(-c4ccccc4)nc(-c4cc(-c5cc6ccccc6c6ccccc56)cc(-n5c6ccccc6c6ccccc65)c4)n3)cccc21]

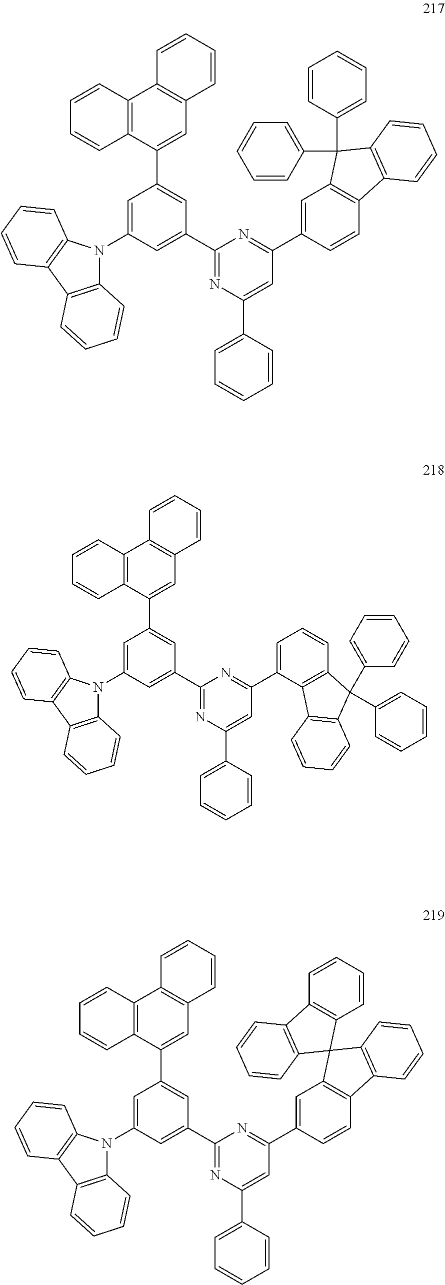 [SMILES: c1ccc(-c2cc(-c3ccc4c(c3)C(c3ccccc3)(c3ccccc3)c3ccccc3-4)nc(-c3cc(-c4cc5ccccc5c5ccccc45)cc(-n4c5ccccc5c5ccccc54)c3)n2)cc1 ; c1ccc(-c2cc(-c3cccc4c3-c3ccccc3C4(c3ccccc3)c3ccccc3)nc(-c3cc(-c4cc5ccccc5c5ccccc45)cc(-n4c5ccccc5c5ccccc54)c3)n2)cc1 ; c1ccc(-c2cc(-c3ccc4c(c3)C3(c5ccccc5-c5ccccc53)c3ccccc3-4)nc(-c3cc(-c4cc5ccccc5c5ccccc45)cc(-n4c5ccccc5c5ccccc54)c3)n2)cc1]

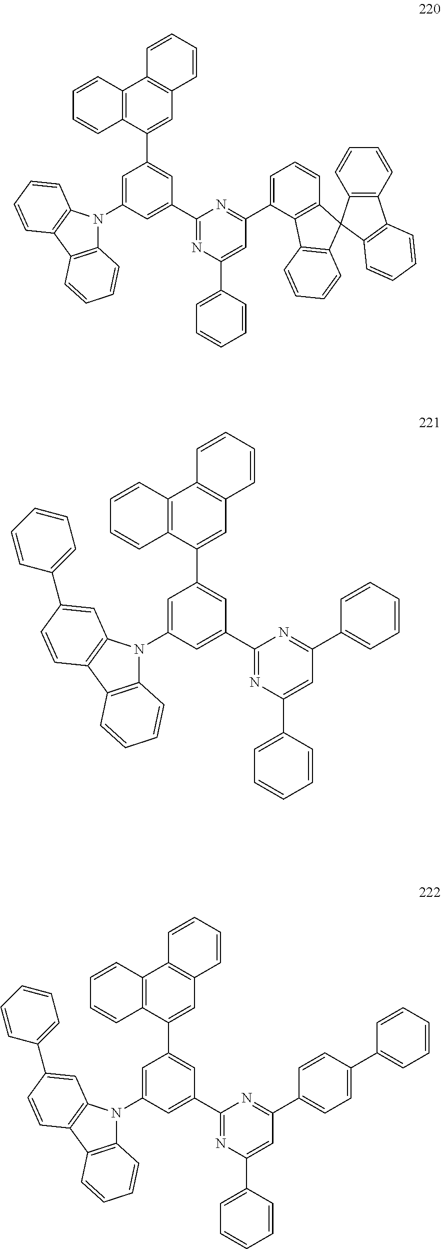 [SMILES: c1ccc(-c2cc(-c3cccc4c3-c3ccccc3C43c4ccccc4-c4ccccc43)nc(-c3cc(-c4cc5ccccc5c5ccccc45)cc(-n4c5ccccc5c5ccccc54)c3)n2)cc1 ; c1ccc(-c2ccc3c4ccccc4n(-c4cc(-c5nc(-c6ccccc6)cc(-c6ccccc6)n5)cc(-c5cc6ccccc6c6ccccc56)c4)c3c2)cc1 ; c1ccc(-c2ccc(-c3cc(-c4ccccc4)nc(-c4cc(-c5cc6ccccc6c6ccccc56)cc(-n5c6ccccc6c6ccc(-c7ccccc7)cc65)c4)n3)cc2)cc1]

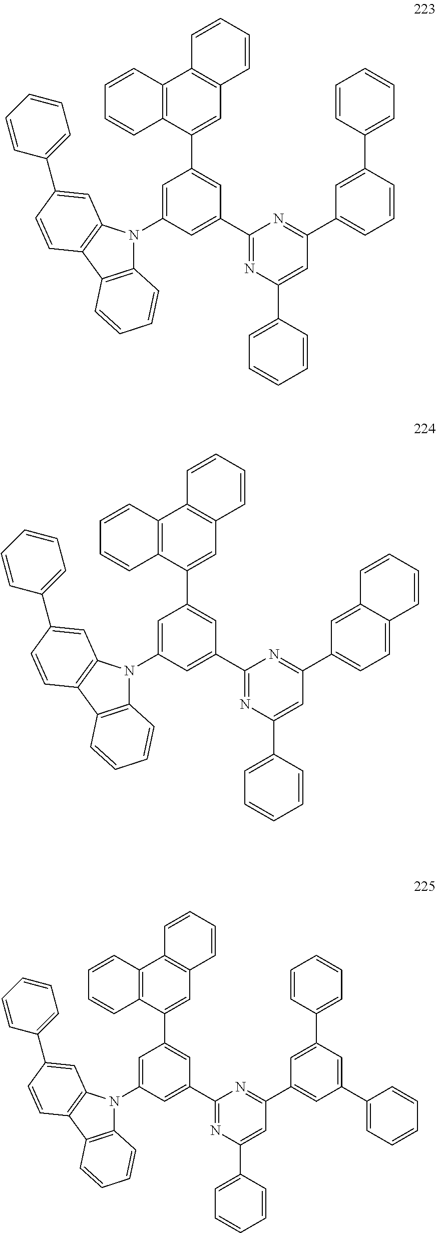 [SMILES: c1ccc(-c2cccc(-c3cc(-c4ccccc4)nc(-c4cc(-c5cc6ccccc6c6ccccc56)cc(-n5c6ccccc6c6ccc(-c7ccccc7)cc65)c4)n3)c2)cc1 ; c1ccc(-c2ccc3c4ccccc4n(-c4cc(-c5nc(-c6ccccc6)cc(-c6ccc7ccccc7c6)n5)cc(-c5cc6ccccc6c6ccccc56)c4)c3c2)cc1 ; c1ccc(-c2cc(-c3ccccc3)cc(-c3cc(-c4ccccc4)nc(-c4cc(-c5cc6ccccc6c6ccccc56)cc(-n5c6ccccc6c6ccc(-c7ccccc7)cc65)c4)n3)c2)cc1]

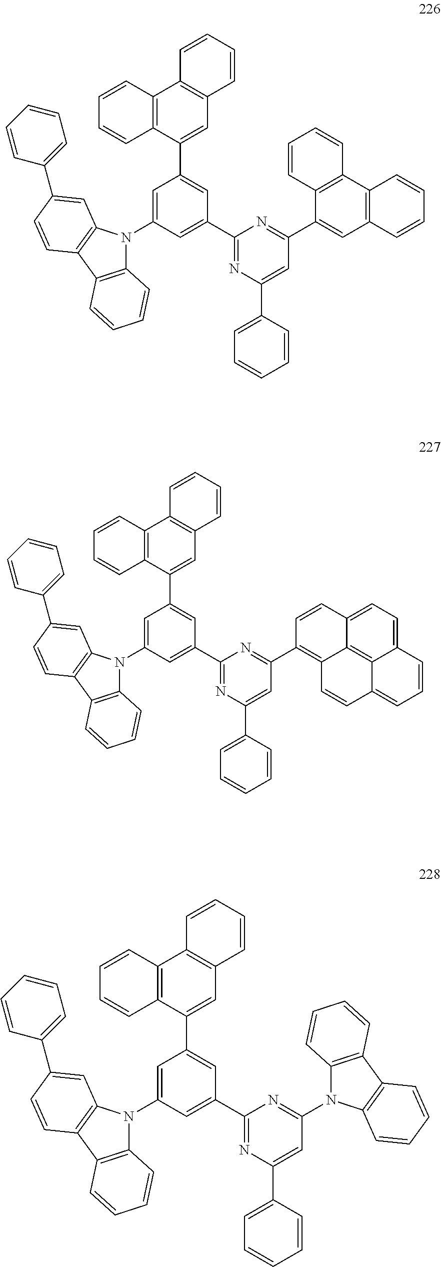 [SMILES: c1ccc(-c2ccc3c4ccccc4n(-c4cc(-c5nc(-c6ccccc6)cc(-c6cc7ccccc7c7ccccc67)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7ccc8cccc9ccc6c7c89)n5)cc(-c5cc6ccccc6c6ccccc56)c4)c3c2)cc1 ; c1ccc(-c2ccc3c4ccccc4n(-c4cc(-c5nc(-c6ccccc6)cc(-n6c7ccccc7c7ccccc76)n5)cc(-c5cc6ccccc6c6ccccc56)c4)c3c2)cc1]

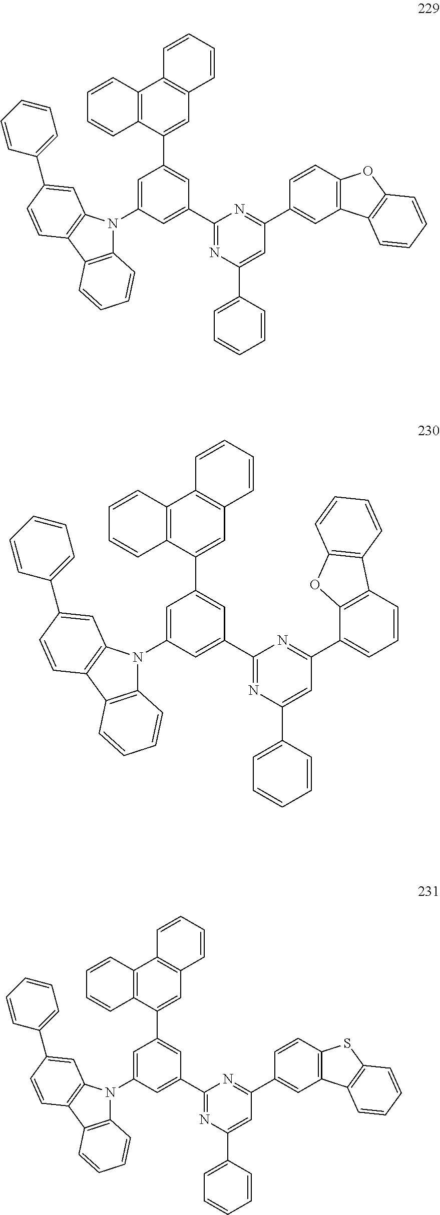 [SMILES: c1ccc(-c2ccc3c4ccccc4n(-c4cc(-c5nc(-c6ccccc6)cc(-c6ccc7oc8ccccc8c7c6)n5)cc(-c5cc6ccccc6c6ccccc56)c4)c3c2)cc1 ; c1ccc(-c2ccc3c4ccccc4n(-c4cc(-c5nc(-c6ccccc6)cc(-c6cccc7c6oc6ccccc67)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7sc8ccccc8c7c6)n5)cc(-c5cc6ccccc6c6ccccc56)c4)c3c2)cc1]

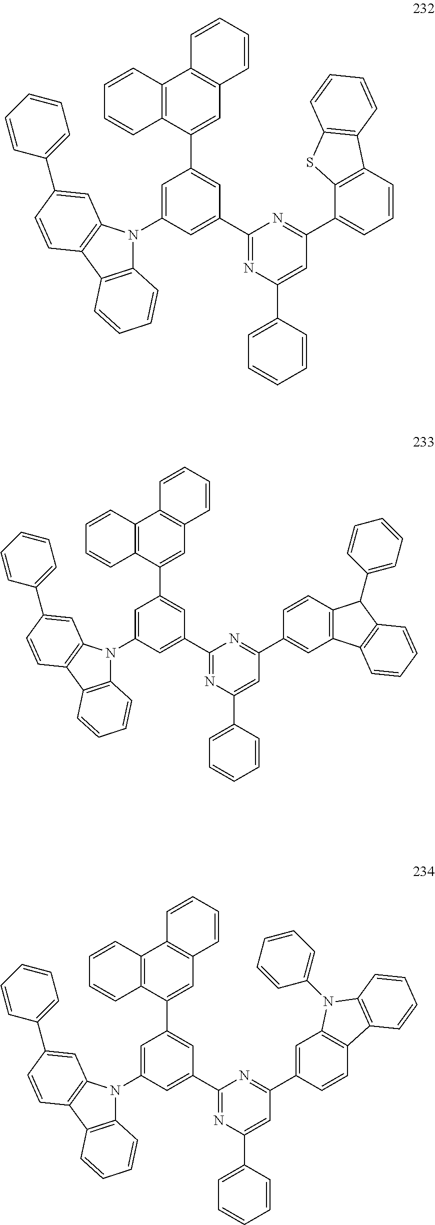 [SMILES: c1ccc(-c2ccc3c4ccccc4n(-c4cc(-c5nc(-c6ccccc6)cc(-c6cccc7c6sc6ccccc67)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7c(c6)-c6ccccc6C7c6ccccc6)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7c8ccccc8n(-c8ccccc8)c7c6)n5)cc(-c5cc6ccccc6c6ccccc56)c4)c3c2)cc1]

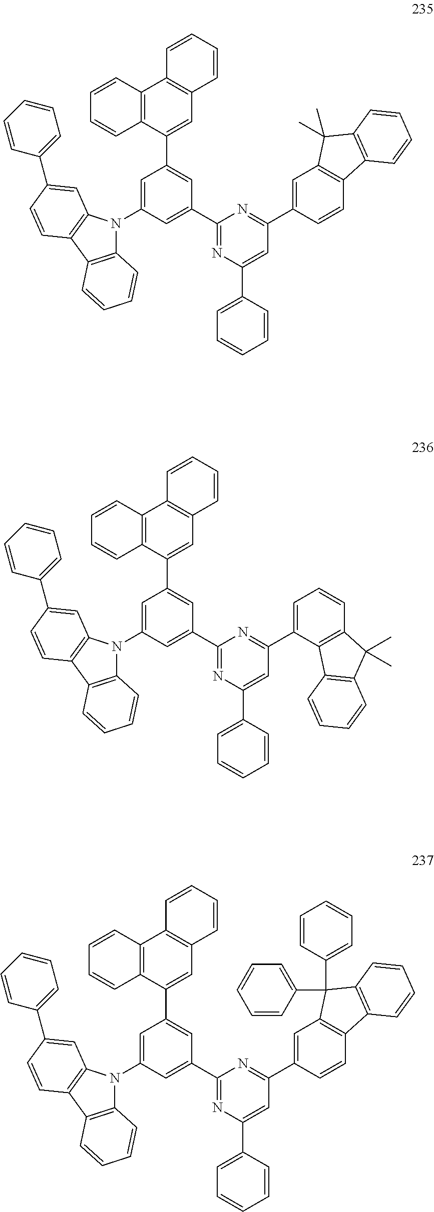 [SMILES: CC1(C)c2ccccc2-c2ccc(-c3cc(-c4ccccc4)nc(-c4cc(-c5cc6ccccc6c6ccccc56)cc(-n5c6ccccc6c6ccc(-c7ccccc7)cc65)c4)n3)cc21 ; CC1(C)c2ccccc2-c2c(-c3cc(-c4ccccc4)nc(-c4cc(-c5cc6ccccc6c6ccccc56)cc(-n5c6ccccc6c6ccc(-c7ccccc7)cc65)c4)n3)cccc21 ; c1ccc(-c2ccc3c4ccccc4n(-c4cc(-c5nc(-c6ccccc6)cc(-c6ccc7c(c6)C(c6ccccc6)(c6ccccc6)c6ccccc6-7)n5)cc(-c5cc6ccccc6c6ccccc56)c4)c3c2)cc1]

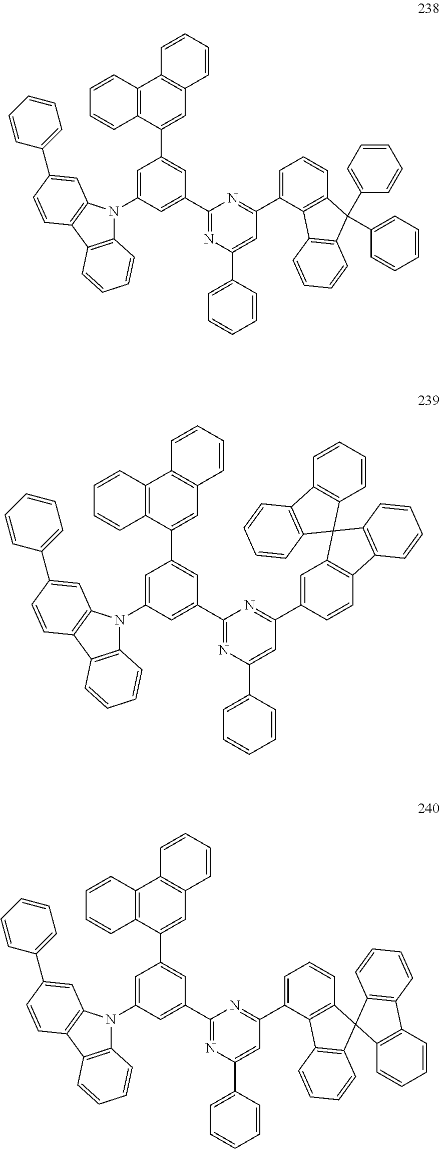 [SMILES: c1ccc(-c2ccc3c4ccccc4n(-c4cc(-c5nc(-c6ccccc6)cc(-c6cccc7c6-c6ccccc6C7(c6ccccc6)c6ccccc6)n5)cc(-c5cc6ccccc6c6ccccc56)c4)c3c2)cc1 ; c1ccc(-c2ccc3c4ccccc4n(-c4cc(-c5nc(-c6ccccc6)cc(-c6ccc7c(c6)C6(c8ccccc8-c8ccccc86)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)cc(-c6cccc7c6-c6ccccc6C76c7ccccc7-c7ccccc76)n5)cc(-c5cc6ccccc6c6ccccc56)c4)c3c2)cc1]

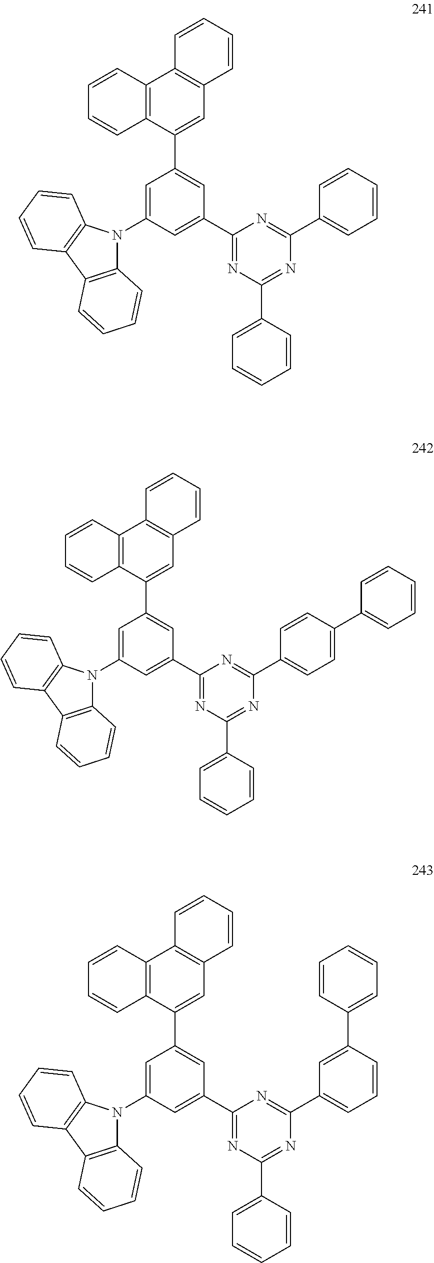 [SMILES: c1ccc(-c2nc(-c3ccccc3)nc(-c3cc(-c4cc5ccccc5c5ccccc45)cc(-n4c5ccccc5c5ccccc54)c3)n2)cc1 ; c1ccc(-c2ccc(-c3nc(-c4ccccc4)nc(-c4cc(-c5cc6ccccc6c6ccccc56)cc(-n5c6ccccc6c6ccccc65)c4)n3)cc2)cc1 ; c1ccc(-c2cccc(-c3nc(-c4ccccc4)nc(-c4cc(-c5cc6ccccc6c6ccccc56)cc(-n5c6ccccc6c6ccccc65)c4)n3)c2)cc1]

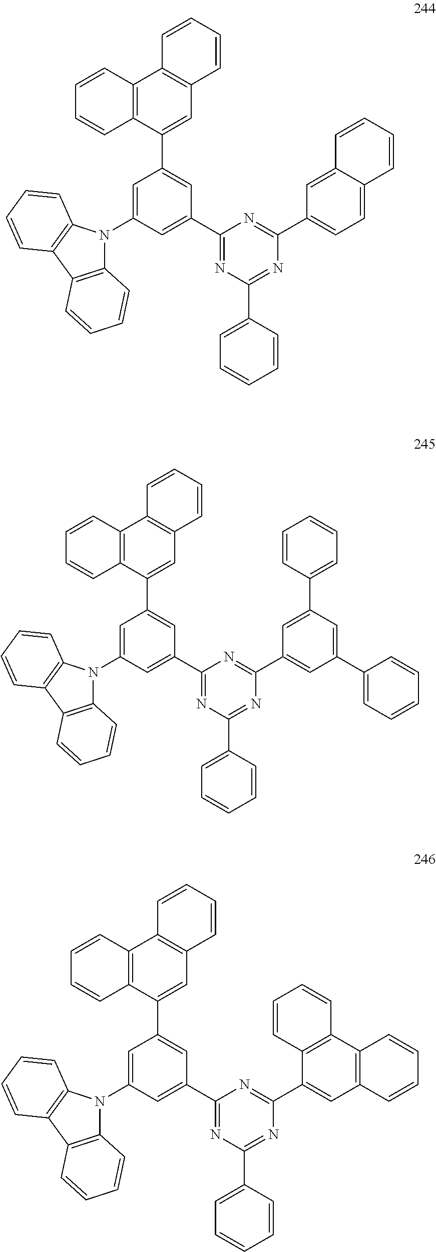 [SMILES: c1ccc(-c2nc(-c3cc(-c4cc5ccccc5c5ccccc45)cc(-n4c5ccccc5c5ccccc54)c3)nc(-c3ccc4ccccc4c3)n2)cc1 ; c1ccc(-c2cc(-c3ccccc3)cc(-c3nc(-c4ccccc4)nc(-c4cc(-c5cc6ccccc6c6ccccc56)cc(-n5c6ccccc6c6ccccc65)c4)n3)c2)cc1 ; c1ccc(-c2nc(-c3cc(-c4cc5ccccc5c5ccccc45)cc(-n4c5ccccc5c5ccccc54)c3)nc(-c3cc4ccccc4c4ccccc34)n2)cc1]

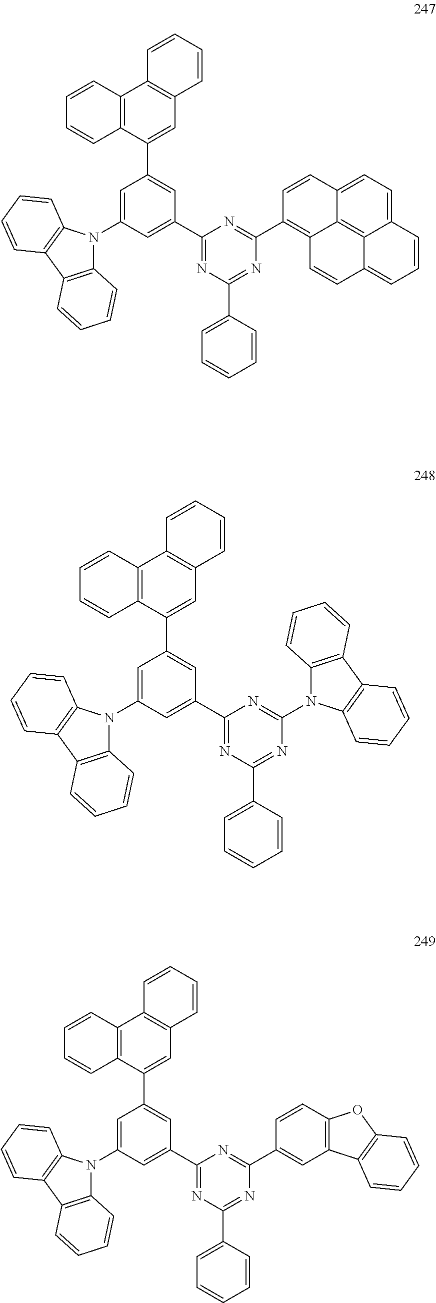 [SMILES: c1ccc(-c2nc(-c3cc(-c4cc5ccccc5c5ccccc45)cc(-n4c5ccccc5c5ccccc54)c3)nc(-c3ccc4ccc5cccc6ccc3c4c56)n2)cc1 ; c1ccc(-c2nc(-c3cc(-c4cc5ccccc5c5ccccc45)cc(-n4c5ccccc5c5ccccc54)c3)nc(-n3c4ccccc4c4ccccc43)n2)cc1 ; c1ccc(-c2nc(-c3cc(-c4cc5ccccc5c5ccccc45)cc(-n4c5ccccc5c5ccccc54)c3)nc(-c3ccc4oc5ccccc5c4c3)n2)cc1]

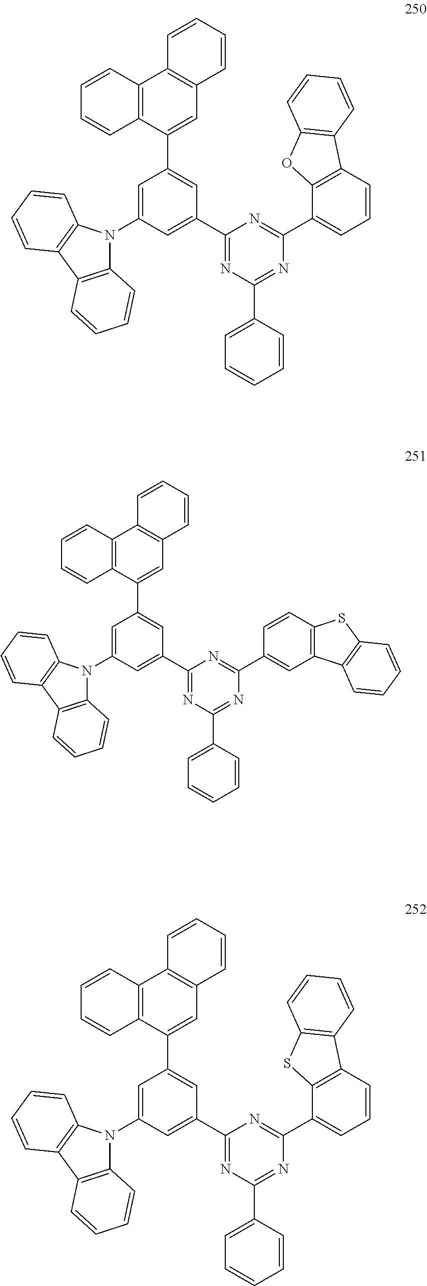 [SMILES: c1ccc(-c2nc(-c3cc(-c4cc5ccccc5c5ccccc45)cc(-n4c5ccccc5c5ccccc54)c3)nc(-c3cccc4c3oc3ccccc34)n2)cc1 ; c1ccc(-c2nc(-c3cc(-c4cc5ccccc5c5ccccc45)cc(-n4c5ccccc5c5ccccc54)c3)nc(-c3ccc4sc5ccccc5c4c3)n2)cc1 ; c1ccc(-c2nc(-c3cc(-c4cc5ccccc5c5ccccc45)cc(-n4c5ccccc5c5ccccc54)c3)nc(-c3cccc4c3sc3ccccc34)n2)cc1]

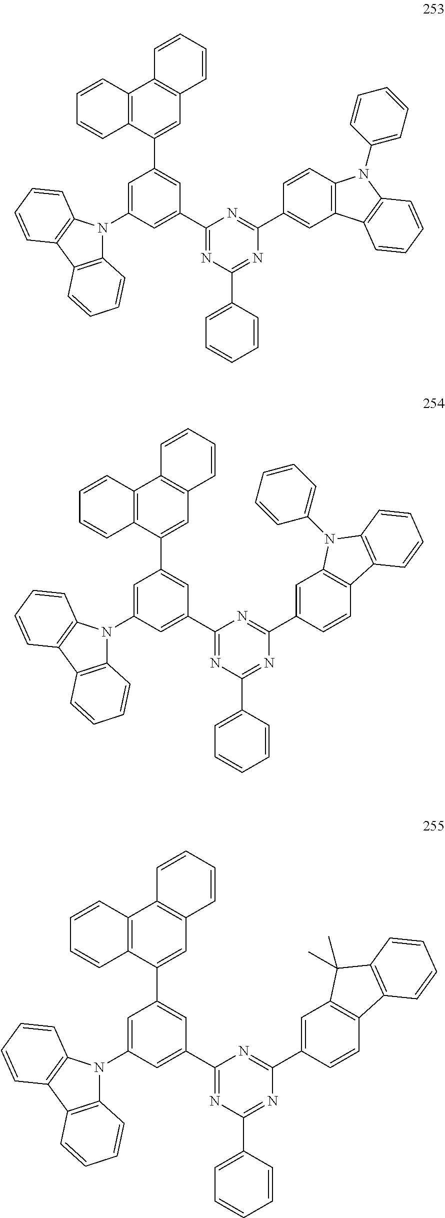 [SMILES: c1ccc(-c2nc(-c3cc(-c4cc5ccccc5c5ccccc45)cc(-n4c5ccccc5c5ccccc54)c3)nc(-c3ccc4c(c3)c3ccccc3n4-c3ccccc3)n2)cc1 ; c1ccc(-c2nc(-c3cc(-c4cc5ccccc5c5ccccc45)cc(-n4c5ccccc5c5ccccc54)c3)nc(-c3ccc4c5ccccc5n(-c5ccccc5)c4c3)n2)cc1 ; CC1(C)c2ccccc2-c2ccc(-c3nc(-c4ccccc4)nc(-c4cc(-c5cc6ccccc6c6ccccc56)cc(-n5c6ccccc6c6ccccc65)c4)n3)cc21]

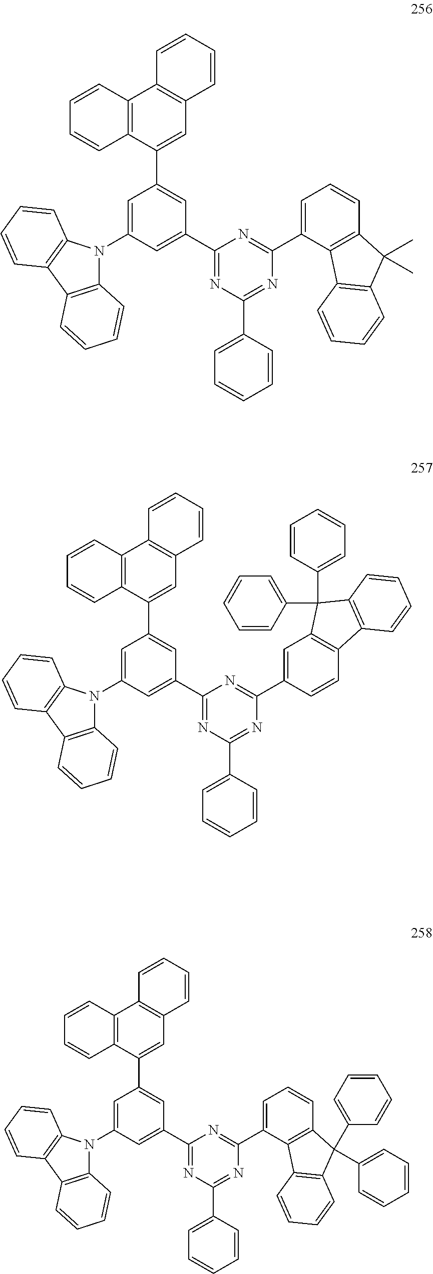 [SMILES: CC1(C)c2ccccc2-c2c(-c3nc(-c4ccccc4)nc(-c4cc(-c5cc6ccccc6c6ccccc56)cc(-n5c6ccccc6c6ccccc65)c4)n3)cccc21 ; c1ccc(-c2nc(-c3cc(-c4cc5ccccc5c5ccccc45)cc(-n4c5ccccc5c5ccccc54)c3)nc(-c3ccc4c(c3)C(c3ccccc3)(c3ccccc3)c3ccccc3-4)n2)cc1 ; c1ccc(-c2nc(-c3cc(-c4cc5ccccc5c5ccccc45)cc(-n4c5ccccc5c5ccccc54)c3)nc(-c3cccc4c3-c3ccccc3C4(c3ccccc3)c3ccccc3)n2)cc1]

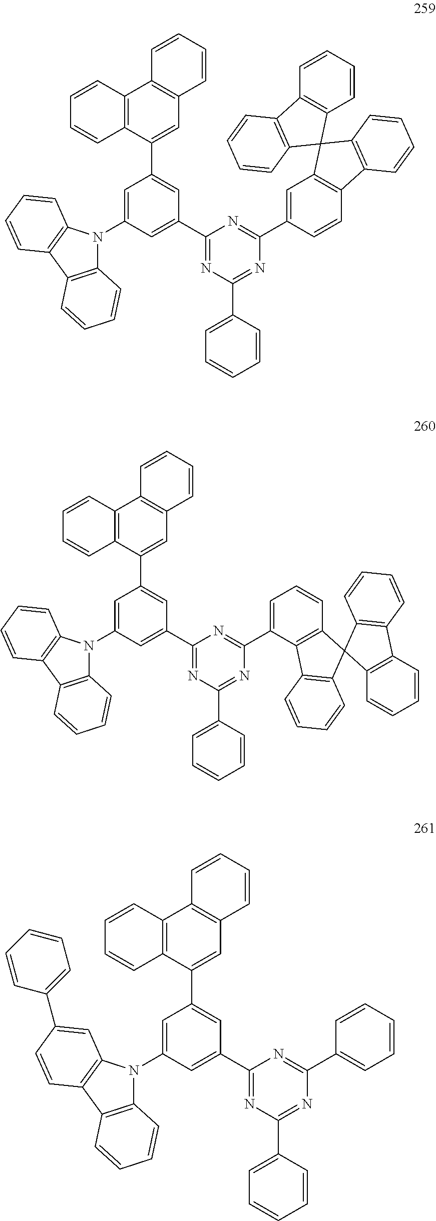 [SMILES: c1ccc(-c2nc(-c3cc(-c4cc5ccccc5c5ccccc45)cc(-n4c5ccccc5c5ccccc54)c3)nc(-c3ccc4c(c3)C3(c5ccccc5-c5ccccc53)c3ccccc3-4)n2)cc1 ; c1ccc(-c2nc(-c3cc(-c4cc5ccccc5c5ccccc45)cc(-n4c5ccccc5c5ccccc54)c3)nc(-c3cccc4c3-c3ccccc3C43c4ccccc4-c4ccccc43)n2)cc1 ; c1ccc(-c2ccc3c4ccccc4n(-c4cc(-c5nc(-c6ccccc6)nc(-c6ccccc6)n5)cc(-c5cc6ccccc6c6ccccc56)c4)c3c2)cc1]

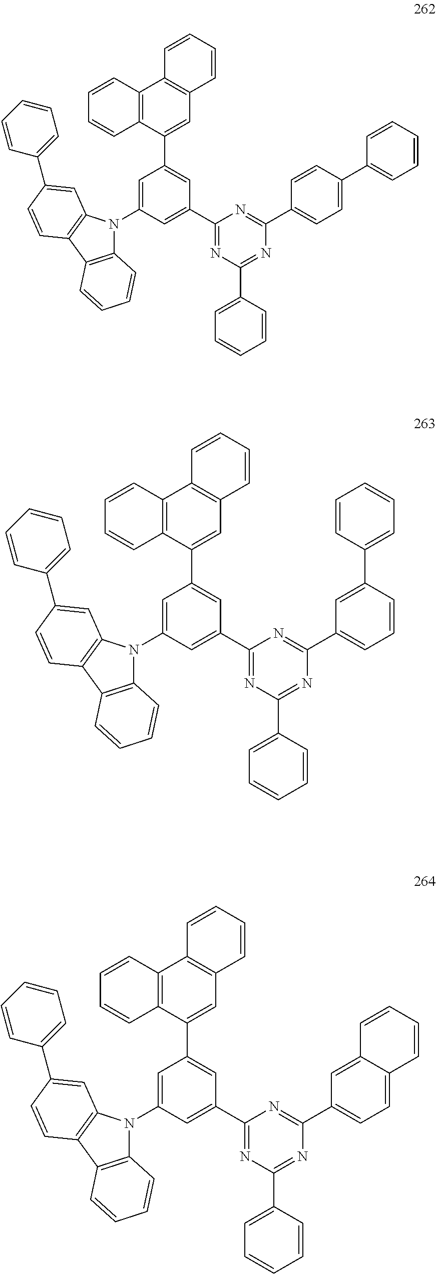 [SMILES: c1ccc(-c2ccc(-c3nc(-c4ccccc4)nc(-c4cc(-c5cc6ccccc6c6ccccc56)cc(-n5c6ccccc6c6ccc(-c7ccccc7)cc65)c4)n3)cc2)cc1 ; c1ccc(-c2cccc(-c3nc(-c4ccccc4)nc(-c4cc(-c5cc6ccccc6c6ccccc56)cc(-n5c6ccccc6c6ccc(-c7ccccc7)cc65)c4)n3)c2)cc1 ; c1ccc(-c2ccc3c4ccccc4n(-c4cc(-c5nc(-c6ccccc6)nc(-c6ccc7ccccc7c6)n5)cc(-c5cc6ccccc6c6ccccc56)c4)c3c2)cc1]

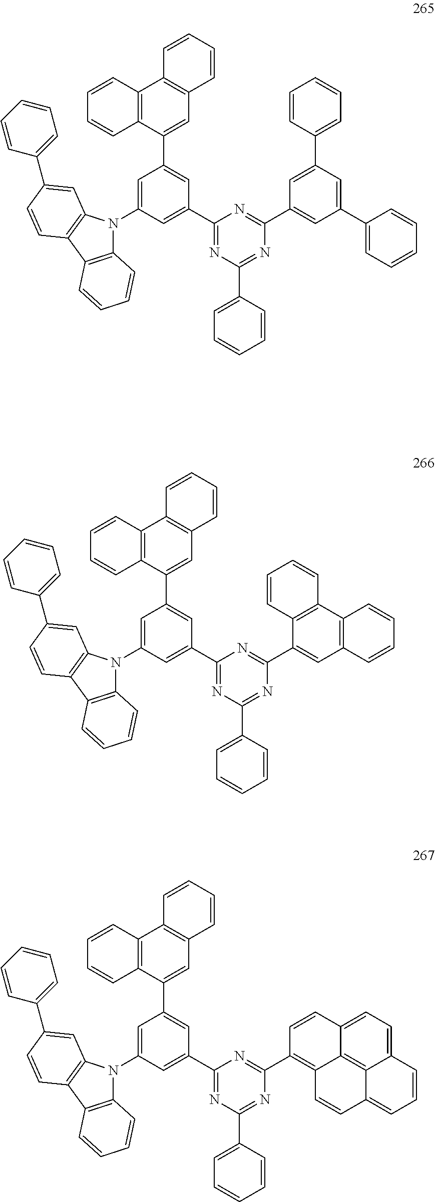 [SMILES: c1ccc(-c2cc(-c3ccccc3)cc(-c3nc(-c4ccccc4)nc(-c4cc(-c5cc6ccccc6c6ccccc56)cc(-n5c6ccccc6c6ccc(-c7ccccc7)cc65)c4)n3)c2)cc1 ; c1ccc(-c2ccc3c4ccccc4n(-c4cc(-c5nc(-c6ccccc6)nc(-c6cc7ccccc7c7ccccc67)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7ccc8cccc9ccc6c7c89)n5)cc(-c5cc6ccccc6c6ccccc56)c4)c3c2)cc1]

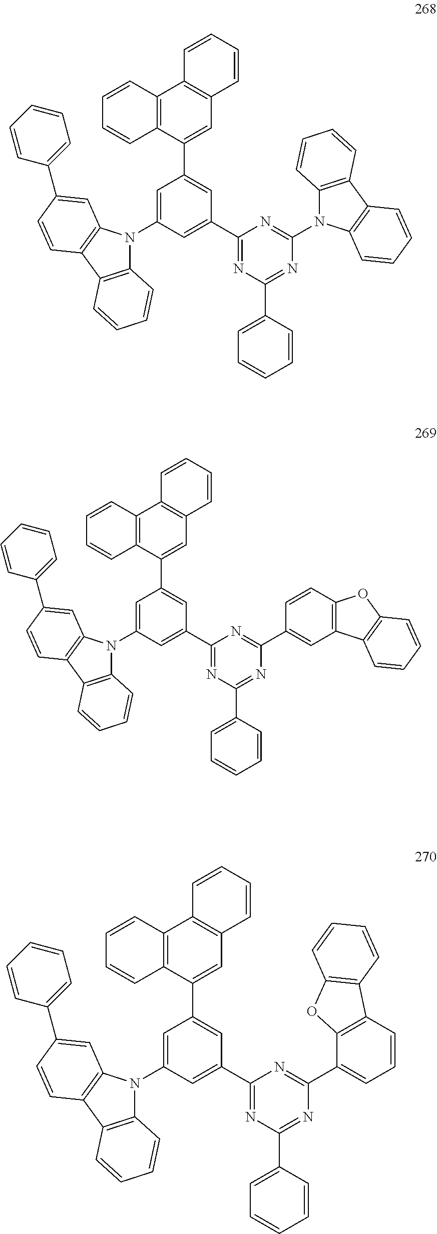 [SMILES: c1ccc(-c2ccc3c4ccccc4n(-c4cc(-c5nc(-c6ccccc6)nc(-n6c7ccccc7c7ccccc76)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7oc8ccccc8c7c6)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6oc6ccccc67)n5)cc(-c5cc6ccccc6c6ccccc56)c4)c3c2)cc1]

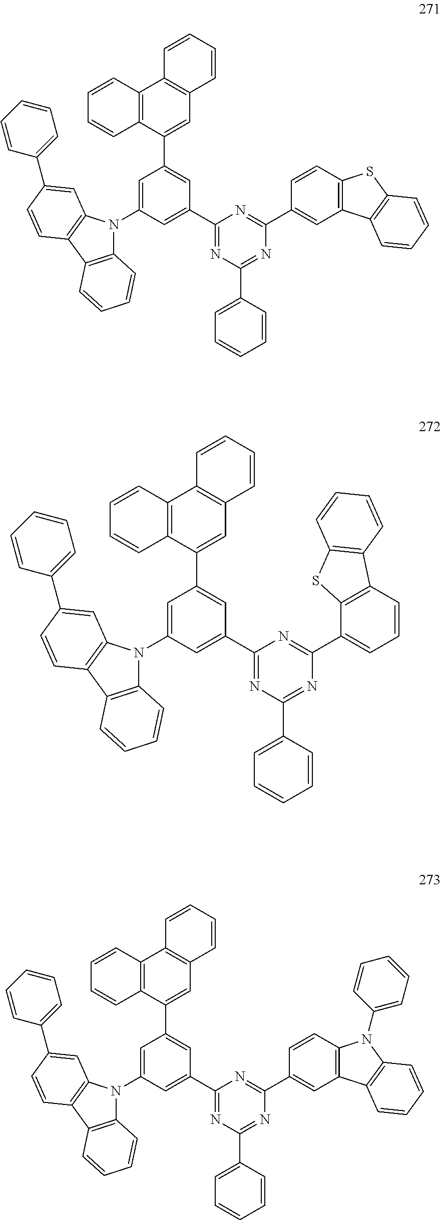 [SMILES: c1ccc(-c2ccc3c4ccccc4n(-c4cc(-c5nc(-c6ccccc6)nc(-c6ccc7sc8ccccc8c7c6)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6sc6ccccc67)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7c(c6)c6ccccc6n7-c6ccccc6)n5)cc(-c5cc6ccccc6c6ccccc56)c4)c3c2)cc1]

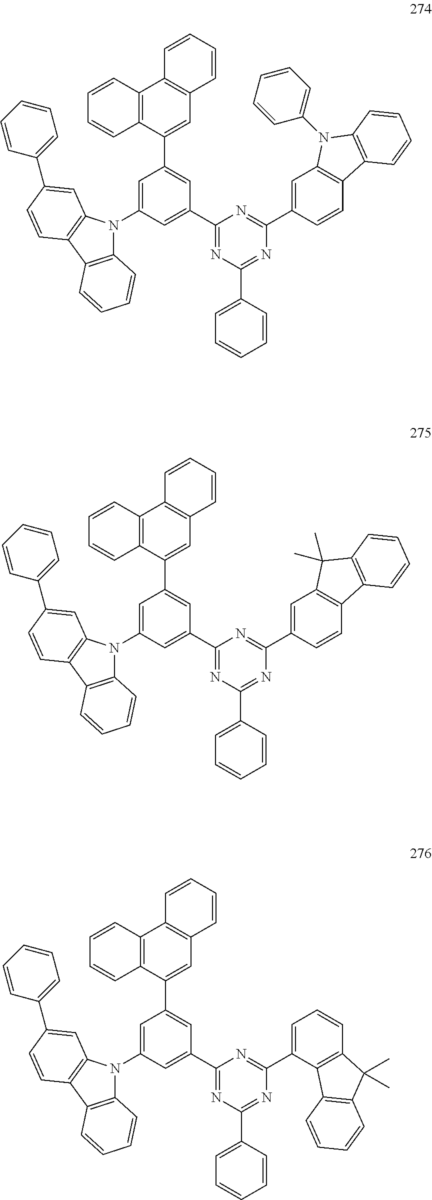 [SMILES: c1ccc(-c2ccc3c4ccccc4n(-c4cc(-c5nc(-c6ccccc6)nc(-c6ccc7c8ccccc8n(-c8ccccc8)c7c6)n5)cc(-c5cc6ccccc6c6ccccc56)c4)c3c2)cc1 ; CC1(C)c2ccccc2-c2ccc(-c3nc(-c4ccccc4)nc(-c4cc(-c5cc6ccccc6c6ccccc56)cc(-n5c6ccccc6c6ccc(-c7ccccc7)cc65)c4)n3)cc21 ; CC1(C)c2ccccc2-c2c(-c3nc(-c4ccccc4)nc(-c4cc(-c5cc6ccccc6c6ccccc56)cc(-n5c6ccccc6c6ccc(-c7ccccc7)cc65)c4)n3)cccc21]

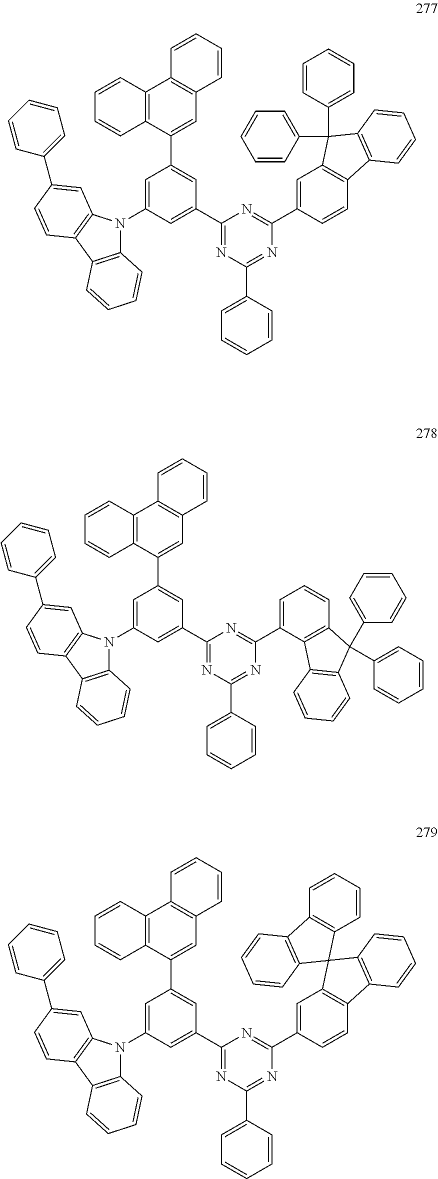 [SMILES: c1ccc(-c2ccc3c4ccccc4n(-c4cc(-c5nc(-c6ccccc6)nc(-c6ccc7c(c6)C(c6ccccc6)(c6ccccc6)c6ccccc6-7)n5)cc(-c5cc6ccccc6c6ccccc56)c4)c3c2)cc1 ; c1ccc(-c2ccc3c4ccccc4n(-c4cc(-c5nc(-c6ccccc6)nc(-c6cccc7c6-c6ccccc6C7(c6ccccc6)c6ccccc6)n5)cc(-c5cc6ccccc6c6ccccc56)c4)c3c2)cc1 ; c1ccc(-c2ccc3c4ccccc4n(-c4cc(-c5nc(-c6ccccc6)nc(-c6ccc7c(c6)C6(c8ccccc8-c8ccccc86)c6ccccc6-7)n5)cc(-c5cc6ccccc6c6ccccc56)c4)c3c2)cc1]

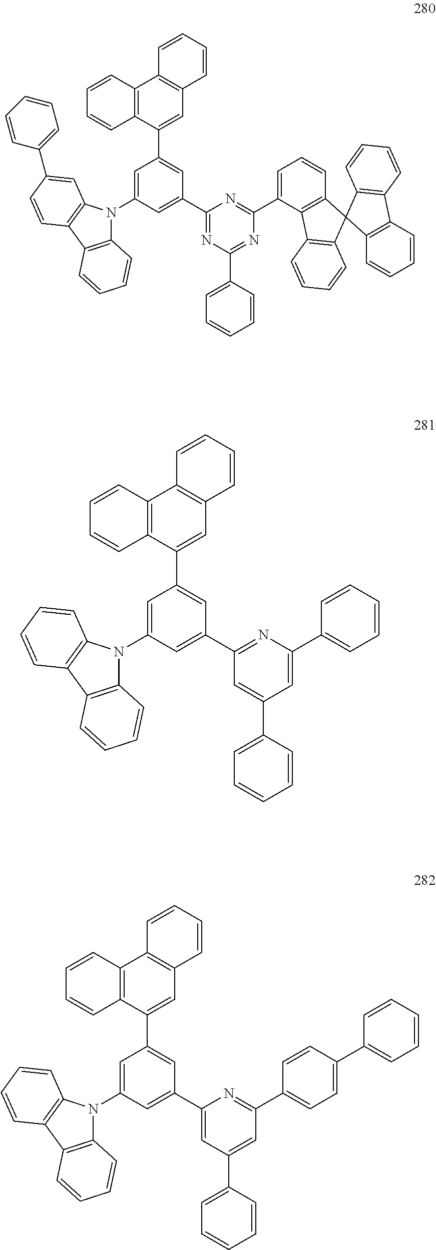 [SMILES: c1ccc(-c2ccc3c4ccccc4n(-c4cc(-c5nc(-c6ccccc6)nc(-c6cccc7c6-c6ccccc6C76c7ccccc7-c7ccccc76)n5)cc(-c5cc6ccccc6c6ccccc56)c4)c3c2)cc1 ; c1ccc(-c2cc(-c3ccccc3)nc(-c3cc(-c4cc5ccccc5c5ccccc45)cc(-n4c5ccccc5c5ccccc54)c3)c2)cc1 ; c1ccc(-c2ccc(-c3cc(-c4ccccc4)cc(-c4cc(-c5cc6ccccc6c6ccccc56)cc(-n5c6ccccc6c6ccccc65)c4)n3)cc2)cc1]

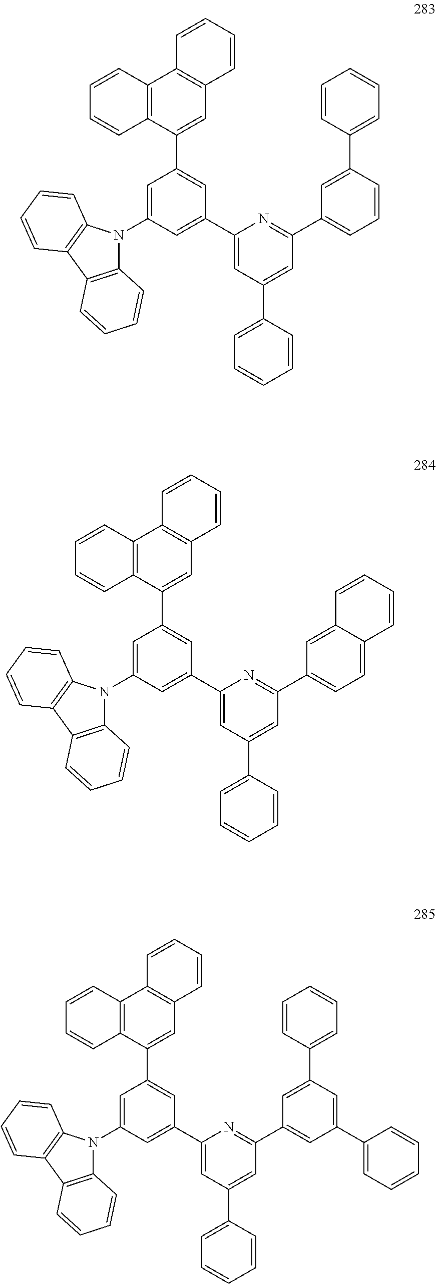 [SMILES: c1ccc(-c2cccc(-c3cc(-c4ccccc4)cc(-c4cc(-c5cc6ccccc6c6ccccc56)cc(-n5c6ccccc6c6ccccc65)c4)n3)c2)cc1 ; c1ccc(-c2cc(-c3cc(-c4cc5ccccc5c5ccccc45)cc(-n4c5ccccc5c5ccccc54)c3)nc(-c3ccc4ccccc4c3)c2)cc1 ; c1ccc(-c2cc(-c3ccccc3)cc(-c3cc(-c4ccccc4)cc(-c4cc(-c5cc6ccccc6c6ccccc56)cc(-n5c6ccccc6c6ccccc65)c4)n3)c2)cc1]

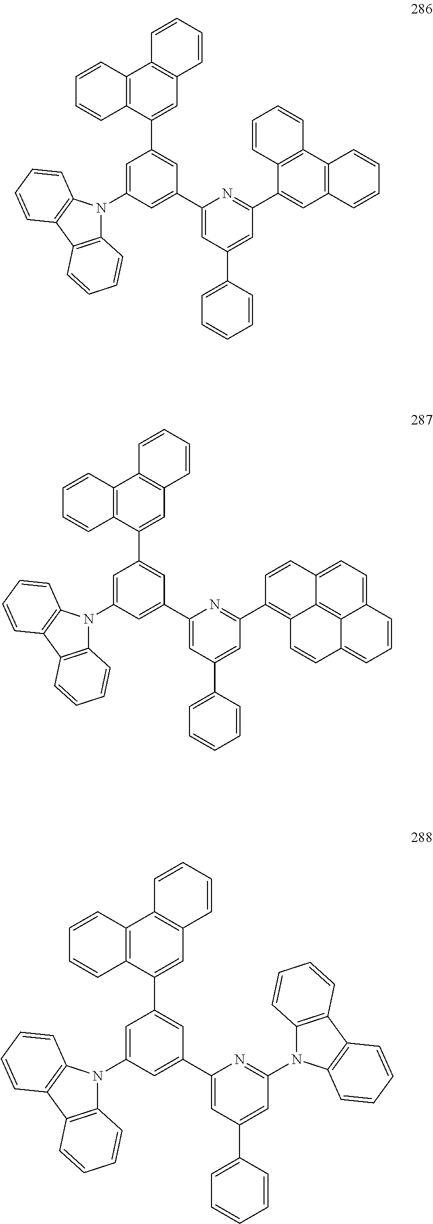 [SMILES: c1ccc(-c2cc(-c3cc(-c4cc5ccccc5c5ccccc45)cc(-n4c5ccccc5c5ccccc54)c3)nc(-c3cc4ccccc4c4ccccc34)c2)cc1 ; c1ccc(-c2cc(-c3cc(-c4cc5ccccc5c5ccccc45)cc(-n4c5ccccc5c5ccccc54)c3)nc(-c3ccc4ccc5cccc6ccc3c4c56)c2)cc1 ; c1ccc(-c2cc(-c3cc(-c4cc5ccccc5c5ccccc45)cc(-n4c5ccccc5c5ccccc54)c3)nc(-n3c4ccccc4c4ccccc43)c2)cc1]

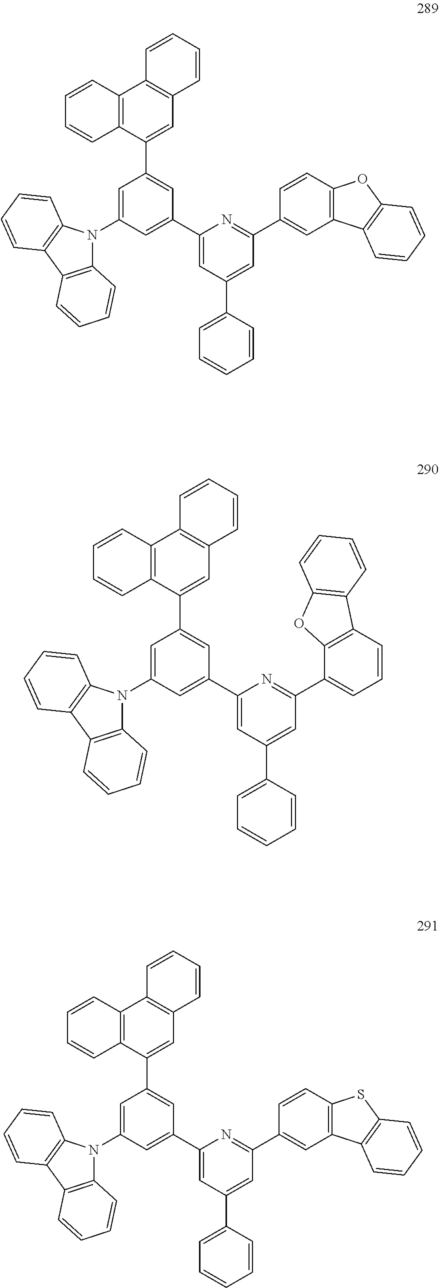 [SMILES: c1ccc(-c2cc(-c3cc(-c4cc5ccccc5c5ccccc45)cc(-n4c5ccccc5c5ccccc54)c3)nc(-c3ccc4oc5ccccc5c4c3)c2)cc1 ; c1ccc(-c2cc(-c3cc(-c4cc5ccccc5c5ccccc45)cc(-n4c5ccccc5c5ccccc54)c3)nc(-c3cccc4c3oc3ccccc34)c2)cc1 ; c1ccc(-c2cc(-c3cc(-c4cc5ccccc5c5ccccc45)cc(-n4c5ccccc5c5ccccc54)c3)nc(-c3ccc4sc5ccccc5c4c3)c2)cc1]

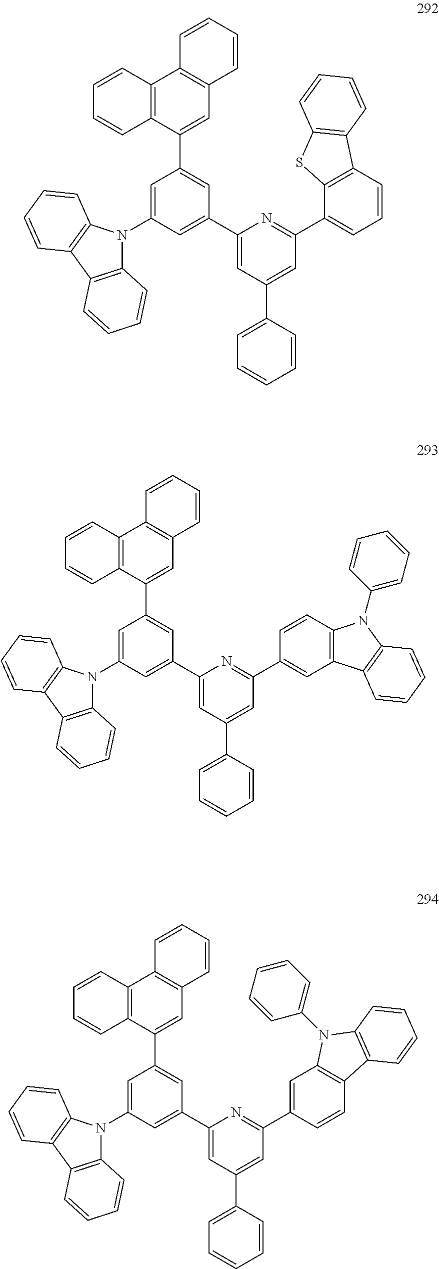 [SMILES: c1ccc(-c2cc(-c3cc(-c4cc5ccccc5c5ccccc45)cc(-n4c5ccccc5c5ccccc54)c3)nc(-c3cccc4c3sc3ccccc34)c2)cc1 ; c1ccc(-c2cc(-c3cc(-c4cc5ccccc5c5ccccc45)cc(-n4c5ccccc5c5ccccc54)c3)nc(-c3ccc4c(c3)c3ccccc3n4-c3ccccc3)c2)cc1 ; c1ccc(-c2cc(-c3cc(-c4cc5ccccc5c5ccccc45)cc(-n4c5ccccc5c5ccccc54)c3)nc(-c3ccc4c5ccccc5n(-c5ccccc5)c4c3)c2)cc1]

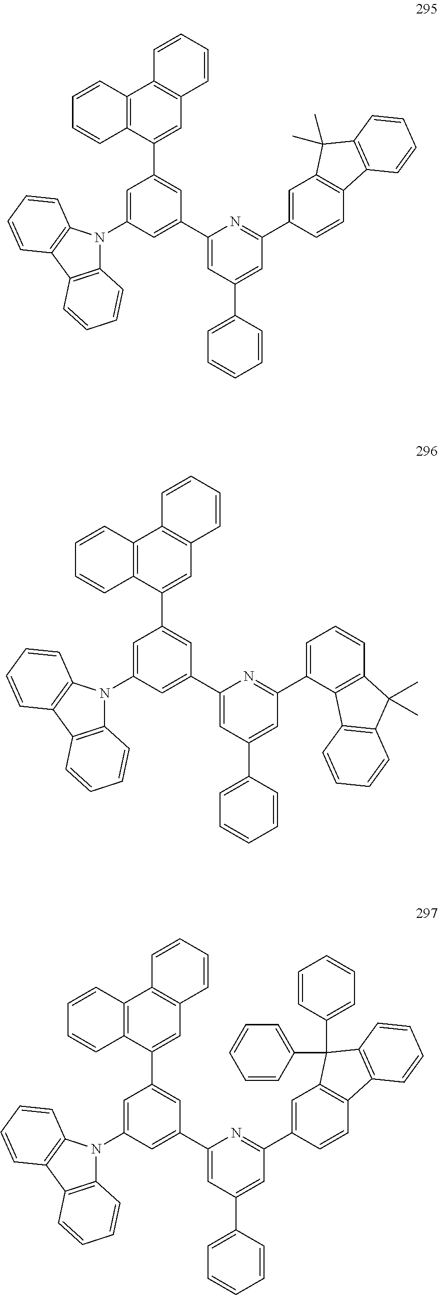 [SMILES: CC1(C)c2ccccc2-c2ccc(-c3cc(-c4ccccc4)cc(-c4cc(-c5cc6ccccc6c6ccccc56)cc(-n5c6ccccc6c6ccccc65)c4)n3)cc21 ; CC1(C)c2ccccc2-c2c(-c3cc(-c4ccccc4)cc(-c4cc(-c5cc6ccccc6c6ccccc56)cc(-n5c6ccccc6c6ccccc65)c4)n3)cccc21 ; c1ccc(-c2cc(-c3cc(-c4cc5ccccc5c5ccccc45)cc(-n4c5ccccc5c5ccccc54)c3)nc(-c3ccc4c(c3)C(c3ccccc3)(c3ccccc3)c3ccccc3-4)c2)cc1]

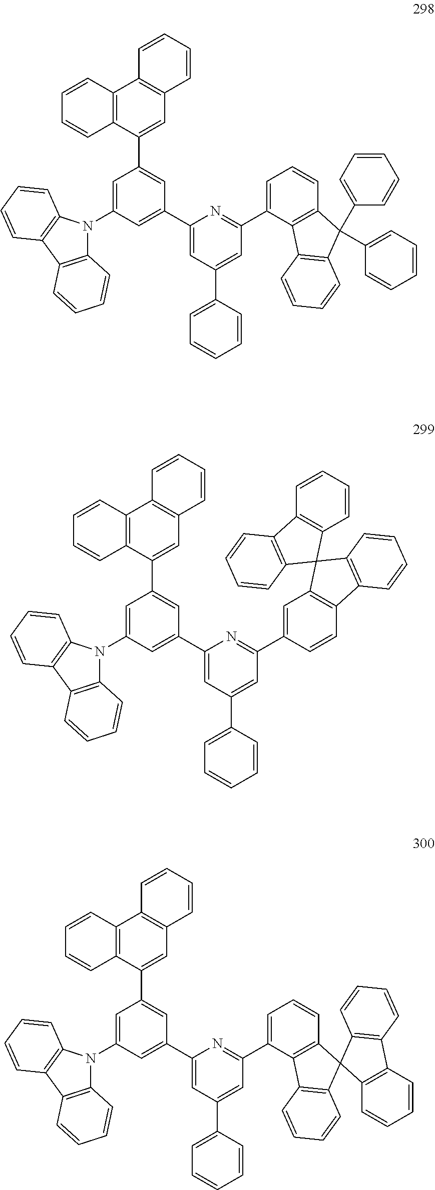 [SMILES: c1ccc(-c2cc(-c3cc(-c4cc5ccccc5c5ccccc45)cc(-n4c5ccccc5c5ccccc54)c3)nc(-c3cccc4c3-c3ccccc3C4(c3ccccc3)c3ccccc3)c2)cc1 ; c1ccc(-c2cc(-c3cc(-c4cc5ccccc5c5ccccc45)cc(-n4c5ccccc5c5ccccc54)c3)nc(-c3ccc4c(c3)C3(c5ccccc5-c5ccccc53)c3ccccc3-4)c2)cc1 ; c1ccc(-c2cc(-c3cc(-c4cc5ccccc5c5ccccc45)cc(-n4c5ccccc5c5ccccc54)c3)nc(-c3cccc4c3-c3ccccc3C43c4ccccc4-c4ccccc43)c2)cc1]

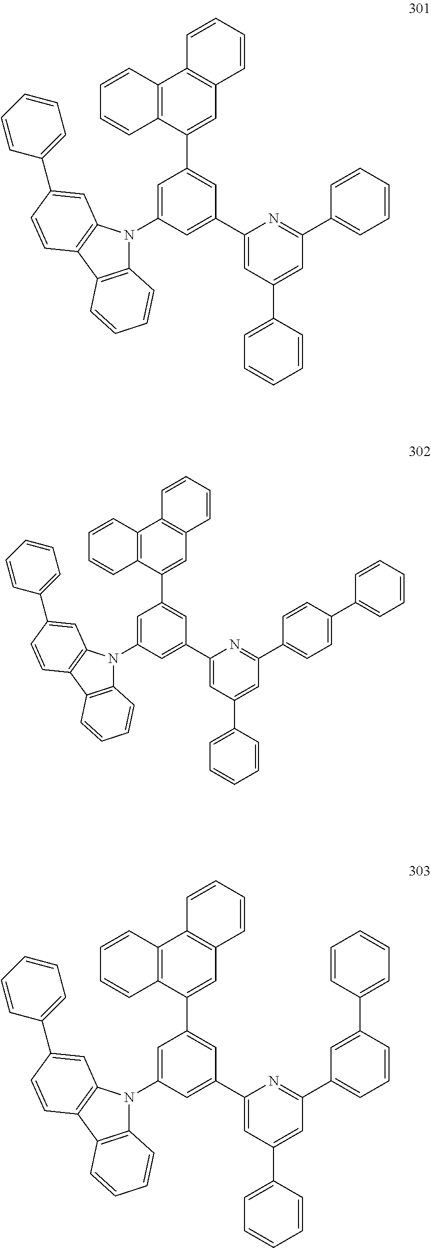 [SMILES: c1ccc(-c2cc(-c3ccccc3)nc(-c3cc(-c4cc5ccccc5c5ccccc45)cc(-n4c5ccccc5c5ccc(-c6ccccc6)cc54)c3)c2)cc1 ; c1ccc(-c2ccc(-c3cc(-c4ccccc4)cc(-c4cc(-c5cc6ccccc6c6ccccc56)cc(-n5c6ccccc6c6ccc(-c7ccccc7)cc65)c4)n3)cc2)cc1 ; c1ccc(-c2cccc(-c3cc(-c4ccccc4)cc(-c4cc(-c5cc6ccccc6c6ccccc56)cc(-n5c6ccccc6c6ccc(-c7ccccc7)cc65)c4)n3)c2)cc1]

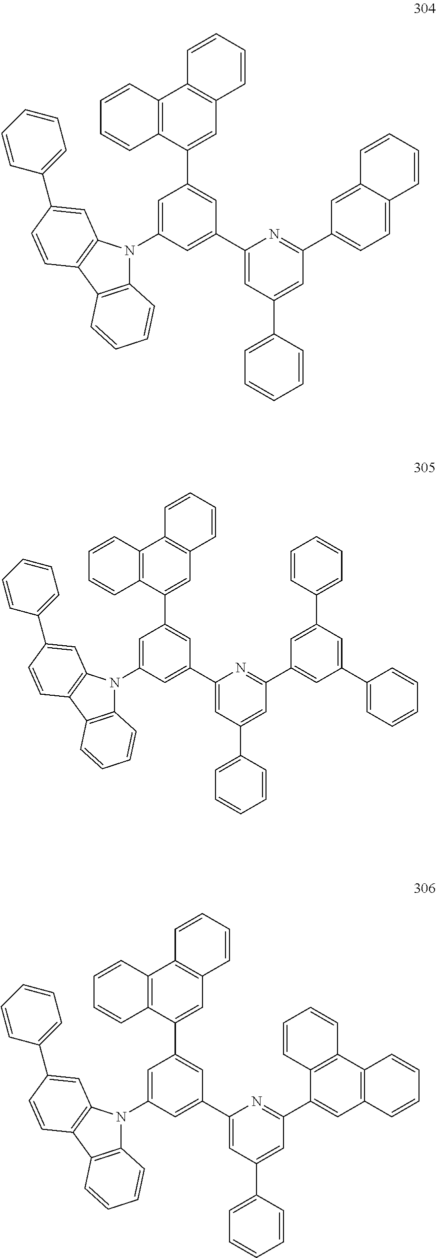 [SMILES: c1ccc(-c2cc(-c3cc(-c4cc5ccccc5c5ccccc45)cc(-n4c5ccccc5c5ccc(-c6ccccc6)cc54)c3)nc(-c3ccc4ccccc4c3)c2)cc1 ; c1ccc(-c2cc(-c3ccccc3)cc(-c3cc(-c4ccccc4)cc(-c4cc(-c5cc6ccccc6c6ccccc56)cc(-n5c6ccccc6c6ccc(-c7ccccc7)cc65)c4)n3)c2)cc1 ; c1ccc(-c2cc(-c3cc(-c4cc5ccccc5c5ccccc45)cc(-n4c5ccccc5c5ccc(-c6ccccc6)cc54)c3)nc(-c3cc4ccccc4c4ccccc34)c2)cc1]

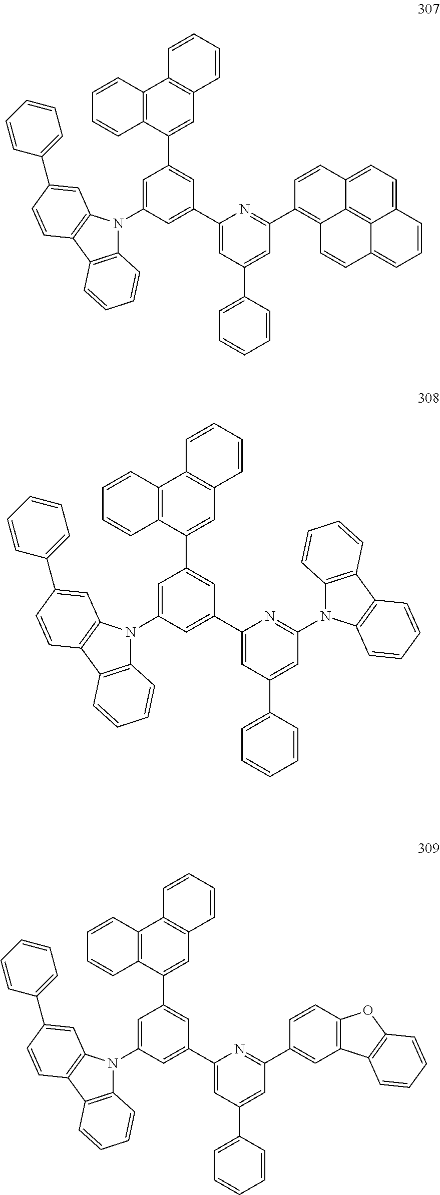 [SMILES: c1ccc(-c2cc(-c3cc(-c4cc5ccccc5c5ccccc45)cc(-n4c5ccccc5c5ccc(-c6ccccc6)cc54)c3)nc(-c3ccc4ccc5cccc6ccc3c4c56)c2)cc1 ; c1ccc(-c2cc(-c3cc(-c4cc5ccccc5c5ccccc45)cc(-n4c5ccccc5c5ccc(-c6ccccc6)cc54)c3)nc(-n3c4ccccc4c4ccccc43)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4oc5ccccc5c4c3)c2)cc1]

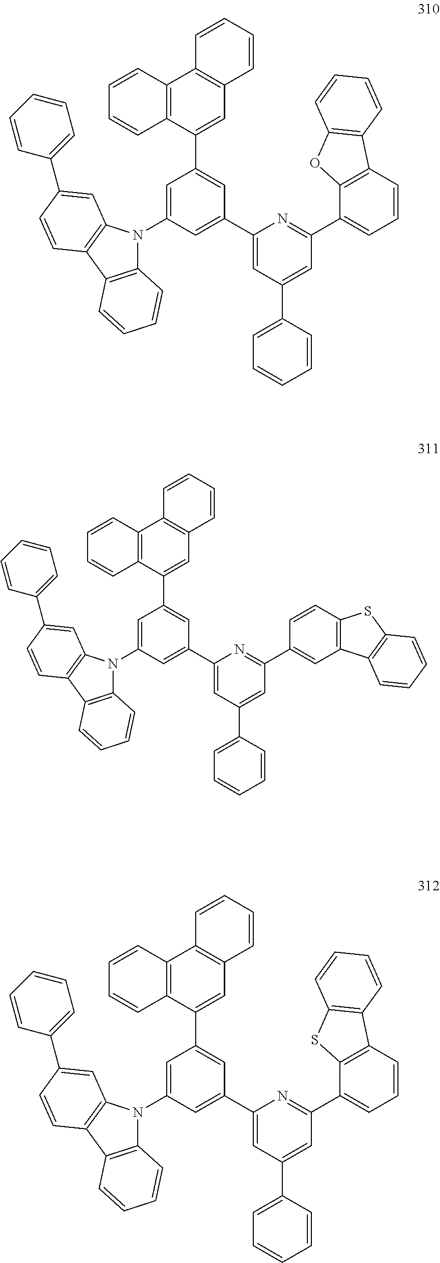 [SMILES: c1ccc(-c2cc(-c3cc(-c4cc5ccccc5c5ccccc45)cc(-n4c5ccccc5c5ccc(-c6ccccc6)cc54)c3)nc(-c3cccc4c3oc3ccccc34)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4sc5ccccc5c4c3)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3sc3ccccc34)c2)cc1]

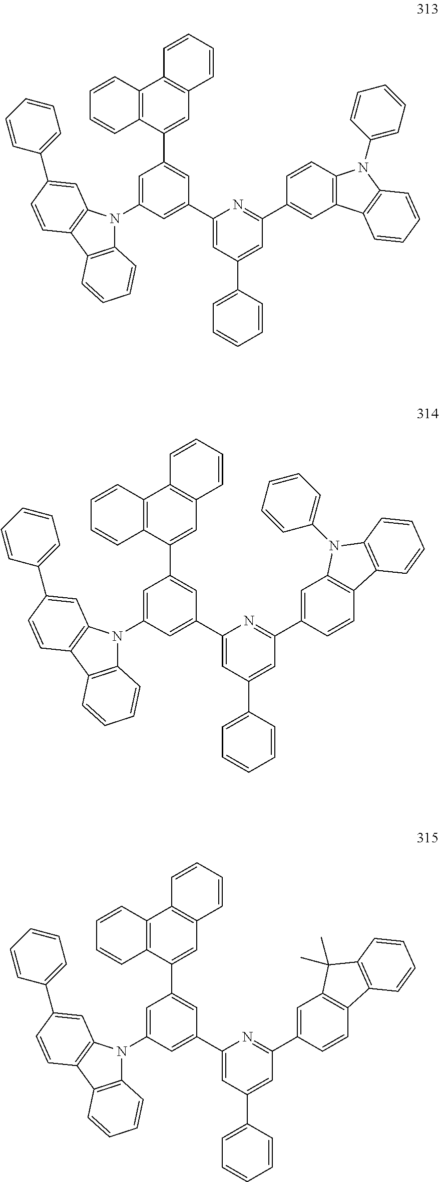 [SMILES: c1ccc(-c2cc(-c3cc(-c4cc5ccccc5c5ccccc45)cc(-n4c5ccccc5c5ccc(-c6ccccc6)cc54)c3)nc(-c3ccc4c(c3)c3ccccc3n4-c3ccccc3)c2)cc1 ; c1ccc(-c2cc(-c3cc(-c4cc5ccccc5c5ccccc45)cc(-n4c5ccccc5c5ccc(-c6ccccc6)cc54)c3)nc(-c3ccc4c5ccccc5n(-c5ccccc5)c4c3)c2)cc1 ; CC1(C)c2ccccc2-c2ccc(-c3cc(-c4ccccc4)cc(-c4cc(-c5cc6ccccc6c6ccccc56)cc(-n5c6ccccc6c6ccc(-c7ccccc7)cc65)c4)n3)cc21]

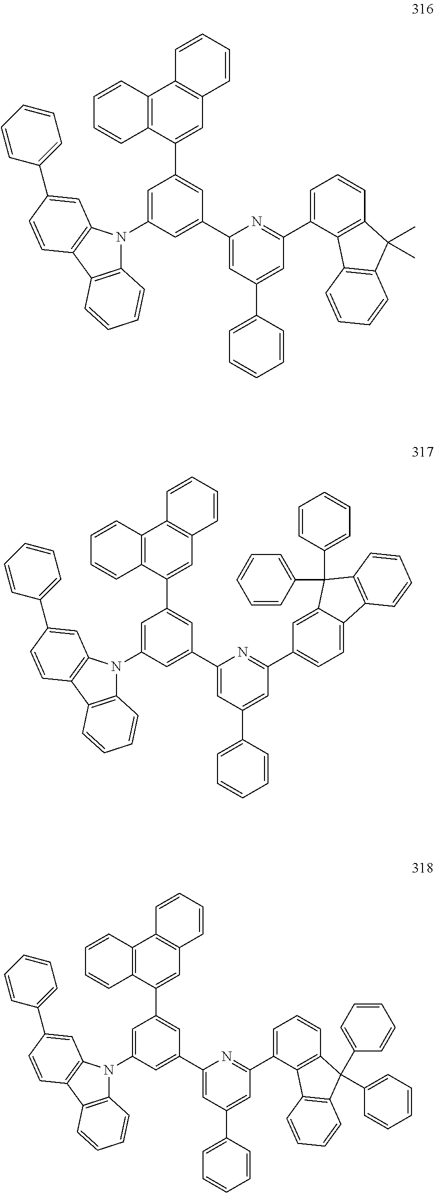 [SMILES: CC1(C)c2ccccc2-c2c(-c3cc(-c4ccccc4)cc(-c4cc(-c5cc6ccccc6c6ccccc56)cc(-n5c6ccccc6c6ccc(-c7ccccc7)cc65)c4)n3)cccc21 ; c1ccc(-c2cc(-c3cc(-c4cc5ccccc5c5ccccc45)cc(-n4c5ccccc5c5ccc(-c6ccccc6)cc54)c3)nc(-c3ccc4c(c3)C(c3ccccc3)(c3ccccc3)c3ccccc3-4)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3-c3ccccc3C4(c3ccccc3)c3ccccc3)c2)cc1]

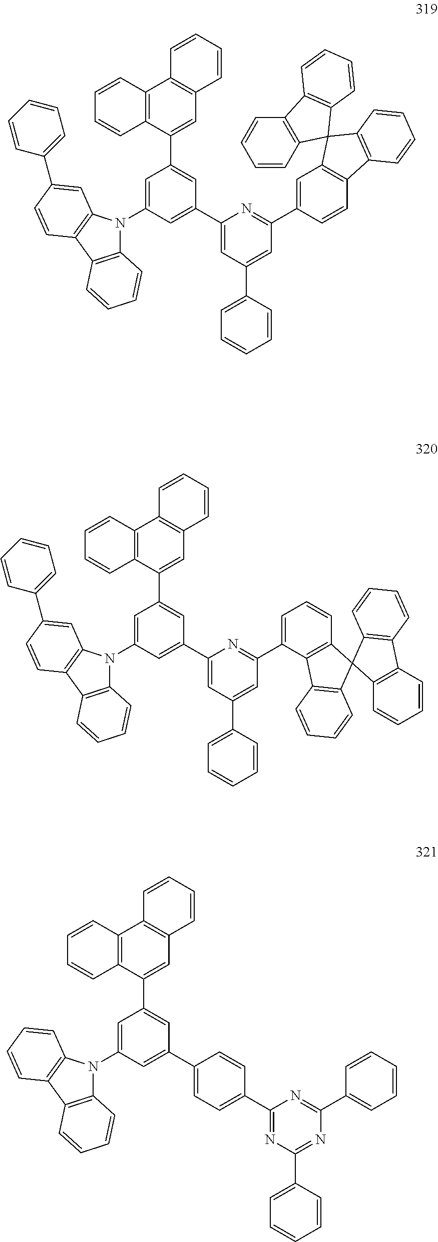 [SMILES: c1ccc(-c2cc(-c3cc(-c4cc5ccccc5c5ccccc45)cc(-n4c5ccccc5c5ccc(-c6ccccc6)cc54)c3)nc(-c3ccc4c(c3)C3(c5ccccc5-c5ccccc53)c3ccccc3-4)c2)cc1 ; c1ccc(-c2cc(-c3cc(-c4cc5ccccc5c5ccccc45)cc(-n4c5ccccc5c5ccc(-c6ccccc6)cc54)c3)nc(-c3cccc4c3-c3ccccc3C43c4ccccc4-c4ccccc43)c2)cc1 ; c1ccc(-c2nc(-c3ccccc3)nc(-c3ccc(-c4cc(-c5cc6ccccc6c6ccccc56)cc(-n5c6ccccc6c6ccccc65)c4)cc3)n2)cc1]

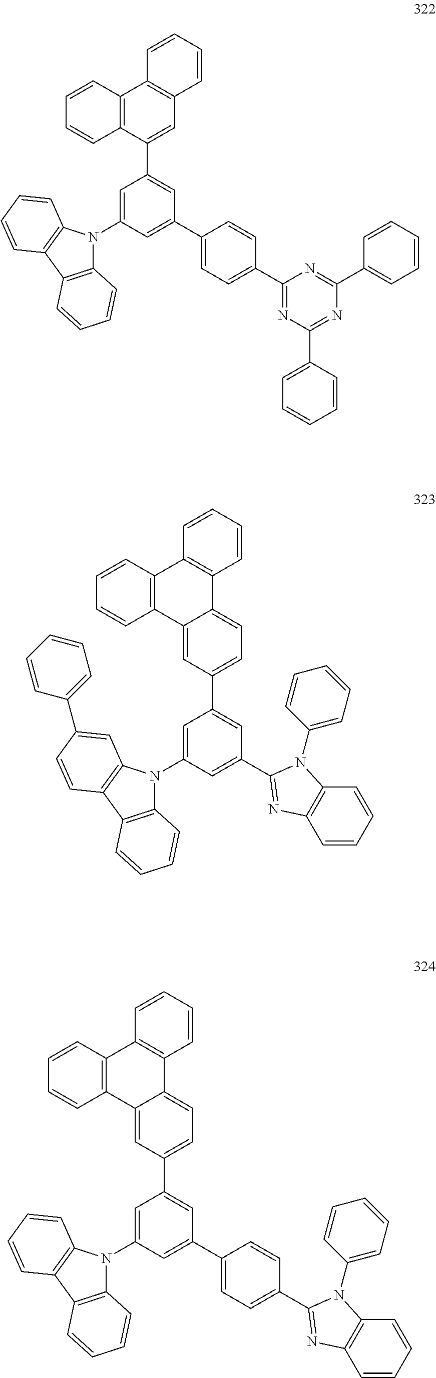 [SMILES: c1ccc(-c2nc(-c3ccccc3)nc(-c3ccc(-c4cc(-c5cc6ccccc6c6ccccc56)cc(-n5c6ccccc6c6ccccc65)c4)cc3)n2)cc1 ; c1ccc(-c2ccc3c4ccccc4n(-c4cc(-c5ccc6c7ccccc7c7ccccc7c6c5)cc(-c5nc6ccccc6n5-c5ccccc5)c4)c3c2)cc1 ; c1ccc(-n2c(-c3ccc(-c4cc(-c5ccc6c7ccccc7c7ccccc7c6c5)cc(-n5c6ccccc6c6ccccc65)c4)cc3)nc3ccccc32)cc1]

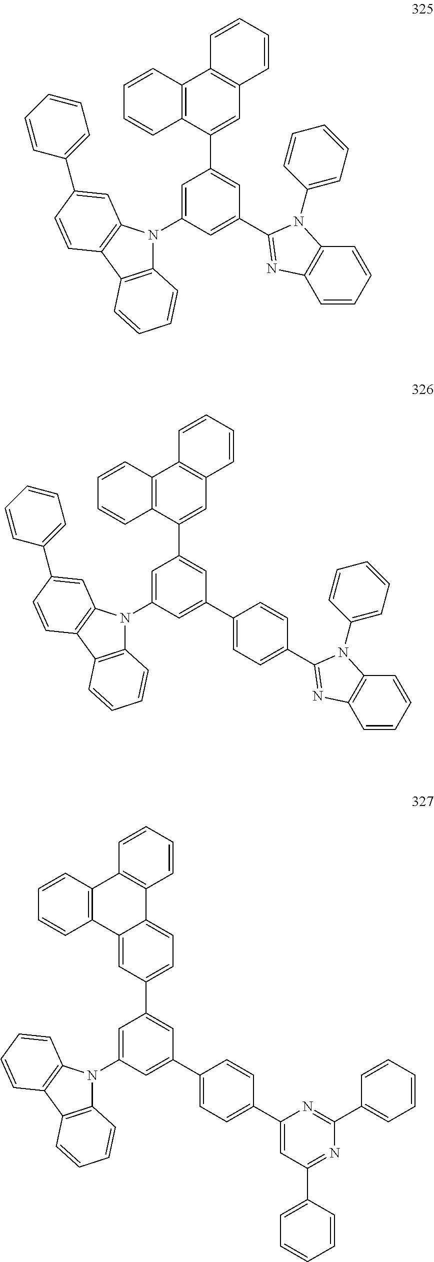 [SMILES: c1ccc(-c2ccc3c4ccccc4n(-c4cc(-c5cc6ccccc6c6ccccc56)cc(-c5nc6ccccc6n5-c5ccccc5)c4)c3c2)cc1 ; c1ccc(-c2ccc3c4ccccc4n(-c4cc(-c5ccc(-c6nc7ccccc7n6-c6ccccc6)cc5)cc(-c5cc6ccccc6c6ccccc56)c4)c3c2)cc1 ; c1ccc(-c2cc(-c3ccc(-c4cc(-c5ccc6c7ccccc7c7ccccc7c6c5)cc(-n5c6ccccc6c6ccccc65)c4)cc3)nc(-c3ccccc3)n2)cc1]

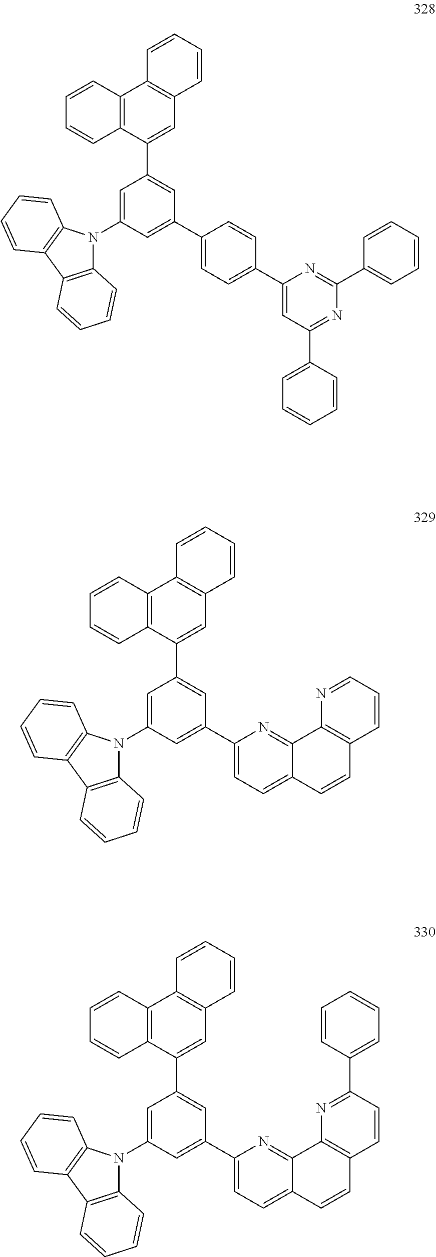 [SMILES: c1ccc(-c2cc(-c3ccc(-c4cc(-c5cc6ccccc6c6ccccc56)cc(-n5c6ccccc6c6ccccc65)c4)cc3)nc(-c3ccccc3)n2)cc1 ; c1ccc2c(c1)cc(-c1cc(-c3ccc4ccc5cccnc5c4n3)cc(-n3c4ccccc4c4ccccc43)c1)c1ccccc12 ; c1ccc(-c2ccc3ccc4ccc(-c5cc(-c6cc7ccccc7c7ccccc67)cc(-n6c7ccccc7c7ccccc76)c5)nc4c3n2)cc1]

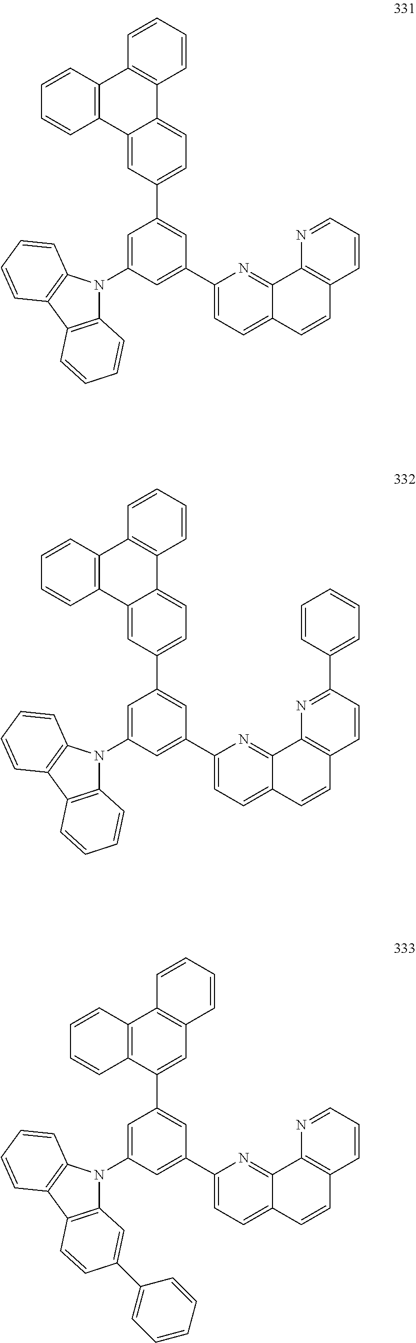 [SMILES: c1cnc2c(c1)ccc1ccc(-c3cc(-c4ccc5c6ccccc6c6ccccc6c5c4)cc(-n4c5ccccc5c5ccccc54)c3)nc12 ; c1ccc(-c2ccc3ccc4ccc(-c5cc(-c6ccc7c8ccccc8c8ccccc8c7c6)cc(-n6c7ccccc7c7ccccc76)c5)nc4c3n2)cc1 ; c1ccc(-c2ccc3c4ccccc4n(-c4cc(-c5ccc6ccc7cccnc7c6n5)cc(-c5cc6ccccc6c6ccccc56)c4)c3c2)cc1]

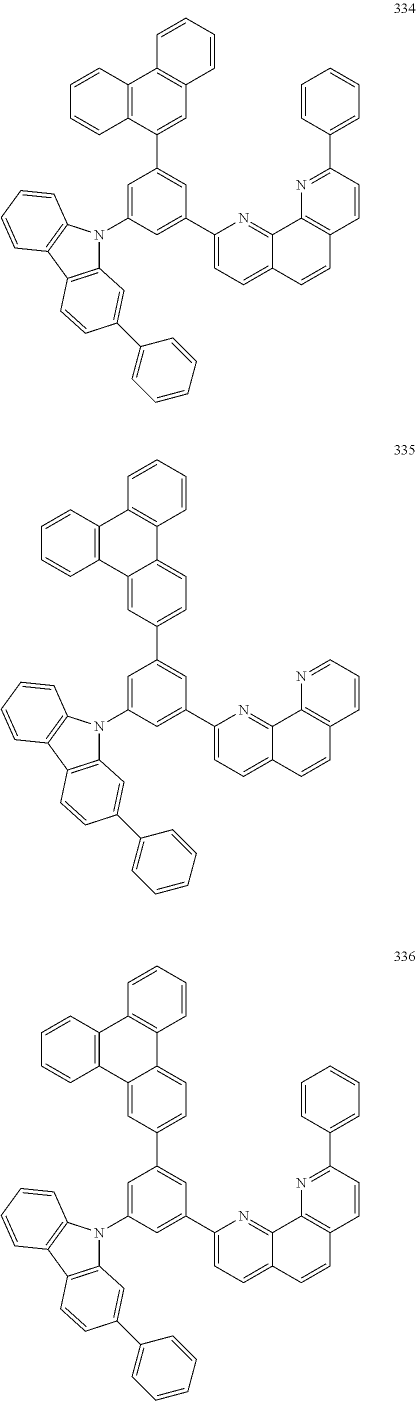 [SMILES: c1ccc(-c2ccc3c4ccccc4n(-c4cc(-c5ccc6ccc7ccc(-c8ccccc8)nc7c6n5)cc(-c5cc6ccccc6c6ccccc56)c4)c3c2)cc1 ; c1ccc(-c2ccc3c4ccccc4n(-c4cc(-c5ccc6c7ccccc7c7ccccc7c6c5)cc(-c5ccc6ccc7cccnc7c6n5)c4)c3c2)cc1 ; c1ccc(-c2ccc3c4ccccc4n(-c4cc(-c5ccc6c7ccccc7c7ccccc7c6c5)cc(-c5ccc6ccc7ccc(-c8ccccc8)nc7c6n5)c4)c3c2)cc1]

7. An organic light emitting device comprising:
an anode, a cathode, and one or more layered organic material layers provided between the anode and the cathode,
wherein one or more layers of the organic material layers include the compound of claim 1.

8. The organic light emitting device of claim 7, wherein the organic material layer includes at least one layer of a hole blocking layer, an electron injecting layer, and an electron transport layer, and at least one layer of the hole blocking layer, the electron injecting layer, and the electron transport layer includes the compound.

9. The organic light emitting device of claim 7, wherein the organic material layer includes a light emitting layer, and the light emitting layer includes the compound.
